(19) United States
(12) Patent Application Publication
(10) Pub. No.: US 2021/0076578 A1
(43) Pub. Date: Mar. 18, 2021

(54) NON-GM IMPROVED TOMATO CROPS AND METHODS FOR OBTAINING CROPS WITH IMPROVED INHERITABLE TRAITS

(71) Applicant: EPIGENETICS LTD., Rehovot (IL)

(72) Inventor: Avner SHENFELD, Rehovot (IL)

(21) Appl. No.: 17/086,437

(22) Filed: Nov. 1, 2020

Related U.S. Application Data
(63) Continuation-in-part of application No. PCT/IB2019/053516, filed on Apr. 30, 2019.
(60) Provisional application No. 62/665,847, filed on May 2, 2018.

(51) Int. Cl. A01G 22/05; A01G 7/04; A23B 7/015

(52) U.S. Cl. CPC A01G 22/05; A23B 7/015; A01G 7/045

(57) ABSTRACT

The present invention discloses a method for producing a commercial non-GM tomato crop exhibiting at least one inheritable improved trait by exposing said population of plants to a predetermined light treatment regime, monitoring at least one trait of said tomato plants comparing to said control population and selecting the plants having the best improved trait.

The tomatoes obtained by the method of this invention exhibit an improved room temperature (17-27° C.) shelf life of at least 45 days after harvesting, as compared to about 10 days for untreated control tomatoes.

### CROSS REFERENCE TO RELATED APPLICATIONS

This application is a Continuation in Part of National Stage Entry of International Application No: PCT/M2019/053516, filed on 30 Apr. 2019, which claims priority to U.S. Provisional Patent Application Ser. No. 62/665,847, filed on 30 Apr. 2019 May 2, 2018, the entire contents of which are hereby incorporated by reference in their entirety.

### TECHNICAL FIELD

The present disclosure provides a non-genetically modified (non-GM) tomato plant and/or a harvestable part thereof, exhibiting at least one inheritable improved trait and methods for obtaining same.

### BACKGROUND ART

Improvement of agricultural methods and productivity is seen as one of the greatest challenges of the 21^{st} century. According to the OECD-FAO report of 2012, agricultural production needs to be increased by 60% over the next 40 years, to meet the rising demand for food. Globally, the scope of area expansion is limited, i.e. the total arable land is projected to be increased by less than 5%. Thus, additional technologies will need to be developed in order to increase food production.

Traditionally, improved crops have been obtained by farmer experimentation with new varieties, plant breeding, purposeful selection, growth and cross pollination.

The first genetically modified crop plant was produced in 1982. Genetically modified crops (GM or GMC) are plants whose DNA was modified using genetic engineering techniques with a view to introduce in the plants a non-natural trait.

Transgenic (GM) plants possess a particular trait not native to the plant, which (inherited) trait is transmitted through the seeds.

While the GM technology has been widely accepted in the US and other countries, the regulatory status of the GM foods varies by country, with some countries banning or restricting them, or permitting them within limiting regulations.

Up until now, the efforts to increase the crop's intrinsic yield potential have mainly focused on exploiting the genetic variability within the crops. New combinations of plants have been mainly produced by traditional breeding techniques and molecular techniques, allowing the exchange of genetic material across species.

The role of epigenetic control mechanisms was much less studied and utilized. Epigenetics refers to the study of changes in gene expression or cellular phenotype, caused by mechanisms other than changes in the underlying DNA sequence.

Epigenetic mechanisms include functionally relevant modifications to the genome that do not involve a change in the nucleotide sequence. Examples of such modifications include DNA methylation and histone modification, both of which serve to regulate gene expression without altering the underlying DNA sequence. In such mechanisms, “non-genetic” factors cause the organism's genes to be expressed differently.

The role of epigenetic control components in plants was demonstrated in some studies and patent documents. US patent application 2013/0117877 A1 relates to methods of finding a DNA methylation profile for a plant with high energy use efficiency. The invention enables the artisan to correlate the DNA methylation profile of a plant with potentially high energy use efficiency. US patent application 2012/0117678 A1 as well as M. Hauben et al. (Proceedings of the National Academy of Sciences of the United States of America, 106, no. 47, 20109-20114, 2009) publication, disclose methods for selecting plants with high energy use efficiency, by monitoring their cellular respiration rate. The cellular respiration rate is determined by measuring NADPH content, ascorbic acid content and/or respiratory chain complex I activity. It is emphasized that the cellular respiration rate measurements taught by US patent application 2012/0117678 A1 and Hauben et al (2009), are performed in vitro, on explants or tissue samples isolated from individual plants. Furthermore, the prior art remains silent on the ability to induce the production of plants with high yield and/or inheritable properties.

Attempts at manipulating the yield of plants and identifying yield genes have been made, with emphasis on the modification of the flowering time in plants (U.S. Pat. No. 8,935,880 and U.S. Patent Application No. 2014/0259905).

The International Patent Application Serial No. WO2017/077539, filed on Nov. 1, 2016 by Shenfeld A., incorporated in its entirety by reference, discloses methods of obtaining various non-GM improved crops with inheritable improved traits.

There is an unmet and long felt need for non-GM tomato crops with improved traits and moreover, for non-GM tomato crops with inheritable improved traits.

### SUMMARY OF INVENTION

This disclosure provides a method for producing a non-genetically modified (non-GM) tomato plant and/or a harvestable part thereof, exhibiting at least one inheritable improved trait. Also provided is a non-GM tomato plant and/or a harvestable part thereof with improved inheritable traits, produced by the method of this disclosure.

It is an object of the present disclosure to provide a method for producing a non-GM tomato plant and/or a harvestable part thereof, exhibiting at least one inheritable improved trait as compared to a control population of untreated tomato plants, said method comprising the steps of: (a) providing a population of a cultivar or line of tomato seeds or plants; (b) exposing said population (a) of a cultivar or line of tomato plants to a predetermined light treatment by irradiating with artificial light, partly in the presence of ambient daylight, every day for a duration of 2 to 6 weeks from sowing, preferably for 30 days from sowing, wherein the predetermined light treatment consists of irradiating with artificial red light of wavelength in the range of from about 600 nm to about 700 nm for a period of about 60 minutes, starting about 30 min. before sunset in the presence of daylight, and continuing about 30 minutes after sunset. (c) growing the tomato plants in greenhouses for 2-3 months after the end of stage (b), then collecting, counting and weighing the tomatoes and collecting their seeds. d) monitoring at least one improved trait of the tomato plants at the end of step (b) and step (c) and comparing to a control population; (e) selecting at the end of the treatment of step (b) the plants exhibiting at least one improved trait as compared to a control population and selecting again at the end of the growing stage of step (c) the plants exhibiting at least one improved trait as compared to a control population; wherein the number of plants selected in the first generation is from about 1% to about 4%, preferably from about 2% to about 3% of the initial plant population provided (a); (f) propagating by sowing the tomato seeds at least one subsequent generation of the plants exhibiting at least one improved trait as compared to a control population; and (g) repeating steps (b) to (f) on 1-7 subsequent generations, preferably at least two subsequent generations; wherein the number of plants selected in step (e) increases from generation to generation, thereby obtaining a tomato crop plant and/or harvestable part thereof exhibiting at least one improved trait, wherein the improved trait is inheritable for at least one more generation, preferably for two or more generations, more preferably for 3 to 5 generations, and even more preferably for 6 to 8 generations.

It is a further object of the present disclosure to provide the method as defined above, wherein steps (b) to (g) are applied to tomato plants or tomato plant parts selected from the group consisting of tomatoes, seeds, seedlings, tissue cultures, calluses, meristems, regenerable cells, protoplasts, potted seedlings, adult tomato plants and any combination thereof.

It is a further object of the present disclosure to provide the method as defined in any of the above, comprising steps of selecting the control population from the group consisting of untreated tomato plants, untreated tomato plants of the same generation, treated tomato plants of the same generation and any combination thereof.

It is a further object of the present disclosure to disclose the method as defined in any of the above, wherein the ambient daylight is characterized by luminous flux units of between about 1.5 to about 3000 lux, particularly from about 100 to about 2000 lux.

It is a further object of the present disclosure to provide the method as defined in any of the above, wherein the step of irradiating the tomato plants with artificial light is applied at a periodicity selected from the group consisting of: at the beginning of sunrise, during dawn, during sunrise, before sunset, during sunset, after sunset or at any combination thereof.

It is a further object of the present disclosure to provide the method as defined in any of the above, comprising steps of irradiating with artificial light the tomato plants of step (a) with red light wavelengths, particularly in the range of about 600 nm to about 700 nm.

It is a further object of the present disclosure to provide the method as defined in any of the above, comprising steps of irradiating with artificial light the tomato plants of step (a) with red light wavelengths every day for a duration of 2 to 6 weeks from sowing, preferably for 30 days, beginning on the sowing day.

It is a further object of the present disclosure to provide the method as defined in any of the above comprising steps of irradiating with artificial light the tomato plants of step (a) with wavelengths selected from the group consisting of red light, blue light, white light, preferably red light and any combination thereof.

It is a further object of the present disclosure to provide the method as defined in any of the above, comprising steps of irradiating with artificial light the tomato plants of step (a) with red light wavelengths, blue light wavelengths, white light wavelengths and any combination thereof, consecutively, simultaneously or interchangeably.

Table 4 in Example 6 discloses the selection of the optimal irradiation wavelength range out of red, blue and white light irradiation, based on best average tomatoes per plant weight at each wavelength.

In some embodiments, there is provided the method of this disclosure, wherein the tomato plants are optimally irradiated with red light wavelength.

It is a further object of the present disclosure to provide the method as defined in any of the above, comprising steps of exposing the tomato crop plants to a predetermined light treatment for about one month under greenhouse conditions and then transferring the selected best plants exhibiting at least one improved trait to field growth conditions or commercial greenhouse growth conditions. At the end of the growing cycle, the best performing plants, based on the selected traits are selected for an additional round of treatment.

It is a further object of the present disclosure to provide the method as defined in any of the above, comprising steps of exposing the plants to a predetermined light treatment regime for about one month under field conditions and then selecting the plants exhibiting at least one improved trait responding best to the treatment. At the end of the growing cycle, the best performing plants, based on the selected traits, are selected for an additional round of treatment.

It is a further object of the present disclosure to provide the method as defined in any of the above, comprising steps of monitoring at least one trait or parameter selected from the group consisting of tomatoes number, tomatoes weight, average seed number, average total seed weight, average single seed weight, plant height, main stem width, stem thickness, plant biomass, number of stems, number of secondary stems, photosynthesis efficiency, photosynthesis rate, nitrogen concentration in leaves and any combination thereof.

It is a further object of the present disclosure to provide the method as defined in any of the above, comprising steps of producing a commercial tomato crop plant exhibiting an increased yield of at least 2% and up to 800% or more of the at least one yield characteristic, as compared to the control population of same.

It is a further object of the present disclosure to provide the method as defined in any of the above, wherein steps (a) to (g) are applied to tomato cultivars, plant lines or plant types selected from the group consisting of M82, Adoration, Alicante, Azoychka, Beefsteak, Better Boy, Big Beef, Big Rainbow, Blaby Special, Black Krim, Brandy wine, Campari, Celebrity, Cherokee Purple, Canario, Early Girl, Enchantment, Ferris Wheel, Flamenco, Fourth of July, Garden Peach, Gardener's delight, German Johnson, Giulietta F1, Granadero, Great White, Green Zebra, Hillbilly, Japanese, Black Trifele, Jersey Boy, Jubilee, Juliet, Kumato, Lilian's Yellow, McDreamy, Matt's Wild Cherry, Micro Tom, Moneymaker, Monterosa, Montserrat, Mortgage Lifter, Mr. Stripey, Pantano Romanesco, Paul Robeson, Plum Tomato, Pumpkin Tomato, Raf Tomato, Rebellion, Red Currant, Roma, Rosa de Barbastro, Rutgers Tomato, San Marzano, Sasha Altai, Stupice, Tiny Tim, Traveller, Arkansas Traveler, Cherry Bambelo, Cherrry Nebula, Santorini, Super Sweet, Tomaccio, Yellow Pear, White Queen.

The experiments described in this disclosure have been carried out on the tomato cultivar M82.

It is a further object of the present disclosure to provide the method as defined in any of the above, comprising steps of producing a commercial tomato crop plant exhibiting an increased average seed number per plant of between about 10% and about 200%, preferably between about 50% and about 80%, as compared to the control population of same. It is a further object of the present disclosure to provide the method as defined in any of the above, comprising steps of producing a commercial tomato crop plant exhibiting improved traits such as an increased average single tomato weight of between about 10% and about 50%, preferably between about 20% and about 40% or from about 40% to about 100% increase as compared to the control population of same.

It is a further object of the present disclosure to provide the method as defined in any of the above, comprising steps of producing a commercial tomato crop plant exhibiting an improved trait such as an increased average total tomato weight per plant of between about 50% and about 200%, preferably between about 80% and about 150% as compared to the control population of same.

It is a further object of the present disclosure to provide the method as defined in any of the above, comprising steps of producing a commercial tomato exhibiting an improved trait such as improved room temperature (17-27° C.) shelf life of at least 45 days after harvesting, as compared to about 10 days for an untreated control tomato.

It is another further object of the present disclosure to provide the tomato obtained as described in this disclosure, wherein exhibiting an improved trait such as improved room temperature (17-27° C.) shelf life of at least 45 days after harvesting, as compared to about 10 days for an untreated control tomato.

It is a further object of the present disclosure to provide a tomato plant part or a harvestable part thereof produced by the method as defined in any of the above.

It is a further object of the present disclosure to provide the method of this disclosure wherein applied to a non-GM tomato plant.

It is a further object of the present disclosure to provide the method of this disclosure wherein applied to a genetically modified (GM) tomato plant.

It is a further object of the present disclosure to provide a plant part or a product thereof derived from the commercial plant as defined in any of the above.

It is further within the scope of this disclosure to provide a method for manipulating the plant's genome expression to increase its energy production efficiency. This may result in a dramatic increase in the number of tomatoes, seeds and biomass of the commercial tomato plant crop. Using the methods of the present disclosure, it is demonstrated that a tomato crop exhibiting increased yield capacity of up to 800% or more, between F1 (generation 1) and F2 (generation 2) or subsequent generation F3, can be produced.

It is a further object of the present disclosure to provide a tomato plant or a harvestable part thereof such as a tomato or a tomato seed, produced by the method as defined in any of the above.

### BRIEF DESCRIPTION OF THE FIGURES

Some embodiments of the disclosure are herein described, by way of example only, with reference to the accompanying Figures. With specific reference now to the drawings in detail, it is stressed that the particulars shown are by way of example and for purposes of illustrative discussion of embodiments of the disclosure. In this regard, the description taken with the Figures makes apparent to those skilled in the art how embodiments of the disclosure may be practiced.

In order to better understand the disclosure and its implementation in practice, a plurality of embodiments will now be described, by way of non-limiting example only, with reference to the accompanying Figures, wherein

FIG. 1 photographically presents red lamps for irradiation of the tomato plants.

FIG. 2 graphically compares the average tomato plant height in F1 treated plants vs control plants.

FIG. 3 graphically compares the average no. of inflorescences per F1 treated tomato plant vs. control plants.

FIG. 4 graphically compares the average weight of tomatoes per F1 treated plant vs. control.

FIG. 5 graphically compares the average weight of tomatoes in F2 treated plants vs control plants.

FIG. 6 graphically compares the total tomato weight in the four best F2 treated tomato plants vs. average weight in control plants.

FIG. 7 presents a photographic illustration of tomatoes with extended shelf life obtained from F2 treated plants.

FIG. 8 graphically compares the average weight of tomatoes from F3 treated tomato plants vs. control.

FIG. 9 presents a photographic illustration of F3 treated tomato plants and control plants.

### DETAILED DESCRIPTION

The following description is provided, alongside all chapters of the present disclosure, so as to enable any person skilled in the art to make use of said disclosure and sets forth the best modes contemplated by the inventor of carrying out this invention. Various modifications, however, will remain apparent to those skilled in the art, since the generic principles of the present disclosure have been defined specifically to provide methods and products thereof for producing and screening for a commercial non-GM tomato plant exhibiting at least one improved trait compared to a control population of same.

It is an object of the present disclosure to provide a method for producing a non-GM tomato plant and/or a harvestable part thereof, exhibiting at least one inheritable improved trait as compared to a control population of untreated tomato plants, said method comprising the steps of: (a) providing a population of a cultivar or line of tomato seeds or plants; (b) exposing said population (a) of a cultivar or line of tomato plants to a predetermined light treatment by irradiating with artificial light, partly in the presence of ambient daylight, every day for a duration of 2 to 6 weeks from sowing, preferably for 30 days from sowing, wherein the predetermined light treatment consists of irradiating with artificial red light of wavelength in the range of from about 600 nm to about 700 nm for a period of about 60 minutes, starting about 30 min. before sunset in the presence of daylight, and continuing about 30 minutes after sunset. (c) growing the tomato plants in greenhouses for 2-3 months after the end of stage (b), then collecting, counting and weighing the tomatoes and collecting their seeds; (d) monitoring at least one improved trait of the tomato plants at the end of step (b) and step (c) and comparing to a control population; (e) selecting at the end of the treatment of step (b) the plants exhibiting at least one improved trait as compared to a control population and selecting again at the end of the growing stage of step (c) the plants exhibiting at least one improved trait as compared to a control population; wherein the number of plants selected in the first generation is from about 1% to about 4%, preferably from about 2% to about 3% of the initial plant population provided (a); (f) propagating by sowing the tomato seeds at least one subsequent generation of the plants exhibiting at least one improved trait as compared to a control population; and (g) repeating steps (b) to (f) on 1-7 subsequent generations, preferably at least two subsequent generations; wherein the number of plants selected in step (e) increases from generation to generation, thereby obtaining a tomato crop plant and/or harvestable part thereof exhibiting at least one improved trait, wherein the improved trait is inheritable for at least one more generation, preferably for two or more generations, more preferably for 3 to 5 generations, and even more preferably for 6 to 8 generations

Examples 1-6 are provided, in which the tomato plants are M82 tomato cultivar plants.

In this way a non-GM tomato plant is produced, exhibiting inheritable improved traits compared to a control population of same.

It is further within the scope of the disclosure to disclose a method for improving tomato plant traits by manipulating environmental factors affecting the plant's growth such as artificial light irradiation.

The technology provided by the present disclosure is suitable for use on genetically modified (GM) crops as well as on non-GM crops (i.e. produced by other techniques such as breeding, grafting etc.), but the method itself is non-GM and does not lead to GM modifications by itself.

It is further within the scope of the disclosure to provide various treatment protocols for affecting tomato crop plants, preferably crop yield, resulting in different extent of trait improvements (such as tomato yield increase, biomass increase, etc.) of the treated tomato plants versus control plants.

It is further within the scope of the disclosure to provide a method for manipulating the various tomato plant inheritable traits by the method detailed in this disclosure.

According to certain aspects, the phenotypic changes in the treated plants are preserved in the next generations.

It is further demonstrated in the present disclosure that by increasing the efficiency of photosynthesis, tomato crop lines characterized by improved traits such as high yield and better growth potential were produced and selected.

According to one embodiment, the present disclosure provides methods to increase the expression of genes which accelerate photosynthesis, by manipulating environmental factors. As a result, improved tomato plant traits (such as an increase in tomato weight, tomato number, plant growth, biomass increase, inheritable phenotypic changes) is achieved.

Without wishing to be bound by theory, the environmental effects on gene expression may be associated with epigenetic mechanisms. It is noted that such genes may be responsive to environmental influence. The improved traits acquired are inheritable over a number of generations (at least one more generation and up to 5-8 generations), without the need for genetical modification.

According to a further embodiment, it is herein demonstrated that the ability to increase photosynthesis rate is associated with increased plant growth and with improved traits such as average number of tomatoes per plant, tomato weight per plant, seed weight per plant, average seed number per plant, average seed weight per plant, plant height, main stem width, stem thickness, average weight of a single seed, plant biomass, number of stems, number of secondary stems, stress resistance, pest resistance, virus resistance, drought tolerance, herbicide tolerance, delayed senescence, modified color, photosynthesis efficiency or rate, nitrogen concentration in the leaves, or any combination thereof.

According to a further embodiment, the improved trait plants of the present disclosure may be non-GM (Non-Genetically Modified) or GM plants. It is noted that modifications in the treatment protocol of the present disclosure may be adapted to each tomato cultivar type.

It is further within the scope of this disclosure that the treatment procedures (i.e. exposure to artificial environmental conditions) as well as the monitoring and evaluation of tomato yield characteristics disclosed in the present disclosure are performed during the tomato plant growth without disrupting the normal growth cycle of the plant. It is herein emphasized that the treatment procedures are external, and in preferred embodiments, are directed to the whole plant. In any case, even when the treatment is done on seeds or plant parts, the treatment is non-invasive. Furthermore, the monitoring or selection or identification of high performing plants does not interrupt or interfere with tomato plant growth.

The traits subject to improvement are selected from the group consisting of average tomato number per plant, weight per plant, average seed number per plant, average seed weight per plant, plant height, main stem width, stem thickness, tomato weight per plant, average weight of a single seed, plant biomass, number of stems, number of secondary stems, stress resistance, pest resistance, virus resistance, drought tolerance, herbicide tolerance, delayed senescence, modified color, photosynthesis efficiency or rate, nitrogen concentration in the leaves or any combination thereof.

In specific embodiments, tomato plants were exposed to irradiation with artificial light of wavelengths selected from red light, blue light, white light and any combination thereof, administered at predetermined protocols, periodicities, cycles and sessions.

In a preferred aspect, the tomato plant irradiation treatment is carried out with red light irradiation, for one hour per day, out of which half an hour before sunset and another half an hour after sunset.

The selection of the tomato plants exhibiting the best improved traits is an important part of the methods of this disclosure.

The treatment method of this disclosure uses in each generation of plant treatment (F1, F2, F3 and F4) two selection stages that differ from each other:

1. The first selection takes place at the end of the irradiation period (b), about one month from the time of sowing. The plants exhibiting at least one improved trait, best responding to the treatment, are selected and transferred to greenhouse growth conditions.

2. The second selection takes place at the end of the growing period (c) in greenhouses (about 2-3 months, depending on the season). This selection is based on the best performing chosen trait (e.g. tomato yield). The plants exhibiting at least one improved trait are selected, tomatoes are collected, counted, weighed and their seeds are collected for the next round of treatment

It was found that three rounds of treatment (F1-F3) lead to the best heredity results.

According to certain aspects of the disclosure, the following conclusions are herein demonstrated: The treated plants of the present disclosure have a significantly increased average yield of up to several hundred percent (i.e. up to 800% or more) greater than the average yield of control plants, as exemplified herein.

The increase in yield and growth of the treated tomato plants appeared not only in the treated generation but continued to the next generation and was maintained without additional treatment. It is noted that the high yield plant may have adapted to the aforementioned major phenotypic changes, over time. The demand for energy is significantly higher in the high yield tomato lines and the tomato plants increase their energy supply to adjust to the new demands but the adaptation continues vertically through several generations.

According to a further embodiment, the method as defined in any of the above, comprises steps of producing a commercial tomato plant exhibiting an increased tomato yield of at least 2% and up to 800%, or more particularly 2%, 5%, 10%, 20%, 30%, 40%, 50%, 60%, 70%, 80%, 90%, 100%, 200%, 300%, 400%, 500%, 600%, 700%, 800%, 900% or more, of said at least one characteristic, as compared to the control population of same.

According to a further embodiment, the method as defined in any of the above, comprises steps of producing a commercial tomato plant exhibiting an increased average total tomato weight per plant of between about 50% and about 200%, preferably between about 80% and about 150% higher than the control population of same.

It is further within the scope of this disclosure to provide the method as defined in any of the above, wherein the method is applied to a non-genetically modified (non-GM) commercial tomato crop plant or to a GM tomato crop plant.

It is further within the scope of this disclosure to provide a plant part such as a tomato or seeds thereof produced by the method as defined in any of the above.

It is further within the scope of this disclosure to disclose the tomato plant as defined in any of the above, wherein the control population is selected from the group consisting of: untreated plants, untreated plants of the same generation, treated plants of the same generation and any combination thereof.

It is further within the scope of this disclosure to disclose the method as defined in any of the above, wherein the optional ambient daylight is characterized by luminous flux units between about 100 to about 2000 lux.

It is further within the scope of this disclosure to disclose the method as defined in any of the above, wherein the step of irradiating with artificial light is applied at a periodicity selected from the group consisting of at the beginning of sunrise, during dawn, during sunrise, before sunset, during sunset, after sunset or any combination thereof.

It is further within the scope of this disclosure to disclose the tomato plant as defined in any of the above, wherein the plant exhibits an increased tomato yield of between about at least 2% and about 800%, or more of the at least one yield characteristic, as compared to the control population of same cultivar.

It is further within the scope of this invention to disclose the tomato plant as defined in any of the above wherein the plant exhibits an increased tomato yield of between about 10% and about 200% as the at least one yield characteristic, as compared to the control population of same cultivar.

It is further within the scope of this disclosure to disclose the tomato plant as defined in any of the above wherein the plant exhibits an increased average tomato seed number per plant of between about 10% and about 100%, preferably between about 50% and about 80% higher than the control population of same cultivar.

It is further within the scope of this disclosure to disclose the tomato plant as defined in any of the above wherein the plant exhibits an increased average single seed weight of between about 10% and about 50%, preferably between about 20% and about 40% higher than the control population of same cultivar.

It is further within the scope of this invention to disclose the tomato plant as defined in any of the above wherein the plant exhibits an increased average total seed weight per plant of between about 50% and about 200%, preferably between about 80% and about 150% higher than the control population of same cultivar.

It is further within the scope of this disclosure to provide a method of screening for tomato plants exhibiting improved traits such as improved tomato yield compared to a control population of same, the method comprising the steps of: (a) providing a population of a cultivar or line of tomato seeds or plants; (b) exposing said population (a) of a cultivar or line of tomato plants to a predetermined light treatment by irradiating with artificial light, partly in the presence of ambient daylight, every day for a duration of 2 to 6 weeks from sowing, preferably for 30 days from sowing, wherein the predetermined light treatment consists of irradiating with artificial red light of wavelength in the range of from about 600 nm to about 700 nm for a period of about 60 minutes, starting about 30 min. before sunset in the presence of daylight, and continuing about 30 minutes after sunset. (c) growing the tomato plants in greenhouses for 2-3 months after the end of stage (b), then collecting, counting and weighing the tomatoes and collecting their seeds. d) monitoring at least one improved trait of the tomato plants at the end of step (b) and step (c) and comparing to a control population; (e) selecting at the end of the treatment of step (b) the plants exhibiting at least one improved trait as compared to a control population and selecting again at the end of the growing stage of step (c) the plants exhibiting at least one improved trait as compared to a control population; wherein the number of plants selected in the first generation is from about 1% to about 4%, preferably from about 2% to about 3% of the initial plant population provided (a); (f) propagating by sowing the tomato seeds at least one subsequent generation of the plants exhibiting at least one improved trait as compared to a control population; and (g) repeating steps (b) to (f) on 1-7 subsequent generations, preferably at least two subsequent generations; wherein the number of plants selected in step (e) increases from generation to generation, thereby obtaining a tomato crop plant and/or harvestable part thereof exhibiting at least one improved trait, wherein the improved trait is inheritable for at least one more generation, preferably for two or more generations, more preferably for 3 to 5 generations, and even more preferably for 6 to 8 generations.

This way the best plants are screened and selected for use in further propagations.

According to a further aspect, the method of screening for commercial tomato plants exhibiting increased yield compared to a control population of same as defined in any of the above, comprises steps of applying the steps as defined above to tomato plants or plant parts selected from the group consisting of tomatoes, seeds, seedlings, tissue cultures, calluses, meristems, regenerable cells, protoplasts, potted seedlings, adult plants and any combination thereof.

It is further within the scope of this disclosure to provide the method of screening for a commercial tomato plant exhibiting increased yield compared to a control population of same as defined in any of the above, wherein the control population is selected from the group consisting of: untreated plants, untreated plants of the same generation, treated plants of the same generation and any combination thereof.

It is further within the scope of this disclosure to provide the method of screening for a commercial tomato plant exhibiting increased tomato yield compared to a control population of same as defined in any of the above, wherein the step of irradiating with artificial light is applied at a periodicity selected from the group consisting of: at the beginning of sunrise, during dawn, during sunrise, before sunset, during sunset, after sunset or any combination thereof.

According to a further aspect, there is provided a method of screening for a commercial tomato plant exhibiting at least one inheritable improved trait (such as improved tomato yield) compared to a control population of same as defined in any of the above comprises the steps (a) to (f) of the method of this disclosure, in several consecutive rounds of treatment and selection. The best non-GM tomato plants exhibiting improved traits compared to a control population or line of plants of the same plant cultivar are screened and selected by this process for use in further propagation.

It is to be understood that the phraseology and terminology employed herein is for the purpose of description and should not be regarded as limiting.

### Definitions

As used herein, the term ‘about’ refers to a value being ±25% of the defined measure.

The term “commercial” or “commercial tomato cultivar” or “commercial crop” or “commercial crop plant” as used herein refers to a tomato cultivar or a line. In specific embodiments such tomato cultivars or lines have been selected for characteristics such as improved yield, flavor, and resistance to disease or environmental stress. In further aspects they were produced by repeated self-fertilization or inbreeding. The term “improved trait” as used herein refers to beneficial new traits acquired as a result of the treatment of the present disclosure by the treated tomato plants as compared to untreated plants of the same.

The term “control population” as used herein refers to crop plants used as reference or in comparison to crop plants of the same variety, cultivar or commercial line that were exposed to the treatment of the present disclosure. Such control population may include untreated plants, untreated plants of the same generation, treated plants of the same generation and any combination thereof. In some embodiments yield parameters have been compared between treated crop plants and a control population.

According to further embodiments, the term ‘control plant’ or ‘control population’ as used in the context of the present disclosure refers to an untreated tomato plant or a plant population of the same cultivar or variety or line of the corresponding treated plant. In one aspect, the controlled plant did not receive additional light radiation treatment on top of normal (ambient) sunlight. In another aspect, ‘control tomato plant’ or ‘control population’ used in the present disclosure refers to a corresponding plant of the same variety or line of the treated plant, exposed to the same number of treatment cycles as the treated plant, but propagated for one less generation than the treated plant. In another aspect, ‘control plant’ or ‘control population’ used in the present disclosure refers to a corresponding plant of the same cultivar, species or variety or line of the treated plant, and of the same generation of the treated plant, exposed to less treatment cycles.

The term ‘monocrop population’ as used herein refers to a population composed of genetically singular tomato crop plants. In other aspects, a monocrop population refers to crop plants of the same variety, particularly to the cultivation of a single crop cultivar.

The term ‘conditioning’ as used herein refers to the application of at least one treatment regime or protocol as defined herein below, to a predetermined tomato cultivar, to achieve improved yield characteristics or parameters.

The term ‘artificial light’ as used herein refers to any lighting that is not sunlight. In general, artificial light refers to lighting with man-made sources, such as fluorescent, tungsten, mercury vapor, sodium vapor, halogen, compact fluorescent, etc. It can be turned on and off. It is within the scope of the present disclosure that irradiation with artificial light includes exposing the plants to visible light wavelengths, corresponding to 380-750 nm and/or 450-495 nm. The following ranges can be used to distinguish between various light sources: violet (about 380-450 nm), blue (about 450-495 nm), green (about 495-570 nm), yellow (about 570-590 nm), orange (about 590-620 nm) and preferably red (620-750 nm).

The term ‘ambient light’ or ‘ambient daylight’ or ‘ambient sunlight’ as used herein refers to available or normal light or sources of light that are available naturally (e.g. the sun or moon). The term usually refers to the combination of all direct and indirect sunlight outdoors during the daytime. This includes direct sunlight, diffuse sky radiation that may be reflected from the Earth and terrestrial objects. It is within the scope of the disclosure that the outdoor illuminance can vary from 120,000 lux to less than 5 lux (even less than 1 lux for extreme cases). More specifically, the radiation treatment of the present disclosure is optionally applied in the presence of ambient light characterized by luminous flux units of between about 1.5 to about 3000 lux, particularly between about 100 to about 2000 lux, more particularly in the range of about 800 lux to about 1800 lux.

The term ‘dawn’ as used herein generally refers to the time that marks the beginning of the twilight before sunrise. In other aspects, the term dawn, include sunrise, particularly a period of about one hour at the beginning of sunrise. The exact dawn time depends on day length.

The term ‘sunset’ as used herein generally refers to the time of sunset defined in astronomy as the moment when the trailing edge of the Sun's disk disappears below the horizon. The exact time of sunset depends on day length. In some aspects it may refer to a period of about 30 minutes to about 75 minutes before sunset.

The term “lux” as used herein refers to the measuring of luminous flux per unit area, i.e. one lux is equal to one lumen per square meter.

The terms ‘dawn’ or ‘sunrise’ and ‘sunset’ are herein defined by light intensity or units of illuminance or luminous emittance or luminous flux per unit area of between about 1.5 to about 75 lux. It is noted that at sunset and sunrise (on a clear sky), ambient outdoor light may reach between about 400 lux and about 1800 lux.

The term ‘treatment’ as used herein refers to implementation of protocol comprising irradiation cycles with artificial light of wavelength ranges between 380 nm to about 750 nm, for duration of between 5 minutes to 2.5 hours per cycle, preferably one hour. A predetermined session may be defined in terms of cycles, for example, there may be sessions of 5 minutes to 2.5 hours per day at a periodicity of 24 hours apart, for duration of 2 to 6 weeks, preferably one month. A treatment cycle is directed to one generation. According to certain aspects, the aforementioned irradiation treatment is optionally applied in the presence of or in addition to normal or ambient daylight. The aforementioned treatment is applied to at least one tomato crop plant or a crop plant part. In another aspect, the irradiation treatment is applied about one hour at the beginning of sunrise, during dawn or before, during or after sunset or a combination thereof (dependent on day length).

It is noted that different combinations of light radiation treatments were applied to be suitable for the genomic structure and expression of each crop. Examples of treatment cycles used in the present disclosure may include the following:

The term ‘protocol A’ as used herein refers to a treatment protocol where the crop plants or part thereof, were irradiated with white light, for one hour at dawn or during sunset, for about one month from the time of sowing. In specific embodiments, applying protocol A treatment resulted in a decreased yield or lower magnitude of yield increase of the treated crops relative to the control crops.

The term “protocol B” or ‘treatment protocol’ as used herein refers to a treatment protocol where the crops were irradiated with red light (wavelength in the range of 600-700 nm), for about one hour before and after sunset, for about one month from the time of sowing. In specific embodiments, applying protocol B, the aforementioned treatment protocol resulted in an increased yield of the treated crops relative to the control crops.

As used herein, the term ‘progeny’ refers to the descendant(s) of a particular plant. Progeny may result from selfing (i.e., the same plant acts as the donor of both male and female gametes) or from crossing of two different plants. The descendant(s) can be, for example, of the F1, the F2, or any subsequent generation. The terms ‘increased yield’ or ‘high yield’ as used herein refers to epigenetically enhanced lines or cultivars of tomato crops that have an increased tomato crop production or increased percentage of usable plant parts, preferably tomatoes, seeds or biomass.

The ‘yield characteristic’ or ‘yield parameter’ includes at least one phenotypic parameter or characteristic selected from the group comprising average tomato number per plant, average tomato weight per plant, average seed number per plant, average seed weight per plant, plant height, main stem width, stem thickness, tomato weight per plant, average weight of a single seed, plant biomass, number of stems, number of secondary stems, photosynthesis efficiency or rate, nitrogen concentration in the leaves or any combination thereof.

The tomato plants produced by the methods provided in this disclosure exhibit enhanced or improved or improved traits such as increased tomato yield or improved measures of at least one of the parameters or characteristics listed above by at least 2% and up to 800% or more relative to a control plant, or control population.

As used herein, the term ‘population’ means a genetically homogeneous or heterogeneous collection of plants sharing a common genetic derivation.

As used herein, the term ‘variety’ or ‘cultivar’ means a group of similar plants that by structural features and performance can be identified from other varieties within the same species. The term ‘variety’ as used herein has identical meaning to the corresponding definition in the International Convention for the Protection of New Varieties of Plants (UPOV treaty), of Dec. 2, 1961, as revised at Geneva on Nov. 10, 1972, on Oct. 23, 1978, and on Mar. 19, 1991.

The term ‘plant cell culture’ or ‘tissue culture’ as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, tomatoes, seeds, seed coat or any combination thereof.

The term ‘plant material’ or ‘plant part’ used herein refers to tomatoes, leaves, stems, roots, root tips, flowers or flower parts, seeds, kernels, pollen, egg cells, zygotes, seed coat, cuttings, explant or any sample derived from a plant, cell or tissue cultures, or any other part or product derived from a plant or plant part and any combination thereof.

In other embodiments, the term ‘plant material’ or ‘plant part’ also refers to tissue culture of regenerable cells or protoplasts obtained from a plant or from a plant part selected from the group consisting of leaves, pollen, embryos, roots, root tips, anthers, flowers, tomatoes and seeds.

### Embodiments

In some embodiments, there is provided a method for producing a non-genetically modified (non-GM) tomato plant and/or a harvestable part thereof, exhibiting at least one inheritable improved trait as compared to a control population of plants of the same plant cultivar or line, the method comprising the steps of:

(a) providing a population of a cultivar or line of tomato seeds or plants;

(b) exposing said population (a) of a cultivar or line of tomato plants to a predetermined light treatment by irradiating with artificial light, partly in the presence of ambient daylight, every day for a duration of 2 to 6 weeks from sowing, preferably for 30 days from sowing, wherein the predetermined light treatment consists of irradiating with artificial red light of wavelength in the range of from about 600 nm to about 700 nm for a period of about 60 minutes, starting about 30 min. before sunset in the presence of daylight, and continuing about 30 minutes after sunset.

(c) growing the tomato plants in greenhouses for 2-3 months after the end of stage (b), then collecting, counting and weighing the tomatoes and collecting their seeds.

(d) monitoring at least one improved trait of the tomato plants at the end of step (b) and step (c) and comparing to a control population;

(e) selecting at the end of the treatment of step (b) the plants exhibiting at least one improved trait as compared to a control population and selecting again at the end of the growing stage of step (c) the plants exhibiting at least one improved trait as compared to a control population; wherein the number of plants selected in the first generation is from about 1% to about 4%, preferably from about 2% to about 3% of the initial plant population provided (a);

(f) propagating by sowing the tomato seeds at least one subsequent generation of the plants exhibiting at least one improved trait as compared to a control population; and

(g) repeating steps (b) to (f) on 1-7 subsequent generations, preferably at least two subsequent generations; wherein the number of plants selected in step (e) increases from generation to generation,

thereby obtaining a tomato crop plant and/or harvestable part thereof exhibiting at least one improved trait, wherein the improved trait is inheritable for at least one more generation, preferably for two or more generations, more preferably for 3 to 5 generations, and even more preferably for 6 to 8 generations.

In some embodiments, there is provided the above method of this disclosure for producing a non-genetically modified (non-GM) tomato plant and/or a harvestable part thereof, exhibiting at least one inheritable improved trait as compared to a control population of plants of the same plant cultivar or line, by monitoring at least one improved trait of said tomato plants, wherein the at least one improved inheritable trait being monitored is selected from the group consisting of tomato yield, tomato number, tomato weight, tomato shelf life, seed number, seed weight, plant height, main stem width, stem thickness, plant biomass, number of stems, number of secondary stems, stress resistance, pest resistance, virus resistance, drought tolerance, herbicide tolerance, delayed senescence, modified color, photosynthesis efficiency, nitrogen concentration in leaves and any combination thereof.

In some other embodiments, there is provided the above method for producing a non-genetically modified (non-GM) tomato plant and/or a harvestable part thereof, by monitoring at least one improved trait of said tomato plants, wherein the at least one improved inheritable trait being monitored is inheritable improved tomato yield.

According to some embodiments, there is provided the above method for producing a non-genetically modified (non-GM) tomato plant and/or a harvestable part thereof, by monitoring at least one improved inheritable trait of said tomato plants, wherein the at least one improved inheritable trait is an increased tomato yield of at least 2% and up to 800%, or more particularly 2%, 5%, 10%, 20%, 30%, 40%, 50%, 60%, 70%, 80%, 90%, 100%, 200%, 300%, 400%, 500%, 600%, 700%, 800% or higher, as compared to the control population of the same tomato cultivar or line.

According to some other embodiments, there is provided the above method for producing a non-genetically modified (non-GM) tomato plant and/or a harvestable part thereof, by monitoring at least one improved inheritable trait of said tomato plants, wherein the at least one inheritable improved trait is the result of an epigenetic effect.

In some embodiments, there is provided the above method for producing a non-genetically modified (non-GM) tomato plant and/or a harvestable part thereof, by monitoring at least one improved inheritable trait of said tomato plants, wherein applied to plants or harvestable plant parts selected from the group consisting of tomatoes, seeds, seedlings, tissue cultures, calluses, meristems, regenerable cells, protoplasts, potted seedlings, adult plants and any combination thereof.

In some other embodiments, there is provided the above method for producing a non-genetically modified (non-GM) tomato plant and/or a harvestable part thereof, by monitoring at least one improved inheritable trait of said tomato plants, wherein said the step of irradiating with artificial light is applied at a periodicity selected from the group consisting of before sunset, during sunset, after sunset and any combination thereof.

According to some embodiments, there is provided the above method for producing a non-genetically modified (non-GM) tomato plant and/or a harvestable part thereof, by monitoring at least one improved inheritable trait of said tomato plants, wherein applied to tomato cultivars, plant lines or plant types selected from the group consisting of M82, Adoration, Alicante, Azoychka, Beefsteak, Better Boy, Big Beef, Big Rainbow, Blaby Special, Black Krim, Brandy wine, Campari, Celebrity, Cherokee Purple, Canario, Early Girl, Enchantment, Ferris Wheel, Flamenco, Fourth of July, Garden Peach, Gardener's delight, German Johnson, Giulietta F1, Granadero, Great White, Green Zebra, Hillbilly, Japanese, Black Trifele, Jersey Boy, Jubilee, Juliet, Kumato, Lilian's Yellow, McDreamy, Matt's Wild Cherry, Micro Tom, Moneymaker, Monterosa, Montserrat, Mortgage Lifter, Mr. Stripey, Pantano Romanesco, Paul Robeson, Plum Tomato, Pumpkin Tomato, Raf Tomato, Rebellion, Red Currant, Roma, Rosa de Barbastro, Rutgers Tomato, San Marzano, Sasha Altai, Stupice, Tiny Tim, Traveller, Arkansas Traveler, Cherry Bambelo, Cherrry Nebula, Santorini, Super Sweet, Tomaccio, Yellow Pear, White Queen and any combination thereof.

According to some other embodiments, there is provided the above method for producing a non-genetically modified (non-GM) tomato plant and/or a harvestable part thereof, by monitoring at least one improved inheritable trait of said tomato plants, wherein applied to a non-genetically modified (non-GM) plant.

According to some embodiments, there is provided the above method for producing a non-genetically modified (non-GM) tomato plant and/or a harvestable part thereof, by monitoring at least one improved inheritable trait of said tomato plants, wherein applied to a genetically modified (GM) plant.

In some embodiments, there is provided a tomato plant or a harvestable tomato plant part, preferably a tomato, having at least one improved inheritable trait, wherein obtained by the method of this disclosure.

In some other embodiments, there is provided a tomato plant exhibiting at least one improved trait compared to a control population of tomato plants of the same cultivar or line, wherein produced by the method of this disclosure and wherein said tomato plant preserves its at least one improved trait for at least one subsequent generation, preferably for two or more generations, more preferably for 3 to 5 generations, and even more preferably for 6 to 8 generations, without exposure to additional light treatment.

According to some embodiments, there is provided a tomato plant or harvestable plant part of this disclosure, wherein selected from the group consisting of tomatoes, seeds, seedlings, tissue cultures, calluses, meristems, regenerable cells, protoplasts, potted seedlings, adult plants and any combination thereof.

According to some other embodiments, there is provided a tomato plant or harvestable plant part of this disclosure, wherein produced by irradiating with artificial light of red wavelengths.

In some embodiments, there is provided a tomato plant or harvestable plant part of this disclosure, wherein the at least one improved inheritable trait is an yield characteristic selected from the group consisting of, tomato number, tomato weight, seeds number, seeds weight, plant height, main stem width, stem thickness, plant biomass, number of stems, number of secondary stems, photosynthesis efficiency, nitrogen concentration in leaves and any combination thereof.

In some other embodiments, there is provided a tomato plant or harvestable plant part of this disclosure, wherein exhibiting as inheritable improved trait an increased tomato yield from about 2% to about 800% or more, as compared to a control population of the same cultivar.

In some other embodiments, there is provided a tomato plant or harvestable plant part of this disclosure, wherein the population of a cultivar or line of tomato seeds or plants provided is a non-genetically modified (non-GM) plant population.

In some embodiments, there is provided a tomato plant or harvestable plant part of this disclosure, wherein the population of a cultivar or line of tomato seeds or plants provided is is a genetically modified (GM) plant population.

According to some embodiments, there is provided a harvestable part of this disclosure, wherein it is a tomato exhibiting as improved trait an improved room temperature (17-27° C.) shelf life of at least 45 days after harvesting, as compared to about 10 days for an untreated control tomato.

According to some other embodiments, there is provided a method of screening for a tomato plant cultivar or line amenable to exhibiting at least one inheritable improved trait by applying the method of this disclosure and selecting the best plants exhibiting at least one inheritable improved trait.

### Examples

The following examples illustrate certain embodiments of the invention but are not meant to limit the scope of the claims in any way. The following examples are put forth so as to provide those of ordinary skill in the art with a complete disclosure and description of how to make and use the described invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, percentages are weight per weight, molecular weight is weight average molecular weight, temperature is in degrees Centigrade, and pressure is at or near atmospheric.

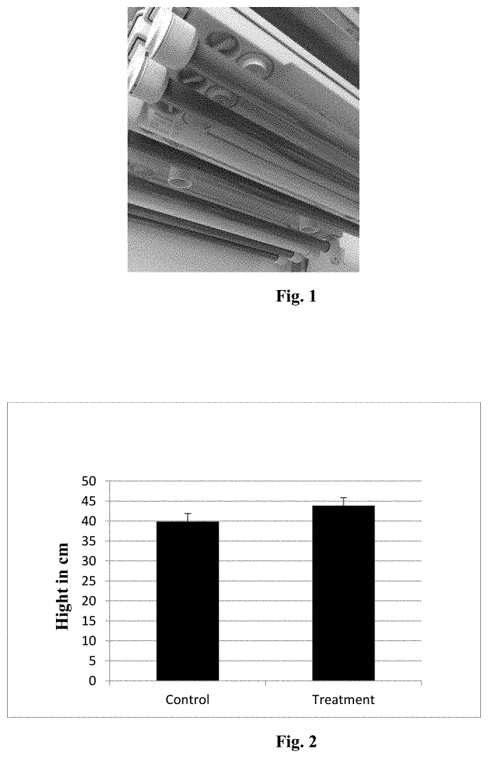

The irradiation treatment in the Examples 1-8 below used red fluorescent lamps of wavelength between 620-750 nm (see FIG. 1) situated at about 50-60 cm above the plants. The luminous flux was between 200-3000 lux.

Reference is made to the following non-limiting examples:

### Example 1: First Tomato Plant Treatment (F1)

Treatment procedure and results of stage F1 (first generation)
1. A population of 150 plants was provided and treated in the F1 treatment. After 30 days of treatment, about 20% of the plant's responded favorably to the treatment (one hour of red irradiation for half an hour before sunset and half an hour into the night, for one month). A few plants responded very well, some had moderate response and the majority did not respond at all in the first treatment stage F1. About 30 (20%) plants exhibiting at least one improved trait were selected. The first selection was based on plant height, volume (biomass) and vigor.
2. The selected plants were transferred to the commercial greenhouse, and planted there until completion of their growth cycle. During this period phenotype measurements were taken.
3. At the end of the growing cycle, tomatoes were collected from each plant, counted and weighted.
4. 3-4 best plants exhibiting an improved trait (in respect of the yield) (10-13% of the 30 plants in first selection, or 2-2.6% of the initial 150 plant population provided) were selected for a second round of treatment (F2) (the same as in F1), and their seeds were collected.
5. The same process was repeated for F2 and then for F3.

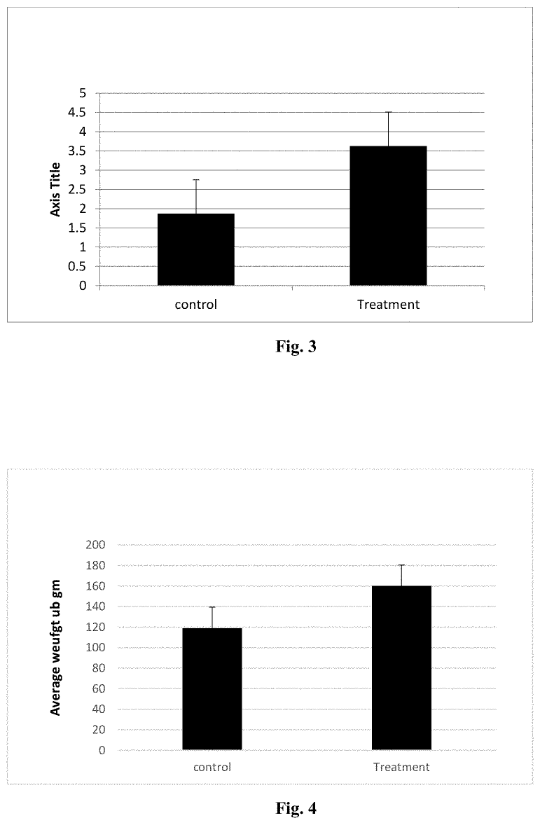

TABLE 1
Tomato plants (line M82) F1 treatment results
(see FIGS. 2, 3 and 4)

| F1 | Average | Average No. of | Average weight |
|---|---|---|---|
| Treatment | Plant Height | Inflorescences | of tomato plants |
| Treatment 29 plants | 43.86 cm | 3.62 | 160 gm |
| Control 15 plants | 39.87 cm | 1.87 | 119 gm |

Summing up, in the first treatment F1, there was not much difference between the average height of the control plants and the average height of the treated plants. However, the few individual responsive plants in F1 selected for further treatment in F2 were significantly higher than the control plants.

### Example 2: Second Tomato Plant Treatment (F2)

Treatment of 150 plants grown from seeds of F1 treatment began on the day of sowing.

The treatment included red irradiation in the evening, for half an hour before sunset and half an hour into the night. Red fluorescent light lamps of wavelength between 620-750 nm (see FIG. 1) situated at about 50-60 cm above the plants were used for irradiation. The luminous flux was between 200-3000 lux.

The 150 plants were irrigated 5 minutes twice a day, altogether 400 mm per growing cycle. Each pot was irrigated separately.

The treated group of plants included 89 plants (59% of the initial population provided of 150 plants) and the control group included 50 plants. The selected plants were grown in 12 cm pots arranged on two benches, four pots in a row on each bench.

18-20 treated plants exhibiting an improved trait (in respect of the yield) (20-22% of the selected 89 plants or 12-13% of the initial 150 plants) were selected and taken to the commercial greenhouse for the next round of treatment. The control plants were taken to the commercial greenhouse as is, without selection.

The treated group of plants showed significant trait improvements in comparison to the control group.

Growing conditions in the tomato greenhouses:
1. Date of sowing: 19.9.17. 150 seeds from F1 treatment were sown in pots, divided in two greenhouses. One tomato seed of the M88 cultivar from F1 was sown in each 12 cm pot.
2. Treatment of the plants sown in pots in greenhouses designated 1-2 started on the day of sowing.
3. Greenhouse No. 1: F2 treatment of tomato plants from 75 F1 pots.
4. Greenhouse No. 2: F2 treatment of tomato plants from 75 F1 pots.
5. Greenhouse No. 3: 50 control untreated plants from F1 mixed seeds control plants.
6. Treatment started at 6:30 PM, ended 7.30 PM, using red light 620-750 nm
7. First selection of the best plants (20-30 plants, (13-20% out of a total of 150 plants in two greenhouses) was based on plant height, volume (biomass) and vigor.
8. Irrigation: 5 min, twice a day
9. Transfer of the selected F2 tomato plants from the treatment greenhouses to commercial greenhouse on 17.10.17
10. Second selection of the best 3-4 plants (2-2.7% out of a total of the initial 150 plants) was done at the end of the growing cycle was based on average tomato yield.

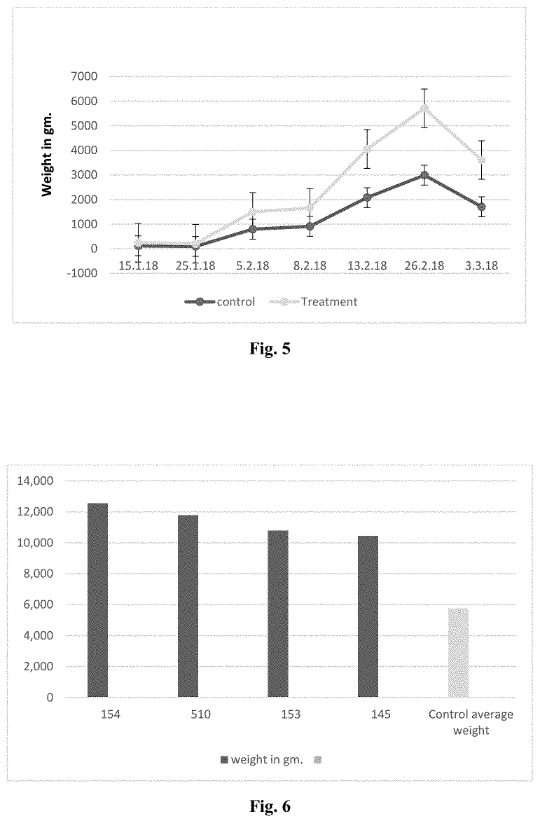

Measurement of Phenotypes
11. Phenotype measurements of the treated tomato plants were taken during the growth cycle and compared to control plants. E.g. plants height, flowering date, tomato ripeness, total weight etc.
12. First flower in the treated group appears about 2 months earlier than in the control plants. The number of inflorescences in the treated plants is 30% to 60% higher than in the control plants. Early flowering and increase in the number of flowers demonstrate important changes in the plant's genome.
13. The volume and the biomass of the treated plants are at least twice the size of the control plants.
14. The height of the treated plants is significantly higher than the control plants. About 76 cm in the treated group versus 55 cm in the control. After two weeks the difference in height was 38.2%.
15. The total average weight of all tomatoes of the control (non-treated) tomato plants was 70,645.8 g.
16. The total average weight of all the tomatoes from treated tomato plants was 115,736 g.
17. The total weight of all tomatoes from the treated plants is 63.8% higher than that of the control plants.
18. The total tomato weight of the four best performing plants was compared to the average weight of the tomatoes from the control plants (non-treated). These four plants were selected for the next round of treatment F3. The total tomato weight from best performing plant was about 118% higher than the average weight of the tomatoes from the control plants.
19. The development of ripe tomatoes is shown in FIG. 5. Ripe tomatoes were collected and total tomato weight in the treated plants versus the total weight of tomatoes from the control plants was compared.

Summary of F2 Results:
1. Significant differences were observed in all measured parameters between the treated plants and the control plants, such as: height, plant volume, biomass, early flowering, number of flowers, yield,
2. The treated plants were in average 38.2% higher than the average control plants. The same with plant volume and increase in biomass.
3. Flowering was earlier in the treated plants vs. control.
4. Ripening of tomatoes is quicker in the treated plants versus the control plants.
5. The number of flowers is at least twice in the treated plants versus in the control plants.
6. The treated group showed a yield increase of 63.8% versus the control group.
7. The yield of the treated plants is higher by 118% than the control average yield.
8. The average tomato weight in the F2 treated plants was about double the average weight of the control. The decline toward the end of the curve is due to the fact that there are reduced number of tomatoes toward the end of the experiment (see FIG. 5).

TABLE 2
Total tomato weight in the four best F2 treated plants
vs. average weight of control plants (see FIG. 6)

| Treated plant no. | Total tomato weight in g. |
|---|---|
| 154 | 12,558 |
| 510 | 11,776 |
| 153 | 10,776 |
| 145 | 10,442 |
| Control plants' average weight | 5,760 |

### Example 3—Tomatoes from F2 Treated Plants Exhibiting Extended Shelf Life

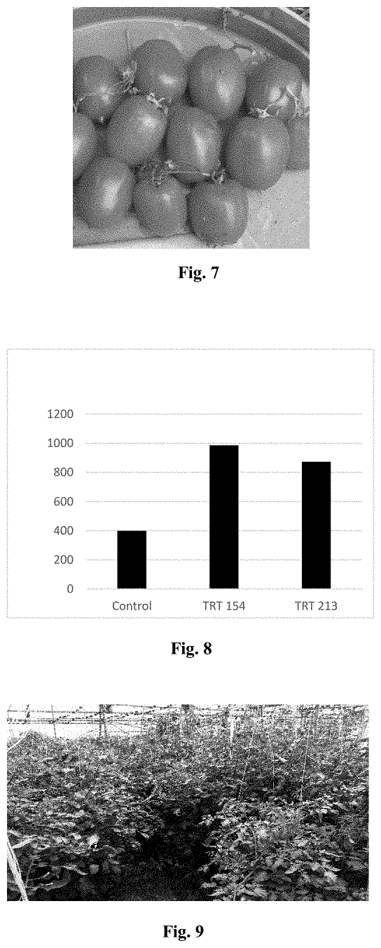

Tomatoes from F2 treated tomato plants which were stored openly at room temperature (between 17-27° C.) in the shade, but without any cooling or addition of preservatives, exhibited an extended shelf life of at least 48 days (see FIG. 7) after harvesting (plant no. BIS 154), while the control tomatoes' shelf life is about 10 days.

### Example 4—F3 Tomato Experiments

The third round of treatment (F3) was performed on seeds and seedlings which received previously two rounds of treatment. The average weight of tomatoes from the two best treated sub-lines TRT 154 and TRT213 (see FIG. 8) were significantly higher than the control which received no treatment at all.

The effect of the F3 treatment can be seen in FIG. 9, which depicts a photograph of the F3 treated tomato plants near the control plants.

### Example 5—Germination Rates of the Seeds from F3 and F4 Treatments

The seed germination rate is the percentage of seeds out of a certain seed population which on sowing, germinate and turn into plants.

Germination rate is an indicator for the fertility of the seeds from treated plants versus the control seeds an indication if our treatment effect in fertility of the treated seeds. The results in Table 3 show that the treatment of this disclosure significantly improved the fertility of the seeds obtained from F3-F4 treatments.

TABLE 3
Germination rates of tomato seeds from F3 and F4 treatments

| | Seeds from | Seeds from | % Difference |
|---|---|---|---|
| Generation | treated plants | control plants | treated and |
| (F) | % germination | % germination | untreated seeds |
| F3 | 78% | 52% | 26% |
| F4 | 89% | 52% | 37% |

### Example 6—Optimal Irradiation Wavelength Range

150 F2 seedlings were divided into 4 groups and were irradiated with light different wavelengths as follows:
1. Group 1 received red light for an hour before sunset for 1 month
2. Group 2 received blue light for one hour before sunset for 1 month.
3. Group 3 received white light of one hour for a month before sunset.
4. Group 4 received no additional irradiation at all for a month.

The average tomato weights obtained by irradiation at red, blue and white wavelength range are detailed in Table 4 below.

TABLE 4
Selection of the optimal irradiation wavelength
range out of red, blue and white light irradiation
based on best average tomatos per plant weight

| | | | | Control |
|---|---|---|---|---|
| Irradiation | Red light | Blue light | White light | (No irrad.) |
| wavelength | Group 1 | Group 2 | Group 3 | Group 4 |
| Av. tomato weight | 160 gm | 145 gm | 123 gm | 119 gm |

### Example 7—RNA Sequencing

In order to determine if the irradiation method disclosed in the present application (one hour of red irradiation {between 620-750 nm} for half an hour before sunset and half an hour into the night, for one month) results in transcriptomic changes, RNA sequencing assay was carried out on tomato plants treated with said irradiation protocol and control plants. The RNA sequencing assay was performed on F3 tomato plants (treated and control plants).

RNA was extracted from a 50-100 mg of tomato (line M82) leaves using Sigma Spectrum Plant Total RNA Kit (Cat #SLCD4163) according to the manufacturer's guidelines. The resultant RNA was eluted in Nuclease free water (Cat #AM9938, Ambion, USA), quantified and analyzed for integrity using standard protocols.

### Library Preparation

RNA sequencing libraries were prepared with Illumina-compatible NEBNext® Ultra™ II Directional RNA Library Prep Kit (New England BioLabs, MA, USA) at Genotypic Technology Pvt. Ltd., Bangalore, India. 500 ng of total RNA was taken for mRNA isolation, fragmentation and priming. Fragmented and primed mRNA was further subjected to first strand synthesis followed by second strand synthesis. The double stranded cDNA was purified using JetSeq Beads (Bioline, Cat #BIO-68031). Purified cDNA was end-repaired, adenylated and ligated to Illumina adapters as per NEBNext® Ultra™ II Directional RNA Library Prep protocol followed by second strand excision using USER enzyme at 37° C. for 15 minutes. Adapter ligated cDNA was purified using JetSeq Beads and was subjected to 11 cycles for Indexing-(98° C. for 30 sec, cycling (98° C. for 10 sec, 65° C. for 75 sec) and 65° C. for 5 min) and enriching the adapter-ligated fragments. Final PCR products (sequencing libraries) were purified with JetSeq Beads, followed by library quality control check. Illumina-compatible sequencing libraries were quantified by Qubitfluorometer (Thermo Fisher Scientific, MA, USA) and fragment size distribution was analyzed on Agilent 2200 TapeStation.

### Illumina Sequencing

The libraries were paired-end sequenced on Illumina HiSeq X Ten sequencer for 150 cycles (Illumina, San Diego, USA) following manufacturer's instructions.

### Analysis Methodology

Raw data pre-processing was done to remove low quality data and adaptor sequences using the Trim Galore tool (www.bioinformatics.babraham.ac.uk/projects/trim_galore/). The high-quality reads were considered for alignment with reference genome [Solanum lycopersicum (solgenomics.net/organism/Solanum_lycopersicum/genome)] using a splice aligner (see Kim, D., Langmead, B., & Salzberg, S. L. hisat2. Nature methods, 2015). Expression calculation was performed at gene level using HTSeq tool (see “HTSeq—a Python framework to work with high-throughput sequencing data”, Anders, S., Pyl, P. T., & Huber, W., Bioinformatics, 31(2), 166-169, 2015). DESeq was used to calculate the differentially expressed genes (see “Differential expression analysis for sequence count data. Genome biology”, Anders, S., & Huber, W., Genome biology, 11(10), R106, 2010). Genes were categorized into upregulated, downregulated and neutrally regulated based on the log 2fold change cutoff of 1 value. This experiment was carried out in triplicates, and was subjected to rigorous and stringent statistical tests and evaluations.

The following tables (Table 5-8) present the genes which are differentially expressed between the treated F3 tomato plants and control plants. Table 5 depicts genes whose expression was upregulated by a 2-4 fold increase in treated plants compared to control plants.

TABLE 5
Upregulated Genes between Treated and Control F3 Tomato Plants
(2-4 fold increase)

| | | Control | Treatment | |
|---|---|---|---|---|
| Gene_ID | log2FoldChange | Expression | Expression | Protein Name |
| Solyc05g051640 | 2.00 | 85.23 | 340.65 | Protein kinase domain- |
| | | | | containing protein |
| Solyc02g036260 | 2.00 | 10.21 | 40.70 | Uncharacterized protein |
| Solyc03g043910 | 1.99 | 5.10 | 20.32 | BSD domain-containing protein |
| Solyc03g093790 | 1.99 | 9.48 | 37.79 | Cyclin |
| Solyc08g078420 | 1.99 | 13.13 | 52.29 | AP2/ERF domain-containing |
| | | | | transcription factor |
| Solyc01g087140 | 1.99 | 6.87 | 27.37 | Uncharacterized protein |
| Solyc03g007720 | 1.99 | 8.41 | 33.29 | Uncharacterized protein |
| Solyc04g082750 | 1.98 | 12.79 | 50.60 | 3Beta_HSD domain-containing |
| | | | | protein |
| Solyc09g084490 | 1.98 | 6.98 | 27.60 | Uncharacterized protein |
| Solyc10g078680 | 1.98 | 10.24 | 40.36 | Uncharacterized protein |
| Solyc05g013870 | 1.98 | 108.42 | 427.21 | Uncharacterized protein |
| Solyc10g083670 | 1.98 | 38.94 | 153.31 | Glyco_trans_2-like domain- |
| | | | | containing protein |
| Solyc01g102720 | 1.97 | 32.57 | 127.98 | Uncharacterized protein |
| Solyc06g052010 | 1.97 | 12.06 | 47.40 | DUF4005 domain-containing |
| | | | | protein |
| Solyc03g033660 | 1.97 | 8.37 | 32.87 | GATA transcription factor |
| Solyc01g107340 | 1.97 | 41.50 | 162.65 | Uncharacterized protein |
| Solyc10g079170 | 1.97 | 25.18 | 98.59 | Protein kinase domain- |
| | | | | containing protein |
| Solve10g075107 | 1.97 | 7.72 | 30.21 | Non-specific lipid-transfer |
| | | | | protein |
| Solyc06g007130 | 1.96 | 129.54 | 505.33 | Uncharacterized protein |
| Solyc10g084250 | 1.96 | 31.12 | 121.33 | Clade VIII lectin receptor |
| | | | | kinase |
| Solyc07g049370 | 1.96 | 37.90 | 147.66 | Glucan endo-1,3-beta-D- |
| | | | | glucosidase (EC 3.2.1.39) |
| Solyc11g018710 | 1.96 | 6.95 | 27.08 | Uncharacterized protein |
| Solyc08g067550 | 1.96 | 5.48 | 21.33 | Non-specific lipid-transfer |
| | | | | protein |
| Solyc01g058270 | 1.96 | 27.80 | 108.09 | Cas1_AcylT domain-containing |
| | | | | protein |
| Solyc06g067980 | 1.96 | 10.23 | 39.75 | Uncharacterized protein |
| Solyc01g094690 | 1.96 | 224.47 | 871.75 | Putative water channel protein |
| Solyc05g055730 | 1.96 | 10.96 | 42.55 | Methylenetetrahydrofolate |
| | | | | reductase (EC 1.5.1.20) |
| Solyc12g006260 | 1.96 | 11.64 | 45.18 | Uncharacterized protein |
| Solyc06g072830 | 1.96 | 25.59 | 99.37 | PWWP domain-containing |
| | | | | protein |
| Solyc01g088780 | 1.95 | 10.58 | 40.97 | Ras-related C3 botulinum toxin |
| | | | | substrate 1 |
| Solyc03g122360 | 1.95 | 64.76 | 250.84 | Uncharacterized protein |
| Solyc08g081480 | 1.95 | 63.16 | 244.55 | Putative polygalacturonase-like |
| | | | | (Fragment) |
| Solyc02g088100 | 1.95 | 211.16 | 816.51 | Expansin |
| Solyc03g005280 | 1.95 | 29.78 | 114.86 | Uncharacterized protein |
| Solyc06g005680 | 1.95 | 47.85 | 184.53 | Putative transcription factor |
| | | | | PCL1-like |
| Solyc01g017600 | 1.95 | 121.31 | 467.46 | Uncharacterized protein |
| Solyc02g069490 | 1.94 | 221.04 | 850.88 | Delta(24)-sterol reductase (EC |
| | | | | 1.3.1.72) |
| Solyc06g007240 | 1.94 | 46.58 | 178.44 | Ubiquitin-like domain- |
| | | | | containing protein |
| Solyc11g010710 | 1.94 | 15.04 | 57.58 | Uncharacterized protein |
| Solyc09g014480 | 1.93 | 27.80 | 106.07 | LRRNT_2 domain-containing |
| | | | | protein |
| Solyc02g083720 | 1.93 | 7.62 | 29.04 | MLO-like protein |
| Solyc08g079690 | 1.93 | 24.52 | 93.38 | Uncharacterized protein |
| Solyc01g109230 | 1.93 | 80.24 | 305.15 | Phorbol-ester/DAG-type |
| | | | | domain-containing protein |
| Solyc12g010030 | 1.93 | 11.71 | 44.53 | Lap17.1a protein |
| Solyc06g084070 | 1.93 | 30.03 | 114.21 | Auxin-responsive protein |
| Solyc06g062460 | 1.93 | 57.95 | 220.08 | BHLH domain-containing |
| | | | | protein |
| Solyc12g005250 | 1.92 | 8.43 | 32.01 | Kinesin-like protein |
| Solyc09g007360 | 1.92 | 45.36 | 171.64 | Uncharacterized protein |
| Solyc11g071750 | 1.92 | 10.90 | 41.19 | Regulator of gene silencing |
| Solyc02g090930 | 1.92 | 16.71 | 63.14 | Uncharacterized protein |
| Solyc06g071330 | 1.92 | 14.96 | 56.43 | Xanthine/uracil permease |
| | | | | family protein |
| Solyc06g069070 | 1.91 | 9.46 | 35.59 | AAI domain-containing protein |
| Solyc03g112540 | 1.91 | 13.54 | 50.86 | Uncharacterized protein |
| Solyc11g012130 | 1.91 | 12.83 | 48.09 | Putative early nodulin-like |
| | | | | protein 1-like |
| Solyc03g111100 | 1.91 | 254.66 | 953.81 | Uncharacterized protein |
| Solyc05g048800 | 1.91 | 9.82 | 36.79 | CAP10 domain-containing |
| | | | | protein |
| Solyc04g074080 | 1.91 | 92.23 | 345.40 | WW domain-containing protein |
| Solyc01g080070 | 1.90 | 160.56 | 600.85 | HMA domain-containing |
| | | | | protein |
| Solyc08g005240 | 1.90 | 12.40 | 46.38 | Uncharacterized protein |
| Solyc01g088400 | 1.90 | 20.80 | 77.64 | Uncharacterized protein |
| Solyc01g087590 | 1.90 | 57.92 | 216.17 | Uncharacterized protein |
| Solyc06g054130 | 1.90 | 30.05 | 111.96 | Uncharacterized protein |
| Solyc04g009980 | 1.90 | 6.99 | 26.03 | Eight-dependent short |
| | | | | hypocotyls 1 |
| Solyc02g083130 | 1.90 | 36.10 | 134.37 | Uncharacterized protein |
| Solyc09g092720 | 1.90 | 265.75 | 988.78 | Uncharacterized protein |
| Solyc04g063390 | 1.90 | 13.11 | 48.78 | chaperone protein dnaJ 10-like |
| Solyc04g005250 | 1.89 | 113.32 | 421.31 | Uncharacterized protein |
| Solyc04g005070 | 1.89 | 32.43 | 120.47 | Putative peptide transporter-like |
| | | | | (Fragment) |
| Solyc01g016370 | 1.89 | 5.86 | 21.75 | Cf-19 |
| Solyc08g076350 | 1.89 | 6.18 | 22.92 | Hydrolase_4 domain-containing |
| | | | | protein |
| Solyc04g079170 | 1.89 | 10.27 | 38.02 | Uncharacterized protein |
| Solyc09g065590 | 1.89 | 51.59 | 190.96 | Putative ovule protein |
| Solyc01g005280 | 1.89 | 7.63 | 28.24 | CRAL-TRIO domain- |
| | | | | containing protein |
| Solyc02g091990 | 1.89 | 10.25 | 37.90 | 1-aminocyclopropane-1- |
| | | | | carboxylate synthase 3 (ACC |
| | | | | synthase 3) (EC 4.4.1.14) (Le- |
| | | | | ACS3) (ACS-3) (S-adenosyl-L- |
| | | | | methionine |
| | | | | methylthioadenosine-lyase 3) |
| Solyc09g007380 | 1.89 | 6.94 | 25.67 | AB hydrolase-1 domain- |
| | | | | containing protein |
| Solyc01g058320 | 1.89 | 141.84 | 524.12 | Uncharacterized protein |
| Solyc09g018020 | 1.88 | 75.71 | 278.90 | Expansin |
| Solyc04g078195 | 1.88 | 530.88 | 1,953.45 | Uncharacterized protein |
| Solyc02g083620 | 1.88 | 17.14 | 62.99 | L-ascorbate peroxidase (EC |
| | | | | 1.11.1.11) |
| Solyc01g079850 | 1.88 | 16.05 | 58.94 | PMR5N domain-containing |
| | | | | protein |
| Solyc09g092760 | 1.87 | 78.11 | 286.35 | Uncharacterized protein |
| Solyc09g030450 | 1.87 | 32.57 | 119.37 | Protein kinase domain- |
| | | | | containing protein |
| Solyc12g009930 | 1.87 | 60.99 | 223.39 | Glycosyltransferase (EC 2.4.1.—) |
| Solyc07g041770 | 1.87 | 16.09 | 58.85 | Uncharacterized protein |
| Solyc11g008680 | 1.87 | 13.87 | 50.72 | Acyl-[acyl-carrier-protein] |
| | | | | desaturase (EC 1.14.19.—) |
| Solyc11g066130 | 1.87 | 106.29 | 387.78 | Osmotin-like protein |
| Solyc03g111810 | 1.87 | 293.17 | 1,069.57 | Uncharacterized protein |
| Solyc05g005760 | 1.87 | 59.38 | 216.55 | Putative NHL domain- |
| | | | | containing protein-like |
| Solyc01g096410 | 1.87 | 53.46 | 194.93 | Phosphatidylinositol 4- |
| | | | | phosphate 5-kinase (EC |
| | | | | 2.7.1.68) |
| Solyc09g014940 | 1.86 | 19.40 | 70.65 | Putative wound-induced protein |
| | | | | 1-like |
| Solyc01g086720 | 1.86 | 13.12 | 47.72 | Uncharacterized protein |
| Solyc03g098190 | 1.86 | 5.78 | 20.97 | Uncharacterized protein |
| Solyc06g075460 | 1.86 | 15.36 | 55.66 | Nucleoside-diphosphate kinase |
| | | | | (EC 2.7.4.6) |
| Solyc03g005960 | 1.86 | 112.80 | 408.14 | Non-specific serine/threonine |
| | | | | protein kinase (EC 2.7.11.1) |
| Solyc10g078330 | 1.85 | 5.54 | 20.02 | Cyclin N-terminal domain- |
| | | | | containing protein |
| Solyc01g091490 | 1.85 | 76.08 | 274.63 | Uncharacterized protein |
| Solyc11g010350 | 1.85 | 7.63 | 27.55 | Copia LTR rider |
| Solyc05g016390 | 1.85 | 14.59 | 52.62 | RanBP2-type domain- |
| | | | | containing protein |
| Solyc01g094230 | 1.85 | 5.44 | 19.61 | Uncharacterized protein |
| Solyc06g076610 | 1.84 | 33.25 | 119.39 | RING-CH-type domain- |
| | | | | containing protein |
| Solyc10g081790 | 1.84 | 5.49 | 19.69 | RING-type domain-containing |
| | | | | protein |
| Solyc11g020960 | 1.84 | 93.26 | 333.07 | Probable proteinase inhibitor |
| Solyc12g008460 | 1.83 | 75.58 | 269.34 | Senescence domain-containing |
| | | | | protein |
| Solyc03g079880 | 1.83 | 11.01 | 39.21 | AAI domain-containing protein |
| Solyc10g009350 | 1.83 | 13.08 | 46.60 | Non-specific serine/threonine |
| | | | | protein kinase (EC 2.7.11.1) |
| Solyc06g160230 | 1.83 | 14.17 | 50.48 | Pyruvate kinase (EC 2.7.1.40) |
| Solyc10g044510 | 1.83 | 12.41 | 44.17 | MLO-like protein |
| Solyc07g047680 | 1.83 | 5.50 | 19.57 | F-box protein SNE |
| Solyc03g122140 | 1.83 | 6.85 | 24.28 | (S)-2-hydroxy-acid oxidase (EC |
| | | | | 1.1.3.15) |
| Solyc07g041230 | 1.82 | 102.70 | 362.83 | Uncharacterized protein |
| Solyc06g062580 | 1.82 | 46.73 | 164.79 | Beta-galactosidase (EC |
| | | | | 3.2.1.23) |
| Solyc04g016000 | 1.82 | 9.49 | 33.45 | HSF_DOMAIN domain- |
| | | | | containing protein |
| Solyc04g150164 | 1.82 | 16.04 | 56.47 | Uncharacterized protein |
| Solyc02g082260 | 1.81 | 94.15 | 331.01 | 3-hydroxy-3-methylglutaryl |
| | | | | coenzyme A reductase (HMG- |
| | | | | CoA reductase) (EC 1.1.1.34) |
| Solyc03g119390 | 1.81 | 57.10 | 200.72 | BHLH domain-containing |
| | | | | protein |
| Solyc02g072500 | 1.81 | 16.79 | 58.87 | Uncharacterized protein |
| Solyc11g010920 | 1.81 | 22.64 | 79.36 | Kinesin motor domain- |
| | | | | containing protein |
| Solyc04g080270 | 1.81 | 12.77 | 44.74 | Uncharacterized protein |
| Solyc05g006160 | 1.81 | 42.49 | 148.68 | Uncharacterized protein |
| Solyc09g075350 | 1.81 | 24.87 | 86.94 | Pectinesterase (EC 3.1.1.1 1) |
| Solyc12g150113 | 1.80 | 19.27 | 67.34 | HMA domain-containing |
| | | | | protein |
| Solyc01g102330 | 1.80 | 62.20 | 217.12 | SASA domain-containing |
| | | | | protein |
| Solyc06g072550 | 1.80 | 6.92 | 24.10 | CASP-like protein |
| Solyc04g079930 | 1.80 | 48.93 | 170.35 | SAP30_Sin3_bdg domain- |
| | | | | containing protein |
| Solyc05g013850 | 1.80 | 291.45 | 1,013.85 | Uncharacterized protein |
| Solyc02g032940 | 1.80 | 45.52 | 158.26 | Uncharacterized protein |
| Solyc01g096320 | 1.79 | 18.47 | 64.07 | Homeobox domain-containing |
| | | | | protein |
| Solyc04g064760 | 1.79 | 99.42 | 344.07 | Serine carboxypeptidase-like 42 |
| Solyc06g076290 | 1.79 | 23.46 | 81.14 | Scarecrow-like protein 30 |
| Solyc08g079890 | 1.79 | 37.13 | 128.05 | Uncharacterized protein |
| Solyc01g091530 | 1.78 | 64.33 | 221.51 | Fasciclin-like arabinogalactan |
| | | | | protein 13 |
| Solyc11g069590 | 1.78 | 31.32 | 107.85 | Receptor-like serine/threonine- |
| | | | | protein kinase NCRK |
| Solyc08g005630 | 1.78 | 34.70 | 119.48 | Long-chain-alcohol oxidase |
| | | | | (EC 1.1.3.20) |
| Solyc06g083080 | 1.78 | 43.89 | 150.94 | Uncharacterized protein |
| Solyc03g113450 | 1.78 | 32.55 | 111.76 | LRR receptor-like |
| | | | | serine/threonine-protein kinase |
| | | | | FEI 2 isoform X4 |
| Solyc05g006020 | 1.78 | 52.63 | 180.58 | Uncharacterized protein |
| Solyc06g072520 | 1.78 | 11.38 | 39.01 | BHLH domain-containing |
| | | | | protein |
| Solyc07g048090 | 1.78 | 50.99 | 174.72 | Putative fasciclin-like |
| | | | | arabinogalactan protein 7-like |
| Solyc11g066820 | 1.77 | 476.31 | 1,629.92 | Putative glucomannan 4-beta- |
| | | | | mannosyltransferase 2-like |
| Solyc08g022240 | 1.77 | 13.85 | 47.32 | Lipase_3 domain-containing |
| | | | | protein |
| Solyc12g049210 | 1.77 | 12.83 | 43.77 | Uncharacterized protein |
| Solyc07g054900 | 1.77 | 7.22 | 24.61 | AB hydrolase-1 domain- |
| | | | | containing protein |
| Solyc02g092050 | 1.77 | 18.91 | 64.46 | Uncharacterized protein |
| Solyc12g008580 | 1.77 | 26.70 | 90.77 | Glucan endo-1,3-beta-D- |
| | | | | glucosidase (EC 3.2.1.39) |
| Solyc03g119940 | 1.76 | 20.00 | 67.96 | Uncharacterized protein |
| Solyc06g072220 | 1.76 | 60.01 | 203.78 | Uncharacterized protein |
| Solyc08g044260 | 1.76 | 118.91 | 403.77 | Sterol desaturase |
| Solyc09g007410 | 1.76 | 57.18 | 194.02 | Uncharacterized protein |
| Solyc06g051350 | 1.76 | 13.91 | 47.19 | PMR5N domain-containing |
| | | | | protein |
| Solyc09g098590 | 1.76 | 41.90 | 142.16 | Sucrose synthase (EC 2.4.1.13) |
| Solyc09g092235 | 1.76 | 14.97 | 50.69 | Uncharacterized protein |
| Solyc03g005910 | 1.76 | 69.89 | 236.49 | Lipase_GDSL domain- |
| | | | | containing protein |
| Solyc03g112930 | 1.76 | 21.93 | 74.13 | Dof-type domain-containing |
| | | | | protein |
| Solyc12g042500 | 1.75 | 6.61 | 22.30 | Uncharacterized protein |
| Solyc10g085670 | 1.75 | 29.44 | 99.30 | Protein kinase domain- |
| | | | | containing protein |
| Solyc06g074070 | 1.75 | 90.02 | 303.45 | Protein kinase domain- |
| | | | | containing protein |
| Solyc06g083580 | 1.75 | 37.52 | 126.32 | Pectate lyase (EC 4.2.2.2) |
| Solyc11g005760 | 1.75 | 6.23 | 20.96 | Hexosyltransferase (EC 2.4.1.—) |
| | | | | (Fragment) |
| Solyc02g063390 | 1.75 | 10.65 | 35.82 | S5A_REDUCTASE domain- |
| | | | | containing protein |
| Solyc12g100180 | 1.75 | 48.76 | 164.05 | Uncharacterized protein |
| Solyc09g074630 | 1.75 | 25.51 | 85.74 | BTB/POZ-like protein |
| Solyc04g016290 | 1.75 | 7.99 | 26.84 | Rho_N domain-containing |
| | | | | protein |
| Solyc11g005090 | 1.75 | 23.81 | 79.96 | Cyclin A1 |
| Solyc12g098630 | 1.75 | 17.55 | 58.93 | Kinesin motor domain- |
| | | | | containing protein |
| Solyc02g063140 | 1.75 | 142.54 | 477.81 | 3-ketoacyl-CoA synthase (EC |
| | | | | 2.3.1.—) |
| Solyc07g054170 | 1.74 | 14.97 | 50.10 | Uncharacterized protein |
| Solyc04g012030 | 1.74 | 41.55 | 139.00 | S-acyltransferase (EC |
| | | | | 2.3.1.225) |
| | | | | (Palmitoyltransferase) |
| Solyc09g097860 | 1.74 | 13.12 | 43.82 | Uncharacterized protein |
| Solyc03g083170 | 1.74 | 9.11 | 30.39 | Uncharacterized protein |
| Solyc09g075960 | 1.74 | 17.84 | 59.50 | Uncharacterized protein |
| Solyc02g087880 | 1.73 | 64.53 | 214.55 | Tubulin alpha chain |
| Solyc12g009270 | 1.73 | 7.33 | 24.37 | PMEI domain-containing |
| | | | | protein |
| Solyc03g119190 | 1.73 | 9.10 | 30.17 | Reticulon-like protein |
| Solyc04g063350 | 1.73 | 57.08 | 189.20 | E1_dh domain-containing |
| | | | | protein |
| Solyc03g078650 | 1.73 | 41.39 | 136.97 | Uncharacterized protein |
| Solyc06g066840 | 1.73 | 84.84 | 280.64 | Putative 2-oxoglutarate- |
| | | | | dependent dioxygenase AOP1.2 |
| Solyc09g065480 | 1.73 | 24.11 | 79.76 | IPPc domain-containing protein |
| Solyc09g075580 | 1.73 | 20.50 | 67.79 | Uncharacterized protein |
| Solyc10g074920 | 1.72 | 41.12 | 135.81 | Mannan endo-1,4-beta- |
| | | | | mannosidase (EC 3.2.1.78) |
| Solyc07g045450 | 1.72 | 18.26 | 60.22 | Uncharacterized protein |
| Solyc04g051244 | 1.72 | 13.15 | 43.36 | Uncharacterized protein |
| Solyc09g010810 | 1.72 | 14.32 | 47.17 | Kinesin-like protein |
| Solyc12g006590 | 1.72 | 17.66 | 58.20 | Uncharacterized protein |
| Solyc06g084450 | 1.72 | 8.33 | 27.42 | Uncharacterized protein |
| Solyc04g007690 | 1.72 | 59.23 | 195.04 | Auxin efflux carrier component |
| Solyc03g114690 | 1.72 | 15.01 | 49.34 | WD_REPEATS_REGION |
| | | | | domain-containing protein |
| Solyc05g053900 | 1.72 | 93.51 | 307.03 | Peptidase A1 domain- |
| | | | | containing protein |
| Solyc03g044060 | 1.71 | 105.91 | 347.40 | Formin-like protein |
| Solyc12g095810 | 1.71 | 31.82 | 104.24 | Uncharacterized protein |
| Solyc08g079060 | 1.71 | 24.08 | 78.80 | Trehalose 6-phosphate |
| | | | | phosphatase (EC 3.1.3.12) |
| Solyc05g009330 | 1.71 | 108.84 | 355.36 | Putative sodium/hydrogen |
| | | | | exchanger 7-like |
| Solyc11g071300 | 1.71 | 8.40 | 27.40 | Uncharacterized protein |
| Solyc07g062543 | 1.71 | 17.78 | 58.01 | MurA transferase |
| Solyc02g065530 | 1.71 | 42.35 | 138.17 | Uncharacterized protein |
| Solyc01g058160 | 1.70 | 9.10 | 29.63 | Uncharacterized protein |
| Solyc10g085070 | 1.70 | 277.35 | 902.90 | UPF0503 protein, chloroplastic |
| Solyc04g077640 | 1.70 | 8.38 | 27.25 | Peptidylprolyl isomerase (EC |
| | | | | 5.2.1.8) |
| Solyc01g103790 | 1.70 | 11.68 | 37.90 | Protein kinase APK1B, |
| | | | | chloroplast |
| Solyc07g066180 | 1.70 | 38.42 | 124.49 | AAA domain-containing |
| | | | | protein |
| Solyc10g009360 | 1.69 | 13.60 | 43.98 | Dof-type domain-containing |
| | | | | protein |
| Solyc02g089550 | 1.69 | 167.89 | 542.66 | Receptor protein kinase |
| Solyc02g078950 | 1.69 | 81.54 | 263.40 | Beta-galactosidase (EC |
| | | | | 3.2.1.23) |
| Solyc12g039070 | 1.69 | 13.42 | 43.31 | Str_synth domain-containing |
| | | | | protein |
| Solyc04g078810 | 1.69 | 42.28 | 136.35 | DNA binding protein |
| Solyc01g010130 | 1.69 | 115.00 | 370.86 | BHLH domain-containing |
| | | | | protein |
| Solyc03g117730 | 1.69 | 10.19 | 32.87 | Tub domain-containing protein |
| Solyc04g005380 | 1.69 | 34.85 | 112.19 | Uncharacterized protein |
| Solyc02g077030 | 1.69 | 52.40 | 168.54 | Lipase_3 domain-containing |
| | | | | protein |
| Solyc05g012790 | 1.69 | 142.97 | 459.79 | S-acyltransferase (EC 2.3.1.225) |
| | | | | (Palmitoyltransferase) |
| Solyc08g066840 | 1.68 | 686.86 | 2,205.37 | Uncharacterized protein |
| Solyc06g066670 | 1.68 | 25.99 | 83.39 | DNA helicase (EC 3.6.4.12) |
| Solyc04g050050 | 1.68 | 54.58 | 175.11 | DUF4005 domain-containing |
| | | | | protein |
| Solyc07g049680 | 1.68 | 9.54 | 30.57 | Condensin complex subunit 2 |
| Solyc04g081755 | 1.68 | 135.34 | 433.83 | Uncharacterized protein |
| Solyc05g160400 | 1.68 | 8.78 | 28.11 | Avr9/Cf-9 rapidly elicited |
| | | | | protein 75 |
| Solyc04g078650 | 1.68 | 81.67 | 261.13 | WOX4 |
| Solyc01g102520 | 1.68 | 24.83 | 79.40 | Uncharacterized protein |
| Solyc01g098980 | 1.68 | 10.28 | 32.86 | Protein kinase domain- |
| | | | | containing protein |
| Solyc06g034390 | 1.68 | 16.72 | 53.45 | Uncharacterized protein |
| Solyc10g074440 | 1.68 | 89.56 | 286.22 | Chitin-binding type-1 domain- |
| | | | | containing protein |
| Solyc07g043480 | 1.68 | 134.13 | 428.55 | Uncharacterized protein |
| Solyc02g069630 | 1.67 | 86.58 | 276.41 | Uncharacterized protein |
| Solyc12g011170 | 1.67 | 8.41 | 26.84 | Phospholipase D (EC 3.1.4.4) |
| Solyc09g015520 | 1.67 | 137.48 | 438.61 | Protein kinase domain- |
| | | | | containing protein |
| Solyc03g120970 | 1.67 | 70.87 | 226.03 | Uncharacterized protein |
| Solyc12g056700 | 1.67 | 36.92 | 117.65 | Uncharacterized protein |
| Solyc03g098450 | 1.67 | 24.40 | 77.51 | NAB domain-containing protein |
| Solyc08g068160 | 1.67 | 16.09 | 51.10 | Flavin-containing |
| | | | | monooxygenase (EC 1.—.—.—) |
| Solyc09g091430 | 1.67 | 88.71 | 281.62 | Pectate lyase (EC 4.2.2.2) |
| Solyc01g099980 | 1.67 | 84.01 | 266.67 | Uncharacterized protein |
| Solyc02g080530 | 1.66 | 844.34 | 2,672.56 | Peroxidase (EC 1.11.1.7) |
| Solyc07g052240 | 1.66 | 12.81 | 40.55 | Uncharacterized protein |
| Solyc11g010120 | 1.66 | 261.76 | 827.75 | Peroxidase (EC 1.11.1.7) |
| Solyc08g006690 | 1.66 | 446.56 | 1,411.75 | PMEI domain-containing |
| | | | | protein |
| Solyc02g077260 | 1.66 | 11.74 | 37.12 | Uncharacterized protein |
| Solyc01g106910 | 1.66 | 31.94 | 100.92 | Uncharacterized protein |
| Solyc06g083370 | 1.66 | 9.13 | 28.82 | Uncharacterized protein |
| Solyc01g098400 | 1.66 | 54.47 | 171.86 | Histidine-containing |
| | | | | phosphotransfer protein |
| Solyc12g017970 | 1.66 | 10.30 | 32.49 | Uncharacterized protein |
| Solyc03g115380 | 1.66 | 76.03 | 239.74 | UDP-glucose 6-dehydrogenase |
| | | | | (EC 1.1.1.22) |
| Solyc08g076300 | 1.66 | 32.01 | 100.84 | Uncharacterized protein |
| Solyc08g065420 | 1.65 | 51.72 | 162.65 | Homeobox domain-containing |
| | | | | protein |
| Solyc08g016720 | 1.65 | 42.91 | 134.83 | 9-cis-epoxycarotenoid |
| | | | | dioxygenase NCED2, |
| | | | | chloroplastic (LeNCED2) |
| | | | | (SlNCED2) (EC 1.13.11.51) |
| | | | | (Nine-cis-epoxycarotenoid |
| | | | | dioxygenase 2) |
| Solyc03g083720 | 1.65 | 29.54 | 92.79 | PMEI domain-containing |
| | | | | protein |
| Solyc05g005580 | 1.65 | 9.44 | 29.63 | Uncharacterized protein |
| Solyc05g056450 | 1.65 | 13.53 | 42.41 | Peptidase A1 domain- |
| | | | | containing protein |
| Solyc07g006230 | 1.65 | 116.03 | 363.34 | NA |
| Solyc04g054500 | 1.65 | 147.29 | 460.93 | HMA domain-containing |
| | | | | protein |
| Solyc09g064750 | 1.65 | 26.61 | 83.28 | Uncharacterized protein |
| Solyc01g150165 | 1.65 | 19.60 | 61.34 | MAPK activating protein |
| Solyc04g077540 | 1.64 | 14.29 | 44.67 | Kinesin-like protein |
| Solyc06g074000 | 1.64 | 55.81 | 174.42 | Peptidase A1 domain- |
| | | | | containing protein |
| Solyc02g072010 | 1.64 | 15.00 | 46.87 | Heat shock factor |
| Solyc08g078410 | 1.64 | 8.07 | 25.21 | Dehydration-responsive |
| | | | | clement binding protein 6 |
| Solyc02g085400 | 1.64 | 42.53 | 132.63 | Uncharacterized protein |
| Solyc11g040340 | 1.64 | 45.92 | 142.99 | Endoglucanase (EC 3.2.1.4) |
| Solyc09g015080 | 1.64 | 146.04 | 454.78 | Sec14-like protein |
| Solyc11g066490 | 1.64 | 35.93 | 111.83 | LEA_2 domain-containing |
| | | | | protein |
| Solyc03g007130 | 1.64 | 14.61 | 45.47 | Uncharacterized protein |
| Solyc04g078470 | 1.64 | 45.31 | 140.98 | CycD3; 3 protein |
| Solyc12g008800 | 1.64 | 114.11 | 354.98 | MYB transcription factor |
| Solyc10g008210 | 1.64 | 9.53 | 29.61 | Uncharacterized protein |
| Solyc09g011070 | 1.64 | 11.33 | 35.19 | Clade XI lectin receptor kinase |
| Solyc08g006520 | 1.64 | 15.41 | 47.89 | Uncharacterized protein |
| Solyc01g091370 | 1.64 | 133.17 | 413.61 | AT-hook motif nuclear- |
| | | | | localized protein |
| Solyc10g076710 | 1.63 | 315.48 | 978.16 | Phosphoinositide phospholipase |
| | | | | C (EC 3.1.4.11) |
| Solyc01g005400 | 1.63 | 17.54 | 54.27 | Calcium ion binding protein |
| Solyc08g067240 | 1.62 | 125.53 | 386.91 | Putative BRCT domain- |
| | | | | containing DNA repair protein- |
| | | | | like |
| Solyc04g014470 | 1.62 | 15.65 | 48.22 | Uncharacterized protein |
| Solyc05g006700 | 1.62 | 15.79 | 48.63 | Uncharacterized protein |
| Solyc08g066450 | 1.62 | 50.08 | 154.24 | Uncharacterized protein |
| Solyc08g067300 | 1.62 | 45.34 | 139.58 | F-box domain-containing |
| | | | | protein |
| Solyc11g008080 | 1.62 | 14.66 | 45.01 | Uncharacterized protein |
| Solyc03g093330 | 1.62 | 107.86 | 330.55 | Protein kinase domain- |
| | | | | containing protein |
| Solyc02g072170 | 1.61 | 11.32 | 34.60 | Uncharacterized protein |
| Solyc04g081810 | 1.61 | 44.47 | 135.85 | Uncharacterized protein |
| Solyc11g010960 | 1.61 | 111.37 | 339.84 | Sinapyl alcohol dehydrogenase |
| | | | | 2 |
| Solyc12g049350 | 1.61 | 22.44 | 68.48 | Uncharacterized protein |
| Solyc11g008780 | 1.61 | 112.93 | 343.99 | Acetohydroxy-acid synthase |
| | | | | small subunit |
| Solyc07g053240 | 1.61 | 23.71 | 72.19 | Uncharacterized protein |
| Solyc01g067295 | 1.60 | 121.57 | 369.29 | CarbpepA_inh domain- |
| | | | | containing protein |
| Solyc03g006970 | 1.60 | 98.73 | 299.86 | SBT2 protein (Subtilisin-like |
| | | | | protease) |
| Solyc01g096220 | 1.60 | 57.33 | 174.10 | Uncharacterized protein |
| Solyc02g093930 | 1.60 | 33.21 | 100.86 | Rad21_Rec8_N domain- |
| | | | | containing protein |
| Solyc04g016060 | 1.60 | 10.56 | 32.07 | Uncharacterized protein |
| Solyc01g095610 | 1.60 | 35.91 | 108.80 | PMR5N domain-containing |
| | | | | protein |
| Solyc11g072840 | 1.60 | 18.25 | 55.28 | Histone H4 |
| Solyc10g079300 | 1.60 | 21.97 | 66.48 | Calcium-transporting ATPase |
| | | | | (EC 7.2.2.10) |
| Solyc06g074850 | 1.60 | 37.72 | 114.06 | Carboxypeptidase (EC 3.4.16.—) |
| Solyc05g009880 | 1.59 | 118.53 | 358.03 | BHLH domain-containing |
| | | | | protein |
| Solyc12g044920 | 1.59 | 46.19 | 139.20 | Phospholipid-transporting |
| | | | | ATPase (EC 7.6.2.1) |
| Solyc02g077430 | 1.59 | 115.71 | 348.52 | Lipase_3 domain-containing |
| | | | | protein |
| Solyc06g034150 | 1.59 | 25.55 | 76.86 | Reverse transcriptase domain- |
| | | | | containing protein |
| Solyc04g078520 | 1.59 | 45.54 | 136.67 | bHLH-MYC_N domain- |
| | | | | containing protein |
| Solyc03g113720 | 1.58 | 33.63 | 100.61 | Response regulatory domain- |
| | | | | containing protein |
| Solyc07g053540 | 1.58 | 10.63 | 31.78 | FAS1 domain-containing |
| | | | | protein |
| Solyc02g090770 | 1.58 | 10.46 | 31.20 | AP2/ERF domain-containing |
| | | | | protein |
| Solyc08g081060 | 1.58 | 90.51 | 270.01 | Uncharacterized protein |
| Solyc10g055540 | 1.58 | 29.29 | 87.33 | uncharacterized protein |
| | | | | LOC107821901 |
| Solyc09g007830 | 1.58 | 61.15 | 182.26 | Cytokinin riboside 5′- |
| | | | | monophosphate |
| | | | | phosphoribohydrolase (EC |
| | | | | 3.2.2.n1) |
| Solyc05g051870 | 1.58 | 160.04 | 476.84 | Uncharacterized protein |
| Solyc03g120420 | 1.57 | 10.99 | 32.69 | Uncharacterized protein |
| Solyc01g090710 | 1.57 | 37.32 | 110.99 | Malate dehydrogenase (EC |
| | | | | 1.1.1.37) |
| Solyc09g009970 | 1.57 | 51.20 | 152.11 | Uncharacterized protein |
| Solyc08g080270 | 1.57 | 26.30 | 78.14 | Uncharacterized protein |
| Solyc10g008780 | 1.57 | 11.67 | 34.64 | TCP domain-containing protein |
| Solyc06g084420 | 1.57 | 116.66 | 346.07 | URRNT_2 domain-containing |
| | | | | protein |
| Solyc07g062260 | 1.57 | 137.34 | 407.30 | BES1_N domain-containing |
| | | | | protein |
| Solyc04g008110 | 1.57 | 38.05 | 112.80 | Histidine kinase (EC 2.7.13.3) |
| Solyc04g008110 | 1.57 | 38.05 | 112.80 | Histidine kinase (EC 2.7.13.3) |
| Solyc03g026120 | 1.57 | 20.77 | 61.56 | Methyltransferase (EC 2.1.1.—) |
| Solyc03g026120 | 1.57 | 20.77 | 61.56 | Methyltransferase (EC 2.1.1.—) |
| Solyc02g069560 | 1.56 | 16.49 | 48.76 | Uncharacterized protein |
| Solyc02g069560 | 1.56 | 16.49 | 48.76 | Uncharacterized protein |
| Solyc02g071790 | 1.56 | 20.33 | 60.06 | Non-specific serine/threonine |
| | | | | protein kinase (EC 2.7.11.1) |
| Solyc02g071790 | 1.56 | 20.33 | 60.06 | Non-specific serine/threonine |
| | | | | protein kinase (EC 2.7.11.1) |
| Solyc02g090480 | 1.56 | 10.65 | 31.41 | Peptidylprolyl isomerase (EC |
| | | | | 5.2.1.8) |
| Solyc02g090480 | 1.56 | 10.65 | 31.41 | Peptidylprolyl isomerase (EC |
| | | | | 5.2.1.8) |
| Solyc02g071720 | 1.56 | 18.23 | 53.78 | Uncharacterized protein |
| Solyc02g071720 | 1.56 | 18.23 | 53.78 | Uncharacterized protein |
| Solyc10g005000 | 1.56 | 51.92 | 153.07 | DUF4005 domain-containing |
| | | | | protein |
| Solyc10g005000 | 1.56 | 51.92 | 153.07 | DUF4005 domain-containing |
| | | | | protein |
| Solyc06g066600 | 1.56 | 53.73 | 158.36 | Putative plastidic glucose |
| | | | | transporter 3-like |
| Solyc06g066600 | 1.56 | 53.73 | 158.36 | Putative plastidic glucose |
| | | | | transporter 3-like |
| Solyc03g093250 | 1.56 | 11.65 | 34.33 | Structural maintenance of |
| | | | | chromosomes protein |
| Solyc03g093250 | 1.56 | 11.65 | 34.33 | Structural maintenance of |
| | | | | chromosomes protein |
| Solyc03g114800 | 1.56 | 19.31 | 56.79 | Sulfotransferase (EC 2.8.2.—) |
| Solyc03g114800 | 1.56 | 19.31 | 56.79 | Sulfotransferase (EC 2.8.2.—) |
| Solyc07g056160 | 1.56 | 51.43 | 151.17 | Cytochrome P450 |
| Solyc07g056160 | 1.56 | 51.43 | 151.17 | Cytochrome P450 |
| Solyc12g100030 | 1.55 | 91.80 | 269.58 | LRRNT_2 domain-containing |
| | | | | protein |
| Solyc12g100030 | 1.55 | 91.80 | 269.58 | LRRNT_2 domain-containing |
| | | | | protein |
| Solyc05g009100 | 1.55 | 47.76 | 140.13 | Protein kinase domain- |
| | | | | containing protein |
| Solyc05g009100 | 1.55 | 47.76 | 140.13 | Protein kinase domain- |
| | | | | containing protein |
| Solyc05g010010 | 1.55 | 67.81 | 198.95 | Uncharacterized protein |
| Solyc05g010010 | 1.55 | 67.81 | 198.95 | Uncharacterized protein |
| Solyc09g160240 | 1.55 | 16.85 | 49.41 | Uncharacterized protein |
| Solyc09g160240 | 1.55 | 16.85 | 49.41 | Uncharacterized protein |
| Solyc12g014420 | 1.55 | 376.16 | 1,102.34 | Glucan endo-1,3-beta-D- |
| | | | | glucosidase (EC 3.2.1.39) |
| Solyc12g014420 | 1.55 | 376.16 | 1,102.34 | Glucan endo-1,3-beta-D- |
| | | | | glucosidase (EC 3.2.1.39) |
| Solyc03g115870 | 1.55 | 176.55 | 517.00 | Putative thioredoxin-like 1-2, |
| | | | | chloroplastic-like |
| Solyc03g115870 | 1.55 | 176.55 | 517.00 | Putative thioredoxin-like 1-2, |
| | | | | chloroplastic-like |
| Solyc04g007650 | 1.55 | 39.02 | 114.17 | 1-phosphatidylinositol-3- |
| | | | | phosphate 5-kinase (EC |
| | | | | 2.7.1.150) |
| Solyc04g007650 | 1.55 | 39.02 | 114.17 | 1-phosphatidylinositol-3- |
| | | | | phosphate 5-kinase (EC |
| | | | | 2.7.1.150) |
| Solyc01g088300 | 1.55 | 121.72 | 355.94 | Uncharacterized protein |
| Solyc01g088300 | 1.55 | 121.72 | 355.94 | Uncharacterized protein |
| Solyc09g064247 | 1.55 | 44.82 | 130.95 | uncharacterized protein |
| | | | | LOC107869380 |
| Solyc09g064247 | 1.55 | 44.82 | 130.95 | uncharacterized protein |
| | | | | LOC107869380 |
| Solyc09g098050 | 1.55 | 10.99 | 32.10 | uncharacterized protein |
| | | | | LOC107865686 |
| Solyc09g098050 | 1.55 | 10.99 | 32.10 | uncharacterized protein |
| | | | | LOC107865686 |
| Solyc02g069040 | 1.55 | 23.41 | 68.35 | DUF4220 domain-containing |
| | | | | protein |
| Solyc02g069040 | 1.55 | 23.41 | 68.35 | DUF4220 domain-containing |
| | | | | protein |
| Solyc04g080750 | 1.54 | 27.38 | 79.86 | uncharacterized protein |
| | | | | LOC107800378 isoform X1 |
| Solyc04g080750 | 1.54 | 27.38 | 79.86 | uncharacterized protein |
| | | | | LOC107800378 isoform X1 |
| Solyc10g080550 | 1.54 | 19.72 | 57.52 | PMR5N domain-containing |
| | | | | protein |
| Solyc10g080550 | 1.54 | 19.72 | 57.52 | PMR5N domain-containing |
| | | | | protein |
| Solyc09g011750 | 1.54 | 94.29 | 274.94 | Receptor protein kinase |
| Solyc09g011750 | 1.54 | 94.29 | 274.94 | Receptor protein kinase |
| Solyc08g066490 | 1.54 | 194.10 | 565.36 | Protein kinase domain- |
| | | | | containing protein |
| Solyc08g066490 | 1.54 | 194.10 | 565.36 | Protein kinase domain- |
| | | | | containing protein |
| Solyc01g107890 | 1.54 | 43.04 | 125.20 | Uncharacterized protein |
| Solyc01g107890 | 1.54 | 43.04 | 125.20 | Uncharacterized protein |
| Solyc12g010020 | 1.54 | 89.73 | 260.89 | CYTOSOL_AP domain- |
| | | | | containing protein |
| Solyc12g010020 | 1.54 | 89.73 | 260.89 | CYTOSOL_AP domain- |
| | | | | containing protein |
| Solyc07g043460 | 1.54 | 332.46 | 966.41 | Uncharacterized protein |
| Solyc07g043460 | 1.54 | 332.46 | 966.41 | Uncharacterized protein |
| Solyc07g009500 | 1.54 | 10.33 | 29.96 | Uncharacterized protein |
| Solyc07g009500 | 1.54 | 10.33 | 29.96 | Uncharacterized protein |
| Solyc04g076100 | 1.53 | 42.30 | 122.43 | Glucose-6-phosphate isomerase |
| | | | | (EC 5.3.1.9) |
| Solyc04g076100 | 1.53 | 42.30 | 122.43 | Glucose-6-phosphate isomerase |
| | | | | (EC 5.3.1.9) |
| Solyc07g052340 | 1.53 | 48.85 | 141.19 | Putative exosome component |
| | | | | 10-like |
| Solyc07g052340 | 1.53 | 48.85 | 141.19 | Putative exosome component |
| | | | | 10-like |
| Solyc12g014500 | 1.53 | 16.14 | 46.59 | Uncharacterized protein |
| Solyc12g014500 | 1.53 | 16.14 | 46.59 | Uncharacterized protein |
| Solyc03g083510 | 1.53 | 26.23 | 75.69 | Protein kinase domain- |
| | | | | containing protein |
| Solyc03g083510 | 1.53 | 26.23 | 75.69 | Protein kinase domain- |
| | | | | containing protein |
| Solyc09g010430 | 1.53 | 16.36 | 47.19 | Uncharacterized protein |
| Solyc09g010430 | 1.53 | 16.36 | 47.19 | Uncharacterized protein |
| Solyc02g091920 | 1.53 | 144.76 | 417.18 | Xyloglucan |
| | | | | endotransglucosylase/hydrolase |
| | | | | (EC 2.4.1.207) |
| Solyc02g091920 | 1.53 | 144.76 | 417.18 | Xyloglucan |
| | | | | endotransglucosylase/hydrolase |
| | | | | (EC 2.4.1.207) |
| Solyc03g026360 | 1.53 | 30.69 | 88.39 | LysM domain-containing |
| | | | | protein |
| Solyc03g026360 | 1.53 | 30.69 | 88.39 | LysM domain-containing |
| | | | | protein |
| Solyc11g011370 | 1.52 | 19.43 | 55.90 | Nudix hydrolase domain- |
| | | | | containing protein |
| Solyc11g011370 | 1.52 | 19.43 | 55.90 | Nudix hydrolase domain- |
| | | | | containing protein |
| Solyc04g008880 | 1.52 | 22.25 | 63.90 | Uncharacterized protein |
| Solyc04g008880 | 1.52 | 22.25 | 63.90 | Uncharacterized protein |
| Solyc09g072800 | 1.52 | 21.60 | 61.97 | Uncharacterized protein |
| Solyc09g072800 | 1.52 | 21.60 | 61.97 | Uncharacterized protein |
| Solyc03g025760 | 1.52 | 33.58 | 96.29 | Uncharacterized protein |
| Solyc03g025760 | 1.52 | 33.58 | 96.29 | Uncharacterized protein |
| Solyc01g107800 | 1.52 | 75.86 | 217.27 | Polysacc_synt_4 domain- |
| | | | | containing protein |
| Solyc01g107800 | 1.52 | 75.86 | 217.27 | Polysacc_synt_4 domain- |
| | | | | containing protein |
| Solyc02g077400 | 1.52 | 14.97 | 42.85 | Ras-related C3 botulinum toxin |
| | | | | substrate 1 |
| Solyc02g077400 | 1.52 | 14.97 | 42.85 | Ras-related C3 botulinum toxin |
| | | | | substrate 1 |
| Solyc10g047240 | 1.52 | 21.88 | 62.62 | Annexin |
| Solyc10g047240 | 1.52 | 21.88 | 62.62 | Annexin |
| Solyc06g069300 | 1.52 | 26.39 | 75.48 | Uncharacterized protein |
| Solyc06g069300 | 1.52 | 26.39 | 75.48 | Uncharacterized protein |
| Solyc01g087500 | 1.51 | 45.84 | 131.01 | DNA topoisomerase 2 (EC |
| | | | | 5.6.22) |
| Solyc01g087500 | 1.51 | 45.84 | 131.01 | DNA topoisomerase 2 (EC |
| | | | | 5.6.22) |
| Solyc02g080610 | 1.51 | 25.56 | 73.02 | ANK_REP_REGION domain- |
| | | | | containing protein |
| Solyc02g080610 | 1.51 | 25.56 | 73.02 | ANK_REP_REGION domain- |
| | | | | containing protein |
| Solyc02g092820 | 1.51 | 32.02 | 91.31 | Putative indole-3-acetic acid |
| | | | | amido synthetase |
| Solyc02g092820 | 1.51 | 32.02 | 91.31 | Putative indole-3-acetic acid |
| | | | | amido synthetase |
| Solyc04g080550 | 1.51 | 29.33 | 83.57 | NmrA domain-containing |
| | | | | protein |
| Solyc04g080550 | 1.51 | 29.33 | 83.57 | NmrA domain-containing |
| | | | | protein |
| Solyc04g077190 | 1.51 | 19.76 | 56.29 | Xylanase Xyn2 |
| Solyc04g077190 | 1.51 | 19.76 | 56.29 | Xylanase Xyn2 |
| Solyc03g005900 | 1.51 | 141.84 | 403.53 | GDSL-lipase protein |
| Solyc03g005900 | 1.51 | 141.84 | 403.53 | GDSL-lipase protein |
| Solyc05g054040 | 1.51 | 17.82 | 50.68 | Transmembrane 9 superfamily |
| | | | | member |
| Solyc05g054040 | 1.51 | 17.82 | 50.68 | Transmembrane 9 superfamily |
| | | | | member |
| Solyc06g008580 | 1.51 | 17.89 | 50.88 | Auxin-responsive protein |
| Solyc06g008580 | 1.51 | 17.89 | 50.88 | Auxin-responsive protein |
| Solyc11g066270 | 1.51 | 131.71 | 374.52 | Xyloglucan |
| | | | | endotransglucosylase/hydrolase |
| | | | | (EC 2.4.1.207) |
| Solyc11g066270 | 1.51 | 131.71 | 374.52 | Xyloglucan |
| | | | | endotransglucosylase/hydrolase |
| | | | | (EC 2.4.1.207) |
| Solyc03g025860 | 1.51 | 12.11 | 34.39 | Longin domain-containing |
| | | | | protein |
| Solyc03g025860 | 1.51 | 12.11 | 34.39 | Longin domain-containing |
| | | | | protein |
| Solyc09g011770 | 1.51 | 80.35 | 228.11 | RING-CH-type domain- |
| | | | | containing protein |
| Solyc09g011770 | 1.51 | 80.35 | 228.11 | RING-CH-type domain- |
| | | | | containing protein |
| Solyc10g039290 | 1.50 | 335.15 | 951.10 | Uncharacterized protein |
| Solyc10g039290 | 1.50 | 335.15 | 951.10 | Uncharacterized protein |
| Solyc03g112840 | 1.50 | 31.11 | 88.21 | Uncharacterized protein |
| Solyc03g112840 | 1.50 | 31.11 | 88.21 | Uncharacterized protein |
| Solyc03g043770 | 1.50 | 88.77 | 251.52 | Protein kinase domain- |
| | | | | containing protein |
| Solyc03g043770 | 1.50 | 88.77 | 251.52 | Protein kinase domain- |
| | | | | containing protein |
| Solyc02g070540 | 1.50 | 137.82 | 390.06 | Uncharacterized protein |
| Solyc02g070540 | 1.50 | 137.82 | 390.06 | Uncharacterized protein |
| Solyc07g045440 | 1.50 | 208.40 | 589.31 | Uncharacterized protein |
| Solyc07g045440 | 1.50 | 208.40 | 589.31 | Uncharacterized protein |
| Solyc11g032090 | 1.50 | 25.97 | 73.42 | Uncharacterized protein |
| Solyc11g032090 | 1.50 | 25.97 | 73.42 | Uncharacterized protein |
| Solyc09g074930 | 1.50 | 97.44 | 275.38 | Uncharacterized protein |
| Solyc09g074930 | 1.50 | 97.44 | 275.38 | Uncharacterized protein |
| Solyc03g115200 | 1.50 | 122.69 | 346.12 | X8 domain-containing protein |
| Solyc03g115200 | 1.50 | 122.69 | 346.12 | X8 domain-containing protein |
| Solyc09g064520 | 1.49 | 19.30 | 54.35 | Protein kinase domain- |
| | | | | containing protein |
| Solyc09g064520 | 1.49 | 19.30 | 54.35 | Protein kinase domain- |
| | | | | containing protein |
| Solyc10g047110 | 1.49 | 17.20 | 48.41 | Peroxidase (EC 1.11.1.7) |
| Solyc10g047110 | 1.49 | 17.20 | 48.41 | Peroxidase (EC 1.11.1.7) |
| Solyc08g007830 | 1.49 | 61.78 | 173.63 | AP2/ERF domain-containing |
| | | | | protein |
| Solyc08g007830 | 1.49 | 61.78 | 173.63 | AP2/ERF domain-containing |
| | | | | protein |
| Solyc03g006990 | 1.49 | 17.22 | 48.25 | Uncharacterized protein |
| Solyc03g006990 | 1.49 | 17.22 | 48.25 | Uncharacterized protein |
| Solyc07g062040 | 1.49 | 41.86 | 117.23 | Protein kinase domain- |
| | | | | containing protein |
| Solyc07g062040 | 1.49 | 41.86 | 117.23 | Protein kinase domain- |
| | | | | containing protein |
| Solyc06g069380 | 1.48 | 149.82 | 418.36 | O-fucosyltransferase family |
| | | | | protein |
| Solyc06g069380 | 1.48 | 149.82 | 418.36 | O-fucosyltransferase family |
| | | | | protein |
| Solyc01g107520 | 1.48 | 64.81 | 180.90 | Uncharacterized protein |
| Solyc01g107520 | 1.48 | 64.81 | 180.90 | Uncharacterized protein |
| Solyc01g096450 | 1.48 | 166.41 | 464.37 | Peptidase A1 domain- |
| | | | | containing protein |
| Solyc01g096450 | 1.48 | 166.41 | 464.37 | Peptidase A1 domain- |
| | | | | containing protein |
| Solyc09g008270 | 1.48 | 55.04 | 153.33 | Uncharacterized protein |
| Solyc09g008270 | 1.48 | 55.04 | 153.33 | Uncharacterized protein |
| Solyc08g014280 | 1.48 | 185.91 | 517.87 | DUF4005 domain-containing |
| | | | | protein |
| Solyc08g014280 | 1.48 | 185.91 | 517.87 | DUF4005 domain-containing |
| | | | | protein |
| Solyc05g012870 | 1.48 | 14.24 | 39.67 | Uncharacterized protein |
| Solyc05g012870 | 1.48 | 14.24 | 39.67 | Uncharacterized protein |
| Solyc02g082050 | 1.48 | 42.28 | 117.74 | ADP-ribosyl cyclase/cyclic |
| | | | | ADP-ribose hydrolase (EC |
| | | | | 3.2.2.6) |
| Solyc02g082050 | 1.48 | 42.28 | 117.74 | ADP-ribosyl cyclase/cyclic |
| | | | | ADP-ribose hydrolase (EC |
| | | | | 3.2.2.6) |
| Solyc05g032680 | 1.48 | 14.91 | 41.46 | Putative 3-hydroxyisobutyryl- |
| | | | | CoA hydrolase 3-like |
| Solyc05g032680 | 1.48 | 14.91 | 41.46 | Putative 3-hydroxyisobutyryl - |
| | | | | CoA hydrolase 3-like |
| Solyc09g008760 | 1.47 | 13.53 | 37.59 | VQ domain-containing protein |
| Solyc09g008760 | 1.47 | 13.53 | 37.59 | VQ domain-containing protein |
| Solyc12g049400 | 1.47 | 42.89 | 119.07 | Tify domain-containing protein |
| Solyc12g049400 | 1.47 | 42.89 | 119.07 | Tify domain-containing protein |
| Solyc02g083120 | 1.47 | 38.44 | 106.71 | Uncharacterized protein |
| Solyc02g083120 | 1.47 | 38.44 | 106.71 | Uncharacterized protein |
| Solyc09g015170 | 1.47 | 49.83 | 138.30 | Systemin receptor SR160 |
| Solyc09g015170 | 1.47 | 49.83 | 138.30 | Systemin receptor SR160 |
| Solyc03g071570 | 1.47 | 25.25 | 69.98 | Pectate lyase (EC 4.2.2.2) |
| Solyc03g071570 | 1.47 | 25.25 | 69.98 | Pectate lyase (EC 4.2.2.2) |
| Solyc07g065210 | 1.47 | 31.47 | 87.23 | Kinesin motor domain- |
| | | | | containing protein |
| Solyc07g065210 | 1.47 | 31.47 | 87.23 | Kinesin motor domain- |
| | | | | containing protein |
| Solyc02g082410 | 1.47 | 24.09 | 66.66 | Sugar transporter |
| Solyc02g082410 | 1.47 | 24.09 | 66.66 | Sugar transporter |
| Solyc07g005770 | 1.47 | 16.44 | 45.48 | Disease resistance protein BS2 |
| Solyc07g005770 | 1.47 | 16.44 | 45.48 | Disease resistance protein BS2 |
| Solyc06g065680 | 1.47 | 29.32 | 81.07 | Cyclin N-terminal domain- |
| | | | | containing protein |
| Solyc06g065680 | 1.47 | 29.32 | 81.07 | Cyclin N-terminal domain- |
| | | | | containing protein |
| Solyc07g049440 | 1.47 | 326.14 | 900.55 | Lipase_GDSL domain- |
| | | | | containing protein |
| Solyc07g049440 | 1.47 | 326.14 | 900.55 | Lipase_GDSL domain- |
| | | | | containing protein |
| Solyc10g005210 | 1.46 | 32.02 | 88.39 | Uncharacterized protein |
| Solyc10g005210 | 1.46 | 32.02 | 88.39 | Uncharacterized protein |
| Solyc07g054580 | 1.46 | 15.00 | 41.38 | Uncharacterized protein |
| Solyc07g054580 | 1.46 | 15.00 | 41.38 | Uncharacterized protein |
| Solyc02g078590 | 1.46 | 78.67 | 216.99 | Gb3_synth domain-containing |
| | | | | protein |
| Solyc02g078590 | 1.46 | 78.67 | 216.99 | Gb3_synth domain-containing |
| | | | | protein |
| Solyc02g092590 | 1.46 | 237.95 | 656.32 | Uncharacterized protein |
| Solyc02g092590 | 1.46 | 237.95 | 656.32 | Uncharacterized protein |
| Solyc07g042990 | 1.46 | 43.99 | 121.25 | Leucine-rich repeat family |
| | | | | protein |
| Solyc07g042990 | 1.46 | 43.99 | 121.25 | Leucine-rich repeat family |
| | | | | protein |
| Solyc01g096170 | 1.46 | 28.15 | 77.30 | Protein kinase domain- |
| | | | | containing protein |
| Solyc01g096170 | 1.46 | 28.15 | 77.30 | Protein kinase domain- |
| | | | | containing protein |
| Solyc08g065430 | 1.46 | 268.24 | 736.33 | Uncharacterized protein |
| Solyc08g065430 | 1.46 | 268.24 | 736.33 | Uncharacterized protein |
| Solyc01g098740 | 1.45 | 19.44 | 53.29 | Protein kinase domain- |
| | | | | containing protein |
| Solyc01g098740 | 1.45 | 19.44 | 53.29 | Protein kinase domain- |
| | | | | containing protein |
| Solyc01g006100 | 1.45 | 20.50 | 56.14 | Uncharacterized protein |
| Solyc01g006100 | 1.45 | 20.50 | 56.14 | Uncharacterized protein |
| Solyc10g078700 | 1.45 | 19.76 | 53.97 | Promoter-binding protein SPL9 |
| Solyc10g078700 | 1.45 | 19.76 | 53.97 | Promoter-binding protein SPL9 |
| Solyc02g084410 | 1.45 | 161.75 | 441.86 | VOC domain-containing protein |
| Solyc02g084410 | 1.45 | 161.75 | 441.86 | VOC domain-containing protein |
| Solyc01g094610 | 1.45 | 17.90 | 48.76 | Med12 domain-containing |
| | | | | protein |
| Solyc01g094610 | 1.45 | 17.90 | 48.76 | Med12 domain-containing |
| | | | | protein |
| Solyc05g050830 | 1.45 | 23.11 | 62.92 | AP2/ERF domain-containing |
| | | | | transcription factor |
| Solyc05g050830 | 1.45 | 23.11 | 62.92 | AP2/ERF domain-containing |
| | | | | transcription factor |
| Solyc01g058170 | 1.44 | 16.38 | 44.55 | Uncharacterized protein |
| Solyc01g058170 | 1.44 | 16.38 | 44.55 | Uncharacterized protein |
| Solyc02g090310 | 1.44 | 49.46 | 134.45 | Dof-type domain-containing |
| | | | | protein |
| Solyc02g090310 | 1.44 | 49.46 | 134.45 | Dof-type domain-containing |
| | | | | protein |
| Solyc08g082070 | 1.44 | 52.01 | 141.29 | Uncharacterized protein |
| Solyc08g082070 | 1.44 | 52.01 | 141.29 | Uncharacterized protein |
| Solyc05g009660 | 1.44 | 77.31 | 209.87 | Putative transcription factor |
| | | | | PERIANTHIA-like |
| Solyc05g009660 | 1.44 | 77.31 | 209.87 | Putative transcription factor |
| | | | | PERIANTHIA-like |
| Solyc03g034010 | 1.44 | 45.40 | 123.24 | Putative U-box domain- |
| | | | | containing protein 5-like |
| Solyc03g034010 | 1.44 | 45.40 | 123.24 | Putative U-box domain- |
| | | | | containing protein 5-like |
| Solyc03g112620 | 1.44 | 150.21 | 407.40 | Uncharacterized protein |
| Solyc03g112620 | 1.44 | 150.21 | 407.40 | Uncharacterized protein |
| Solyc08g080730 | 1.44 | 39.04 | 105.80 | Uncharacterized protein |
| Solyc08g080730 | 1.44 | 39.04 | 105.80 | Uncharacterized protein |
| Solyc07g018070 | 1.44 | 212.57 | 576.10 | Uncharacterized protein |
| Solyc07g018070 | 1.44 | 212.57 | 576.10 | Uncharacterized protein |
| Solyc01g111840 | 1.44 | 69.65 | 188.64 | Uncharacterized protein |
| Solyc01g111840 | 1.44 | 69.65 | 188.64 | Uncharacterized protein |
| Solyc05g005865 | 1.44 | 18.57 | 50.24 | Bet_v_1 domain-containing |
| | | | | protein |
| Solyc05g005865 | 1.44 | 18.57 | 50.24 | Bet_v_1 domain-containing |
| | | | | protein |
| Solyc03g093800 | 1.44 | 1,115.09 | 3,017.25 | Uncharacterized protein |
| Solyc03g093800 | 1.44 | 1,115.09 | 3,017.25 | Uncharacterized protein |
| Solyc03g097750 | 1.44 | 26.30 | 71.15 | Uncharacterized protein |
| Solyc03g097750 | 1.44 | 26.30 | 71.15 | Uncharacterized protein |
| Solyc08g021820 | 1.44 | 170.64 | 461.45 | Auxin-responsive protein |
| Solyc08g021820 | 1.44 | 170.64 | 461.45 | Auxin-responsive protein |
| Solyc02g091470 | 1.43 | 38.69 | 104.50 | Protein kinase domain- |
| | | | | containing protein |
| Solyc02g091470 | 1.43 | 38.69 | 104.50 | Protein kinase domain- |
| | | | | containing protein |
| Solyc05g052670 | 1.43 | 188.82 | 509.62 | Uncharacterized protein |
| Solyc05g052670 | 1.43 | 188.82 | 509.62 | Uncharacterized protein |
| Solyc10g084290 | 1.43 | 15.73 | 42.42 | Uncharacterized protein |
| Solyc10g084290 | 1.43 | 15.73 | 42.42 | Uncharacterized protein |
| Solyc07g064970 | 1.43 | 171.52 | 462.46 | Uncharacterized protein |
| Solyc07g064970 | 1.43 | 171.52 | 462.46 | Uncharacterized protein |
| Solyc11g007930 | 1.43 | 17.57 | 47.34 | Histone H2B |
| Solyc11g007930 | 1.43 | 17.57 | 47.34 | Histone H2B |
| Solyc08g080780 | 1.43 | 363.61 | 979.49 | C2 NT-type domain-containing |
| | | | | protein |
| Solyc08g080780 | 1.43 | 363.61 | 979.49 | C2 NT-type domain-containing |
| | | | | protein |
| Solyc04g008820 | 1.43 | 27.41 | 73.82 | HMG box domain-containing |
| | | | | protein |
| Solyc04g008820 | 1.43 | 27.41 | 73.82 | HMG box domain-containing |
| | | | | protein |
| Solyc09g014160 | 1.43 | 85.14 | 228.69 | Uncharacterized protein |
| Solyc09g014160 | 1.43 | 85.14 | 228.69 | Uncharacterized protein |
| Solyc07g045010 | 1.42 | 29.43 | 78.99 | Uncharacterized protein |
| Solyc07g045010 | 1.42 | 29.43 | 78.99 | Uncharacterized protein |
| Solyc12g013570 | 1.42 | 15.35 | 41.20 | Uncharacterized protein |
| Solyc12g013570 | 1.42 | 15.35 | 41.20 | Uncharacterized protein |
| Solyc02g087980 | 1.42 | 36.43 | 97.71 | Structural maintenance of |
| | | | | chromosomes protein |
| Solyc02g087980 | 1.42 | 36.43 | 97.71 | Structural maintenance of |
| | | | | chromosomes protein |
| Solyc04g016590 | 1.42 | 42.00 | 112.45 | Putative ovule protein |
| Solyc04g016590 | 1.42 | 42.00 | 112.45 | Putative ovule protein |
| Solyc06g062830 | 1.42 | 20.11 | 53.82 | Uncharacterized protein |
| Solyc06g062830 | 1.42 | 20.11 | 53.82 | Uncharacterized protein |
| Solyc02g080900 | 1.42 | 40.98 | 109.61 | GUB_WAK_bind domain- |
| | | | | containing protein |
| Solyc02g080900 | 1.42 | 40.98 | 109.61 | GUB_WAK_bind domain- |
| | | | | containing protein |
| Solyc01g079750 | 1.42 | 23.72 | 63.45 | Protein kinase domain- |
| | | | | containing protein |
| Solyc01g079750 | 1.42 | 23.72 | 63.45 | Protein kinase domain- |
| | | | | containing protein |
| Solyc06g068770 | 1.42 | 69.39 | 185.43 | Putative ovule protein |
| Solyc06g068770 | 1.42 | 69.39 | 185.43 | Putative ovule protein |
| Solyc02g082930 | 1.42 | 675.58 | 1,804.69 | Acidic 27 kDa endochitinase |
| | | | | (EC 3.2.1.14) |
| Solyc02g082930 | 1.42 | 675.58 | 1,804.69 | Acidic 27 kDa endochitinase |
| | | | | (EC 3.2.1.14) |
| Solyc12g099260 | 1.42 | 94.21 | 251.55 | ATP citrate synthase (EC |
| | | | | 2.3.3.8) |
| Solyc12g099260 | 1.42 | 94.21 | 251.55 | ATP citrate synthase (EC |
| | | | | 2.3.3.8) |
| Solyc04g016480 | 1.42 | 22.72 | 60.67 | Uncharacterized protein |
| Solyc04g016480 | 1.42 | 22.72 | 60.67 | Uncharacterized protein |
| Solyc04g078000 | 1.42 | 62.90 | 167.92 | Uncharacterized protein |
| Solyc04g078000 | 1.42 | 62.90 | 167.92 | Uncharacterized protein |
| Solyc12g009800 | 1.42 | 62.43 | 166.57 | Purple acid phosphatase (EC |
| | | | | 3.1.3.2) |
| Solyc12g009800 | 1.42 | 62.43 | 166.57 | Purple acid phosphatase (EC |
| | | | | 3.1.3.2) |
| Solyc09g007340 | 1.41 | 54.44 | 145.08 | Uncharacterized protein |
| Solyc09g007340 | 1.41 | 54.44 | 145.08 | Uncharacterized protein |
| Solyc03g032190 | 1.41 | 19.00 | 50.62 | Putative G2/mitotic-specific |
| | | | | cyclin-1 |
| Solyc03g032190 | 1.41 | 19.00 | 50.62 | Putative G2/mitotic-specific |
| | | | | cyclin-1 |
| Solyc04g080940 | 1.41 | 790.89 | 2,105.35 | WAT1-related protein |
| Solyc04g080940 | 1.41 | 790.89 | 2,105.35 | WAT1-related protein |
| Solyc01g105800 | 1.41 | 423.66 | 1,126.85 | Mads box protein |
| Solyc01g105800 | 1.41 | 423.66 | 1,126.85 | Mads box protein |
| Solyc02g030480 | 1.41 | 74.25 | 197.30 | PKS_ER domain-containing |
| | | | | protein |
| Solyc02g030480 | 1.41 | 74.25 | 197.30 | PKS_ER domain-containing |
| | | | | protein |
| Solyc01g090760 | 1.41 | 22.29 | 59.23 | GATA transcription factor |
| Solyc01g090760 | 1.41 | 22.29 | 59.23 | GATA transcription factor |
| Solyc04g008370 | 1.41 | 22.27 | 59.09 | Uncharacterized protein |
| Solyc04g008370 | 1.41 | 22.27 | 59.09 | Uncharacterized protein |
| Solyc09g082370 | 1.41 | 113.52 | 301.18 | IPPc domain-containing protein |
| Solyc09g082370 | 1.41 | 113.52 | 301.18 | IPPc domain-containing protein |
| Solyc10g045380 | 1.41 | 86.21 | 228.71 | Uncharacterized protein |
| Solyc10g045380 | 1.41 | 86.21 | 228.71 | Uncharacterized protein |
| Solyc05g015490 | 1.41 | 439.75 | 1,166.15 | AAI domain-containing protein |
| Solyc05g015490 | 1.41 | 439.75 | 1,166.15 | AAI domain-containing protein |
| Solyc06g074110 | 1.40 | 232.24 | 614.84 | BHLH domain-containing |
| | | | | protein |
| Solyc06g074110 | 1.40 | 232.24 | 614.84 | BHLH domain-containing |
| | | | | protein |
| Solyc10g086660 | 1.40 | 197.81 | 522.71 | PRONE domain-containing |
| | | | | protein |
| Solyc10g086660 | 1.40 | 197.81 | 522.71 | PRONE domain-containing |
| | | | | protein |
| Solyc09g083400 | 1.40 | 90.93 | 240.28 | TPX2 domain-containing |
| | | | | protein |
| Solyc09g083400 | 1.40 | 90.93 | 240.28 | TPX2 domain-containing |
| | | | | protein |
| Solyc06g082000 | 1.40 | 17.96 | 47.45 | Phosphoinositide phospholipase |
| | | | | C (EC 3.1.4.11) |
| Solyc06g082000 | 1.40 | 17.96 | 47.45 | Phosphoinositide phospholipase |
| | | | | C (EC 3.1.4.11) |
| Solyc08g075870 | 1.40 | 492.68 | 1,301.49 | Methyltransferase (EC 2.1.1.—) |
| Solyc08g075870 | 1.40 | 492.68 | 1,301.49 | Methyltransferase (EC 2.1.1.—) |
| Solyc06g053830 | 1.40 | 161.12 | 425.32 | Auxin-responsive protein |
| Solyc06g053830 | 1.40 | 161.12 | 425.32 | Auxin-responsive protein |
| Solyc09g065760 | 1.40 | 187.42 | 494.53 | Uncharacterized protein |
| Solyc09g065760 | 1.40 | 187.42 | 494.53 | Uncharacterized protein |
| Solyc03g114390 | 1.40 | 52.65 | 138.92 | Uncharacterized protein |
| Solyc03g114390 | 1.40 | 52.65 | 138.92 | Uncharacterized protein |
| Solyc05g026240 | 1.40 | 63.89 | 168.44 | Putative leucine-rich repeat |
| | | | | extensin-like protein 4-like |
| Solyc05g026240 | 1.40 | 63.89 | 168.44 | Putative leucine-rich repeat |
| | | | | extensin-like protein 4-like |
| Solyc12g013650 | 1.40 | 143.31 | 377.70 | Uncharacterized protein |
| Solyc12g013650 | 1.40 | 143.31 | 377.70 | Uncharacterized protein |
| Solyc05g023670 | 1.40 | 26.26 | 69.10 | YDG domain-containing |
| | | | | protein |
| Solyc05g023670 | 1.40 | 26.26 | 69.10 | YDG domain-containing |
| | | | | protein |
| Solyc04g064660 | 1.40 | 18.24 | 47.98 | Uncharacterized protein |
| Solyc04g064660 | 1.40 | 18.24 | 47.98 | Uncharacterized protein |
| Solyc04g072570 | 1.39 | 506.58 | 1,331.61 | Non-specific serine/threonine |
| | | | | protein kinase (EC 2.7.11.1) |
| Solyc04g072570 | 1.39 | 506.58 | 1,331.61 | Non-specific serine/threonine |
| | | | | protein kinase (EC 2.7.11.1) |
| Solyc11g022590 | 1.39 | 231.04 | 606.35 | Uncharacterized protein |
| Solyc11g022590 | 1.39 | 231.04 | 606.35 | Uncharacterized protein |
| Solyc02g092970 | 1.39 | 314.60 | 825.64 | Uncharacterized protein |
| Solyc02g092970 | 1.39 | 314.60 | 825.64 | Uncharacterized protein |
| Solyc06g035720 | 1.39 | 77.07 | 202.01 | Ubiquitin-like domain- |
| | | | | containing protein |
| Solyc06g035720 | 1.39 | 77.07 | 202.01 | Ubiquitin-like domain- |
| | | | | containing protein |
| Solyc08g081940 | 1.39 | 150.83 | 394.68 | Protein kinase domain- |
| | | | | containing protein |
| Solyc08g081940 | 1.39 | 150.83 | 394.68 | Protein kinase domain- |
| | | | | containing protein |
| Solyc04g055230 | 1.39 | 21.92 | 57.36 | Uncharacterized protein |
| Solyc04g055230 | 1.39 | 21.92 | 57.36 | Uncharacterized protein |
| Solyc03g071750 | 1.39 | 54.08 | 141.45 | Non-specific serine/threonine |
| | | | | protein kinase (EC 2.7.11.1) |
| Solyc03g071750 | 1.39 | 54.08 | 141.45 | Non-specific serine/threonine |
| | | | | protein kinase (EC 2.7.11.1) |
| Solyc04g015470 | 1.38 | 153.39 | 399.20 | Phosphatidylinositol 4- |
| | | | | phosphate 5-kinase (EC |
| | | | | 2.7.1.68) |
| Solyc04g015470 | 1.38 | 153.39 | 399.20 | Phosphatidylinositol 4- |
| | | | | phosphate 5-kinase (EC |
| | | | | 2.7.1.68) |
| Solyc12g010480 | 1.38 | 19.31 | 50.24 | Uncharacterized protein |
| Solyc12g010480 | 1.38 | 19.31 | 50.24 | Uncharacterized protein |
| Solyc01g057310 | 1.38 | 26.25 | 68.30 | Uncharacterized protein |
| Solyc01g057310 | 1.38 | 26.25 | 68.30 | Uncharacterized protein |
| Solyc10g078380 | 1.38 | 25.97 | 67.45 | BHLH domain-containing |
| | | | | protein |
| Solyc10g078380 | 1.38 | 25.97 | 67.45 | BHLH domain-containing |
| | | | | protein |
| Solyc06g084430 | 1.37 | 56.16 | 145.64 | Histone H2A |
| Solyc06g084430 | 1.37 | 56.16 | 145.64 | Histone H2A |
| Solyc01g108670 | 1.37 | 47.88 | 123.97 | Kinesin motor domain- |
| | | | | containing protein |
| Solyc01g108670 | 1.37 | 47.88 | 123.97 | Kinesin motor domain- |
| | | | | containing protein |
| Solyc10g081300 | 1.37 | 28.34 | 73.34 | Uncharacterized protein |
| Solyc10g081300 | 1.37 | 28.34 | 73.34 | Uncharacterized protein |
| Solyc10g086640 | 1.37 | 24.22 | 62.69 | Homeobox domain-containing |
| | | | | protein |
| Solyc10g086640 | 1.37 | 24.22 | 62.69 | Homeobox domain-containing |
| | | | | protein |
| Solyc07g008570 | 1.37 | 64.41 | 166.56 | Purple acid phosphatase (EC |
| | | | | 3.1.3.2) |
| Solyc07g008570 | 1.37 | 64.41 | 166.56 | Purple acid phosphatase (EC |
| | | | | 3.1.3.2) |
| Solyc06g059730 | 1.37 | 21.17 | 54.74 | Auxin efflux carrier component |
| Solyc06g059730 | 1.37 | 21.17 | 54.74 | Auxin efflux carrier component |
| Solyc03g082840 | 1.37 | 35.39 | 91.49 | Dof-type domain-containing |
| | | | | protein |
| Solyc03g082840 | 1.37 | 35.39 | 91.49 | Dof-type domain-containing |
| | | | | protein |
| Solyc01g108190 | 1.37 | 407.56 | 1,053.28 | Uncharacterized protein |
| Solyc01g108190 | 1.37 | 407.56 | 1,053.28 | Uncharacterized protein |
| Solyc10g078340 | 1.37 | 190.04 | 491.06 | Cyclin N-terminal domain- |
| | | | | containing protein |
| Solyc10g078340 | 1.37 | 190.04 | 491.06 | Cyclin N-terminal domain- |
| | | | | containing protein |
| Solyc01g006370 | 1.37 | 311.57 | 804.65 | 1,3-beta-glucan synthase (EC |
| | | | | 2.4.1.34) |
| Solyc01g006370 | 1.37 | 311.57 | 804.65 | 1,3-beta-glucan synthase (EC |
| | | | | 2.4.1.34) |
| Solyc02g089760 | 1.37 | 47.47 | 122.54 | ATP binding protein |
| Solyc02g089760 | 1.37 | 47.47 | 122.54 | ATP binding protein |
| Solyc07g006910 | 1.36 | 56.46 | 145.33 | TPT domain-containing protein |
| Solyc07g006910 | 1.36 | 56.46 | 145.33 | TPT domain-containing protein |
| Solyc01g066820 | 1.36 | 34.35 | 88.38 | Kinesin-like protein |
| Solyc01g066820 | 1.36 | 34.35 | 88.38 | Kinesin-like protein |
| Solyc07g007190 | 1.36 | 19.13 | 49.20 | Uncharacterized protein |
| Solyc07g007190 | 1.36 | 19.13 | 49.20 | Uncharacterized protein |
| Solyc09g056170 | 1.36 | 39.10 | 100.37 | Endonuclease-reverse |
| | | | | transcriptase HmRTE-e01 |
| Solyc09g056170 | 1.36 | 39.10 | 100.37 | Endonuclease-reverse |
| | | | | transcriptase HmRTE-e01 |
| Solyc12g044200 | 1.36 | 124.51 | 319.55 | AAA domain-containing |
| | | | | protein |
| Solyc12g044200 | 1.36 | 124.51 | 319.55 | AAA domain-containing |
| | | | | protein |
| Solyc06g066650 | 1.36 | 59.41 | 152.43 | PRONE domain-containing |
| | | | | protein |
| Solyc06g066650 | 1.36 | 59.41 | 152.43 | PRONE domain-containing |
| | | | | protein |
| Solyc06g009150 | 1.36 | 22.73 | 58.32 | Uncharacterized protein |
| Solyc06g009150 | 1.36 | 22.73 | 58.32 | Uncharacterized protein |
| Solyc08g078500 | 1.36 | 85.88 | 220.28 | Uncharacterized protein |
| Solyc08g078500 | 1.36 | 85.88 | 220.28 | Uncharacterized protein |
| Solyc05g006830 | 1.36 | 198.96 | 509.83 | Thioredoxin |
| Solyc05g006830 | 1.36 | 198.96 | 509.83 | Thioredoxin |
| Solyc09g072750 | 1.36 | 2,682.21 | 6,865.88 | Male specific sperm protein |
| Solyc09g072750 | 1.36 | 2,682.21 | 6,865.88 | Male specific sperm protein |
| Solyc06g050350 | 1.36 | 165.49 | 423.45 | Uncharacterized protein |
| Solyc06g050350 | 1.36 | 165.49 | 423.45 | Uncharacterized protein |
| Solyc12g014040 | 1.35 | 32.17 | 82.28 | Chloroplast protein HCF243 |
| Solyc12g014040 | 1.35 | 32.17 | 82.28 | Chloroplast protein HCF243 |
| Solyc09g090670 | 1.35 | 26.51 | 67.81 | TUDc domain-containing |
| | | | | protein |
| Solyc09g090670 | 1.35 | 26.51 | 67.81 | TUDc domain-containing |
| | | | | protein |
| Solyc03g019710 | 1.35 | 49.27 | 125.92 | Uncharacterized protein |
| Solyc03g019710 | 1.35 | 49.27 | 125.92 | Uncharacterized protein |
| Solyc01g066680 | 1.35 | 1,568.57 | 4,004.31 | Uncharacterized protein |
| Solyc01g066680 | 1.35 | 1,568.57 | 4,004.31 | Uncharacterized protein |
| Solyc04g079570 | 1.35 | 52.03 | 132.81 | Dof-type domain-containing |
| | | | | protein |
| Solyc04g079570 | 1.35 | 52.03 | 132.81 | Dof-type domain-containing |
| | | | | protein |
| Solyc05g054210 | 1.35 | 247.15 | 629.24 | Uncharacterized protein |
| Solyc05g054210 | 1.35 | 247.15 | 629.24 | Uncharacterized protein |
| Solyc01g065510 | 1.35 | 93.72 | 238.46 | Uncharacterized protein |
| Solyc01g065510 | 1.35 | 93.72 | 238.46 | Uncharacterized protein |
| Solyc02g088820 | 1.35 | 71.32 | 181.26 | Carboxvpcptidasc (EC 3.4.16.—) |
| Solyc02g088820 | 1.35 | 71.32 | 181.26 | Carboxypeptidase (EC 3.4.16.—) |
| Solyc01g098800 | 1.35 | 461.88 | 1,173.56 | Uncharacterized protein |
| Solyc01g098800 | 1.35 | 461.88 | 1,173.56 | Uncharacterized protein |
| Solyc05g005320 | 1.34 | 229.54 | 582.74 | Uncharacterized protein |
| Solyc05g005320 | 1.34 | 229.54 | 582.74 | Uncharacterized protein |
| Solyc10g050160 | 1.34 | 62.88 | 159.43 | Caffeoyl-CoA O- |
| | | | | methyltransferase (EC |
| | | | | 2.1.1.104) |
| Solyc10g050160 | 1.34 | 62.88 | 159.43 | Caffeoyl-CoA O- |
| | | | | methyltransferase (EC |
| | | | | 2.1.1.104) |
| Solyc12g006860 | 1.34 | 98.68 | 250.10 | Uncharacterized protein |
| Solyc12g006860 | 1.34 | 98.68 | 250.10 | Uncharacterized protein |
| Solyc02g090360 | 1.34 | 702.44 | 1,778.41 | Multicopper oxidase |
| Solyc02g090360 | 1.34 | 702.44 | 1,778.41 | Multicopper oxidase |
| Solyc03g031730 | 1.34 | 22.64 | 57.25 | Uncharacterized protein |
| Solyc03g031730 | 1.34 | 22.64 | 57.25 | Uncharacterized protein |
| Solyc03g083130 | 1.34 | 65.20 | 164.59 | Uncharacterized protein |
| Solyc03g083130 | 1.34 | 65.20 | 164.59 | Uncharacterized protein |
| Solyc09g055890 | 1.34 | 36.87 | 93.05 | Uncharacterized protein |
| Solyc09g055890 | 1.34 | 36.87 | 93.05 | Uncharacterized protein |
| Solyc04g074410 | 1.33 | 277.14 | 698.40 | Uncharacterized protein |
| Solyc04g074410 | 1.33 | 277.14 | 698.40 | Uncharacterized protein |
| Solyc08g006765 | 1.33 | 196.33 | 493.99 | N-acetyltransferase domain- |
| | | | | containing protein |
| Solyc08g006765 | 1.33 | 196.33 | 493.99 | N-acetyltransferase domain- |
| | | | | containing protein |
| Solyc11g020290 | 1.33 | 28.10 | 70.69 | WD_REPEATS_REGION |
| | | | | domain-containing protein |
| Solyc11g020290 | 1.33 | 28.10 | 70.69 | WD_REPEATS_REGION |
| | | | | domain-containing protein |
| Solyc03g044480 | 1.33 | 75.65 | 190.22 | Cyclin-dependent kinase |
| | | | | inhibitor |
| Solyc03g044480 | 1.33 | 75.65 | 190.22 | Cyclin-dependent kinase |
| | | | | inhibitor |
| Solyc03g114310 | 1.33 | 38.37 | 96.43 | Putative serine/threonine- |
| | | | | protein kinase HT1-like |
| Solyc03g114310 | 1.33 | 38.37 | 96.43 | Putative serine/threonine- |
| | | | | protein kinase HT1-like |
| Solyc08g079740 | 1.33 | 27.77 | 69.77 | Putative LRR receptor-like |
| | | | | serine/threonine-protein kinase- |
| | | | | like |
| Solyc08g079740 | 1.33 | 27.77 | 69.77 | Putative LRR receptor-like |
| | | | | serine/threonine-protein kinase- |
| | | | | like |
| Solyc10g008910 | 1.33 | 104.84 | 263.14 | Histone H3 |
| Solyc10g008910 | 1.33 | 104.84 | 263.14 | Histone H3 |
| Solyc12g006790 | 1.33 | 145.46 | 364.95 | AGO10 |
| Solyc12g006790 | 1.33 | 145.46 | 364.95 | AGO10 |
| Solyc08g082250 | 1.33 | 367.24 | 920.57 | Endoglucanasc (EC 3.2.1.4) |
| Solyc08g082250 | 1.33 | 367.24 | 920.57 | Endoglucanase (EC 3.2.1.4) |
| Solyc07g016170 | 1.32 | 76.19 | 190.79 | Putative L-arabinokinase-like |
| Solyc07g016170 | 1.32 | 76.19 | 190.79 | Putative L-arabinokinase-like |
| Solyc04g072760 | 1.32 | 26.58 | 66.51 | Sulfate transporter 1.3-like |
| Solyc04g072760 | 1.32 | 26.58 | 66.51 | Sulfate transporter 1.3-like |
| Solyc02g085180 | 1.32 | 35.86 | 89.73 | AB hydrolase-1 domain- |
| | | | | containing protein |
| Solyc02g085180 | 1.32 | 35.86 | 89.73 | AB hydrolase-1 domain- |
| | | | | containing protein |
| Solyc08g080400 | 1.32 | 31.37 | 78.47 | GRAS domain-containing |
| | | | | protein |
| Solyc08g080400 | 1.32 | 31.37 | 78.47 | GRAS domain-containing |
| | | | | protein |
| Solyc06g009780 | 1.32 | 69.87 | 174.80 | Kinesin motor domain- |
| | | | | containing protein |
| Solyc06g009780 | 1.32 | 69.87 | 174.80 | Kinesin motor domain- |
| | | | | containing protein |
| Solyc06g072230 | 1.32 | 131.34 | 328.55 | Uncharacterized protein |
| Solyc06g072230 | 1.32 | 131.34 | 328.55 | Uncharacterized protein |
| Solyc01g090400 | 1.32 | 27.44 | 68.63 | Uncharacterized protein |
| Solyc01g090400 | 1.32 | 27.44 | 68.63 | Uncharacterized protein |
| Solyc01g099650 | 1.32 | 22.29 | 55.66 | Formin-like protein |
| Solyc01g099650 | 1.32 | 22.29 | 55.66 | Formin-like protein |
| Solyc05g009470 | 1.32 | 672.95 | 1,677.58 | Uncharacterized protein |
| Solyc05g009470 | 1.32 | 672.95 | 1,677.58 | Uncharacterized protein |
| Solyc09g064820 | 1.32 | 99.55 | 248.04 | Uncharacterized protein |
| Solyc09g064820 | 1.32 | 99.55 | 248.04 | Uncharacterized protein |
| Solyc01g080380 | 1.32 | 66.92 | 166.73 | S-adenosylmethionine |
| | | | | decarboxylase alpha chain (EC |
| | | | | 4.1.1.50) (S- |
| | | | | adenosylmethionine |
| | | | | decarboxylase beta chain) (S- |
| | | | | adenosylmethionine |
| | | | | decarboxylase proenzyme) |
| Solyc01g080380 | 1.32 | 66.92 | 166.73 | S-adenosylmethionine |
| | | | | decarboxylase alpha chain (EC |
| | | | | 4.1.1.50) (S- |
| | | | | adenosylmethionine |
| | | | | decarboxylase beta chain) (S- |
| | | | | adenosylmethionine |
| | | | | decarboxylase proenzyme) |
| Solyc04g050920 | 1.32 | 422.05 | 1,050.89 | Uncharacterized protein |
| Solyc04g050920 | 1.32 | 422.05 | 1,050.89 | Uncharacterized protein |
| Solyc08g083230 | 1.32 | 31.50 | 78.41 | Uncharacterized protein |
| Solyc08g083230 | 1.32 | 31.50 | 78.41 | Uncharacterized protein |
| Solyc08g081380 | 1.31 | 64.67 | 160.86 | BTB domain-containing protein |
| Solyc08g081380 | 1.31 | 64.67 | 160.86 | BTB domain-containing protein |
| Solyc03g112390 | 1.31 | 39.62 | 98.45 | Uncharacterized protein |
| Solyc03g112390 | 1.31 | 39.62 | 98.45 | Uncharacterized protein |
| Solyc02g084390 | 1.31 | 105.49 | 261.69 | Kinesin motor domain- |
| | | | | containing protein |
| Solyc02g084390 | 1.31 | 105.49 | 261.69 | Kinesin motor domain- |
| | | | | containing protein |
| Solyc04g079480 | 1.31 | 118.45 | 293.65 | Uncharacterized protein |
| Solyc04g079480 | 1.31 | 118.45 | 293.65 | Uncharacterized protein |
| Solyc03g113060 | 1.31 | 614.80 | 1,522.54 | Uncharacterized protein |
| Solyc03g113060 | 1.31 | 614.80 | 1,522.54 | Uncharacterized protein |
| Solyc09g072753 | 1.31 | 1,496.98 | 3,704.80 | Male specific sperm protein |
| Solyc09g072753 | 1.31 | 1,496.98 | 3,704.80 | Male specific sperm protein |
| Solyc05g005010 | 1.31 | 220.28 | 544.89 | Uncharacterized protein |
| Solyc05g005010 | 1.31 | 220.28 | 544.89 | Uncharacterized protein |
| Solyc06g074730 | 1.31 | 33.62 | 83.12 | AGO5 |
| Solyc06g074730 | 1.31 | 33.62 | 83.12 | AGO5 |
| Solyc04g074730 | 1.30 | 230.73 | 569.70 | Arabinogalactan-protein |
| Solyc04g074730 | 1.30 | 230.73 | 569.70 | Arabinogalactan-protein |
| Solyc01g065500 | 1.30 | 70.49 | 173.96 | Uncharacterized protein |
| Solyc01g065500 | 1.30 | 70.49 | 173.96 | Uncharacterized protein |
| Solyc03g096870 | 1.30 | 23.78 | 58.63 | Putative cell division cycle |
| | | | | 20.1, cofactor of APC complex- |
| | | | | like |
| Solyc03g096870 | 1.30 | 23.78 | 58.63 | Putative cell division cycle |
| | | | | 20.1, cofactor of APC complex- |
| | | | | like |
| Solyc06g064490 | 1.30 | 49.27 | 121.28 | Uimonene cyclase |
| Solyc06g064490 | 1.30 | 49.27 | 121.28 | Uimonene cyclase |
| Solyc02g065490 | 1.30 | 112.00 | 275.66 | Homeobox domain-containing |
| | | | | protein |
| Solyc02g065490 | 1.30 | 112.00 | 275.66 | Homeobox domain-containing |
| | | | | protein |
| Solyc10g084280 | 1.30 | 200.60 | 493.64 | Uncharacterized protein |
| Solyc10g084280 | 1.30 | 200.60 | 493.64 | Uncharacterized protein |
| Solyc06g061010 | 1.30 | 60.02 | 147.56 | FEZ-type domain-containing |
| | | | | protein |
| Solyc06g061010 | 1.30 | 60.02 | 147.56 | FLZ-type domain-containing |
| | | | | protein |
| Solyc03g031800 | 1.30 | 80.67 | 198.23 | Xyloglucan |
| | | | | endotransglucosylase/hydrolase |
| | | | | (EC 2.4.1.207) |
| Solyc03g031800 | 1.30 | 80.67 | 198.23 | Xyloglucan |
| | | | | endotransglucosylase/hydrolase |
| | | | | (EC 2.4.1.207) |
| Solyc01g081210 | 1.30 | 36.10 | 88.63 | Uncharacterized protein |
| Solyc01g081210 | 1.30 | 36.10 | 88.63 | Uncharacterized protein |
| Solyc06g068520 | 1.29 | 277.92 | 681.57 | Hydroxyproline-rich systemin |
| | | | | (Defense-signaling |
| | | | | glycopeptide hormone) |
| | | | | [Cleaved into: HypSys I |
| | | | | (TomHypSys I); HypSys II |
| | | | | (TomHypSys II); HypSys III |
| | | | | (TomHypSys III)] |
| Solyc06g068520 | 1.29 | 277.92 | 681.57 | Hydroxyproline-rich systemin |
| | | | | (Defense-signaling |
| | | | | glycopeptide hormone) |
| | | | | [Cleaved into: HypSys I |
| | | | | (TomHypSys I); HypSys II |
| | | | | (TomHypSys II); HypSys III |
| | | | | (TomHypSys III)] |
| Solyc07g052980 | 1.29 | 255.65 | 626.80 | Xyloglucan |
| | | | | endotransglucosylase/hydrolase |
| | | | | (EC 2.4.1.207) |
| Solyc07g052980 | 1.29 | 255.65 | 626.80 | Xyloglucan |
| | | | | endotransglucosylase/hydrolase |
| | | | | (EC 2.4.1.207) |
| Solyc09g074300 | 1.29 | 54.47 | 133.52 | Histone H2A |
| Solyc09g074300 | 1.29 | 54.47 | 133.52 | Histone H2A |
| Solyc01g098500 | 1.29 | 618.45 | 1,516.05 | Sugar-porter family protein 5 |
| Solyc01g098500 | 1.29 | 618.45 | 1,516.05 | Sugar-porter family protein 5 |
| Solyc08g077060 | 1.29 | 335.57 | 822.38 | Uncharacterized protein |
| Solyc08g077060 | 1.29 | 335.57 | 822.38 | Uncharacterized protein |
| Solyc01g090240 | 1.29 | 212.03 | 519.09 | AvrPto-dependent Pto- |
| | | | | interacting protein 3 |
| Solyc01g090240 | 1.29 | 212.03 | 519.09 | AvrPto-dependent Pto- |
| | | | | interacting protein 3 |
| Solyc01g079110 | 1.29 | 53.88 | 131.77 | uncharacterized protein |
| | | | | LOC107849419 |
| Solyc01g079110 | 1.29 | 53.88 | 131.77 | uncharacterized protein |
| | | | | LOC107849419 |
| Solyc08g082860 | 1.29 | 50.87 | 124.39 | Trehalase (EC 3.2.1.28) (Alpha- |
| | | | | trehalose glucohydrolase) |
| Solyc08g082860 | 1.29 | 50.87 | 124.39 | Trehalase (EC 3.2.1.28) (Alpha- |
| | | | | trehalose glucohydrolase) |
| Solyc08g041710 | 1.29 | 220.56 | 538.41 | Uncharacterized protein |
| Solyc08g041710 | 1.29 | 220.56 | 538.41 | Uncharacterized protein |
| Solyc01g105010 | 1.29 | 114.05 | 278.37 | Non-specific lipid-transfer |
| | | | | protein |
| Solyc01g105010 | 1.29 | 114.05 | 278.37 | Non-specific lipid-transfer |
| | | | | protein |
| Solyc06g076270 | 1.29 | 36.48 | 88.98 | HTH myb-type domain- |
| | | | | containing protein |
| Solyc06g076270 | 1.29 | 36.48 | 88.98 | HTH myb-type domain- |
| | | | | containing protein |
| Solyc06g065670 | 1.29 | 236.82 | 577.58 | Uncharacterized protein |
| Solyc06g065670 | 1.29 | 236.82 | 577.58 | Uncharacterized protein |
| Solyc05g056120 | 1.28 | 273.74 | 666.96 | GATA transcription factor |
| Solyc05g056120 | 1.28 | 273.74 | 666.96 | GATA transcription factor |
| Solyc09g075810 | 1.28 | 104.13 | 253.62 | Phytocyanin domain-containing |
| | | | | protein |
| Solyc09g075810 | 1.28 | 104.13 | 253.62 | Phytocyanin domain-containing |
| | | | | protein |
| Solyc01g109180 | 1.28 | 72.50 | 176.49 | 4-coumarate--CoA ligase (EC |
| | | | | 6.2.1.12) |
| Solyc01g109180 | 1.28 | 72.50 | 176.49 | 4-coumarate--CoA ligase (EC |
| | | | | 6.2.1.12) |
| Solyc12g014490 | 1.28 | 173.26 | 421.72 | Uncharacterized protein |
| Solyc12g014490 | 1.28 | 173.26 | 421.72 | Uncharacterized protein |
| Solyc02g065170 | 1.28 | 30.33 | 73.73 | Uncharacterized protein |
| Solyc02g065170 | 1.28 | 30.33 | 73.73 | Uncharacterized protein |
| Solyc01g057260 | 1.28 | 171.03 | 415.60 | Uncharacterized protein |
| Solyc01g057260 | 1.28 | 171.03 | 415.60 | Uncharacterized protein |
| Solyc09g090400 | 1.28 | 33.54 | 81.51 | Uncharacterized protein |
| Solyc09g090400 | 1.28 | 33.54 | 81.51 | Uncharacterized protein |
| Solyc10g055630 | 1.28 | 128.44 | 311.88 | Putative aquaporin PIP2-2 |
| Solyc10g055630 | 1.28 | 128.44 | 311.88 | Putative aquaporin PIP2-2 |
| Solyc07g045080 | 1.28 | 50.38 | 122.21 | Putative 2-alkenal reductase |
| | | | | (NADP(+)-dependent)-like |
| Solyc07g045080 | 1.28 | 50.38 | 122.21 | Putative 2-alkenal reductase |
| | | | | (NADP(+)-dependent)-like |
| Solyc08g006310 | 1.28 | 102.23 | 247.85 | Glyco_trans_2-like domain- |
| | | | | containing protein |
| Solyc08g006310 | 1.28 | 102.23 | 247.85 | Glyco_trans_2-like domain- |
| | | | | containing protein |
| Solyc07g049320 | 1.28 | 83.65 | 202.76 | Uncharacterized protein |
| Solyc07g049320 | 1.28 | 83.65 | 202.76 | Uncharacterized protein |
| Solyc07g055740 | 1.28 | 202.59 | 490.95 | Str_synth domain-containing |
| | | | | protein |
| Solyc07g055740 | 1.28 | 202.59 | 490.95 | Str_synth domain-containing |
| | | | | protein |
| Solyc08g006270 | 1.27 | 77.71 | 188.00 | Uncharacterized protein |
| Solyc08g006270 | 1.27 | 77.71 | 188.00 | Uncharacterized protein |
| Solyc09g008370 | 1.27 | 145.19 | 351.21 | Nodulin-like domain-containing |
| | | | | protein |
| Solyc09g008370 | 1.27 | 145.19 | 351.21 | Nodulin-like domain-containing |
| | | | | protein |
| Solyc03g006670 | 1.27 | 27.79 | 67.21 | CYCUIN domain-containing |
| | | | | protein |
| Solyc03g006670 | 1.27 | 27.79 | 67.21 | CYCUIN domain-containing |
| | | | | protein |
| Solyc09g009380 | 1.27 | 35.33 | 85.27 | Protein kinase domain- |
| | | | | containing protein |
| Solyc09g009380 | 1.27 | 35.33 | 85.27 | Protein kinase domain- |
| | | | | containing protein |
| Solyc09g007480 | 1.27 | 49.35 | 119.01 | Uncharacterized protein |
| Solyc09g007480 | 1.27 | 49.35 | 119.01 | Uncharacterized protein |
| Solyc11g066970 | 1.27 | 58.09 | 139.98 | RAB6-interacting golgin |
| Solyc11g066970 | 1.27 | 58.09 | 139.98 | RAB6-interacting golgin |
| Solyc10g086030 | 1.27 | 98.22 | 236.62 | Uncharacterized protein |
| Solyc10g086030 | 1.27 | 98.22 | 236.62 | Uncharacterized protein |
| Solyc02g092240 | 1.27 | 60.62 | 146.02 | Uncharacterized protein |
| Solyc02g092240 | 1.27 | 60.62 | 146.02 | Uncharacterized protein |
| Solyc02g079520 | 1.27 | 31.52 | 75.93 | EF-hand domain-containing |
| | | | | protein |
| Solyc02g079520 | 1.27 | 31.52 | 75.93 | EF-hand domain-containing |
| | | | | protein |
| Solyc12g006997 | 1.27 | 74.84 | 180.23 | HMA domain-containing |
| | | | | protein |
| Solyc12g006997 | 1.27 | 74.84 | 180.23 | HMA domain-containing |
| | | | | protein |
| Solyc07g056310 | 1.27 | 49.26 | 118.54 | X8 domain-containing protein |
| Solyc07g056310 | 1.27 | 49.26 | 118.54 | X8 domain-containing protein |
| Solyc02g037530 | 1.27 | 256.21 | 616.44 | Auxin response factor |
| Solyc02g037530 | 1.27 | 256.21 | 616.44 | Auxin response factor |
| Solyc06g008765 | 1.27 | 53.28 | 128.15 | NBS-LRR resistance protein- |
| | | | | like protein |
| Solyc06g008765 | 1.27 | 53.28 | 128.15 | NBS-LRR resistance protein- |
| | | | | like protein |
| Solyc04g054290 | 1.27 | 27.80 | 66.87 | DYW_deaminase domain- |
| | | | | containing protein |
| Solyc04g054290 | 1.27 | 27.80 | 66.87 | DYW_deaminase domain- |
| | | | | containing protein |
| Solyc04g072890 | 1.27 | 261.85 | 629.32 | WD_REPEATS_REGION |
| | | | | domain-containing protein |
| Solyc04g072890 | 1.27 | 261.85 | 629.32 | WD_REPEATS_REGION |
| | | | | domain-containing protein |
| Solyc08g081890 | 1.26 | 159.49 | 383.16 | Uncharacterized protein |
| Solyc08g081890 | 1.26 | 159.49 | 383.16 | Uncharacterized protein |
| Solyc04g008430 | 1.26 | 114.75 | 275.67 | Protein kinase domain- |
| | | | | containing protein |
| Solyc04g008430 | 1.26 | 114.75 | 275.67 | Protein kinase domain- |
| | | | | containing protein |
| Solyc09g011170 | 1.26 | 1,093.82 | 2,621.75 | BHLH domain-containing |
| | | | | protein |
| Solyc09g011170 | 1.26 | 1,093.82 | 2,621.75 | BHLH domain-containing |
| | | | | protein |
| Solyc02g081420 | 1.26 | 27.82 | 66.59 | Uncharacterized protein |
| Solyc02g081420 | 1.26 | 27.82 | 66.59 | Uncharacterized protein |
| Solyc03g119080 | 1.26 | 1,059.28 | 2,534.06 | Putative beta-glucosidase 44- |
| | | | | like |
| Solyc03g119080 | 1.26 | 1,059.28 | 2,534.06 | Putative beta-glucosidase 44- |
| | | | | like |
| Solyc02g078760 | 1.26 | 45.63 | 109.01 | Uncharacterized protein |
| Solyc02g078760 | 1.26 | 45.63 | 109.01 | Uncharacterized protein |
| Solyc06g071540 | 1.26 | 40.06 | 95.63 | Uncharacterized protein |
| Solyc06g071540 | 1.26 | 40.06 | 95.63 | Uncharacterized protein |
| Solyc01g080100 | 1.25 | 57.67 | 137.62 | Uncharacterized protein |
| Solyc01g080100 | 1.25 | 57.67 | 137.62 | Uncharacterized protein |
| Solyc02g083190 | 1.25 | 66.64 | 158.78 | F-box domain-containing |
| | | | | protein |
| Solyc02g083190 | 1.25 | 66.64 | 158.78 | F-box domain-containing |
| | | | | protein |
| Solyc05g014370 | 1.25 | 37.43 | 89.18 | Plant mitotic spindle assembly |
| | | | | checkpoint protein mad2 |
| Solyc05g014370 | 1.25 | 37.43 | 89.18 | Plant mitotic spindle assembly |
| | | | | checkpoint protein mad2 |
| Solyc02g078780 | 1.25 | 189.10 | 450.28 | Protein kinase domain- |
| | | | | containing protein |
| Solyc02g078780 | 1.25 | 189.10 | 450.28 | Protein kinase domain- |
| | | | | containing protein |
| Solyc11g072610 | 1.25 | 27.71 | 65.98 | Uncharacterized protein |
| Solyc11g072610 | 1.25 | 27.71 | 65.98 | Uncharacterized protein |
| Solyc03g083560 | 1.25 | 172.66 | 410.99 | Uncharacterized protein |
| Solyc03g083560 | 1.25 | 172.66 | 410.99 | Uncharacterized protein |
| Solyc01g099120 | 1.25 | 73.21 | 174.25 | AB hydrolase-1 domain- |
| | | | | containing protein |
| Solyc01g099120 | 1.25 | 73.21 | 174.25 | AB hydrolase-1 domain- |
| | | | | containing protein |
| Solyc06g072845 | 1.25 | 123.91 | 294.53 | Uncharacterized protein |
| Solyc06g072845 | 1.25 | 123.91 | 294.53 | Uncharacterized protein |
| Solyc08g083110 | 1.25 | 71.07 | 168.52 | Uncharacterized protein |
| Solyc08g083110 | 1.25 | 71.07 | 168.52 | Uncharacterized protein |
| Solyc05g026180 | 1.24 | 51.52 | 122.06 | NAC domain-containing protein |
| Solyc05g026180 | 1.24 | 51.52 | 122.06 | NAC domain-containing protein |
| Solyc11g073010 | 1.24 | 27.29 | 64.57 | Exocyst subunit Exo70 family |
| | | | | protein |
| Solyc11g073010 | 1.24 | 27.29 | 64.57 | Exocyst subunit Exo70 family |
| | | | | protein |
| Solyc10g086650 | 1.24 | 77.95 | 184.36 | Uncharacterized protein |
| Solyc10g086650 | 1.24 | 77.95 | 184.36 | Uncharacterized protein |
| Solyc09g014380 | 1.24 | 565.21 | 1,336.67 | Auxin transporter-like protein 1 |
| Solyc09g014380 | 1.24 | 565.21 | 1,336.67 | Auxin transporter-like protein 1 |
| Solyc07g056270 | 1.24 | 86.66 | 204.86 | Protein kinase domain- |
| | | | | containing protein |
| Solyc07g056270 | 1.24 | 86.66 | 204.86 | Protein kinase domain- |
| | | | | containing protein |
| Solyc02g092980 | 1.24 | 83.31 | 196.86 | CycD3; 1 protein (Cyclin D3.1) |
| Solyc02g092980 | 1.24 | 83.31 | 196.86 | CycD3; 1 protein (Cyclin D3.1) |
| Solyc01g104720 | 1.24 | 444.21 | 1,049.30 | Uncharacterized protein |
| Solyc01g104720 | 1.24 | 444.21 | 1,049.30 | Uncharacterized protein |
| Solyc03g020040 | 1.24 | 455.19 | 1,075.03 | Uncharacterized protein |
| Solyc03g020040 | 1.24 | 455.19 | 1,075.03 | Uncharacterized protein |
| Solyc01g056850 | 1.24 | 79.35 | 187.26 | CTP synthase (EC 6.3.4.2) |
| | | | | (UTP--ammonia ligase) |
| Solyc01g056850 | 1.24 | 79.35 | 187.26 | CTP synthase (EC 6.3.4.2) |
| | | | | (UTP--ammonia ligase) |
| Solyc03g093460 | 1.24 | 97.00 | 228.89 | Protein kinase domain- |
| | | | | containing protein |
| Solyc03g093460 | 1.24 | 97.00 | 228.89 | Protein kinase domain- |
| | | | | containing protein |
| Solyc03g117270 | 1.24 | 47.02 | 110.89 | F-box domain-containing |
| | | | | protein |
| Solyc03g117270 | 1.24 | 47.02 | 110.89 | F-box domain-containing |
| | | | | protein |
| Solyc01g109720 | 1.24 | 27.29 | 64.34 | AB hydrolase-1 domain- |
| | | | | containing protein |
| Solyc01g109720 | 1.24 | 27.29 | 64.34 | AB hydrolase-1 domain- |
| | | | | containing protein |
| Solyc03g007485 | 1.24 | 53.33 | 125.75 | Protein kinase domain- |
| | | | | containing protein |
| Solyc03g007485 | 1.24 | 53.33 | 125.75 | Protein kinase domain- |
| | | | | containing protein |
| Solyc03g083900 | 1.24 | 71.86 | 169.38 | Uncharacterized protein |
| Solyc03g083900 | 1.24 | 71.86 | 169.38 | Uncharacterized protein |
| Solyc02g084880 | 1.24 | 53.92 | 127.09 | BHUH domain-containing |
| | | | | protein |
| Solyc02g084880 | 1.24 | 53.92 | 127.09 | BHUH domain-containing |
| | | | | protein |
| Solyc04g082110 | 1.23 | 137.00 | 322.37 | PRONE domain-containing |
| | | | | protein |
| Solyc04g082110 | 1.23 | 137.00 | 322.37 | PRONE domain-containing |
| | | | | protein |
| Solyc09g090910 | 1.23 | 439.87 | 1,034.74 | Auxin-responsive protein |
| Solyc09g090910 | 1.23 | 439.87 | 1,034.74 | Auxin-responsive protein |
| Solyc11g067300 | 1.23 | 53.00 | 124.64 | Uncharacterized protein |
| Solyc11g067300 | 1.23 | 53.00 | 124.64 | Uncharacterized protein |
| Solyc05g009820 | 1.23 | 105.37 | 246.92 | Hexosyltransferase (EC 2.4.1.—) |
| Solyc05g009820 | 1.23 | 105.37 | 246.92 | Hexosyltransferase (EC 2.4.1.—) |
| Solyc12g035190 | 1.23 | 37.61 | 88.06 | Anthranilate |
| | | | | phosphoribosyltransferase (EC |
| | | | | 2.4.2.18) |
| Solyc12g035190 | 1.23 | 37.61 | 88.06 | Anthranilate |
| | | | | phosphoribosyltransferase (EC |
| | | | | 2.4.2.18) |
| Solyc04g055160 | 1.23 | 39.54 | 92.44 | DUF4057 domain-containing |
| | | | | protein |
| Solyc04g055160 | 1.23 | 39.54 | 92.44 | DUF4057 domain-containing |
| | | | | protein |
| Solyc04g050990 | 1.22 | 115.36 | 269.37 | Uncharacterized protein |
| Solyc04g050990 | 1.22 | 115.36 | 269.37 | Uncharacterized protein |
| Solyc06g048620 | 1.22 | 50.65 | 118.22 | Protein kinase domain- |
| | | | | containing protein |
| Solyc06g048620 | 1.22 | 50.65 | 118.22 | Protein kinase domain- |
| | | | | containing protein |
| Solyc03g044150 | 1.22 | 671.10 | 1,566.17 | Subtilisin-like protease |
| Solyc03g044150 | 1.22 | 671.10 | 1,566.17 | Subtilisin-like protease |
| Solyc05g007930 | 1.22 | 78.16 | 182.26 | Galectin domain-containing |
| | | | | protein |
| Solyc05g007930 | 1.22 | 78.16 | 182.26 | Galectin domain-containing |
| | | | | protein |
| Solyc05g013560 | 1.22 | 937.83 | 2,184.41 | Uncharacterized protein |
| Solyc05g013560 | 1.22 | 937.83 | 2,184.41 | Uncharacterized protein |
| Solyc11g006250 | 1.22 | 99.01 | 230.24 | Lipase_GDSL domain- |
| | | | | containing protein |
| Solyc11g006250 | 1.22 | 99.01 | 230.24 | Lipase_GDSL domain- |
| | | | | containing protein |
| Solyc03g121610 | 1.22 | 46.75 | 108.68 | Receptor-like serine/threonine- |
| | | | | protein kinase ALE2 |
| Solyc03g121610 | 1.22 | 46.75 | 108.68 | Receptor-like serine/threonine- |
| | | | | protein kinase ALE2 |
| Solyc01g079950 | 1.22 | 312.34 | 725.24 | Peptidase A1 domain- |
| | | | | containing protein |
| Solyc01g079950 | 1.22 | 312.34 | 725.24 | Peptidase A1 domain- |
| | | | | containing protein |
| Solyc03g006830 | 1.22 | 203.71 | 473.01 | uncharacterized protein |
| | | | | LOC104232043 |
| Solyc03g006830 | 1.22 | 203.71 | 473.01 | uncharacterized protein |
| | | | | LOC104232043 |
| Solyc02g077630 | 1.21 | 166.50 | 386.05 | Protein kinase domain- |
| | | | | containing protein |
| Solyc02g077630 | 1.21 | 166.50 | 386.05 | Protein kinase domain- |
| | | | | containing protein |
| Solyc01g011105 | 1.21 | 53.98 | 124.67 | Uncharacterized protein |
| Solyc01g011105 | 1.21 | 53.98 | 124.67 | Uncharacterized protein |
| Solyc06g066180 | 1.21 | 85.77 | 198.09 | Uncharacterized protein |
| Solyc06g066180 | 1.21 | 85.77 | 198.09 | Uncharacterized protein |
| Solyc01g066740 | 1.20 | 113.55 | 261.64 | WD_REPEATS_REGION |
| | | | | domain-containing protein |
| Solyc01g066740 | 1.20 | 113.55 | 261.64 | WD_REPEATS_REGION |
| | | | | domain-containing protein |
| Solyc10g009170 | 1.20 | 91.05 | 209.78 | Secretory carrier-associated |
| | | | | membrane protein (Secretory |
| | | | | carrier membrane protein) |
| Solyc10g009170 | 1.20 | 91.05 | 209.78 | Secretory carrier-associated |
| | | | | membrane protein (Secretory |
| | | | | carrier membrane protein) |
| Solyc02g068120 | 1.20 | 102.24 | 235.29 | DCD domain-containing protein |
| Solyc02g068120 | 1.20 | 102.24 | 235.29 | DCD domain-containing protein |
| Solyc01g100030 | 1.20 | 59.11 | 135.96 | Deoxyuridine 5′-triphosphate |
| | | | | nucleotidohydrolase (dUTPase) |
| | | | | (EC 3.6.1.23) (dUTP |
| | | | | pyrophosphatase) |
| Solyc01g100030 | 1.20 | 59.11 | 135.96 | Deoxyuridine 5′-triphosphate |
| | | | | nucleotidohydrolase (dUTPase) |
| | | | | (EC 3.6.1.23) (dUTP |
| | | | | pyrophosphatase) |
| Solyc02g076670 | 1.20 | 77.19 | 177.33 | HTH myb-type domain- |
| | | | | containing protein |
| Solyc02g076670 | 1.20 | 77.19 | 177.33 | HTH myb-type domain- |
| | | | | containing protein |
| Solyc01g096040 | 1.20 | 935.79 | 2,148.79 | Peptidase A1 domain- |
| | | | | containing protein |
| Solyc01g096040 | 1.20 | 935.79 | 2,148.79 | Peptidase A1 domain- |
| | | | | containing protein |
| Solyc03g098200 | 1.20 | 104.31 | 239.47 | homeobox-leucine zipper |
| | | | | protein HDG11-like isoform X1 |
| Solyc03g098200 | 1.20 | 104.31 | 239.47 | homeobox-leucine zipper |
| | | | | protein HDG11-like isoform X1 |
| Solyc10g080340 | 1.20 | 55.52 | 127.44 | Protein DETOXIFICATION |
| | | | | (Multidrug and toxic compound |
| | | | | extrusion protein) |
| Solyc10g080340 | 1.20 | 55.52 | 127.44 | Protein DETOXIFICATION |
| | | | | (Multidrug and toxic compound |
| | | | | extrusion protein) |
| Solyc12g006240 | 1.20 | 711.73 | 1,633.61 | Uncharacterized protein |
| Solyc12g006240 | 1.20 | 711.73 | 1,633.61 | Uncharacterized protein |
| Solyc01g100210 | 1.20 | 165.32 | 379.34 | Hexosyltransferase (EC 2.4.1.—) |
| Solyc01g100210 | 1.20 | 165.32 | 379.34 | Hexosyltransferase (EC 2.4.1.—) |
| Solyc09g055900 | 1.20 | 105.65 | 242.31 | Lipoxygenase (EC 1.13.11.—) |
| Solyc09g055900 | 1.20 | 105.65 | 242.31 | Lipoxygenase (EC 1.13.11.—) |
| Solyc11g071890 | 1.20 | 39.49 | 90.53 | Bac_surface_Ag domain- |
| | | | | containing protein |
| Solyc11g071890 | 1.20 | 39.49 | 90.53 | Bac_surface_Ag domain- |
| | | | | containing protein |
| Solyc09g014350 | 1.20 | 109.48 | 250.81 | PlsC domain-containing protein |
| Solyc09g014350 | 1.20 | 109.48 | 250.81 | PlsC domain-containing protein |
| Solyc06g075010 | 1.19 | 231.69 | 530.29 | Uncharacterized protein |
| Solyc06g075010 | 1.19 | 231.69 | 530.29 | Uncharacterized protein |
| Solyc02g023950 | 1.19 | 34.82 | 79.64 | Protein kinase domain- |
| | | | | containing protein |
| Solyc02g023950 | 1.19 | 34.82 | 79.64 | Protein kinase domain- |
| | | | | containing protein |
| Solyc06g035700 | 1.19 | 66.90 | 152.84 | AP2/ERF domain-containing |
| | | | | protein |
| Solyc06g035700 | 1.19 | 66.90 | 152.84 | AP2/ERF domain-containing |
| | | | | protein |
| Solyc06g068880 | 1.19 | 64.63 | 147.54 | Carboxypeptidase (EC 3.4.16.—) |
| Solyc06g068880 | 1.19 | 64.63 | 147.54 | Carboxypeptidase (EC 3.4.16.—) |
| Solyc11g066670 | 1.19 | 77.80 | 177.59 | Glycosyltransferase (EC 2.4.1.—) |
| Solyc11g066670 | 1.19 | 77.80 | 177.59 | Glycosyltransferase (EC 2.4.1.—) |
| Solyc06g050710 | 1.19 | 36.28 | 82.75 | PAM2 domain-containing |
| | | | | protein |
| Solyc06g050710 | 1.19 | 36.28 | 82.75 | PAM2 domain-containing |
| | | | | protein |
| Solyc02g063350 | 1.19 | 183.91 | 419.51 | Uncharacterized protein |
| Solyc02g063350 | 1.19 | 183.91 | 419.51 | Uncharacterized protein |
| Solyc12g044410 | 1.19 | 619.77 | 1,413.62 | DNA binding protein |
| Solyc12g044410 | 1.19 | 619.77 | 1,413.62 | DNA binding protein |
| Solyc10g080840 | 1.19 | 337.13 | 767.24 | Uncharacterized protein |
| Solyc10g080840 | 1.19 | 337.13 | 767.24 | Uncharacterized protein |
| Solyc04g008650 | 1.19 | 114.85 | 261.21 | Inactive leucine-rich repeat |
| | | | | receptor-like serinethreonine- |
| | | | | protein kinase |
| Solyc04g008650 | 1.19 | 114.85 | 261.21 | Inactive leucine-rich repeat |
| | | | | receptor-like serinethreonine- |
| | | | | protein kinase |
| Solyc07g007340 | 1.19 | 97.52 | 221.72 | Nodulin-like domain-containing |
| | | | | protein |
| Solyc07g007340 | 1.19 | 97.52 | 221.72 | Nodulin-like domain-containing |
| | | | | protein |
| Solyc12g038343 | 1.18 | 43.50 | 98.82 | uncharacterized protein |
| | | | | LOC104236329 |
| Solyc12g038343 | 1.18 | 43.50 | 98.82 | uncharacterized protein |
| | | | | LOC104236329 |
| Solyc00g500023 | 1.18 | 95.17 | 216.08 | ATP-dependent Clp protease |
| | | | | proteolytic subunit |
| Solyc00g500023 | 1.18 | 95.17 | 216.08 | ATP-dependent Clp protease |
| | | | | proteolytic subunit |
| Solyc02g084610 | 1.18 | 153.84 | 349.01 | Uncharacterized protein |
| Solyc02g084610 | 1.18 | 153.84 | 349.01 | Uncharacterized protein |
| Solyc09g082890 | 1.18 | 152.50 | 345.84 | Calcium-transporting ATPase |
| | | | | (EC 7.2.2.10) |
| Solyc09g082890 | 1.18 | 152.50 | 345.84 | Calcium-transporting ATPase |
| | | | | (EC 7.2.2.10) |
| Solyc08g067030 | 1.18 | 315.43 | 715.03 | Uncharacterized protein |
| Solyc08g067030 | 1.18 | 315.43 | 715.03 | Uncharacterized protein |
| Solyc02g078720 | 1.18 | 79.65 | 180.47 | Putative CTTNBP2 N-terminal- |
| | | | | like (Putative ovule protein) |
| Solyc02g078720 | 1.18 | 79.65 | 180.47 | Putative CTTNBP2 N-terminal- |
| | | | | like (Putative ovule protein) |
| Solyc09g097840 | 1.18 | 36.50 | 82.56 | Uncharacterized protein |
| Solyc09g097840 | 1.18 | 36.50 | 82.56 | Uncharacterized protein |
| Solyc10g009380 | 1.17 | 34.02 | 76.80 | Uncharacterized protein |
| Solyc10g009380 | 1.17 | 34.02 | 76.80 | Uncharacterized protein |
| Solyc05g005960 | 1.17 | 114.81 | 258.81 | Uncharacterized protein |
| Solyc05g005960 | 1.17 | 114.81 | 258.81 | Uncharacterized protein |
| Solyc03g121970 | 1.17 | 290.57 | 654.43 | Uncharacterized protein |
| Solyc03g121970 | 1.17 | 290.57 | 654.43 | Uncharacterized protein |
| Solyc04g078740 | 1.17 | 166.48 | 374.76 | Uncharacterized protein |
| Solyc04g078740 | 1.17 | 166.48 | 374.76 | Uncharacterized protein |
| Solyc04g081240 | 1.17 | 183.07 | 411.65 | Auxin response factor |
| Solyc04g081240 | 1.17 | 183.07 | 411.65 | Auxin response factor |
| Solyc09g005200 | 1.17 | 144.79 | 325.35 | Uncharacterized protein |
| Solyc09g005200 | 1.17 | 144.79 | 325.35 | Uncharacterized protein |
| Solyc02g084640 | 1.17 | 271.72 | 610.50 | Aldehyde dehydrogenase |
| Solyc02g084640 | 1.17 | 271.72 | 610.50 | Aldehyde dehydrogenase |
| Solyc06g074420 | 1.17 | 170.01 | 381.79 | Uncharacterized protein |
| Solyc06g074420 | 1.17 | 170.01 | 381.79 | Uncharacterized protein |
| Solyc02g092580 | 1.17 | 63.47 | 142.51 | Peroxidase (EC 1.11.1.7) |
| | | | | (Fragment) |
| Solyc02g092580 | 1.17 | 63.47 | 142.51 | Peroxidase (EC 1.11.1.7) |
| | | | | (Fragment) |
| Solyc12g055840 | 1.17 | 404.51 | 908.18 | Uncharacterized protein |
| Solyc12g055840 | 1.17 | 404.51 | 908.18 | Uncharacterized protein |
| Solyc08g160350 | 1.17 | 45.34 | 101.66 | Uncharacterized protein |
| Solyc08g160350 | 1.17 | 45.34 | 101.66 | Uncharacterized protein |
| Solyc06g076400 | 1.16 | 538.07 | 1,203.70 | Protein-serine/threonine |
| | | | | phosphatase (EC 3.1.3.16) |
| Solyc06g076400 | 1.16 | 538.07 | 1,203.70 | Protein-serine/threonine |
| | | | | phosphatase (EC 3.1.3.16) |
| Solyc10g085620 | 1.16 | 258.68 | 578.02 | HTH myb-type domain- |
| | | | | containing protein |
| Solyc10g085620 | 1.16 | 258.68 | 578.02 | HTH myb-type domain- |
| | | | | containing protein |
| Solyc09g010510 | 1.16 | 60.24 | 134.50 | Uncharacterized protein |
| Solyc09g010510 | 1.16 | 60.24 | 134.50 | Uncharacterized protein |
| Solyc03g118790 | 1.16 | 40.14 | 89.61 | Uncharacterized protein |
| Solyc03g118790 | 1.16 | 40.14 | 89.61 | Uncharacterized protein |
| Solyc09g075550 | 1.16 | 49.37 | 110.20 | Uncharacterized protein |
| Solyc09g075550 | 1.16 | 49.37 | 110.20 | Uncharacterized protein |
| Solyc02g092810 | 1.16 | 165.50 | 369.24 | RING-type domain-containing |
| | | | | protein |
| Solyc02g092810 | 1.16 | 165.50 | 369.24 | RING-type domain-containing |
| | | | | protein |
| Solyc02g081730 | 1.16 | 85.76 | 191.25 | 3Beta_HSD domain-containing |
| | | | | protein |
| Solyc02g081730 | 1.16 | 85.76 | 191.25 | 3Beta_HSD domain-containing |
| | | | | protein |
| Solyc06g050800 | 1.16 | 58.01 | 129.32 | Amino acid transporter |
| Solyc06g050800 | 1.16 | 58.01 | 129.32 | Amino acid transporter |
| Solyc01g102980 | 1.16 | 44.00 | 98.07 | ZF-HD dimerization-type |
| | | | | domain-containing protein |
| Solyc01g102980 | 1.16 | 44.00 | 98.07 | ZF-HD dimerization-type |
| | | | | domain-containing protein |
| Solyc02g078890 | 1.15 | 43.70 | 97.28 | Uncharacterized protein |
| Solyc02g078890 | 1.15 | 43.70 | 97.28 | Uncharacterized protein |
| Solyc01g088250 | 1.15 | 263.48 | 586.38 | DUF4005 domain-containing |
| | | | | protein |
| Solyc01g088250 | 1.15 | 263.48 | 586.38 | DUF4005 domain-containing |
| | | | | protein |
| Solyc08g008380 | 1.15 | 117.97 | 262.54 | Auxin response factor |
| Solyc08g008380 | 1.15 | 117.97 | 262.54 | Auxin response factor |
| Solyc02g071610 | 1.15 | 234.75 | 522.22 | GDSL-lipase 1 |
| Solyc02g071610 | 1.15 | 234.75 | 522.22 | GDSL-lipase 1 |
| Solyc09g092320 | 1.15 | 65.38 | 145.41 | RRM domain-containing |
| | | | | protein |
| Solyc09g092320 | 1.15 | 65.38 | 145.41 | RRM domain-containing |
| | | | | protein |
| Solyc08g083130 | 1.15 | 50.57 | 112.45 | Homeobox domain-containing |
| | | | | protein |
| Solyc08g083130 | 1.15 | 50.57 | 112.45 | Homeobox domain-containing |
| | | | | protein |
| Solyc10g075070 | 1.15 | 5,287.51 | 11,756.14 | Non-specific lipid-transfer |
| | | | | protein |
| Solyc10g075070 | 1.15 | 5,287.51 | 11,756.14 | Non-specific lipid-transfer |
| | | | | protein |
| Solyc05g050210 | 1.15 | 40.11 | 89.17 | Putative GDSL esterase/lipase- |
| | | | | like (Fragment) |
| Solyc05g050210 | 1.15 | 40.11 | 89.17 | Putative GDSL esterase/lipase- |
| | | | | like (Fragment) |
| Solyc03g005060 | 1.15 | 46.40 | 103.09 | C2 NT-type domain-containing |
| | | | | protein |
| Solyc03g005060 | 1.15 | 46.40 | 103.09 | C2 NT-type domain-containing |
| | | | | protein |
| Solyc10g078530 | 1.15 | 615.93 | 1,365.24 | PNPUA domain-containing |
| | | | | protein |
| Solyc10g078530 | 1.15 | 615.93 | 1,365.24 | PNPUA domain-containing |
| | | | | protein |
| Solyc09g082810 | 1.15 | 463.17 | 1,026.56 | NA |
| Solyc09g082810 | 1.15 | 463.17 | 1,026.56 | NA |
| Solyc08g061560 | 1.15 | 149.42 | 331.16 | Protein kinase domain- |
| | | | | containing protein |
| Solyc08g061560 | 1.15 | 149.42 | 331.16 | Protein kinase domain- |
| | | | | containing protein |
| Solyc02g071080 | 1.15 | 54.25 | 120.22 | Uncharacterized protein |
| Solyc02g071080 | 1.15 | 54.25 | 120.22 | Uncharacterized protein |
| Solyc04g015280 | 1.15 | 114.23 | 252.78 | RING-type domain-containing |
| | | | | protein |
| Solyc04g015280 | 1.15 | 114.23 | 252.78 | RING-type domain-containing |
| | | | | protein |
| Solyc08g067370 | 1.14 | 1,386.17 | 3,064.98 | Uncharacterized protein |
| Solyc08g067370 | 1.14 | 1,386.17 | 3,064.98 | Uncharacterized protein |
| Solyc03g119210 | 1.14 | 88.91 | 196.50 | AAI domain-containing protein |
| Solyc03g119210 | 1.14 | 88.91 | 196.50 | AAI domain-containing protein |
| Solyc02g082420 | 1.14 | 155.84 | 344.23 | RING-type domain-containing |
| | | | | protein |
| Solyc02g082420 | 1.14 | 155.84 | 344.23 | RING-type domain-containing |
| | | | | protein |
| Solyc09g098380 | 1.14 | 141.14 | 311.30 | vacuolar amino acid transporter |
| | | | | 1 |
| Solyc09g098380 | 1.14 | 141.14 | 311.30 | vacuolar amino acid transporter |
| | | | | 1 |
| Solyc08g079460 | 1.14 | 318.99 | 702.88 | Protein kinase domain- |
| | | | | containing protein |
| Solyc08g079460 | 1.14 | 318.99 | 702.88 | Protein kinase domain- |
| | | | | containing protein |
| Solyc07g005680 | 1.14 | 77.98 | 171.73 | Carboxypeptidase (EC 3.4.16.—) |
| Solyc07g005680 | 1.14 | 77.98 | 171.73 | Carboxypeptidase (EC 3.4.16.—) |
| Solyc06g048950 | 1.14 | 216.89 | 476.94 | Protein kinase domain- |
| | | | | containing protein |
| Solyc06g048950 | 1.14 | 216.89 | 476.94 | Protein kinase domain- |
| | | | | containing protein |
| Solyc02g077850 | 1.14 | 234.44 | 515.30 | Malectin_like domain- |
| | | | | containing protein |
| Solyc02g077850 | 1.14 | 234.44 | 515.30 | Malectin_like domain- |
| | | | | containing protein |
| Solyc02g079290 | 1.14 | 47.10 | 103.46 | SP2G |
| Solyc02g079290 | 1.14 | 47.10 | 103.46 | SP2G |
| Solyc01g108240 | 1.13 | 264.14 | 580.06 | AP2/ERF domain-containing |
| | | | | protein |
| Solyc01g108240 | 1.13 | 264.14 | 580.06 | AP2/ERF domain-containing |
| | | | | protein |
| Solyc07g055930 | 1.13 | 618.03 | 1,357.22 | Hexosyltransferase (EC 2.4.1.—) |
| Solyc07g055930 | 1.13 | 618.03 | 1,357.22 | Hexosyltransferase (EC 2.4.1.—) |
| Solyc03g080180 | 1.13 | 514.50 | 1,128.52 | Uncharacterized protein |
| Solyc03g080180 | 1.13 | 514.50 | 1,128.52 | Uncharacterized protein |
| Solyc11g013330 | 1.13 | 78.11 | 171.24 | Uncharacterized protein |
| Solyc11g013330 | 1.13 | 78.11 | 171.24 | Uncharacterized protein |
| Solyc10g079280 | 1.13 | 74.80 | 163.98 | Phosphate/phosphoenolpyruvate |
| | | | | translocator |
| Solyc10g079280 | 1.13 | 74.80 | 163.98 | Phosphate/phosphoenolpyruvate |
| | | | | translocator |
| Solyc03g120120 | 1.13 | 115.50 | 253.06 | Uncharacterized protein |
| Solyc03g120120 | 1.13 | 115.50 | 253.06 | Uncharacterized protein |
| Solyc03g031890 | 1.13 | 81.46 | 178.48 | Uncharacterized protein |
| Solyc03g031890 | 1.13 | 81.46 | 178.48 | Uncharacterized protein |
| Solyc12g011420 | 1.13 | 112.45 | 246.14 | Uncharacterized protein |
| Solyc12g011420 | 1.13 | 112.45 | 246.14 | Uncharacterized protein |
| Solyc07g014640 | 1.13 | 47.11 | 102.93 | Integrase catalytic domain- |
| | | | | containing protein |
| Solyc07g014640 | 1.13 | 47.11 | 102.93 | Integrase catalytic domain- |
| | | | | containing protein |
| Solyc12g006470 | 1.13 | 1,243.45 | 2,714.58 | Uncharacterized protein |
| Solyc12g006470 | 1.13 | 1,243.45 | 2,714.58 | Uncharacterized protein |
| Solyc03g123620 | 1.13 | 1,079.75 | 2,356.99 | Pectinesterase (EC 3.1.1.11) |
| Solyc03g123620 | 1.13 | 1,079.75 | 2,356.99 | Pectinesterase (EC 3.1.1.11) |
| Solyc02g092450 | 1.12 | 85.61 | 186.64 | Calcium-transporting ATPase |
| | | | | (EC 7.2.2.10) |
| Solyc02g092450 | 1.12 | 85.61 | 186.64 | Calcium-transporting ATPase |
| | | | | (EC 7.2.2.10) |
| Solyc03g095490 | 1.12 | 355.39 | 774.78 | Protein kinase domain- |
| | | | | containing protein |
| Solyc03g095490 | 1.12 | 355.39 | 774.78 | Protein kinase domain- |
| | | | | containing protein |
| Solyc06g082780 | 1.12 | 184.52 | 402.05 | Calcium uniporter protein |
| Solyc06g082780 | 1.12 | 184.52 | 402.05 | Calcium uniporter protein |
| Solyc07g007550 | 1.12 | 180.53 | 393.34 | Uncharacterized protein |
| Solyc07g007550 | 1.12 | 180.53 | 393.34 | Uncharacterized protein |
| Solyc03g006550 | 1.12 | 42.49 | 92.45 | Putative geranyllinalool |
| | | | | synthase |
| Solyc03g006550 | 1.12 | 42.49 | 92.45 | Putative geranyllinalool |
| | | | | synthase |
| Solyc04g007000 | 1.12 | 212.10 | 461.32 | RAV1 |
| Solyc04g007000 | 1.12 | 212.10 | 461.32 | RAV1 |
| Solyc10g080760 | 1.12 | 42.11 | 91.57 | Nudix hydrolase domain- |
| | | | | containing protein |
| Solyc10g080760 | 1.12 | 42.11 | 91.57 | Nudix hydrolase domain- |
| | | | | containing protein |
| Solyc08g080320 | 1.12 | 356.21 | 774.17 | Uncharacterized protein |
| Solyc08g080320 | 1.12 | 356.21 | 774.17 | Uncharacterized protein |
| Solyc11g017410 | 1.12 | 102.89 | 223.60 | Uncharacterized protein |
| Solyc11g017410 | 1.12 | 102.89 | 223.60 | Uncharacterized protein |
| Solyc03g097380 | 1.12 | 443.71 | 963.98 | Metal ion binding protein |
| Solyc03g097380 | 1.12 | 443.71 | 963.98 | Metal ion binding protein |
| Solyc06g069400 | 1.12 | 92.05 | 199.79 | Uncharacterized protein |
| Solyc06g069400 | 1.12 | 92.05 | 199.79 | Uncharacterized protein |
| Solyc10g078160 | 1.12 | 109.77 | 238.23 | Uncharacterized protein |
| Solyc10g078160 | 1.12 | 109.77 | 238.23 | Uncharacterized protein |
| Solyc08g008340 | 1.12 | 82.38 | 178.76 | Nucleoside diphosphate kinase |
| | | | | (EC 2.7.4.6) |
| Solyc08g008340 | 1.12 | 82.38 | 178.76 | Nucleoside diphosphate kinase |
| | | | | (EC 2.7.4.6) |
| Solyc06g050170 | 1.12 | 514.29 | 1,115.55 | Potassium transporter |
| Solyc06g050170 | 1.12 | 514.29 | 1,115.55 | Potassium transporter |
| Solyc03g117550 | 1.12 | 70.52 | 152.89 | Protein kinase domain- |
| | | | | containing protein |
| Solyc03g117550 | 1.12 | 70.52 | 152.89 | Protein kinase domain- |
| | | | | containing protein |
| Solyc05g051660 | 1.12 | 180.36 | 391.00 | Acylsugar acylhydrolase 1 |
| Solyc05g051660 | 1.12 | 180.36 | 391.00 | Acylsugar acylhydrolase 1 |
| Solyc12g005120 | 1.11 | 439.25 | 950.39 | Uncharacterized protein |
| Solyc12g005120 | 1.11 | 439.25 | 950.39 | Uncharacterized protein |
| Solyc06g053640 | 1.11 | 64.38 | 139.29 | RING-type domain-containing |
| | | | | protein |
| Solyc06g053640 | 1.11 | 64.38 | 139.29 | RING-type domain-containing |
| | | | | protein |
| Solyc06g084460 | 1.11 | 139.43 | 301.34 | Shikimate dehydrogenase (EC |
| | | | | 1.1.1.25) |
| Solyc06g084460 | 1.11 | 139.43 | 301.34 | Shikimate dehydrogenase (EC |
| | | | | 1.1.1.25) |
| Solyc07g045180 | 1.11 | 152.22 | 328.95 | Uncharacterized protein |
| Solyc07g045180 | 1.11 | 152.22 | 328.95 | Uncharacterized protein |
| Solyc01g067800 | 1.11 | 228.56 | 493.04 | RING-CH-type domain- |
| | | | | containing protein |
| Solyc01g067800 | 1.11 | 228.56 | 493.04 | RING-CH-type domain- |
| | | | | containing protein |
| Solyc01g098910 | 1.11 | 728.84 | 1,571.59 | Uncharacterized protein |
| Solyc01g098910 | 1.11 | 728.84 | 1,571.59 | Uncharacterized protein |
| Solyc02g031770 | 1.11 | 78.40 | 168.90 | Uncharacterized protein |
| Solyc02g031770 | 1.11 | 78.40 | 168.90 | Uncharacterized protein |
| Solyc07g064820 | 1.11 | 169.62 | 365.37 | Protein kinase domain- |
| | | | | containing protein |
| Solyc07g064820 | 1.11 | 169.62 | 365.37 | Protein kinase domain- |
| | | | | containing protein |
| Solyc12g098360 | 1.11 | 46.27 | 99.67 | Protein arginine |
| | | | | methyltransferase NDUFAF7 |
| | | | | (EC 2.1.1.320) |
| Solyc12g098360 | 1.11 | 46.27 | 99.67 | Protein arginine |
| | | | | methyltransferase NDUFAF7 |
| | | | | (EC 2.1.1.320) |
| Solyc02g065780 | 1.11 | 67.89 | 146.06 | Uncharacterized protein |
| Solyc02g065780 | 1.11 | 67.89 | 146.06 | Uncharacterized protein |
| Solyc04g008040 | 1.10 | 141.28 | 303.74 | Myosin heavy chain, clone |
| Solyc04g008040 | 1.10 | 141.28 | 303.74 | Myosin heavy chain, clone |
| Solyc01g099410 | 1.10 | 338.03 | 724.21 | Histone H2A.1 (LeH2A-1) |
| Solyc01g099410 | 1.10 | 338.03 | 724.21 | Histone H2A.1 (LeH2A-1) |
| Solyc10g076820 | 1.10 | 45.61 | 97.60 | Uncharacterized protein |
| Solyc10g076820 | 1.10 | 45.61 | 97.60 | Uncharacterized protein |
| Solyc04g082610 | 1.10 | 260.18 | 556.63 | Glutamate receptor |
| Solyc04g082610 | 1.10 | 260.18 | 556.63 | Glutamate receptor |
| Solyc09g083380 | 1.10 | 191.52 | 409.75 | Histone H1F |
| Solyc09g083380 | 1.10 | 191.52 | 409.75 | Histone H1F |
| Solyc09g089740 | 1.10 | 59.66 | 127.51 | Uncharacterized protein |
| Solyc09g089740 | 1.10 | 59.66 | 127.51 | Uncharacterized protein |
| Solyc02g070000 | 1.10 | 171.54 | 366.46 | Protein kinase domain- |
| | | | | containing protein |
| Solyc02g070000 | 1.10 | 171.54 | 366.46 | Protein kinase domain- |
| | | | | containing protein |
| Solyc02g080310 | 1.09 | 82.74 | 176.65 | Putative beta-glucosidase 47- |
| | | | | like |
| Solyc02g080310 | 1.09 | 82.74 | 176.65 | Putative beta-glucosidase 47- |
| | | | | like |
| Solyc04g005410 | 1.09 | 116.12 | 247.59 | Uncharacterized protein |
| Solyc04g005410 | 1.09 | 116.12 | 247.59 | Uncharacterized protein |
| Solyc08g061970 | 1.09 | 879.74 | 1,875.11 | Putative spermine synthase |
| Solyc08g061970 | 1.09 | 879.74 | 1,875.11 | Putative spermine synthase |
| Solyc10g080390 | 1.09 | 226.75 | 483.11 | Protein kinase domain- |
| | | | | containing protein |
| Solyc10g080390 | 1.09 | 226.75 | 483.11 | Protein kinase domain- |
| | | | | containing protein |
| Solyc09g074340 | 1.09 | 54.11 | 115.14 | Uncharacterized protein |
| Solyc09g074340 | 1.09 | 54.11 | 115.14 | Uncharacterized protein |
| Solyc05g054530 | 1.09 | 96.45 | 205.17 | Uncharacterized protein |
| Solyc05g054530 | 1.09 | 96.45 | 205.17 | Uncharacterized protein |
| Solyc03g093130 | 1.09 | 299.54 | 636.87 | Xyloglucan |
| | | | | endotransglucosylase/hydrolase |
| | | | | (EC 2.4.1.207) |
| Solyc03g093130 | 1.09 | 299.54 | 636.87 | Xyloglucan |
| | | | | endotransglucosylase/hydrolase |
| | | | | (EC 2.4.1.207) |
| Solyc03g096670 | 1.09 | 107.22 | 227.90 | Protein-serine/threonine |
| | | | | phosphatase (EC 3.1.3.16) |
| Solyc03g096670 | 1.09 | 107.22 | 227.90 | Protein-serine/threonine |
| | | | | phosphatase (EC 3.1.3.16) |
| Solyc01g081120 | 1.09 | 119.33 | 253.14 | Uncharacterized protein |
| Solyc01g081120 | 1.09 | 119.33 | 253.14 | Uncharacterized protein |
| Solyc10g075103 | 1.08 | 300.49 | 637.07 | Non-specific lipid-transfer |
| | | | | protein 2 (LTP 2) |
| Solyc10g075103 | 1.08 | 300.49 | 637.07 | Non-specific lipid-transfer |
| | | | | protein 2 (LTP 2) |
| Solyc02g087410 | 1.08 | 50.24 | 106.51 | Uncharacterized protein |
| Solyc02g087410 | 1.08 | 50.24 | 106.51 | Uncharacterized protein |
| Solyc01g005590 | 1.08 | 51.63 | 109.40 | NA |
| Solyc01g005590 | 1.08 | 51.63 | 109.40 | NA |
| Solyc01g103590 | 1.08 | 129.35 | 273.91 | Lactoylglutathione lyase |
| Solyc01g103590 | 1.08 | 129.35 | 273.91 | Lactoylglutathione lyase |
| Solyc01g007130 | 1.08 | 231.35 | 489.88 | Protein kinase domain- |
| | | | | containing protein |
| Solyc01g007130 | 1.08 | 231.35 | 489.88 | Protein kinase domain- |
| | | | | containing protein |
| Solyc01g095460 | 1.08 | 432.93 | 916.69 | BZIP domain-containing |
| | | | | protein |
| Solyc01g095460 | 1.08 | 432.93 | 916.69 | BZIP domain-containing |
| | | | | protein |
| Solyc04g015600 | 1.08 | 97.43 | 206.29 | Protein kinase domain- |
| | | | | containing protein |
| Solyc04g015600 | 1.08 | 97.43 | 206.29 | Protein kinase domain- |
| | | | | containing protein |
| Solyc07g053890 | 1.08 | 73.02 | 154.54 | Diacylglycerol O- |
| | | | | acyltransferase (EC 2.3.1.20) |
| Solyc07g053890 | 1.08 | 73.02 | 154.54 | Diacylglycerol O- |
| | | | | acyltransferase (EC 2.3.1.20) |
| Solyc10g076790 | 1.08 | 126.35 | 267.27 | LAX4 protein |
| Solyc10g076790 | 1.08 | 126.35 | 267.27 | LAX4 protein |
| Solyc05g016180 | 1.08 | 50.83 | 107.49 | Uncharacterized protein |
| Solyc05g016180 | 1.08 | 50.83 | 107.49 | Uncharacterized protein |
| Solyc09g007510 | 1.08 | 67.08 | 141.85 | Clade VIII lectin receptor |
| | | | | kinase |
| Solyc09g007510 | 1.08 | 67.08 | 141.85 | Clade VIII lectin receptor |
| | | | | kinase |
| Solyc08g076310 | 1.08 | 117.75 | 248.86 | Uncharacterized protein |
| Solyc08g076310 | 1.08 | 117.75 | 248.86 | Uncharacterized protein |
| Solyc10g008270 | 1.08 | 223.03 | 470.88 | BHLH domain-containing |
| | | | | protein |
| Solyc10g008270 | 1.08 | 223.03 | 470.88 | BHLH domain-containing |
| | | | | protein |
| Solyc11g069190 | 1.08 | 1,851.33 | 3,906.43 | Auxin response factor |
| Solyc11g069190 | 1.08 | 1,851.33 | 3,906.43 | Auxin response factor |
| Solyc01g008940 | 1.08 | 190.68 | 401.95 | Uncharacterized protein |
| Solyc01g008940 | 1.08 | 190.68 | 401.95 | Uncharacterized protein |
| Solyc09g061860 | 1.08 | 122.99 | 259.15 | Glyco_transf_28 domain- |
| | | | | containing protein |
| Solyc09g061860 | 1.08 | 122.99 | 259.15 | Glyco_transf_28 domain- |
| | | | | containing protein |
| Solyc01g099290 | 1.07 | 52.75 | 111.09 | Cullin domain-containing |
| | | | | protein |
| Solyc01g099290 | 1.07 | 52.75 | 111.09 | Cullin domain-containing |
| | | | | protein |
| Solyc04g080640 | 1.07 | 249.92 | 525.95 | Uncharacterized protein |
| Solyc04g080640 | 1.07 | 249.92 | 525.95 | Uncharacterized protein |
| Solyc02g079750 | 1.07 | 294.05 | 618.42 | NAD(P)H dehydrogenase |
| | | | | (quinone) (EC 1.6.5.2) |
| Solyc02g079750 | 1.07 | 294.05 | 618.42 | NAD(P)H dehydrogenase |
| | | | | (quinone) (EC 1.6.5.2) |
| Solyc10g074820 | 1.07 | 59.09 | 124.24 | Uncharacterized protein |
| Solyc10g074820 | 1.07 | 59.09 | 124.24 | Uncharacterized protein |
| Solyc03g121440 | 1.07 | 230.21 | 483.91 | Protein kinase domain- |
| | | | | containing protein |
| Solyc03g121440 | 1.07 | 230.21 | 483.91 | Protein kinase domain- |
| | | | | containing protein |
| Solyc08g081550 | 1.07 | 813.65 | 1,709.40 | 1-amino-cyclopropane-1- |
| | | | | carboxylate synthase (EC |
| | | | | 4.4.1.14) |
| Solyc08g081550 | 1.07 | 813.65 | 1,709.40 | 1-amino-cyclopropane-1- |
| | | | | carboxylate synthase (EC |
| | | | | 4.4.1.14) |
| Solyc09g064530 | 1.07 | 133.36 | 280.12 | Auxin-responsive protein |
| Solyc09g064530 | 1.07 | 133.36 | 280.12 | Auxin-responsive protein |
| Solyc07g054630 | 1.07 | 56.98 | 119.42 | Uncharacterized protein |
| Solyc07g054630 | 1.07 | 56.98 | 119.42 | Uncharacterized protein |
| Solyc04g008060 | 1.07 | 380.76 | 797.54 | Protein CCC1 |
| Solyc04g008060 | 1.07 | 380.76 | 797.54 | Protein CCC1 |
| Solyc01g111570 | 1.07 | 155.50 | 325.67 | Protein kinase domain- |
| | | | | containing protein |
| Solyc01g111570 | 1.07 | 155.50 | 325.67 | Protein kinase domain- |
| | | | | containing protein |
| Solyc04g074110 | 1.07 | 88.51 | 185.36 | SASA domain-containing |
| | | | | protein |
| Solyc04g074110 | 1.07 | 88.51 | 185.36 | SASA domain-containing |
| | | | | protein |
| Solyc09g082870 | 1.06 | 230.67 | 482.57 | Calcium-transporting ATPase |
| | | | | (EC 7.2.2.10) |
| Solyc09g082870 | 1.06 | 230.67 | 482.57 | Calcium-transporting ATPase |
| | | | | (EC 7.2.2.10) |
| Solyc04g051660 | 1.06 | 160.69 | 336.10 | Chloroplast lumen common |
| | | | | family protein |
| Solyc04g051660 | 1.06 | 160.69 | 336.10 | Chloroplast lumen common |
| | | | | family protein |
| Solyc12g010590 | 1.06 | 286.73 | 599.73 | Diacylglycerol O- |
| | | | | acyltransferase (EC 2.3.1.20) |
| Solyc12g010590 | 1.06 | 286.73 | 599.73 | Diacylglycerol O- |
| | | | | acyltransferase (EC 2.3.1.20) |
| Solyc08g079090 | 1.06 | 114.43 | 239.06 | Putative cupredoxin |
| Solyc08g079090 | 1.06 | 114.43 | 239.06 | Putative cupredoxin |
| Solyc01g100080 | 1.06 | 144.47 | 301.78 | Single-stranded DNA-binding |
| | | | | protein, mitochondrial |
| Solyc01g100080 | 1.06 | 144.47 | 301.78 | Single-stranded DNA-binding |
| | | | | protein, mitochondrial |
| Solyc03g117800 | 1.06 | 1,183.97 | 2,473.09 | Uncharacterized protein |
| Solyc03g117800 | 1.06 | 1,183.97 | 2,473.09 | Uncharacterized protein |
| Solyc09g065630 | 1.06 | 95.85 | 200.19 | BIG SEEDS 2 |
| Solyc09g065630 | 1.06 | 95.85 | 200.19 | BIG SEEDS 2 |
| Solyc03g112100 | 1.06 | 105.46 | 220.23 | High-affinity nitrate transporter |
| Solyc03g112100 | 1.06 | 105.46 | 220.23 | High-affinity nitrate transporter |
| Solyc03g032180 | 1.06 | 148.86 | 310.25 | Uncharacterized protein |
| Solyc03g032180 | 1.06 | 148.86 | 310.25 | Uncharacterized protein |
| Solyc02g077720 | 1.06 | 56.12 | 116.85 | Uncharacterized protein |
| Solyc02g077720 | 1.06 | 56.12 | 116.85 | Uncharacterized protein |
| Solyc11g065490 | 1.06 | 488.01 | 1,015.55 | Uncharacterized protein |
| Solyc11g065490 | 1.06 | 488.01 | 1,015.55 | Uncharacterized protein |
| Solyc12g014440 | 1.06 | 298.96 | 622.12 | Putative ovule protein |
| Solyc12g014440 | 1.06 | 298.96 | 622.12 | Putative ovule protein |
| Solyc05g013690 | 1.06 | 148.20 | 308.25 | Uncharacterized protein |
| Solyc05g013690 | 1.06 | 148.20 | 308.25 | Uncharacterized protein |
| Solyc12g007140 | 1.06 | 68.61 | 142.66 | Uncharacterized protein |
| Solyc12g007140 | 1.06 | 68.61 | 142.66 | Uncharacterized protein |
| Solyc03g119960 | 1.06 | 86.36 | 179.48 | NPH3 domain-containing |
| | | | | protein |
| Solyc03g119960 | 1.06 | 86.36 | 179.48 | NPH3 domain-containing |
| | | | | protein |
| Solyc10g005690 | 1.05 | 60.03 | 124.67 | Chloride channel protein |
| Solyc10g005690 | 1.05 | 60.03 | 124.67 | Chloride channel protein |
| Solyc07g005775 | 1.05 | 311.11 | 645.81 | Putative ovule protein |
| Solyc07g005775 | 1.05 | 311.11 | 645.81 | Putative ovule protein |
| Solyc07g066040 | 1.05 | 59.29 | 122.93 | Uncharacterized protein |
| Solyc07g066040 | 1.05 | 59.29 | 122.93 | Uncharacterized protein |
| Solyc03g026070 | 1.05 | 125.50 | 259.89 | Uncharacterized protein |
| Solyc03g026070 | 1.05 | 125.50 | 259.89 | Uncharacterized protein |
| Solyc11g072860 | 1.05 | 148.78 | 308.00 | Histone H4 |
| Solyc11g072860 | 1.05 | 148.78 | 308.00 | Histone H4 |
| Solyc12g006530 | 1.05 | 226.60 | 468.49 | Uncharacterized protein |
| Solyc12g006530 | 1.05 | 226.60 | 468.49 | Uncharacterized protein |
| Solyc08g036640 | 1.05 | 97.70 | 201.94 | Tify domain-containing protein |
| Solyc08g036640 | 1.05 | 97.70 | 201.94 | Tify domain-containing protein |
| Solyc02g067830 | 1.05 | 99.99 | 206.60 | Protein kinase domain- |
| | | | | containing protein |
| Solyc02g067830 | 1.05 | 99.99 | 206.60 | Protein kinase domain- |
| | | | | containing protein |
| Solyc02g080480 | 1.05 | 186.00 | 384.23 | Protein DETOXIFICATION |
| | | | | (Multidrug and toxic compound |
| | | | | extrusion protein) |
| Solyc02g080480 | 1.05 | 186.00 | 384.23 | Protein DETOXIFICATION |
| | | | | (Multidrug and toxic compound |
| | | | | extrusion protein) |
| Solyc11g071800 | 1.05 | 189.97 | 392.09 | Str_synth domain-containing |
| | | | | protein |
| Solyc11g071800 | 1.05 | 189.97 | 392.09 | Str_synth domain-containing |
| | | | | protein |
| Solyc06g065690 | 1.04 | 168.49 | 347.45 | Serine/threonine protein |
| | | | | phosphatase 2A regulatory |
| | | | | subunit |
| Solyc06g065690 | 1.04 | 168.49 | 347.45 | Serine/threonine protein |
| | | | | phosphatase 2A regulatory |
| | | | | subunit |
| Solyc07g047830 | 1.04 | 162.37 | 334.43 | Uncharacterized protein |
| Solyc07g047830 | 1.04 | 162.37 | 334.43 | Uncharacterized protein |
| Solyc10g050970 | 1.04 | 504.45 | 1,038.49 | AP2/ERF domain-containing |
| | | | | protein |
| Solyc10g050970 | 1.04 | 504.45 | 1,038.49 | AP2/ERF domain-containing |
| | | | | protein |
| Solyc11g069990 | 1.04 | 124.29 | 255.87 | Disease resistance protein |
| | | | | (Fragment) |
| Solyc11g069990 | 1.04 | 124.29 | 255.87 | Disease resistance protein |
| | | | | (Fragment) |
| Solyc10g009290 | 1.04 | 66.45 | 136.38 | BHLH domain-containing |
| | | | | protein |
| Solyc10g009290 | 1.04 | 66.45 | 136.38 | BHLH domain-containing |
| | | | | protein |
| Solyc12g008470 | 1.04 | 102.49 | 210.29 | Cytochrome B5-like protein |
| Solyc12g008470 | 1.04 | 102.49 | 210.29 | Cytochrome B5-like protein |
| Solyc07g042300 | 1.04 | 105.57 | 216.61 | Dirigent protein |
| Solyc07g042300 | 1.04 | 105.57 | 216.61 | Dirigent protein |
| Solyc05g005950 | 1.04 | 422.14 | 865.56 | Uncharacterized protein |
| Solyc05g005950 | 1.04 | 422.14 | 865.56 | Uncharacterized protein |
| Solyc09g010210 | 1.04 | 133.10 | 272.82 | Endoglucanase (EC 3.2.1.4) |
| Solyc09g010210 | 1.04 | 133.10 | 272.82 | Endoglucanase (EC 3.2.1.4) |
| Solyc01g095470 | 1.03 | 84.61 | 173.31 | Glucose-6-phosphate 1- |
| | | | | epimerase (EC 5.1.3.15) |
| Solyc01g095470 | 1.03 | 84.61 | 173.31 | Glucose-6-phosphate 1- |
| | | | | epimerase (EC 5.1.3.15) |
| Solyc01g111210 | 1.03 | 70.92 | 145.22 | Uncharacterized protein |
| Solyc01g111210 | 1.03 | 70.92 | 145.22 | Uncharacterized protein |
| Solyc01g079580 | 1.03 | 207.81 | 425.41 | TPR_REGION domain- |
| | | | | containing protein |
| Solyc01g079580 | 1.03 | 207.81 | 425.41 | TPR_REGION domain- |
| | | | | containing protein |
| Solyc02g070260 | 1.03 | 388.75 | 795.50 | Uncharacterized protein |
| Solyc02g070260 | 1.03 | 388.75 | 795.50 | Uncharacterized protein |
| Solyc09g082510 | 1.03 | 869.56 | 1,778.74 | NAB domain-containing protein |
| Solyc09g082510 | 1.03 | 869.56 | 1,778.74 | NAB domain-containing protein |
| Solyc06g071320 | 1.03 | 99.08 | 202.53 | T10O24.15, related |
| Solyc06g071320 | 1.03 | 99.08 | 202.53 | T10O24.15, related |
| Solyc09g075480 | 1.03 | 80.68 | 164.90 | Uncharacterized protein |
| Solyc09g075480 | 1.03 | 80.68 | 164.90 | Uncharacterized protein |
| Solyc09g089860 | 1.03 | 363.00 | 740.57 | BHLH domain-containing |
| | | | | protein |
| Solyc09g089860 | 1.03 | 363.00 | 740.57 | BHLH domain-containing |
| | | | | protein |
| Solyc03g115000 | 1.03 | 384.03 | 783.18 | Uncharacterized protein |
| Solyc03g115000 | 1.03 | 384.03 | 783.18 | Uncharacterized protein |
| Solyc12g010730 | 1.03 | 104.72 | 213.36 | Uncharacterized protein |
| Solyc12g010730 | 1.03 | 104.72 | 213.36 | Uncharacterized protein |
| Solyc07g008900 | 1.03 | 369.17 | 752.18 | Uncharacterized protein |
| Solyc07g008900 | 1.03 | 369.17 | 752.18 | Uncharacterized protein |
| Solyc11g066430 | 1.03 | 87.07 | 177.41 | Histone H2B |
| Solyc11g066430 | 1.03 | 87.07 | 177.41 | Histone H2B |
| Solyc09g072860 | 1.03 | 75.67 | 154.04 | Uncharacterized protein |
| Solyc09g072860 | 1.03 | 75.67 | 154.04 | Uncharacterized protein |
| Solyc01g102350 | 1.02 | 619.33 | 1,259.02 | Pectin acetylesterase (EC 3.1.1.—) |
| Solyc01g102350 | 1.02 | 619.33 | 1,259.02 | Pectin acetylesterase (EC 3.1.1.—) |
| Solyc02g082140 | 1.02 | 439.87 | 893.35 | Glycosyltransferase family 92 |
| | | | | protein (EC 2.4.1.—) |
| Solyc02g082140 | 1.02 | 439.87 | 893.35 | Glycosyltransferase family 92 |
| | | | | protein (EC 2.4.1.—) |
| Solyc04g071610 | 1.02 | 2,171.19 | 4,407.68 | Ci21A protein |
| Solyc04g071610 | 1.02 | 2,171.19 | 4,407.68 | Ci21A protein |
| Solyc05g052410 | 1.02 | 140.49 | 284.98 | Dehydration responsive element |
| | | | | binding protein 2A-like protein |
| Solyc05g052410 | 1.02 | 140.49 | 284.98 | Dehydration responsive element |
| | | | | binding protein 2A-like protein |
| Solyc04g054880 | 1.02 | 106.88 | 216.73 | Uncharacterized protein |
| Solyc04g054880 | 1.02 | 106.88 | 216.73 | Uncharacterized protein |
| Solyc02g083340 | 1.02 | 387.75 | 786.17 | Phospholipase D (EC 3.1.4.4) |
| Solyc02g083340 | 1.02 | 387.75 | 786.17 | Phospholipase D (EC 3.1.4.4) |
| Solyc06g062400 | 1.02 | 87.84 | 177.95 | Uncharacterized protein |
| Solyc06g062400 | 1.02 | 87.84 | 177.95 | Uncharacterized protein |
| Solyc01g074000 | 1.02 | 63.39 | 128.40 | Protein kinase domain- |
| | | | | containing protein |
| Solyc01g074000 | 1.02 | 63.39 | 128.40 | Protein kinase domain- |
| | | | | containing protein |
| Solyc01g100760 | 1.01 | 164.85 | 332.86 | Uncharacterized protein |
| Solyc01g100760 | 1.01 | 164.85 | 332.86 | Uncharacterized protein |
| Solyc09g089930 | 1.01 | 147.28 | 297.36 | Transcription factor TSRF1 |
| Solyc09g089930 | 1.01 | 147.28 | 297.36 | Transcription factor TSRF 1 |
| Solyc01g096370 | 1.01 | 173.30 | 349.81 | BHLH domain-containing |
| | | | | protein |
| Solyc01g096370 | 1.01 | 173.30 | 349.81 | BHLH domain-containing |
| | | | | protein |
| Solyc01g109590 | 1.01 | 170.38 | 343.67 | Protein kinase domain- |
| | | | | containing protein |
| Solyc01g109590 | 1.01 | 170.38 | 343.67 | Protein kinase domain- |
| | | | | containing protein |
| Solyc04g051470 | 1.01 | 95.29 | 191.98 | Uncharacterized protein |
| Solyc04g051470 | 1.01 | 95.29 | 191.98 | Uncharacterized protein |
| Solyc05g018510 | 1.01 | 469.83 | 946.27 | Putative ABC transporter G |
| | | | | family member 32-like |
| Solyc05g018510 | 1.01 | 469.83 | 946.27 | Putative ABC transporter G |
| | | | | family member 32-like |
| Solyc05g008900 | 1.01 | 417.92 | 841.50 | Putative coproporphyrinogen- |
| | | | | III oxidase, aerobic-like |
| Solyc05g008900 | 1.01 | 417.92 | 841.50 | Putative coproporphyrinogen- |
| | | | | III oxidase, aerobic-like |
| Solyc11g021360 | 1.01 | 303.00 | 609.75 | Uncharacterized protein |
| Solyc11g021360 | 1.01 | 303.00 | 609.75 | Uncharacterized protein |
| Solyc12g014310 | 1.01 | 125.41 | 252.37 | Uncharacterized protein |
| Solyc12g014310 | 1.01 | 125.41 | 252.37 | Uncharacterized protein |
| Solyc04g071660 | 1.00 | 77.59 | 155.67 | Uncharacterized protein |
| Solyc04g071660 | 1.00 | 77.59 | 155.67 | Uncharacterized protein |
| Solyc02g088190 | 1.00 | 73.42 | 147.27 | Uncharacterized protein |
| Solyc02g088190 | 1.00 | 73.42 | 147.27 | Uncharacterized protein |
| Solyc10g045430 | 1.00 | 93.59 | 187.71 | Uncharacterized protein |
| Solyc10g045430 | 1.00 | 93.59 | 187.71 | Uncharacterized protein |
| Solyc06g071290 | 1.00 | 210.33 | 421.62 | Betaine-aldehyde |
| | | | | dehydrogenase, chloroplast, |
| | | | | putative |
| Solyc06g071290 | 1.00 | 210.33 | 421.62 | Betaine-aldehyde |
| | | | | dehydrogenase, chloroplast, |
| | | | | putative |
| Solyc01g106330 | 1.00 | 110.33 | 221.05 | Glutamyl-tRNA(Gln) |
| | | | | amidotransferase subunit A |
| Solyc01g106330 | 1.00 | 110.33 | 221.05 | Glutamyl-tRNA(Gln) |
| | | | | amidotransferase subunit A |
| Solyc07g007270 | 1.00 | 345.10 | 691.39 | Uncharacterized protein |
| Solyc07g007270 | 1.00 | 345.10 | 691.39 | Uncharacterized protein |
| Solyc07g064030 | 1.00 | 109.95 | 220.06 | Kinesin-like protein |
| Solyc07g064030 | 1.00 | 109.95 | 220.06 | Kinesin-like protein |
| Solyc01g100530 | 1.00 | 141.20 | 282.50 | Uncharacterized protein |
| Solyc01g100530 | 1.00 | 141.20 | 282.50 | Uncharacterized protein |

Table 6 depicts genes whose expression was upregulated by more than a 4-fold increase in treated plants compared to control plants.

TABLE 6
Unregulated Genes between Treated and Control F3 Tomato Plants (>4 fold increase)

| | | Control | Treatment | |
|---|---|---|---|---|
| Gene_ID | log2FoldChange | Expression | Expression | Protein Name |
| Solyc12g011010 | 5.39 | 2.20 | 92.49 | Uncharacterized protein |
| Solyc02g085110 | 5.24 | 0.74 | 27.86 | Laccase (EC 1.10.3.2) |
| | | | | (Benzenediol: oxygen |
| | | | | oxidoreductase) (Diphenol |
| | | | | oxidase) (Urishiol oxidase) |
| Solyc11g007870 | 5.07 | 0.73 | 24.49 | Uncharacterized protein |
| Solyc11g011300 | 4.94 | 0.37 | 11.36 | Rhamnogalacturonan |
| | | | | endolyase (EC 4.2.2.23) |
| Solyc01g109270 | 4.85 | 0.36 | 10.33 | Uncharacterized protein |
| Solyc04g076640 | 4.84 | 0.37 | 10.60 | Uncharacterized protein |
| | | | | (Fragment) |
| Solyc09g011970 | 4.75 | 0.37 | 9.94 | Laccase (EC 1.10.3.2) |
| | | | | (Benzenediol: oxygen |
| | | | | oxidoreductase) (Diphenol |
| | | | | oxidase) (Urishiol oxidase) |
| Solyc11g008830 | 4.70 | 0.36 | 9.30 | LOB domain-containing |
| | | | | protein 6 |
| Solyc04g054720 | 4.68 | 0.36 | 9.17 | Exonuclease/nucleic acid |
| | | | | binding |
| Solyc00g500095 | 4.67 | 25.37 | 644.34 | Phenylalanine ammonia-lyase |
| | | | | (PAL) (EC 4.3.1.24) |
| Solyc01g058100 | 4.59 | 0.37 | 8.91 | Uncharacterized protein |
| Solyc03g005210 | 4.57 | 0.37 | 8.77 | AAI domain-containing protein |
| Solyc03g033550 | 4.56 | 1.11 | 26.20 | Uncharacterized protein |
| Solyc04g071500 | 4.50 | 0.37 | 8.33 | Uncharacterized protein |
| Solyc07g052180 | 4.49 | 0.74 | 16.53 | NA |
| Solyc02g070070 | 4.45 | 1.11 | 24.15 | Reticuline oxidase |
| Solyc01g150144 | 4.44 | 0.37 | 8.00 | Epoxide hydrolase 1 |
| Solyc12g087940 | 4.42 | 1.08 | 23.28 | Peptidase A1 domain- |
| | | | | containing protein |
| Solyc12g005310 | 4.42 | 7.00 | 149.60 | Uncharacterized protein |
| Solyc09g008360 | 4.37 | 0.37 | 7.65 | DUF1624 domain-containing |
| | | | | protein |
| Solyc11g069840 | 4.37 | 0.74 | 15.29 | Uncharacterized protein |
| Solyc06g005070 | 4.37 | 1.08 | 22.42 | Uncharacterized protein |
| Solyc01g095250 | 4.37 | 0.73 | 15.02 | Glyco_hydro_18 domain- |
| | | | | containing protein |
| Solyc12g100150 | 4.37 | 0.73 | 15.01 | LOB domain-containing |
| | | | | protein |
| Solyc07g064990 | 4.35 | 4.37 | 88.96 | Uncharacterized protein |
| Solyc04g071680 | 4.32 | 0.37 | 7.36 | Protein kinase domain- |
| | | | | containing protein |
| Solyc07g064120 | 4.26 | 1.11 | 21.27 | Uncharacterized protein |
| Solyc10g007790 | 4.17 | 0.36 | 6.42 | TPR domain containing protein |
| Solyc12g088510 | 4.16 | 0.36 | 6.41 | Uncharacterized protein |
| Solyc09g075880 | 4.16 | 1.08 | 19.35 | Uncharacterized protein |
| Solyc06g053410 | 4.15 | 0.36 | 6.36 | Uncharacterized protein |
| Solyc08g081780 | 4.15 | 0.74 | 13.11 | Dirigent protein |
| Solyc09g064230 | 4.12 | 0.37 | 6.42 | Synaptotagmin |
| Solyc01g058540 | 4.11 | 0.37 | 6.37 | WRKY domain-containing |
| | | | | protein |
| Solyc06g054660 | 4.08 | 0.37 | 6.23 | Uncharacterized protein |
| Solyc03g034100 | 4.06 | 1.83 | 30.60 | Transcription repressor (Ovate |
| | | | | family protein) |
| Solyc05g023846 | 4.05 | 0.36 | 5.91 | Uncharacterized protein |
| Solyc08g074630 | 4.04 | 2.55 | 42.06 | Polyphenol oxidase F, |
| | | | | chloroplastic (PPO) (EC |
| | | | | 1.10.3.1) (Catechol oxidase) |
| Solyc02g072440 | 4.04 | 1.80 | 29.65 | Protein kinase domain- |
| | | | | containing protein |
| Solyc04g015540 | 4.04 | 0.73 | 11.95 | Uncharacterized protein |
| Solyc08g080870 | 4.01 | 0.37 | 5.95 | Putative copper-transporting |
| | | | | ATPase HMA5 |
| Solyc03g119350 | 3.99 | 0.74 | 11.76 | Uncharacterized protein |
| Solyc08g081790 | 3.99 | 1.10 | 17.37 | Dirigent protein |
| Solyc03g096030 | 3.98 | 2.20 | 34.76 | Protein ESKIMO 1-like |
| Solyc04g007823 | 3.98 | 1.44 | 22.70 | Bet_v_1 domain-containing |
| | | | | protein |
| Solyc02g030460 | 3.98 | 0.71 | 11.25 | Myb-like domain-containing |
| | | | | protein |
| Solyc11g068890 | 3.97 | 0.71 | 11.19 | LEA_2 domain-containing |
| | | | | protein |
| Solyc02g085630 | 3.96 | 0.37 | 5.76 | Homeobox domain-containing |
| | | | | protein |
| Solyc05g012240 | 3.95 | 0.36 | 5.52 | Uncharacterized protein |
| Solyc06g083720 | 3.94 | 0.36 | 5.49 | Uncharacterized protein |
| Solyc09g065490 | 3.93 | 0.37 | 5.64 | Uncharacterized protein |
| Solyc08g074683 | 3.92 | 0.71 | 10.81 | Polyphenol oxidase B, |
| | | | | chloroplastic (PPO) (EC |
| | | | | 1.10.3.1) (Catechol oxidase) |
| Solyc11g065840 | 3.92 | 0.37 | 5.58 | Uncharacterized protein |
| Solyc01g094870 | 3.92 | 2.18 | 32.90 | Uncharacterized protein |
| Solyc03g121170 | 3.91 | 1.10 | 16.46 | Uncharacterized protein |
| Solyc05g007300 | 3.91 | 0.37 | 5.55 | HVA22-like protein |
| Solyc08g014430 | 3.90 | 0.74 | 11.00 | Formin-like protein |
| Solyc03g120060 | 3.88 | 0.37 | 5.45 | Copia LTR rider |
| Solyc07g056360 | 3.87 | 0.37 | 5.41 | Uncharacterized protein |
| Solyc09g010260 | 3.87 | 0.37 | 5.39 | PMR5N domain-containing |
| | | | | protein |
| Solyc10g076830 | 3.86 | 1.48 | 21.41 | Laccase (EC 1.10.3.2) |
| | | | | (Benzenediol: oxygen |
| | | | | oxidoreductase) (Diphenol |
| | | | | oxidase) (Urishiol oxidase) |
| Solyc02g072490 | 3.84 | 2.58 | 37.12 | O-fucosyltransferase family |
| | | | | protein |
| Solyc09g075740 | 3.84 | 1.07 | 15.39 | Uncharacterized protein |
| Solyc07g055950 | 3.81 | 2.91 | 40.90 | Uncharacterized protein |
| Solyc07g049240 | 3.81 | 0.36 | 5.01 | Peroxidase (EC 1.11.1.7) |
| Solyc06g035580 | 3.80 | 8.06 | 112.68 | GMC_OxRdtase_N domain- |
| | | | | containing protein |
| Solyc05g015320 | 3.79 | 3.29 | 45.62 | PLE |
| Solyc09g083435 | 3.79 | 14.36 | 199.18 | Uncharacterized protein |
| Solyc01g111330 | 3.79 | 1.82 | 25.14 | Uncharacterized protein |
| Solyc01g091050 | 3.77 | 19.31 | 264.01 | Pectinesterase (EC 3.1.1.11) |
| Solyc10g076650 | 3.77 | 0.36 | 4.88 | Uncharacterized protein |
| Solyc09g062970 | 3.77 | 2.20 | 30.06 | NA |
| Solyc08g080280 | 3.76 | 0.37 | 4.99 | Uncharacterized protein |
| Solyc01g091320 | 3.75 | 17.35 | 234.16 | Fatty acid hydroxylase domain- |
| | | | | containing protein |
| Solyc02g086430 | 3.75 | 2.20 | 29.57 | Uncharacterized protein |
| Solyc01g010710 | 3.74 | 1.85 | 24.60 | Carboxypeptidase (EC 3.4.16.—) |
| Solyc08g061010 | 3.73 | 0.37 | 4.89 | Uncharacterized protein |
| Solyc08g076820 | 3.70 | 4.03 | 52.30 | Hop-interacting protein |
| | | | | THI018 |
| Solyc11g020630 | 3.69 | 0.71 | 9.23 | Uncharacterized protein |
| Solyc10g049270 | 3.69 | 1.10 | 14.11 | Uncharacterized protein |
| Solyc11g031950 | 3.68 | 2.55 | 32.60 | Polysacc_synt_4 domain- |
| | | | | containing protein |
| Solyc05g009580 | 3.66 | 0.73 | 9.19 | Uncharacterized protein |
| Solyc07g052100 | 3.65 | 0.74 | 9.27 | Uncharacterized protein |
| Solyc03g095750 | 3.65 | 1.85 | 23.19 | Uncharacterized protein |
| Solyc09g005630 | 3.63 | 2.93 | 36.35 | PMR5N domain-containing |
| | | | | protein |
| Solyc04g064610 | 3.59 | 1.81 | 21.84 | Bidirectional sugar transporter |
| | | | | SWEET |
| Solyc02g062170 | 3.58 | 2.18 | 26.15 | Uncharacterized protein |
| Solyc01g103700 | 3.58 | 1.11 | 13.24 | Uncharacterized protein |
| Solyc06g060320 | 3.58 | 1.10 | 13.09 | Uncharacterized protein |
| Solyc12g049500 | 3.57 | 1.48 | 17.55 | Uncharacterized protein |
| Solyc01g110180 | 3.55 | 1.83 | 21.45 | Uncharacterized protein |
| Solyc03g006010 | 3.54 | 1.11 | 12.85 | Uncharacterized protein |
| Solyc03g044600 | 3.53 | 0.74 | 8.55 | Uncharacterized protein |
| Solyc01g103360 | 3.53 | 5.89 | 68.18 | Polysacc_synt_4 domain- |
| | | | | containing protein |
| Solyc11g033281 | 3.53 | 1.83 | 21.16 | Uncharacterized protein |
| Solyc09g008380 | 3.52 | 5.81 | 66.89 | Pectate lyase (EC 4.2.2.2) |
| Solyc06g009190 | 3.52 | 145.74 | 1,673.93 | Pectinesterase (EC 3.1.1.11) |
| Solyc12g007040 | 3.52 | 1.10 | 12.58 | Uncharacterized protein |
| Solyc03g006840 | 3.51 | 5.10 | 58.00 | Uncharacterized protein |
| Solyc01g057165 | 3.49 | 1.07 | 12.08 | Uncharacterized protein |
| Solyc10g083580 | 3.49 | 1.46 | 16.49 | Phytosulfokine-beta |
| Solyc10g009480 | 3.49 | 2.22 | 24.93 | Uncharacterized protein |
| Solyc11g056680 | 3.49 | 2.51 | 28.25 | LRRNT_2 domain-containing |
| | | | | protein |
| Solyc03g070440 | 3.49 | 4.80 | 53.85 | Protein COBRA |
| Solyc05g005980 | 3.48 | 7.28 | 81.41 | Putative peptide transporter- |
| | | | | like |
| Solyc11g071520 | 3.47 | 1.85 | 20.52 | Uncharacterized protein |
| Solyc05g009270 | 3.47 | 6.58 | 73.05 | 3-ketoacyl-CoA synthase (EC |
| | | | | 2.3.1.—) |
| Solyc03g006545 | 3.47 | 0.73 | 8.05 | Putative geranyllinalool |
| | | | | synthase |
| Solyc01g091180 | 3.46 | 5.12 | 56.45 | Uncharacterized protein |
| Solyc05g010080 | 3.43 | 7.71 | 82.81 | Uncharacterized protein |
| Solyc01g090870 | 3.42 | 0.74 | 7.89 | OVATE domain-containing |
| | | | | protein |
| Solyc02g089210 | 3.42 | 6.98 | 74.50 | Uncharacterized protein |
| Solyc03g034380 | 3.41 | 1.82 | 19.36 | AAI domain-containing protein |
| Solyc10g006500 | 3.37 | 2.94 | 30.47 | Hexosyltransferase (EC 2.4.1.—) |
| Solyc03g026040 | 3.36 | 3.61 | 37.02 | Protein kinase domain- |
| | | | | containing protein |
| Solyc02g078040 | 3.35 | 16.45 | 167.86 | Uncharacterized protein |
| Solyc12g089050 | 3.35 | 1.48 | 15.03 | MBOAT_2 domain-containing |
| | | | | protein |
| Solyc09g007420 | 3.34 | 4.42 | 44.81 | Glycosyltransferases (EC 2.4.—.—) |
| Solyc06g067910 | 3.34 | 1.45 | 14.69 | Uncharacterized protein |
| Solyc08g066810 | 3.33 | 1.48 | 14.90 | Mannan endo-1,4-beta- |
| | | | | mannosidase (EC 3.2.1.78) |
| Solyc07g005900 | 3.33 | 1.48 | 14.88 | Uncharacterized protein |
| Solyc09g009670 | 3.33 | 0.71 | 7.18 | TFIID_20 kDa domain- |
| | | | | containing protein |
| Solyc03g053130 | 3.32 | 0.74 | 7.39 | Str_synth domain-containing |
| | | | | protein |
| Solyc09g091390 | 3.32 | 1.11 | 11.07 | Uncharacterized protein |
| Solyc02g084210 | 3.31 | 1.10 | 10.88 | GDP-mannose 4,6-dehydratase |
| | | | | (EC 4.2.1.47) |
| Solyc08g067940 | 3.31 | 0.73 | 7.21 | Uncharacterized protein |
| Solyc03g114380 | 3.29 | 2.56 | 25.13 | Kinesin-like protein |
| Solyc09g075630 | 3.29 | 1.48 | 14.48 | Uncharacterized protein |
| Solyc09g066300 | 3.29 | 1.10 | 10.73 | RING-type domain-containing |
| | | | | protein |
| Solyc09g007140 | 3.29 | 4.74 | 46.34 | SHSP domain-containing |
| | | | | protein |
| Solyc04g015010 | 3.29 | 1.11 | 10.83 | Uncharacterized protein |
| Solyc03g025790 | 3.29 | 6.88 | 67.15 | Copper ion binding protein |
| Solyc03g083770 | 3.28 | 714.41 | 6,947.78 | PMEI domain-containing |
| | | | | protein |
| Solyc04g074810 | 3.27 | 0.74 | 7.13 | BHLH domain-containing |
| | | | | protein |
| Solyc05g008895 | 3.26 | 53.09 | 507.92 | Nonspecific lipid-transfer |
| | | | | protein |
| Solyc04g051420 | 3.25 | 7.36 | 70.19 | Uncharacterized protein |
| Solyc02g094050 | 3.25 | 8.75 | 83.44 | Uncharacterized protein |
| Solyc12g088220 | 3.25 | 1.11 | 10.51 | Branched-chain-amino-acid |
| | | | | aminotransferase (EC 2.6.1.42) |
| Solyc10g085610 | 3.24 | 9.47 | 89.51 | Uncharacterized protein |
| Solyc06g061023 | 3.24 | 16.40 | 154.78 | Putative cytochrome P450 |
| Solyc06g068910 | 3.23 | 10.22 | 95.66 | Protein kinase domain- |
| | | | | containing protein |
| Solyc05g052400 | 3.22 | 4.33 | 40.51 | Laccase (EC 1.10.3.2) |
| | | | | (Benzenediol: oxygen |
| | | | | oxidoreductase) (Diphenol |
| | | | | oxidase) (Urishiol oxidase) |
| Solyc11g069250 | 3.22 | 6.93 | 64.52 | FAS1 domain-containing |
| | | | | protein |
| Solyc06g082960 | 3.22 | 2.58 | 24.00 | Uncharacterized protein |
| Solyc03g114430 | 3.21 | 1.08 | 10.04 | Uncharacterized protein |
| Solyc06g150127 | 3.21 | 0.74 | 6.83 | NA |
| Solyc07g055100 | 3.20 | 1.83 | 16.88 | 6b-interacting protein 1 |
| Solyc03g123390 | 3.20 | 3.30 | 30.30 | AB hydrolase-1 domain- |
| | | | | containing protein |
| Solyc10g009240 | 3.19 | 30.98 | 283.67 | 3-ketoacyl-CoA synthase (EC |
| | | | | 2.3.1.—) |
| Solyc05g008670 | 3.19 | 1.10 | 10.00 | Uncharacterized protein |
| Solyc02g066970 | 3.18 | 2.58 | 23.49 | Uncharacterized protein |
| Solyc09g011960 | 3.18 | 4.06 | 36.82 | Laccase (EC 1.10.3.2) |
| | | | | (Benzenediol: oxygen |
| | | | | oxidoreductase) (Diphenol |
| | | | | oxidase) (Urishiol oxidase) |
| Solyc06g062370 | 3.18 | 17.27 | 156.44 | Uncharacterized protein |
| Solyc03g033280 | 3.18 | 1.08 | 9.80 | Uncharacterized protein |
| Solyc09g089540 | 3.17 | 6.19 | 55.84 | Uncharacterized protein |
| Solyc05g008890 | 3.17 | 3.64 | 32.86 | Reticulon-like protein |
| Solyc06g075220 | 3.16 | 9.49 | 84.88 | FAS1 domain-containing |
| | | | | protein |
| Solyc07g054310 | 3.15 | 0.74 | 6.57 | Uncharacterized protein |
| Solyc09g065570 | 3.15 | 1.10 | 9.75 | Protein kinase domain- |
| | | | | containing protein |
| Solyc06g150133 | 3.15 | 5.48 | 48.68 | Cysteine proteinase inhibitor |
| | | | | (Fragment) |
| Solyc06g071930 | 3.15 | 2.18 | 19.35 | Uncharacterized protein |
| Solyc09g008750 | 3.15 | 2.55 | 22.56 | VQ domain-containing protein |
| Solyc04g005320 | 3.14 | 1.83 | 16.20 | Transcription factor, K-box |
| Solyc01g066457 | 3.14 | 3.69 | 32.50 | AB hydrolase-1 domain- |
| | | | | containing protein |
| Solyc10g075100 | 3.13 | 10.61 | 92.72 | Non-specific lipid-transfer |
| | | | | protein |
| Solyc11g069970 | 3.13 | 1.83 | 16.02 | Uncharacterized protein |
| Solyc03g083180 | 3.13 | 1.82 | 15.89 | Uncharacterized protein |
| Solyc08g016270 | 3.12 | 11.64 | 101.50 | Uncharacterized protein |
| Solyc03g007050 | 3.12 | 28.00 | 244.04 | Protein kinase domain- |
| | | | | containing protein |
| Solyc08g006020 | 3.12 | 0.74 | 6.44 | Transcription factor |
| Solyc06g034360 | 3.12 | 0.74 | 6.40 | Pectinesterase (EC 3.1.1.11) |
| Solyc01g090120 | 3.10 | 2.52 | 21.66 | Phytocyanin domain- |
| | | | | containing protein |
| Solyc02g091230 | 3.09 | 0.73 | 6.17 | RING-CH-type domain- |
| | | | | containing protein |
| Solyc04g007820 | 3.08 | 6.16 | 52.08 | Bet_v_1 domain-containing |
| | | | | protein |
| Solyc11g011000 | 3.08 | 1.44 | 12.19 | Uncharacterized protein |
| Solyc06g051680 | 3.07 | 80.29 | 675.84 | Elf4 domain-containing protein |
| Solyc09g009860 | 3.07 | 2.57 | 21.62 | Glutaredoxin domain- |
| | | | | containing protein |
| Solyc07g042390 | 3.07 | 2.57 | 21.61 | PMEI domain-containing |
| | | | | protein |
| Solyc10g076880 | 3.07 | 3.29 | 27.62 | SHSP domain-containing |
| | | | | protein |
| Solyc06g034340 | 3.07 | 2.55 | 21.41 | NAC domain-containing |
| | | | | protein |
| Solyc04g079990 | 3.07 | 4.75 | 39.90 | Uncharacterized protein |
| Solyc02g069440 | 3.07 | 4.03 | 33.79 | LOB domain-containing |
| | | | | protein |
| Solyc02g032840 | 3.05 | 2.56 | 21.24 | NA |
| Solyc07g053530 | 3.05 | 1.08 | 8.99 | FAS1 domain-containing |
| | | | | protein |
| Solyc09g090550 | 3.04 | 4.73 | 39.00 | Arp2/3 complex 34 kDa |
| | | | | subunit |
| Solyc02g079070 | 3.04 | 2.57 | 21.10 | Uncharacterized protein |
| Solyc03g098790 | 3.03 | 2.22 | 18.13 | Cathepsin D Inhibitor |
| Solyc02g081270 | 3.03 | 5.17 | 42.09 | NAC domain-containing |
| | | | | protein |
| Solyc11g069540 | 3.03 | 6.24 | 50.80 | TPT domain-containing protein |
| Solyc06g036460 | 3.02 | 3.26 | 26.55 | Uncharacterized protein |
| Solyc08g014380 | 3.01 | 1.43 | 11.54 | Pectin acetylesterase (EC |
| | | | | 3.1.1.—) |
| Solyc09g014240 | 3.01 | 19.03 | 153.22 | Laccase (EC 1.10.3.2) |
| | | | | (Benzenediol: oxygen |
| | | | | oxidoreductase) (Diphenol |
| | | | | oxidase) (Urishiol oxidase) |
| Solyc08g080070 | 3.01 | 4.04 | 32.47 | Uncharacterized protein |
| Solyc10g005550 | 3.01 | 1.08 | 8.71 | R2r3-myb transcription factor |
| Solyc06g071610 | 2.99 | 4.39 | 34.97 | Uncharacterized protein |
| Solyc02g085120 | 2.99 | 9.16 | 72.69 | Laccase (EC 1.10.3.2) |
| | | | | (Benzenediol: oxygen |
| | | | | oxidoreductase) (Diphenol |
| | | | | oxidase) (Urishiol oxidase) |
| Solyc05g005540 | 2.99 | 2.88 | 22.83 | BURP domain-containing |
| | | | | protein |
| Solyc10g076570 | 2.99 | 1.85 | 14.62 | PMR5N domain-containing |
| | | | | protein |
| Solyc10g076730 | 2.98 | 8.00 | 63.25 | PMEI domain-containing |
| | | | | protein |
| Solyc08g067505 | 2.97 | 2.93 | 23.00 | AAI domain-containing protein |
| Solyc03g083050 | 2.97 | 7.35 | 57.68 | Uncharacterized protein |
| Solyc10g075110 | 2.97 | 73.55 | 577.08 | Non-specific lipid-transfer |
| | | | | protein |
| Solyc12g005300 | 2.97 | 30.86 | 241.34 | Uncharacterized protein |
| Solyc03g114560 | 2.97 | 16.26 | 127.05 | Uncharacterized protein |
| Solyc08g081710 | 2.96 | 3.30 | 25.64 | Uncharacterized protein |
| Solyc09g075360 | 2.96 | 12.40 | 96.28 | Endoglucanase (EC 3.2.1.4) |
| Solyc01g005290 | 2.95 | 19.76 | 152.91 | SEC14 cytosolic factor/ |
| | | | | phosphoglyceride transfer |
| | | | | family protein |
| Solyc06g053440 | 2.95 | 1.79 | 13.82 | Uncharacterized protein |
| Solyc05g005570 | 2.95 | 48.87 | 377.86 | Uncharacterized protein |
| Solyc01g068140 | 2.95 | 2.20 | 17.03 | Uncharacterized protein |
| Solyc02g036430 | 2.95 | 3.28 | 25.31 | NAC domain-containing |
| | | | | protein |
| Solyc01g103070 | 2.95 | 1.82 | 14.04 | Uncharacterized protein |
| Solyc03g098060 | 2.94 | 2.58 | 19.82 | ZF-HD dimerization-type |
| | | | | domain-containing protein |
| Solyc01g006660 | 2.94 | 6.27 | 48.12 | Uncharacterized protein |
| Solyc10g048190 | 2.94 | 1.10 | 8.39 | EVE domain-containing |
| | | | | protein |
| Solyc05g056620 | 2.94 | 2.89 | 22.16 | APETALA1 protein |
| Solyc07g014730 | 2.93 | 17.26 | 131.78 | Uncharacterized protein |
| Solyc11g066120 | 2.93 | 1.07 | 8.16 | Uncharacterized protein |
| Solyc02g087470 | 2.93 | 16.34 | 124.39 | Uncharacterized protein |
| Solyc02g072240 | 2.91 | 25.31 | 190.09 | Cellulose synthase (EC |
| | | | | 2.4.1.12) |
| Solyc10g055260 | 2.90 | 5.43 | 40.55 | LAX5 protein |
| Solyc03g118370 | 2.90 | 36.76 | 274.21 | Carboxypeptidase (EC 3.4.16.—) |
| Solyc04g079560 | 2.90 | 65.29 | 486.16 | Aa_trans domain-containing |
| | | | | protein |
| Solyc03g114830 | 2.89 | 14.61 | 108.56 | MADS-box domain-containing |
| | | | | protein |
| Solyc10g085190 | 2.89 | 5.49 | 40.62 | Fe2OG dioxygenase domain- |
| | | | | containing protein |
| Solyc10g079530 | 2.89 | 1.47 | 10.84 | Uncharacterized protein |
| Solyc06g054460 | 2.88 | 3.63 | 26.78 | S-adenosylmethionine |
| | | | | decarboxylase proenzyme (EC |
| | | | | 4.1.1.50) |
| Solyc06g062430 | 2.88 | 9.13 | 67.28 | Inositol oxygenase (EC |
| | | | | 1.13.99.1) (Myo-inositol |
| | | | | oxygenase) |
| Solyc09g005940 | 2.88 | 1.46 | 10.78 | Fatty acyl-CoA reductase (EC |
| | | | | 1.2.1.84) |
| Solyc02g089130 | 2.87 | 6.62 | 48.30 | COBRA-like protein |
| Solyc04g050730 | 2.87 | 8.00 | 58.35 | Lipase_GDSL domain- |
| | | | | containing protein |
| Solyc09g084480 | 2.87 | 2.93 | 21.35 | Type I serine protease inhibitor |
| Solyc09g057950 | 2.86 | 3.24 | 23.48 | Asparagine--tRNA ligase (EC |
| | | | | 6.1.1.22) |
| Solyc02g070660 | 2.86 | 1.08 | 7.85 | Cytochrome b561 domain- |
| | | | | containing protein |
| Solyc06g064820 | 2.86 | 1.10 | 7.93 | Lipase_GDSL domain- |
| | | | | containing protein |
| Solyc01g060450 | 2.85 | 1.44 | 10.42 | Uncharacterized protein |
| Solyc05g014000 | 2.85 | 17.92 | 129.51 | Pectate lyase (EC 4.2.2.2) |
| Solyc03g120380 | 2.85 | 22.28 | 160.80 | Auxin-responsive protein |
| Solyc08g078840 | 2.85 | 30.32 | 218.75 | Uncharacterized protein |
| Solyc12g044630 | 2.85 | 3.26 | 23.46 | Profilin-1 |
| Solyc09g072820 | 2.84 | 36.03 | 258.11 | Protein kinase domain- |
| | | | | containing protein |
| Solyc03g120790 | 2.84 | 3.29 | 23.49 | OVATE domain-containing |
| | | | | protein |
| Solyc08g066260 | 2.83 | 1.11 | 7.88 | Uncharacterized protein |
| Solyc09g009010 | 2.83 | 6.92 | 49.10 | Putative glucomannan 4-beta- |
| | | | | mannosyltransferase 9-like |
| Solyc03g079960 | 2.82 | 2.89 | 20.41 | Uncharacterized protein |
| Solyc05g052340 | 2.81 | 1.11 | 7.78 | Laccase (EC 1.10.3.2) |
| | | | | (Benzenediol: oxygen |
| | | | | oxidoreductase) (Diphenol |
| | | | | oxidase) (Urishiol oxidase) |
| Solyc09g098060 | 2.81 | 2.18 | 15.31 | Uncharacterized protein |
| Solyc10g008710 | 2.81 | 5.17 | 36.27 | Lipase_GDSL domain- |
| | | | | containing protein |
| Solyc06g053760 | 2.81 | 9.54 | 66.82 | t-SNARE coiled-coil homology |
| | | | | domain-containing protein |
| Solyc03g098795 | 2.80 | 14.01 | 97.81 | Uncharacterized protein |
| Solyc01g090310 | 2.80 | 1.10 | 7.64 | ERF1 |
| Solyc12g005480 | 2.79 | 3.66 | 25.33 | Uncharacterized protein |
| Solyc05g050090 | 2.79 | 16.93 | 117.05 | Cytochrome b5 heme-binding |
| | | | | domain-containing protein |
| Solyc07g007240 | 2.79 | 7.30 | 50.33 | CarbpepA_inh domain- |
| | | | | containing protein |
| Solyc04g045565 | 2.78 | 34.82 | 239.57 | Retrotransposon-related protein |
| Solyc06g071640 | 2.78 | 1.83 | 12.61 | Alliinase_C domain-containing |
| | | | | protein |
| Solyc01g100890 | 2.78 | 2.18 | 14.97 | WAT1-related protein |
| Solyc02g071200 | 2.78 | 3.29 | 22.57 | X8 domain-containing protein |
| Solyc06g066020 | 2.78 | 11.73 | 80.47 | Auxin-responsive protein |
| Solyc11g007330 | 2.77 | 5.82 | 39.84 | RAB6-interacting golgin |
| Solyc06g068040 | 2.77 | 13.90 | 95.04 | Uncharacterized protein |
| Solyc03g115770 | 2.77 | 33.62 | 229.24 | Uncharacterized protein |
| Solyc08g067000 | 2.76 | 10.28 | 69.79 | Uncharacterized protein |
| Solyc07g043400 | 2.76 | 7.63 | 51.60 | Uncharacterized protein |
| Solyc12g042580 | 2.74 | 33.55 | 223.81 | Phytocyanin domain- |
| | | | | containing protein |
| Solyc02g093740 | 2.73 | 2.86 | 18.99 | Uncharacterized protein |
| Solyc07g053590 | 2.73 | 4.01 | 26.64 | NAC domain-containing |
| | | | | protein |
| Solyc06g007960 | 2.72 | 7.68 | 50.66 | Uncharacterized protein |
| Solyc04g013200 | 2.72 | 1.85 | 12.17 | Uncharacterized protein |
| Solyc07g065110 | 2.72 | 2.18 | 14.36 | AAI domain-containing protein |
| Solyc01g109750 | 2.72 | 2.20 | 14.52 | t-SNARE coiled-coil homology |
| | | | | domain-containing protein |
| Solyc11g069390 | 2.71 | 2.54 | 16.65 | Uncharacterized protein |
| Solyc02g067930 | 2.71 | 4.01 | 26.29 | Glycerol-3-phosphate |
| | | | | dehydrogenase [NAD(+)] (EC |
| | | | | 1.1.1.8) |
| Solyc01g090730 | 2.71 | 10.63 | 69.52 | SBP-type domain-containing |
| | | | | protein |
| Solyc03g006710 | 2.71 | 3.27 | 21.36 | EF-hand domain-containing |
| | | | | protein |
| Solyc08g075240 | 2.71 | 2.16 | 14.06 | Long-chain-alcohol oxidase |
| | | | | (EC 1.1.3.20) |
| Solyc02g086180 | 2.70 | 31.72 | 206.70 | Fatty acid hydroxylase domain- |
| | | | | containing protein |
| Solyc01g091850 | 2.70 | 10.22 | 66.33 | Uncharacterized protein |
| Solyc06g150109 | 2.70 | 1.45 | 9.42 | Mads-box protein agl42 |
| Solyc05g010680 | 2.70 | 26.57 | 172.14 | Hydrolase_4 domain- |
| | | | | containing protein |
| Solyc03g111280 | 2.69 | 1.81 | 11.69 | Uncharacterized protein |
| Solyc04g074740 | 2.69 | 2.55 | 16.40 | Phytocyanin domain- |
| | | | | containing protein |
| Solyc10g008410 | 2.68 | 17.64 | 113.32 | Uncharacterized protein |
| Solyc10g007820 | 2.68 | 4.35 | 27.83 | HVA22-like protein |
| Solyc06g069790 | 2.68 | 17.17 | 109.92 | gibberellin-regulated protein 6- |
| | | | | like |
| Solyc07g064050 | 2.68 | 6.62 | 42.37 | Hexosyltransferase (EC 2.4.1.—) |
| Solyc03g025680 | 2.68 | 1.44 | 9.22 | Uncharacterized protein |
| Solyc06g050920 | 2.66 | 12.39 | 78.34 | PMR5N domain-containing |
| | | | | protein |
| Solyc12g010700 | 2.66 | 2.94 | 18.57 | Uncharacterized protein |
| Solyc09g010995 | 2.65 | 1.82 | 11.42 | Laccase (EC 1.10.3.2) |
| | | | | (Benzenediol: oxygen |
| | | | | oxidoreductase) (Diphenol |
| | | | | oxidase) (Urishiol oxidase) |
| Solyc02g071400 | 2.65 | 1.43 | 8.96 | Uncharacterized protein |
| Solyc10g049580 | 2.65 | 59.29 | 371.28 | NA |
| Solyc05g012350 | 2.64 | 2.89 | 18.08 | Hydrolase_4 domain- |
| | | | | containing protein |
| Solyc01g086920 | 2.64 | 114.26 | 713.66 | LRRNT_2 domain-containing |
| | | | | protein |
| Solyc03g006810 | 2.63 | 5.51 | 34.17 | Peroxidase (EC 1.11.1.7) |
| Solyc06g007140 | 2.63 | 3.99 | 24.70 | Uncharacterized protein |
| Solyc12g094700 | 2.63 | 11.04 | 68.21 | Uncharacterized protein |
| Solyc09g075790 | 2.63 | 5.08 | 31.40 | 4-coumarate--CoA ligase (EC |
| | | | | 6.2.1.12) |
| Solyc09g008320 | 2.63 | 28.25 | 174.34 | Xyloglucan |
| | | | | endotransglucosylase/hydrolase |
| | | | | (EC 2.4.1.207) |
| Solyc06g074040 | 2.62 | 5.50 | 33.93 | LEA_2 domain-containing |
| | | | | protein |
| Solyc04g014360 | 2.62 | 42.43 | 260.68 | BHLH domain-containing |
| | | | | protein |
| Solyc01g107260 | 2.62 | 5.53 | 33.94 | Uncharacterized protein |
| Solyc06g043150 | 2.62 | 7.31 | 44.89 | Cell cycle switch 52B |
| Solyc07g005840 | 2.61 | 34.09 | 208.73 | Cellulose synthase (EC |
| | | | | 2.4.1.12) |
| Solyc02g080330 | 2.61 | 1.48 | 9.04 | Uncharacterized protein |
| Solyc03g113680 | 2.61 | 99.30 | 606.65 | Uncharacterized protein |
| Solyc10g006920 | 2.61 | 1.45 | 8.87 | CBS domain-containing |
| | | | | protein |
| Solyc11g069730 | 2.61 | 31.47 | 191.72 | Uncharacterized protein |
| Solyc06g062380 | 2.61 | 1.44 | 8.77 | Uncharacterized protein |
| Solyc10g150149 | 2.60 | 1.46 | 8.91 | Uncharacterized protein |
| Solyc04g009110 | 2.60 | 1.43 | 8.69 | Uncharacterized protein |
| Solyc01g096980 | 2.60 | 8.00 | 48.40 | SHSP domain-containing |
| | | | | protein |
| Solyc10g076720 | 2.59 | 6.56 | 39.58 | Uncharacterized protein |
| Solyc12g014360 | 2.59 | 15.31 | 92.35 | PMR5N domain-containing |
| | | | | protein |
| Solyc06g073587 | 2.59 | 6.91 | 41.59 | Cysteine proteinase inhibitor |
| | | | | (Fragment) |
| Solyc11g008810 | 2.59 | 24.94 | 150.06 | Beta-hexosaminidase (EC |
| | | | | 3.2.1.52) |
| Solyc11g045410 | 2.59 | 2.22 | 13.31 | Uncharacterized protein |
| Solyc10g053910 | 2.59 | 5.84 | 35.05 | Endoplasmic reticulum |
| | | | | transmembrane protein |
| Solyc02g084850 | 2.58 | 15.53 | 92.88 | NA |
| Solyc10g005460 | 2.58 | 2.54 | 15.17 | Uncharacterized protein |
| Solyc02g068400 | 2.58 | 6.22 | 37.15 | Pectate_lyase_3 domain- |
| | | | | containing protein |
| Solyc06g052050 | 2.57 | 1.45 | 8.65 | Uncharacterized protein |
| Solyc04g008850 | 2.57 | 28.91 | 172.00 | Peptidase A1 domain- |
| | | | | containing protein |
| Solyc06g083650 | 2.57 | 45.38 | 269.28 | Lipase_GDSL domain- |
| | | | | containing protein |
| Solyc04g080520 | 2.57 | 13.18 | 78.12 | Uncharacterized protein |
| Solyc06g073350 | 2.57 | 1.48 | 8.74 | Uncharacterized protein |
| Solyc10g048030 | 2.56 | 34.51 | 204.04 | Bet_v_1 domain-containing |
| | | | | protein |
| Solyc05g050240 | 2.56 | 2.57 | 15.20 | UBC core domain-containing |
| | | | | protein |
| Solyc11g005820 | 2.56 | 1.81 | 10.66 | PMEI domain-containing |
| | | | | protein |
| Solyc07g049300 | 2.55 | 7.68 | 44.98 | Endoglucanase (EC 3.2.1.4) |
| Solyc03g026050 | 2.55 | 17.14 | 100.17 | Uncharacterized protein |
| Solyc04g009150 | 2.54 | 1.82 | 10.62 | Uncharacterized protein |
| Solyc09g092750 | 2.54 | 1.82 | 10.61 | NA |
| Solyc10g080870 | 2.54 | 8.35 | 48.54 | Uncharacterized protein |
| Solyc10g086310 | 2.54 | 8.40 | 48.77 | AtRab GTP-binding protein |
| Solyc07g063350 | 2.54 | 20.40 | 118.36 | Uncharacterized protein |
| Solyc02g080100 | 2.54 | 1.81 | 10.50 | LEA_2 domain-containing |
| | | | | protein |
| Solyc01g106540 | 2.54 | 4.37 | 25.34 | DUF295 domain-containing |
| | | | | protein |
| Solyc12g096110 | 2.53 | 5.11 | 29.53 | protein BREVIS RADIX-like |
| Solyc04g070960 | 2.53 | 2.20 | 12.74 | Dof zinc finger protein 9 |
| Solyc05g052650 | 2.53 | 2.94 | 17.00 | Uncharacterized protein |
| Solyc08g062760 | 2.52 | 16.48 | 94.77 | Uncharacterized protein |
| Solyc06g005130 | 2.52 | 12.78 | 73.40 | Dof-type domain-containing |
| | | | | protein |
| Solyc02g063030 | 2.52 | 52.61 | 302.10 | Uncharacterized protein |
| Solyc01g109370 | 2.52 | 15.69 | 90.02 | 1-acylglycerol-3-phosphate O- |
| | | | | acyltransferase (EC 2.3.1.51) |
| Solyc01g109430 | 2.52 | 6.18 | 35.46 | deSI-like protein At4g17486 |
| Solyc06g068130 | 2.52 | 4.69 | 26.90 | TPR_REGION domain- |
| | | | | containing protein |
| Solyc03g098290 | 2.51 | 27.50 | 156.71 | Sucrose synthase (EC 2.4.1.13) |
| Solyc04g080420 | 2.50 | 25.90 | 146.90 | Uncharacterized protein |
| Solyc09g075900 | 2.50 | 124.65 | 704.08 | PABS domain-containing |
| | | | | protein |
| Solyc03g078830 | 2.49 | 13.18 | 74.12 | Uncharacterized protein |
| Solyc11g010690 | 2.49 | 53.93 | 302.43 | Uncharacterized protein |
| Solyc11g007880 | 2.48 | 2.95 | 16.50 | Self-pruning interacting protein |
| | | | | 1 |
| Solyc06g069590 | 2.48 | 1.83 | 10.22 | Remorin_C domain-containing |
| | | | | protein |
| Solyc02g078850 | 2.48 | 17.55 | 97.69 | Uncharacterized protein |
| Solyc12g015800 | 2.47 | 8.81 | 48.89 | RING-type domain-containing |
| | | | | protein |
| Solyc04g077740 | 2.47 | 4.00 | 22.14 | Uncharacterized protein |
| Solyc08g075080 | 2.47 | 6.91 | 38.21 | Ring finger protein |
| Solyc08g061180 | 2.47 | 2.20 | 12.17 | Uncharacterized protein |
| Solyc01g111310 | 2.46 | 190.01 | 1,047.01 | LAX2 protein |
| Solyc04g010200 | 2.46 | 2.18 | 12.01 | ABC transporter domain- |
| | | | | containing protein |
| Solyc00g500254 | 2.46 | 2.17 | 11.94 | 50S ribosomal protein L20, |
| | | | | chloroplastic |
| Solyc07g054560 | 2.46 | 6.24 | 34.34 | Uncharacterized protein |
| Solyc07g008010 | 2.46 | 8.10 | 44.53 | Uncharacterized protein |
| Solyc08g067180 | 2.46 | 41.28 | 226.78 | Uncharacterized protein |
| Solyc11g008880 | 2.45 | 4.37 | 23.92 | CRIB domain-containing |
| | | | | protein |
| Solyc03g005020 | 2.45 | 11.64 | 63.52 | Lipase |
| Solyc09g008860 | 2.45 | 20.09 | 109.61 | Protein kinase domain- |
| | | | | containing protein |
| Solyc06g006110 | 2.45 | 84.08 | 458.26 | Vacuolar cation/proton |
| | | | | exchanger |
| Solyc02g078130 | 2.44 | 12.11 | 65.85 | BHLH domain-containing |
| | | | | protein |
| Solyc10g086620 | 2.43 | 48.91 | 264.20 | Uncharacterized protein |
| Solyc11g069115 | 2.43 | 2.55 | 13.73 | Uncharacterized protein |
| Solyc01g100980 | 2.43 | 81.90 | 440.17 | Uncharacterized protein |
| Solyc03g120950 | 2.43 | 3.30 | 17.72 | Uncharacterized protein |
| Solyc02g091850 | 2.43 | 3.27 | 17.59 | NA |
| Solyc08g083240 | 2.42 | 26.22 | 140.74 | DUF4005 domain-containing |
| | | | | protein |
| Solyc08g006710 | 2.42 | 7.68 | 41.18 | Uncharacterized protein |
| Solyc10g074550 | 2.42 | 3.62 | 19.41 | NA |
| Solyc12g096980 | 2.42 | 22.21 | 119.01 | Auxin-responsive protein |
| Solyc09g065620 | 2.42 | 4.05 | 21.70 | Uncharacterized protein |
| Solyc08g079880 | 2.42 | 15.07 | 80.60 | p69C protein |
| Solyc09g072690 | 2.42 | 25.03 | 133.81 | Uncharacterized protein |
| Solyc08g079270 | 2.42 | 1.82 | 9.74 | Uncharacterized protein |
| Solyc09g007660 | 2.42 | 6.60 | 35.23 | FAS1 domain-containing |
| | | | | protein |
| Solyc03g120290 | 2.42 | 5.15 | 27.47 | Uncharacterized protein |
| Solyc03g020030 | 2.42 | 21.87 | 116.70 | Uncharacterized protein |
| Solyc08g080890 | 2.41 | 5.88 | 31.32 | Uncharacterized protein |
| Solyc03g046200 | 2.41 | 6.20 | 33.00 | Endo-1,3(4)-beta-glucanase |
| | | | | (EC 3.2.1.6) |
| Solyc03g116370 | 2.41 | 2.17 | 11.53 | AtEB1b |
| Solyc05g009990 | 2.41 | 10.18 | 54.14 | Uncharacterized protein |
| Solyc03g118780 | 2.41 | 17.07 | 90.66 | Uncharacterized protein |
| Solyc11g069210 | 2.40 | 2.17 | 11.47 | Uncharacterized protein |
| Solyc05g055680 | 2.40 | 11.72 | 61.83 | Putative JHL23J11.5-like |
| Solyc11g006430 | 2.40 | 1.83 | 9.65 | Glutaredoxin domain- |
| | | | | containing protein |
| Solyc01g091230 | 2.39 | 27.09 | 142.37 | Leucine-rich repeat receptor |
| | | | | kinase |
| Solyc01g079570 | 2.39 | 10.52 | 55.27 | Alpha-L- |
| | | | | arabinofuranosidase/beta-D- |
| | | | | xylosidase |
| Solyc01g079370 | 2.39 | 2.19 | 11.51 | GRAS domain-containing |
| | | | | protein |
| Solyc01g006240 | 2.39 | 7.68 | 40.30 | Jacalin-type lectin domain- |
| | | | | containing protein |
| Solyc10g084320 | 2.39 | 86.58 | 453.84 | Uncharacterized protein |
| Solyc04g078710 | 2.38 | 5.10 | 26.59 | Uncharacterized protein |
| Solyc07g017730 | 2.38 | 11.70 | 60.91 | Glucan endo-1,3-beta-D- |
| | | | | glucosidase (EC 3.2.1.39) |
| Solyc02g079480 | 2.38 | 5.51 | 28.69 | FAD-linked oxidoreductase 1 |
| Solyc04g006980 | 2.38 | 8.74 | 45.48 | Uncharacterized protein |
| Solyc09g008530 | 2.38 | 5.48 | 28.46 | C2 NT-type domain-containing |
| | | | | protein |
| Solyc04g074820 | 2.37 | 4.00 | 20.74 | Zinc finger protein |
| Solyc09g160260 | 2.37 | 4.06 | 21.03 | F-box family protein |
| Solyc02g081440 | 2.37 | 7.74 | 40.07 | Uncharacterized protein |
| Solyc12g019480 | 2.37 | 12.06 | 62.34 | Formin-like protein |
| Solyc08g062940 | 2.37 | 3.30 | 17.03 | DUF4005 domain-containing |
| | | | | protein |
| Solyc03g122070 | 2.37 | 2.56 | 13.19 | Uncharacterized protein |
| Solyc03g031530 | 2.36 | 6.59 | 33.81 | Uncharacterized protein |
| Solyc03g118510 | 2.36 | 59.88 | 306.65 | Protein kinase domain- |
| | | | | containing protein |
| Solyc02g070210 | 2.35 | 22.30 | 114.06 | Uncharacterized protein |
| Solyc03g117250 | 2.35 | 67.16 | 343.21 | Flotillin-like |
| Solyc11g013270 | 2.35 | 12.43 | 63.44 | O-fucosyltransferase family |
| | | | | protein |
| Solyc11g008650 | 2.35 | 2.94 | 14.96 | Uncharacterized protein |
| Solyc01g104200 | 2.34 | 7.24 | 36.66 | Fatty acyl-CoA reductase (EC |
| | | | | 1.2.1.84) |
| Solyc07g043420 | 2.34 | 357.55 | 1,810.06 | 2-oxoglutarate-dependent |
| | | | | dioxygenase homolog |
| | | | | (Fragment) |
| Solyc02g062960 | 2.34 | 2.93 | 14.83 | Homeobox domain-containing |
| | | | | protein |
| Solyc01g009220 | 2.34 | 8.75 | 44.23 | SHSP domain-containing |
| | | | | protein |
| Solyc01g080750 | 2.34 | 15.03 | 75.93 | TOG domain-containing |
| | | | | protein |
| Solyc06g065550 | 2.34 | 3.61 | 18.23 | Expansin-like EG45 domain- |
| | | | | containing protein |
| Solyc09g075770 | 2.34 | 4.40 | 22.20 | 4-coumarat--CoA ligase (EC |
| | | | | 6.2.1.12) |
| Solyc08g014190 | 2.34 | 7.29 | 36.80 | Uncharacterized protein |
| Solyc09g074470 | 2.33 | 8.42 | 42.47 | NAB domain-containing |
| | | | | protein |
| Solyc08g066050 | 2.33 | 5.87 | 29.61 | Non-specific serine/threonine |
| | | | | protein kinase (EC 2.7.11.1) |
| Solyc02g077330 | 2.33 | 69.27 | 348.89 | Lipase_GDSL domain- |
| | | | | containing protein |
| Solyc01g111740 | 2.33 | 6.95 | 34.97 | Uncharacterized protein |
| Solyc10g086520 | 2.33 | 4.35 | 21.85 | Expansin |
| Solyc04g007470 | 2.33 | 22.31 | 111.91 | B box-type domain-containing |
| | | | | protein |
| Solyc12g036470 | 2.33 | 24.18 | 121.29 | BHLH domain-containing |
| | | | | protein |
| Solyc11g061960 | 2.32 | 13.93 | 69.68 | Protein kinase domain- |
| | | | | containing protein |
| Solyc09g059550 | 2.32 | 20.34 | 101.71 | Uncharacterized protein |
| Solyc02g088320 | 2.32 | 12.85 | 63.94 | DUF1336 domain-containing |
| | | | | protein |
| Solyc08g014580 | 2.31 | 2.17 | 10.78 | MSP domain-containing |
| | | | | protein |
| Solyc01g104400 | 2.31 | 28.48 | 141.71 | Phytocyanin domain- |
| | | | | containing protein |
| Solyc07g065880 | 2.31 | 36.83 | 182.89 | Uncharacterized protein |
| Solyc08g083270 | 2.31 | 4.05 | 20.08 | Hop-interacting protein |
| | | | | THI143 |
| Solyc04g063200 | 2.31 | 2.55 | 12.61 | NA |
| Solyc03g115690 | 2.31 | 5.80 | 28.68 | Uncharacterized protein |
| Solyc12g096105 | 2.31 | 2.92 | 14.43 | Uncharacterized protein |
| Solyc02g062760 | 2.30 | 7.30 | 36.00 | GATA transcription factor |
| Solyc01g106650 | 2.30 | 4.76 | 23.47 | Xyloglucan |
| | | | | endotransglucosylase/hydrolase |
| | | | | (EC 2.4.1.207) |
| Solyc02g081120 | 2.30 | 7.39 | 36.33 | Uncharacterized protein |
| Solyc07g052910 | 2.30 | 5.47 | 26.88 | Uncharacterized protein |
| Solyc02g089520 | 2.29 | 36.30 | 177.44 | Uncharacterized protein |
| Solyc01g099150 | 2.28 | 17.83 | 86.87 | Lipoxygenase (EC 1.13.11.—) |
| Solyc02g090220 | 2.28 | 11.66 | 56.76 | Dof-type domain-containing |
| | | | | protein |
| Solyc02g064945 | 2.28 | 2.95 | 14.37 | Polysacc_synt_4 domain- |
| | | | | containing protein |
| Solyc04g015620 | 2.28 | 14.25 | 69.37 | Uncharacterized protein |
| Solyc03g121470 | 2.28 | 2.56 | 12.44 | Phospholipase D (EC 3.1.4.4) |
| Solyc04g005470 | 2.28 | 6.19 | 30.08 | Remorin_C domain-containing |
| | | | | protein |
| Solyc02g083640 | 2.28 | 8.77 | 42.51 | TPR_REGION domain- |
| | | | | containing protein |
| Solyc02g071700 | 2.28 | 10.55 | 51.17 | Uncharacterized protein |
| Solyc08g078680 | 2.28 | 4.01 | 19.44 | CRAL-TRIO domain- |
| | | | | containing protein |
| Solyc04g076400 | 2.27 | 4.04 | 19.53 | PHD_Oberon domain- |
| | | | | containing protein |
| Solyc08g079200 | 2.27 | 59.64 | 288.42 | AAI domain-containing protein |
| Solyc02g079980 | 2.27 | 59.06 | 285.42 | Uncharacterized protein |
| Solyc06g035430 | 2.27 | 7.29 | 35.16 | AT-hook motif nuclear- |
| | | | | localized protein |
| Solyc02g087440 | 2.27 | 22.73 | 109.54 | Uncharacterized protein |
| Solyc01g007870 | 2.27 | 3.29 | 15.84 | Uncharacterized protein |
| Solyc10g080540 | 2.27 | 13.52 | 65.11 | Homeobox domain-containing |
| | | | | protein |
| Solyc09g082760 | 2.27 | 180.82 | 870.96 | Uncharacterized protein |
| Solyc02g089880 | 2.27 | 11.73 | 56.41 | Hexosyltransferase (EC 2.4.1.—) |
| Solyc03g110890 | 2.27 | 2.19 | 10.54 | Polysacc_synt_4 domain- |
| | | | | containing protein |
| Solyc02g080190 | 2.26 | 15.36 | 73.59 | NTF2 domain-containing |
| | | | | protein |
| Solyc05g007760 | 2.26 | 4.41 | 21.07 | SET domain-containing protein |
| Solyc07g053700 | 2.25 | 8.79 | 41.83 | Reticulon-like protein |
| Solyc09g061930 | 2.25 | 92.07 | 436.51 | Protein kinase domain- |
| | | | | containing protein |
| Solyc05g009950 | 2.24 | 33.55 | 158.94 | Cytochrome B561 family |
| | | | | protein |
| Solyc01g009340 | 2.24 | 18.96 | 89.79 | DUF4005 domain-containing |
| | | | | protein |
| Solyc04g078270 | 2.24 | 8.77 | 41.42 | Uncharacterized protein |
| Solyc01g066860 | 2.24 | 43.22 | 203.97 | Uncharacterized protein |
| Solyc02g071310 | 2.24 | 26.63 | 125.67 | Uncharacterized protein |
| Solyc07g041290 | 2.24 | 4.01 | 18.93 | Uncharacterized protein |
| Solyc01g097000 | 2.24 | 57.89 | 272.64 | Uncharacterized protein |
| Solyc03g113210 | 2.24 | 25.47 | 119.93 | Uncharacterized protein |
| Solyc01g080290 | 2.23 | 12.85 | 60.38 | Uncharacterized protein |
| Solyc03g114240 | 2.23 | 12.35 | 57.92 | BURP domain-containing |
| | | | | protein |
| Solyc02g083630 | 2.23 | 28.56 | 133.90 | PEROXIDASE_4 domain- |
| | | | | containing protein |
| Solyc08g081450 | 2.23 | 12.51 | 58.57 | Uncharacterized protein |
| Solyc05g006870 | 2.23 | 313.71 | 1,467.54 | Thioredoxin |
| Solyc04g077490 | 2.23 | 86.33 | 403.86 | AINTEGUMENTA homolog |
| Solyc07g047710 | 2.22 | 4.37 | 20.41 | X8 domain-containing protein |
| Solyc03g115890 | 2.22 | 15.04 | 70.00 | Expansin |
| Solyc05g007870 | 2.22 | 3.31 | 15.40 | R2R3 MYB |
| Solyc04g058080 | 2.22 | 2.92 | 13.57 | X8 domain-containing protein |
| Solyc09g060110 | 2.21 | 40.41 | 187.60 | Kinase |
| Solyc10g005010 | 2.21 | 2.57 | 11.93 | NAC domain-containing |
| | | | | protein |
| Solyc10g078370 | 2.21 | 51.08 | 236.52 | Auxin efflux carrier component |
| Solyc01g107730 | 2.21 | 10.21 | 47.22 | CycD3; 2 protein |
| Solyc11g065190 | 2.21 | 14.23 | 65.79 | UBC core domain-containing |
| | | | | protein |
| Solyc03g116280 | 2.21 | 2.91 | 13.43 | Fe2OG dioxygenase domain- |
| | | | | containing protein |
| Solyc09g150105 | 2.21 | 9.96 | 45.97 | Laccase (EC 1.10.3.2) |
| | | | | (Benzenediol: oxygen |
| | | | | oxidoreductase) (Diphenol |
| | | | | oxidase) (Urishiol oxidase) |
| Solyc11g066010 | 2.20 | 12.41 | 57.22 | Uncharacterized protein |
| Solyc03g120890 | 2.20 | 13.95 | 64.32 | GATA transcription factor |
| Solyc09g048980 | 2.20 | 22.30 | 102.74 | Uncharacterized protein |
| Solyc12g087850 | 2.20 | 2.57 | 11.84 | DNA binding protein |
| Solyc08g042100 | 2.20 | 9.86 | 45.32 | Uncharacterized protein |
| Solyc05g008300 | 2.20 | 19.77 | 90.75 | Chloroplast-targeted copper |
| | | | | chaperone |
| Solyc02g077420 | 2.20 | 25.95 | 119.05 | Lipase_3 domain-containing |
| | | | | protein |
| Solyc03g095820 | 2.19 | 12.35 | 56.53 | PMR5N domain-containing |
| | | | | protein |
| Solyc06g075130 | 2.19 | 5.49 | 25.11 | Uncharacterized protein |
| Solyc08g005540 | 2.19 | 24.36 | 111.40 | Uncharacterized protein |
| Solyc05g054590 | 2.19 | 43.58 | 199.23 | UDP-glucuronate |
| | | | | decarboxylase 1 |
| Solyc02g083890 | 2.19 | 19.08 | 87.21 | Uncharacterized protein |
| Solyc01g094090 | 2.18 | 2.89 | 13.14 | Uncharacterized protein |
| Solyc01g068075 | 2.18 | 13.87 | 62.76 | NA |
| Solyc10g055790 | 2.18 | 6.62 | 29.91 | Chitin-binding type-1 domain- |
| | | | | containing protein |
| Solyc12g099590 | 2.18 | 5.87 | 26.52 | Uncharacterized protein |
| Solyc07g018290 | 2.17 | 58.61 | 264.42 | Uncharacterized protein |
| Solyc04g082710 | 2.17 | 102.48 | 461.49 | Uncharacterized protein |
| Solyc09g098490 | 2.17 | 9.86 | 44.25 | Uncharacterized protein |
| Solyc07g063690 | 2.16 | 16.20 | 72.56 | EF-hand domain-containing |
| | | | | protein |
| Solyc06g076220 | 2.16 | 124.36 | 556.47 | Expansin |
| Solyc06g068360 | 2.16 | 9.90 | 44.27 | AP2/ERF domain-containing |
| | | | | protein |
| Solyc01g109330 | 2.16 | 8.04 | 35.89 | ResB domain-containing |
| | | | | protein |
| Solyc07g053450 | 2.16 | 4.01 | 17.91 | BZIP domain-containing |
| | | | | protein |
| Solyc06g084080 | 2.16 | 3.27 | 14.60 | GB1/RHD3-type G domain- |
| | | | | containing protein |
| Solyc08g083220 | 2.15 | 13.92 | 61.90 | Uncharacterized protein |
| Solyc07g042520 | 2.15 | 68.32 | 303.43 | Sucrose synthase (EC 2.4.1.13) |
| Solyc11g013300 | 2.15 | 8.05 | 35.75 | Thaumatin |
| Solyc08g042030 | 2.15 | 5.51 | 24.47 | Kinesin motor domain- |
| | | | | containing protein |
| Solyc04g078590 | 2.15 | 8.43 | 37.35 | Protein kinase domain- |
| | | | | containing protein |
| Solyc10g086320 | 2.15 | 3.60 | 15.91 | Phosphatidylinositol 4- |
| | | | | phosphate 5-kinase (EC |
| | | | | 2.7.1.68) |
| Solyc06g054630 | 2.14 | 9.48 | 41.90 | Transcriptional factor TINY |
| Solyc06g073710 | 2.14 | 2.91 | 12.84 | Uncharacterized protein |
| Solyc03g033860 | 2.14 | 4.69 | 20.72 | Uncharacterized protein |
| Solyc01g096150 | 2.14 | 37.95 | 167.57 | Uncharacterized protein |
| Solyc03g121680 | 2.14 | 6.97 | 30.76 | Uncharacterized protein |
| Solyc02g081300 | 2.14 | 27.44 | 121.14 | Sucrose synthase (EC 2.4.1.13) |
| Solyc02g068040 | 2.14 | 66.58 | 293.78 | Putative ovule protein |
| Solyc01g066570 | 2.14 | 56.51 | 248.45 | FLZ-type domain-containing |
| | | | | protein |
| Solyc12g056710 | 2.13 | 12.35 | 54.10 | Uncharacterized protein |
| Solyc12g098930 | 2.13 | 6.54 | 28.62 | Protein-serine/threonine kinase |
| | | | | (EC 2.7.11.—) |
| Solyc12g056200 | 2.12 | 5.78 | 25.19 | Uncharacterized protein |
| Solyc10g055780 | 2.12 | 29.66 | 129.24 | Glyco_hydro_19_cat domain- |
| | | | | containing protein |
| Solyc04g077570 | 2.12 | 9.16 | 39.76 | Uncharacterized protein |
| Solyc07g048050 | 2.11 | 10.85 | 47.00 | Uncharacterized protein |
| Solyc07g007755 | 2.11 | 244.84 | 1,058.69 | Defensin protein |
| Solyc06g072350 | 2.11 | 395.59 | 1,709.18 | CASP-like protein |
| Solyc04g074470 | 2.11 | 130.51 | 563.55 | Uncharacterized protein |
| Solyc11g031960 | 2.11 | 139.94 | 604.20 | Lipase_GDSL domain- |
| | | | | containing protein |
| Solyc04g082970 | 2.11 | 9.86 | 42.54 | Uncharacterized protein |
| Solyc11g071730 | 2.11 | 9.44 | 40.74 | Putative ovule protein |
| Solyc11g007730 | 2.11 | 22.74 | 97.96 | PNP_UDP_1 domain- |
| | | | | containing protein |
| Solyc10g049420 | 2.11 | 8.03 | 34.58 | Uncharacterized protein |
| Solyc06g066240 | 2.11 | 23.93 | 102.96 | Uncharacterized protein |
| Solyc09g072660 | 2.10 | 13.19 | 56.49 | FAD-binding PCMH-type |
| | | | | domain-containing protein |
| Solyc06g062880 | 2.10 | 26.32 | 112.67 | Uncharacterized protein |
| Solyc02g084720 | 2.10 | 249.42 | 1,066.47 | Beta-galactosidase (EC |
| | | | | 3.2.1.23) |
| Solyc07g053900 | 2.09 | 45.21 | 193.15 | Uncharacterized protein |
| Solyc05g050540 | 2.09 | 20.75 | 88.46 | Laccase (EC 1.10.3.2) |
| | | | | (Benzenediol: oxygen |
| | | | | oxidoreductase) (Diphenol |
| | | | | oxidase) (Urishiol oxidase) |
| Solyc07g065530 | 2.09 | 20.04 | 85.42 | Uncharacterized protein |
| Solyc01g101260 | 2.09 | 25.32 | 107.47 | Uncharacterized protein |
| Solyc02g024070 | 2.08 | 141.44 | 599.55 | Uncharacterized protein |
| Solyc03g121080 | 2.08 | 5.47 | 23.14 | Uncharacterized protein |
| Solyc07g048110 | 2.08 | 7.35 | 31.03 | Putative ovule protein |
| | | | | (Fragment) |
| Solyc09g014490 | 2.07 | 8.09 | 34.01 | Uncharacterized protein |
| Solyc12g014130 | 2.07 | 24.17 | 101.55 | Uncharacterized protein |
| Solyc04g073940 | 2.07 | 4.40 | 18.44 | Uncharacterized protein |
| Solyc04g081790 | 2.07 | 6.91 | 28.96 | Uncharacterized protein |
| Solyc08g042025 | 2.06 | 5.48 | 22.84 | Retrovirus-related Pol |
| | | | | polyprotein from transposon |
| | | | | TNT 1-94 |
| Solyc01g006680 | 2.06 | 169.96 | 706.76 | Uncharacterized protein |
| Solyc11g005730 | 2.05 | 5.11 | 21.12 | Uncharacterized protein |
| Solyc05g047580 | 2.05 | 5.43 | 22.41 | Terpene cyclase/mutase family |
| | | | | member (EC 5.4.99.—) |
| Solyc07g041970 | 2.04 | 137.37 | 566.78 | Uncharacterized protein |
| Solyc06g051830 | 2.04 | 3.67 | 15.07 | Uncharacterized protein |
| Solyc07g006020 | 2.04 | 10.28 | 42.23 | Uncharacterized protein |
| Solyc12g056190 | 2.04 | 4.74 | 19.45 | Uncharacterized protein |
| Solyc04g016330 | 2.04 | 26.95 | 110.55 | Glycerol-3-phosphate |
| | | | | dehydrogenase [NAD(+)] (EC |
| | | | | 1.1.1.8) |
| Solyc11g069650 | 2.03 | 14.28 | 58.37 | Disease resistance protein (NB- |
| | | | | LRR disease resistance protein) |
| Solyc05g013860 | 2.03 | 15.67 | 64.05 | Uncharacterized protein |
| Solyc09g065475 | 2.03 | 12.81 | 52.25 | NA |
| Solyc09g042710 | 2.03 | 30.73 | 125.23 | Uncharacterized protein |
| Solyc01g106750 | 2.03 | 10.19 | 41.51 | Uncharacterized protein |
| Solyc01g098720 | 2.02 | 4.01 | 16.33 | BHLH domain-containing |
| | | | | protein |
| Solyc02g088210 | 2.02 | 18.27 | 74.21 | SPX domain-containing protein |
| Solyc12g010050 | 2.02 | 52.40 | 212.77 | Uncharacterized protein |
| Solyc06g071480 | 2.02 | 28.87 | 117.18 | Dof-type domain-containing |
| | | | | protein |
| Solyc03g007230 | 2.02 | 52.12 | 211.46 | Protein-serine/threonine |
| | | | | phosphatase (EC 3.1.3.16) |
| Solyc05g052680 | 2.02 | 33.47 | 135.70 | Uncharacterized protein |
| Solyc06g008360 | 2.02 | 5.12 | 20.76 | NAC domain-containing |
| | | | | protein |
| Solyc03g115120 | 2.02 | 5.47 | 22.15 | Uncharacterized protein |
| Solyc11g045610 | 2.02 | 23.01 | 93.21 | Protein kinase domain- |
| | | | | containing protein |
| Solyc06g061027 | 2.02 | 14.22 | 57.60 | Putative cytochrome P450 |
| Solyc06g008590 | 2.02 | 12.79 | 51.80 | Auxin-responsive protein |
| Solyc04g082430 | 2.02 | 15.42 | 62.35 | Cyclin N-terminal domain- |
| | | | | containing protein |
| Solyc06g054320 | 2.02 | 27.62 | 111.66 | Dirigent protein |
| Solyc02g089050 | 2.02 | 45.55 | 184.13 | DUF4378 domain-containing |
| | | | | protein |
| Solyc11g021060 | 2.01 | 46.39 | 187.48 | Proteinase inhibitor type-2 TR8 |
| | | | | (Proteinase inhibitor type II |
| | | | | TR8) |
| Solyc02g069680 | 2.01 | 5.52 | 22.32 | Vacuolar protein sorting- |
| | | | | associated protein 2-like |
| | | | | protein 2 |
| Solyc11g042580 | 2.01 | 5.88 | 23.75 | AP2/ERF domain-containing |
| | | | | protein |
| Solyc01g091000 | 2.01 | 4.01 | 16.19 | Uncharacterized protein |
| Solyc07g054690 | 2.01 | 5.15 | 20.74 | Uncharacterized protein |
| Solyc07g063970 | 2.01 | 6.92 | 27.87 | C2H2-type domain-containing |
| | | | | protein |
| Solyc02g067710 | 2.01 | 4.00 | 16.11 | TOG domain-containing |
| | | | | protein |
| Solyc11g012040 | 2.01 | 14.65 | 58.86 | Uncharacterized protein |
| Solyc01g006670 | 2.01 | 61.83 | 248.37 | UDP-glucosyltransferase |

Table 7 depicts genes whose expression was downregulated by a 2-4 fold decrease in treated plants compared to control plants.

TABLE 7
Downregulated Genes between Treated and Control F3 Tomato Plants (2-4 fold decrease)

| | | Control | Treatment | |
|---|---|---|---|---|
| Gene_ID | log2FoldChange | Expression | Expression | Protein Name |
| Solyc11g010380 | −2.00 | 1,459.51 | 364.37 | Protein DETOXIFICATION |
| | | | | (Multidrug and toxic |
| | | | | compound extrusion protein) |
| Solyc01g106820 | −2.00 | 904.74 | 225.89 | Sterol regulatory element- |
| | | | | binding protein site 2 protease |
| Solyc05g010000 | −2.00 | 28.09 | 7.02 | Uncharacterized protein |
| Solyc02g091590 | −2.00 | 31.31 | 7.85 | Symbiosis receptor-like kinase |
| Solyc05g012740 | −1.99 | 79.02 | 19.83 | NB-ARC domain-containing |
| | | | | protein |
| Solyc04g064870 | −1.99 | 2,019.19 | 506.68 | Uncharacterized protein |
| Solyc09g005610 | −1.99 | 16.41 | 4.13 | Transcription factor HBP-1b |
| Solyc07g061870 | −1.98 | 18.65 | 4.72 | Uncharacterized protein |
| Solyc08g067630 | −1.98 | 3,440.93 | 872.22 | Uncharacterized protein |
| Solyc02g005601 | −1.98 | 70.58 | 17.89 | Peptidase A1 domain- |
| | | | | containing protein |
| Solyc01g100910 | −1.98 | 118.16 | 29.97 | WAT1-related protein |
| Solyc05g010040 | −1.97 | 931.67 | 237.05 | Uncharacterized protein |
| Solyc05g012660 | −1.97 | 25.02 | 6.38 | Uncharacterized protein |
| Solyc08g007430 | −1.97 | 51.48 | 13.13 | Uncharacterized protein |
| Solyc01g009690 | −1.97 | 1,582.29 | 404.43 | SC0A |
| Solyc10g076550 | −1.96 | 653.12 | 167.76 | Uncharacterized protein |
| Solyc04g072370 | −1.95 | 27.85 | 7.21 | Uncharacterized protein |
| Solyc02g094860 | −1.95 | 15.37 | 3.99 | Uncharacterized protein |
| Solyc01g100460 | −1.94 | 120.82 | 31.42 | Anaerobic basic leucine zipper |
| | | | | protein |
| Solyc06g036420 | −1.94 | 64.07 | 16.75 | Protein SGT1-like protein B- |
| | | | | like |
| Solyc10g005080 | −1.93 | 470.17 | 123.14 | HTH myb-type domain- |
| | | | | containing protein |
| Solyc02g065240 | −1.93 | 3,162.54 | 829.93 | Uncharacterized protein |
| Solyc05g009750 | −1.93 | 17.65 | 4.65 | NB-ARC domain-containing |
| | | | | protein |
| Solyc02g062340 | −1.93 | 5,326.92 | 1,402.53 | Fructose-bisphosphate aldolase |
| | | | | (EC 4.1.2.13) |
| Solyc01g080870 | −1.92 | 547.03 | 144.14 | Uncharacterized protein |
| Solyc04g078340 | −1.92 | 44.84 | 11.83 | Uncharacterized protein |
| Solyc07g039570 | −1.92 | 80.96 | 21.42 | BHLH domain-containing |
| | | | | protein |
| Solyc01g100020 | −1.92 | 90.63 | 23.99 | Phospholipase (EC 3.1.4.4) |
| Solyc12g040740 | −1.92 | 28.40 | 7.52 | Hcr2-0B |
| Solyc11g020040 | −1.92 | 1,417.66 | 375.52 | Uncharacterized protein |
| Solyc09g005090 | −1.91 | 357.46 | 95.12 | Verticillium wilt disease |
| | | | | resistance protein |
| Solyc08g078900 | −1.91 | 2,049.65 | 546.02 | Arachidonic acid-induced |
| | | | | DEA1 |
| Solyc12g045035 | −1.91 | 1,074.86 | 286.47 | Fatty acid desaturase |
| Solyc03g044790 | −1.91 | 1,538.28 | 410.23 | Methylesterase |
| Solyc01g104740 | −1.91 | 92.33 | 24.63 | HTH cro/C1-type domain- |
| | | | | containing protein |
| Solyc03g033500 | −1.91 | 1,609.02 | 429.31 | Putative cold shock domain- |
| | | | | containing protein 3-like |
| Solyc04g071900 | −1.91 | 1,106.85 | 295.36 | Peroxidase (EC 1.11.1.7) |
| Solyc06g064720 | −1.91 | 17.61 | 4.70 | NB-ARC domain-containing |
| | | | | protein |
| Solyc07g061990 | −1.90 | 2,391.26 | 638.61 | Solanesyl-diphosphate |
| | | | | synthase 2, chloroplastic |
| Solyc03g113430 | −1.90 | 3,963.42 | 1,059.19 | Nitrate transporter |
| Solyc09g091910 | −1.90 | 131.84 | 35.24 | Purple acid phosphatase (EC |
| | | | | 3.1.3.2) |
| Solyc10g083690 | −1.90 | 508.84 | 136.07 | Uncharacterized protein |
| Solyc07g043150 | −1.90 | 316.56 | 84.97 | Glycosyltransferase (EC 2.4.1.—) |
| Solyc07g056210 | −1.89 | 945.09 | 254.11 | Uncharacterized protein |
| Solyc01g096720 | −1.89 | 31.98 | 8.61 | MFS domain-containing |
| | | | | protein |
| Solyc09g014820 | −1.89 | 18.23 | 4.93 | Uncharacterized protein |
| Solyc08g082380 | −1.89 | 78.20 | 21.14 | Uncharacterized protein |
| Solyc09g064380 | −1.88 | 172.50 | 46.72 | TPR_REGION domain- |
| | | | | containing protein |
| Solyc02g085940 | −1.88 | 2,524.06 | 686.32 | Uncharacterized protein |
| Solyc03g094120 | −1.88 | 613.46 | 167.09 | Uncharacterized protein |
| Solyc10g018340 | −1.88 | 106.78 | 29.11 | Uncharacterized protein |
| Solyc02g087070 | −1.87 | 30.73 | 8.41 | Uncharacterized protein |
| Solyc01g087150 | −1.87 | 49.08 | 13.45 | Protein DETOXIFICATION |
| | | | | (Multidrug and toxic |
| | | | | compound extrusion protein) |
| Solyc05g013380 | −1.87 | 4,524.36 | 1,240.76 | AlaT1 |
| Solyc03g119810 | −1.86 | 301.67 | 83.18 | Uncharacterized protein |
| Solyc02g150128 | −1.86 | 768.19 | 212.33 | Protein kinase domain- |
| | | | | containing protein |
| Solyc02g021680 | −1.85 | 56.59 | 15.68 | WRKY domain-containing |
| | | | | protein |
| Solyc09g083200 | −1.85 | 2,348.36 | 653.10 | Uncharacterized protein |
| Solyc03g116630 | −1.84 | 689.97 | 192.06 | Uncharacterized protein |
| Solyc07g066550 | −1.84 | 25.63 | 7.14 | Non-specific serine/threonine |
| | | | | protein kinase (EC 2.7.11.1) |
| Solyc09g089680 | −1.84 | 179.38 | 50.09 | Fe2OG dioxygenase domain- |
| | | | | containing protein |
| Solyc12g010410 | −1.84 | 403.50 | 112.73 | Uncharacterized protein |
| Solyc05g014550 | −1.84 | 639.94 | 178.88 | TLC domain-containing |
| | | | | protein |
| Solyc05g010720 | −1.84 | 204.91 | 57.30 | Uncharacterized protein |
| Solyc05g055990 | −1.84 | 119.03 | 33.29 | Uncharacterized protein |
| Solyc11g008820 | −1.84 | 96.20 | 26.94 | Endoglucanase (EC 3.2.1.4) |
| Solyc09g011270 | −1.84 | 495.64 | 138.80 | Uncharacterized protein |
| Solyc06g068960 | −1.84 | 1,915.01 | 536.70 | Uncharacterized protein |
| Solyc10g008160 | −1.83 | 35.00 | 9.82 | Golden 2-like 2 transcription |
| | | | | factor (Golden2-like protein) |
| Solyc12g009480 | −1.83 | 1,391.73 | 390.92 | SPX domain-containing protein |
| Solyc01g107760 | −1.83 | 122.19 | 34.39 | UDPGT domain-containing |
| | | | | protein |
| Solyc03g111540 | −1.83 | 248.55 | 70.04 | Uncharacterized protein |
| Solyc12g096550 | −1.83 | 3,972.48 | 1,120.51 | Rieske domain-containing |
| | | | | protein |
| Solyc07g048070 | −1.82 | 870.24 | 246.11 | Cytochrome b561 and |
| | | | | DOMON domain-containing |
| | | | | protein |
| Solyc08g023510 | −1.82 | 66.58 | 18.85 | Uncharacterized protein |
| Solyc01g089890 | −1.82 | 725.29 | 205.53 | PRA1 family protein |
| Solyc08g016220 | −1.82 | 424.69 | 120.63 | LRRNT_2 domain-containing |
| | | | | protein |
| Solyc11g013810 | −1.81 | 4,122.90 | 1,173.20 | Nitrate reductase |
| Solyc12g045037 | −1.81 | 652.13 | 185.70 | Fatty acid desaturase |
| Solyc03g044830 | −1.81 | 49.17 | 14.04 | WD_REPEATS_REGION |
| | | | | domain-containing protein |
| Solyc01g008620 | −1.80 | 7,241.49 | 2,075.45 | Uncharacterized protein |
| Solyc01g068630 | −1.80 | 67.96 | 19.53 | Putative suppressor of |
| | | | | disruption of TFIIS-like |
| Solyc12g045020 | −1.80 | 1,726.13 | 496.69 | Uncharacterized protein |
| Solyc07g008140 | −1.79 | 1,057.83 | 305.39 | Cucumber peeling cupredoxin |
| Solyc01g087770 | −1.79 | 51.73 | 14.95 | Peptidase_S8 domain- |
| | | | | containing protein |
| Solyc06g064610 | −1.79 | 62.14 | 18.01 | Uncharacterized protein |
| Solyc01g057680 | −1.79 | 853.82 | 247.46 | Hcr2-0B |
| Solyc01g109140 | −1.79 | 92.15 | 26.72 | 9-divinyl ether synthase |
| | | | | (LeDES) (EC 4.2.1.121) |
| | | | | (Colneleate synthase) |
| | | | | (Cytochrome P450 74D1) |
| Solyc07g040710 | −1.78 | 511.94 | 148.59 | Calmodulin binding protein |
| Solyc10g051140 | −1.78 | 189.09 | 55.18 | Uncharacterized protein |
| Solyc03g025350 | −1.78 | 570.44 | 166.53 | CitMHS domain-containing |
| | | | | protein |
| Solyc11g072470 | −1.78 | 215.47 | 62.91 | LOB domain-containing |
| | | | | protein |
| Solyc07g063770 | −1.78 | 528.62 | 154.41 | Receptor-like serine/threonine- |
| | | | | protein kinase (EC 2.7.11.1) |
| Solyc05g053600 | −1.78 | 987.55 | 288.49 | Uncharacterized protein |
| Solyc01g014140 | −1.77 | 204.92 | 60.06 | Hcr9-OR2A |
| Solyc05g021090 | −1.77 | 294.38 | 86.41 | NAC domain-containing |
| | | | | protein |
| Solyc09g011140 | −1.77 | 407.15 | 119.71 | Tropinone reductase 1 |
| Solyc08g068870 | −1.76 | 14,667.06 | 4,326.44 | Peptidase A1 domain- |
| | | | | containing protein |
| Solyc02g067050 | −1.76 | 47.28 | 13.95 | Uncharacterized protein |
| Solyc03g025720 | −1.76 | 1,262.08 | 372.74 | 4-coumarate--CoA ligase (EC |
| | | | | 6.2.1.12) |
| Solyc07g006500 | −1.75 | 3,583.23 | 1,065.35 | Trehalose-6-phosphate |
| | | | | synthase |
| Solyc11g007970 | −1.75 | 168.83 | 50.25 | 4-coumarate--CoA ligase (EC |
| | | | | 6.2.1.12) |
| Solyc12g099930 | −1.75 | 4,097.60 | 1,219.58 | Uncharacterized protein |
| Solyc02g071470 | −1.75 | 756.50 | 225.26 | Uncharacterized protein |
| Solyc08g007460 | −1.75 | 3,152.56 | 939.16 | AAI domain-containing protein |
| Solyc11g008250 | −1.75 | 3,853.89 | 1,148.88 | ATP binding protein |
| Solyc08g016210 | −1.74 | 3,505.50 | 1,048.59 | Uncharacterized protein |
| Solyc06g076520 | −1.74 | 2,883.54 | 862.56 | 17.7 kD class I small heat |
| | | | | shock protein (Type I small |
| | | | | heat shock protein 17.7 kDa I2I |
| | | | | isoform) |
| Solyc05g012180 | −1.74 | 169.32 | 50.75 | AB hydrolase-1 domain- |
| | | | | containing protein |
| Solyc10g007570 | −1.74 | 106.27 | 31.88 | Fe2OG dioxygenase domain- |
| | | | | containing protein |
| Solyc01g073820 | −1.74 | 66.77 | 20.05 | Uncharacterized protein |
| Solyc01g005470 | −1.73 | 2,155.20 | 649.56 | Uncharacterized protein |
| Solyc12g009770 | −1.73 | 33.21 | 10.03 | LRRNT_2 domain-containing |
| | | | | protein |
| Solyc04g014400 | −1.72 | 20,144.94 | 6,105.99 | Rhamnogalacturonan |
| | | | | endolyase (EC 4.2.2.23) |
| Solyc07g056540 | −1.71 | 35,909.26 | 10,951.87 | (S)-2-hydroxy-acid oxidase |
| | | | | (EC 1.1.3.15) |
| Solyc03g098100 | −1.71 | 100.56 | 30.72 | Aldo_ket_red domain- |
| | | | | containing protein |
| Solyc04g051800 | −1.71 | 15,784.31 | 4,826.17 | Uncharacterized protein |
| Solyc04g009640 | −1.71 | 119.78 | 36.66 | Protein kinase domain- |
| | | | | containing protein |
| Solyc09g011810 | −1.71 | 4,121.55 | 1,261.47 | D-fructose-1,6-bisphosphate 1- |
| | | | | phosphohydrolase (EC |
| | | | | 3.1.3.11) |
| Solyc12g042010 | −1.71 | 76.58 | 23.46 | BHLH domain-containing |
| | | | | protein |
| Solyc04g074000 | −1.71 | 5,423.42 | 1,662.60 | Uncharacterized protein |
| Solyc12g005610 | −1.71 | 107.43 | 32.93 | LRRNT_2 domain-containing |
| | | | | protein |
| Solyc10g051020 | −1.70 | 5,515.22 | 1,691.91 | CYP72A54 |
| Solyc09g090570 | −1.70 | 1,086.89 | 334.10 | Uncharacterized protein |
| Solyc07g066330 | −1.70 | 937.46 | 288.20 | NAC domain-containing |
| | | | | protein |
| Solyc01g006550 | −1.70 | 5,390.91 | 1,658.37 | Receptor-like protein Cf-9 |
| | | | | homolog |
| Solyc07g056100 | −1.70 | 30.75 | 9.46 | Uncharacterized protein |
| Solyc02g084970 | −1.70 | 232.62 | 71.70 | acidPPc domain-containing |
| | | | | protein |
| Solyc10g086250 | −1.69 | 156.17 | 48.27 | MYB-like transcriptional factor |
| | | | | AN2 (Fragment) |
| Solyc09g092490 | −1.69 | 69.02 | 21.37 | Uncharacterized protein |
| Solyc10g051120 | −1.69 | 346.04 | 107.15 | Mitochondrial pyruvate carrier |
| Solyc07g051820 | −1.69 | 1,551.56 | 480.44 | uncharacterized protein |
| | | | | LOC104222458 |
| Solyc05g012230 | −1.69 | 259.35 | 80.31 | RNA binding protein |
| Solyc00g500069 | −1.69 | 59.57 | 18.45 | Uncharacterized protein |
| | | | | (Fragment) |
| Solyc06g073260 | −1.69 | 3,184.54 | 986.39 | NAD dependent |
| | | | | epimerase/dehydratase |
| Solyc08g083190 | −1.69 | 249.47 | 77.39 | AB hydrolase-1 domain- |
| | | | | containing protein |
| Solyc10g085860 | −1.68 | 90.76 | 28.28 | Glycosyltransferase (EC 2.4.1.—) |
| Solyc01g007960 | −1.68 | 265.16 | 82.69 | Uncharacterized protein |
| Solyc08g062220 | −1.68 | 1,689.09 | 527.06 | Glycosyltransferase (EC 2.4.1.—) |
| Solyc08g078870 | −1.68 | 2,592.53 | 811.33 | Putative 14 kDa proline-rich |
| | | | | protein DC2.15-like |
| Solyc04g011770 | −1.67 | 48.45 | 15.19 | Uncharacterized protein |
| Solyc01g009700 | −1.67 | 2,749.82 | 862.56 | SC0A |
| Solyc01g008500 | −1.67 | 859.32 | 270.20 | Protein kinase domain- |
| | | | | containing protein |
| Solyc03g098320 | −1.67 | 111.74 | 35.14 | HTH myb-type domain- |
| | | | | containing protein |
| Solyc04g040180 | −1.67 | 2,025.38 | 637.01 | Methyltransf_11 domain- |
| | | | | containing protein |
| Solyc08g076780 | −1.67 | 39.13 | 12.32 | Uncharacterized protein |
| Solyc07g055490 | −1.67 | 405.66 | 127.77 | Uncharacterized protein |
| Solyc01g095080 | −1.66 | 55.19 | 17.44 | 1-aminocyclopropane-1- |
| | | | | carboxylate synthase 2 (ACC |
| | | | | synthase 2) (EC 4.4.1.14) (Le- |
| | | | | ACS2) (ACS-2) (S-adenosyl- |
| | | | | L-methionine |
| | | | | methylthioadenosine-lyase 2) |
| Solyc04g082120 | −1.66 | 4,193.75 | 1,329.46 | Prolyl oligopeptidase (EC |
| | | | | 3.4.21.26) |
| Solyc04g082250 | −1.66 | 11,576.64 | 3,672.31 | AAA domain-containing |
| | | | | protein |
| Solyc05g009090 | −1.66 | 2,093.92 | 664.47 | Protein kinase domain- |
| | | | | containing protein |
| Solyc04g040160 | −1.66 | 750.45 | 238.17 | Rieske domain-containing |
| | | | | protein |
| Solyc09g082550 | −1.66 | 190.74 | 60.56 | STAS domain-containing |
| | | | | protein |
| Solyc02g084440 | −1.65 | 38,192.35 | 12,152.03 | Fructose-bisphosphate aldolase |
| | | | | (EC 4.1.2.13) (Fragment) |
| Solyc02g079530 | −1.65 | 139.84 | 44.53 | Receptor-like serine/threonine- |
| | | | | protein kinase (EC 2.7.11.1) |
| Solyc08g065220 | −1.65 | 10,057.26 | 3,212.48 | Glycine cleavage system P |
| | | | | protein (EC 1.4.4.2) |
| Solyc03g078310 | −1.65 | 32.79 | 10.48 | Uncharacterized protein |
| Solyc07g005210 | −1.64 | 1,361.45 | 436.87 | Temperature-induced lipocalin |
| Solyc06g050950 | −1.64 | 38.27 | 12.28 | LOB domain-containing |
| | | | | protein |
| Solyc03g112010 | −1.64 | 167.36 | 53.79 | Uncharacterized protein |
| Solyc07g055260 | −1.64 | 1,437.10 | 462.65 | Uncharacterized protein |
| Solyc09g007270 | −1.63 | 702.04 | 226.29 | PEROXIDASE_4 domain- |
| | | | | containing protein |
| Solyc08g080630 | −1.63 | 63.28 | 20.41 | Ethylene-responsive proteinase |
| | | | | inhibitor 1 (Ethylene- |
| | | | | responsive proteinase inhibitor |
| | | | | I) |
| Solyc01g097570 | −1.63 | 37.66 | 12.16 | Uncharacterized protein |
| Solyc08g075550 | −1.63 | 1,357.70 | 438.79 | Ubiquinol oxidase (EC |
| | | | | 1.10.3.11) |
| Solyc11g066800 | −1.63 | 513.53 | 166.26 | Aa_trans domain-containing |
| | | | | protein |
| Solyc11g005860 | −1.63 | 32.24 | 10.44 | Aminotran_5 domain- |
| | | | | containing protein |
| Solyc04g014990 | −1.62 | 117.58 | 38.15 | Glycosyltransferase (EC 2.4.1.—) |
| Solyc04g071780 | −1.62 | 1,685.02 | 548.31 | Uncharacterized protein |
| Solyc02g082760 | −1.62 | 5,973.82 | 1,948.22 | Catalase (EC 1.11.1.6) |
| Solyc04g074030 | −1.61 | 45.99 | 15.03 | Uncharacterized protein |
| Solyc08g062360 | −1.61 | 2,488.80 | 814.92 | ANK_REP_REGION domain- |
| | | | | containing protein |
| Solyc07g042470 | −1.61 | 40.85 | 13.38 | Uncharacterized protein |
| Solyc01g150158 | −1.61 | 64.52 | 21.14 | Uncharacterized protein |
| Solyc07g020870 | −1.61 | 73.13 | 23.97 | RING-type E3 ubiquitin |
| | | | | transferase (EC 2.3.2.27) |
| Solyc07g063730 | −1.61 | 268.32 | 88.08 | Receptor-like serine/threonine- |
| | | | | protein kinase (EC 2.7.11.1) |
| Solyc04g074350 | −1.60 | 950.93 | 312.97 | Uncharacterized protein |
| Solyc09g015020 | −1.60 | 30.87 | 10.16 | SHSP domain-containing |
| | | | | protein |
| Solyc05g012820 | −1.60 | 151.57 | 49.90 | Uncharacterized protein |
| Solyc02g070020 | −1.60 | 365.38 | 120.33 | Uncharacterized protein |
| Solyc03g114890 | −1.60 | 189.22 | 62.34 | COBRA-like protein |
| Solyc08g067960 | −1.60 | 9,084.05 | 2,994.81 | Uncharacterized protein |
| Solyc03g115920 | −1.60 | 2,517.31 | 830.30 | RBR-type E3 ubiquitin |
| | | | | transferase |
| Solyc12g005620 | −1.60 | 371.81 | 123.01 | Ethylene-inducing xylanase |
| | | | | (Ethylene-inducing xylanase |
| | | | | (Fragment)) (Fragment) |
| Solyc07g056240 | −1.59 | 349.81 | 115.80 | Uncharacterized protein |
| Solyc03g044910 | −1.59 | 1,318.57 | 437.32 | Uncharacterized protein |
| Solyc01g150176 | −1.59 | 5,428.15 | 1,803.25 | Snakin-2 |
| Solyc08g081490 | −1.59 | 506.60 | 168.34 | Putative polygalacturonase-like |
| | | | | (Fragment) |
| Solyc08g066270 | −1.59 | 139.60 | 46.43 | Protein kinase domain- |
| | | | | containing protein |
| Solyc07g065660 | −1.59 | 64.53 | 21.49 | GOLD domain-containing |
| | | | | protein |
| Solyc02g093270 | −1.58 | 1,090.49 | 363.52 | Caffeoyl-CoA O- |
| | | | | methyltransferase (EC |
| | | | | 2.1.1.104) (Fragment) |
| Solyc11g056620 | −1.58 | 857.40 | 285.93 | Uncharacterized protein |
| Solyc10g076460 | −1.58 | 91.35 | 30.50 | HTH myb-type domain- |
| | | | | containing protein |
| Solyc00g500406 | −1.58 | 77.64 | 26.04 | ADP-ribosyl cyclase/cyclic |
| | | | | ADP-ribose hydrolase (EC |
| | | | | 3.2.2.6) |
| Solyc03g098740 | −1.57 | 487.71 | 164.09 | Uncharacterized protein |
| Solyc08g060920 | −1.57 | 1,919.37 | 646.17 | SPX domain-containing protein |
| Solyc09g009590 | −1.57 | 344.22 | 116.25 | Stress-associated protein 8 |
| Solyc05g012430 | −1.57 | 87.68 | 29.62 | LRRNT_2 domain-containing |
| | | | | protein |
| Solyc05g041870 | −1.56 | 46.24 | 15.63 | DOMON domain-containing |
| | | | | protein |
| Solyc07g009380 | −1.56 | 2,206.36 | 747.11 | Xyloglucan |
| | | | | endotransglucosylase/hydrolase |
| | | | | (EC 2.4.1.207) |
| Solyc02g093420 | −1.56 | 176.65 | 59.82 | Uncharacterized protein |
| Solyc07g055320 | −1.56 | 16,326.01 | 5,533.69 | RING-type domain-containing |
| | | | | protein |
| Solyc12g006805 | −1.56 | 455.00 | 154.30 | Soul heme-binding family |
| | | | | protein |
| Solyc08g077380 | −1.56 | 61.81 | 21.00 | Uncharacterized protein |
| Solyc07g049550 | −1.56 | 206.93 | 70.31 | 1-aminocyclopropane-1- |
| | | | | carboxylate oxidase 4 (ACC |
| | | | | oxidase 4) (EC 1.14.17.4) |
| | | | | (Ethylene-forming enzyme) |
| | | | | (EFE) (Protein pHTOM5) |
| Solyc10g005030 | −1.56 | 1,729.58 | 588.31 | Uncharacterized protein |
| Solyc04g012185 | −1.55 | 99.61 | 33.92 | Protein kinase domain- |
| | | | | containing protein |
| Solyc01g009705 | −1.55 | 161.35 | 54.94 | Uncharacterized protein |
| Solyc08g080090 | −1.55 | 4,120.55 | 1,403.99 | Protein STAY-GREEN 1, |
| | | | | chloroplastic (LeSGR1) |
| | | | | (S1SGR1) (Protein GREEN |
| | | | | FLESH) |
| Solyc09g075330 | −1.55 | 71.13 | 24.27 | Pectinesterase (EC 3.1.1.11) |
| Solyc04g079700 | −1.55 | 71.85 | 24.56 | WD_REPEATS_REGION |
| | | | | domain-containing protein |
| Solyc07g049530 | −1.55 | 14,938.44 | 5,109.31 | Putative 1-aminocyclopropane- |
| | | | | 1-carboxylate oxidase 4-like |
| Solyc01g162830 | −1.54 | 475.16 | 162.85 | Nicotianamine synthase (EC |
| | | | | 2.5.1.43) (Chloronerva) (S- |
| | | | | adenosyl-L-methionine:S- |
| | | | | adenosyl-L-methionine:S- |
| | | | | adenosyl-methionine 3-amino- |
| | | | | 3-carboxypropyltransferase) |
| Solyc02g092890 | −1.54 | 54.33 | 18.63 | Uncharacterized protein |
| Solyc03g093890 | −1.54 | 466.95 | 160.16 | Uncharacterized protein |
| Solyc11g011360 | −1.54 | 153.84 | 52.79 | Nudix hydrolase domain- |
| | | | | containing protein |
| Solyc08g008630 | −1.54 | 2,435.12 | 835.97 | Uncharacterized protein |
| Solyc02g077290 | −1.54 | 561.46 | 193.06 | Glutamate receptor |
| Solyc11g017280 | −1.54 | 952.85 | 327.67 | Protein kinase domain- |
| | | | | containing protein |
| Solyc05g056470 | −1.54 | 76.76 | 26.41 | ABC transporter domain- |
| | | | | containing protein |
| Solyc06g036110 | −1.54 | 1,412.23 | 486.29 | C2 domain-containing protein |
| Solyc04g072050 | −1.53 | 304.99 | 105.38 | Uncharacterized protein |
| Solyc11g066440 | −1.53 | 3,520.53 | 1,216.97 | Rieske domain-containing |
| | | | | protein |
| Solyc04g072240 | −1.53 | 1,071.94 | 371.08 | GFO_IDH_MocA domain- |
| | | | | containing protein |
| Solyc07g065900 | −1.53 | 256.14 | 88.82 | Multifunctional fusion protein |
| | | | | [Includes: Uridine kinase (EC |
| | | | | 2.7.1.48); Fructose- |
| | | | | bisphosphate aldolase (EC |
| | | | | 4.1.2.13)] |
| Solyc01g111630 | −1.52 | 7,166.91 | 2,498.55 | Uncharacterized protein |
| Solyc08g067310 | −1.52 | 522.52 | 182.18 | Non-specific serine/threonine |
| | | | | protein kinase (EC 2.7.11.1) |
| Solyc03g116910 | −1.52 | 330.83 | 115.37 | Cinnamoyl-CoA reductase (EC |
| | | | | 1.2.1.44) |
| Solyc05g051850 | −1.52 | 5,897.45 | 2,061.64 | Inositol-3-phosphate synthase |
| | | | | (EC 5.5.1.4) |
| Solyc11g068940 | −1.52 | 46.80 | 16.36 | RING-type E3 ubiquitin |
| | | | | transferase (EC 2.3.2.27) |
| Solyc01g106620 | −1.52 | 198.18 | 69.33 | PR1 protein |
| Solyc06g062670 | −1.51 | 2,053.37 | 719.37 | C2H2-type domain-containing |
| | | | | protein |
| Solyc08g029000 | −1.51 | 237.76 | 83.38 | Lipoxygenase (EC 1.13.11.—) |
| Solyc06g060340 | −1.51 | 1,120.22 | 393.36 | Photosystem II 22 kDa protein, |
| | | | | chloroplastic (CP22) |
| Solyc11g008600 | −1.51 | 39.52 | 13.89 | Uncharacterized protein |
| Solyc03g119590 | −1.51 | 325.44 | 114.53 | Uncharacterized protein |
| Solyc09g059240 | −1.51 | 312.07 | 109.86 | Uncharacterized protein |
| Solyc12g009730 | −1.50 | 663.59 | 233.90 | LRRNT_2 domain-containing |
| | | | | protein |
| Solyc06g011270 | −1.50 | 34.95 | 12.33 | Uncharacterized protein |
| Solyc04g150172 | −1.50 | 42.96 | 15.16 | SANT domain-containing |
| | | | | protein |
| Solyc10g161270 | −1.50 | 192.13 | 67.84 | Uncharacterized protein |
| Solyc12g008650 | −1.50 | 116.49 | 41.16 | Inositol oxygenase (EC |
| | | | | 1.13.99.1) (Myo-inositol |
| | | | | oxygenase) |
| Solyc10g076540 | −1.50 | 291.06 | 102.87 | Uncharacterized protein |
| Solyc10g075150 | −1.50 | 49,611.02 | 17,548.47 | Uncharacterized protein |
| Solyc08g079230 | −1.50 | 291.34 | 103.15 | AAI domain-containing protein |
| Solyc03g117860 | −1.50 | 652.05 | 230.98 | 4-coumarate--CoA ligase (EC |
| | | | | 6.2.1.12) |
| Solyc07g052780 | −1.50 | 51.80 | 18.35 | T1R domain-containing protein |
| Solyc01g006555 | −1.49 | 9,938.51 | 3,526.93 | Lipoxygenase |
| Solyc03g046270 | −1.49 | 108.25 | 38.51 | Xyloglucan backbone O- |
| | | | | acetyltransferase 1 |
| Solyc01g110360 | −1.48 | 5,539.13 | 1,979.78 | Fructose-bisphosphate aldolase |
| | | | | (EC 4.1.2.13) |
| Solyc12g006050 | −1.48 | 69.35 | 24.88 | Uncharacterized protein |
| Solyc11g005430 | −1.48 | 69.31 | 24.87 | WAT1-related protein |
| Solyc10g086410 | −1.48 | 1,141.50 | 409.76 | Uncharacterized protein |
| Solyc09g011520 | −1.47 | 40.93 | 14.73 | Uncharacterized protein |
| Solyc10g006970 | −1.47 | 1,888.32 | 679.89 | Thioredoxin domain- |
| | | | | containing protein |
| Solyc04g011480 | −1.47 | 46.94 | 16.93 | CASP-like protein |
| Solyc01g102280 | −1.47 | 119.54 | 43.14 | Uncharacterized protein |
| Solyc12g009520 | −1.47 | 491.46 | 177.43 | Uncharacterized protein |
| Solyc03g097370 | −1.47 | 2,050.67 | 740.90 | Uncharacterized protein |
| Solyc01g060020 | −1.47 | 252.07 | 91.08 | Glucan endo-1,3-beta- |
| | | | | glucosidase B (EC 3.2.1.39) |
| | | | | ((1->3)-beta-glucan |
| | | | | endohydrolase B) ((1->3)-beta- |
| | | | | glucanase B) (Basic beta-1,3- |
| | | | | glucanase) (Beta-1,3- |
| | | | | endoglucanase B) |
| Solyc03g123680 | −1.47 | 394.54 | 142.61 | RING-type domain-containing |
| | | | | protein |
| Solyc09g075820 | −1.46 | 4,539.54 | 1,645.26 | Putative sugar transport protein |
| | | | | 13-like |
| Solyc08g076220 | −1.46 | 14,828.40 | 5,374.68 | Phosphoribulokinase (EC |
| | | | | 2.7.1.19) |
| Solyc04g040130 | −1.46 | 1,026.55 | 372.24 | Uncharacterized protein |
| Solyc10g086710 | −1.46 | 172.67 | 62.62 | Uncharacterized protein |
| Solyc12g005660 | −1.46 | 345.81 | 125.43 | Hop-interacting protein |
| | | | | THI121 |
| Solyc08g076290 | −1.46 | 4,169.44 | 1,512.54 | LrgB-like family protein |
| Solyc09g010530 | −1.46 | 152.09 | 55.24 | Na_H_Exchanger domain- |
| | | | | containing protein |
| Solyc04g077500 | −1.46 | 1,845.15 | 671.84 | Uncharacterized protein |
| Solyc05g007630 | −1.46 | 101.01 | 36.78 | NB-ARC domain-containing |
| | | | | protein |
| Solyc04g009030 | −1.46 | 12,313.91 | 4,485.25 | Glyceraldehyde-3-phosphate |
| | | | | dehydrogenase (EC 1.2.1.—) |
| Solyc01g094140 | −1.46 | 37.17 | 13.55 | Uncharacterized protein |
| Solyc02g083810 | −1.46 | 11,367.40 | 4,143.24 | Ferredoxin--NADP reductase, |
| | | | | leaf-type isozyme, |
| | | | | chloroplastic |
| Solyc05g005480 | −1.45 | 1,491.57 | 545.66 | 2-methylene-furan-3-one |
| | | | | reductase (EC 1.3.1.105) |
| | | | | (Enone oxidoreductase) (S1EO) |
| Solyc05g010180 | −1.45 | 540.10 | 197.67 | Amino_oxidase domain- |
| | | | | containing protein |
| Solyc02g091560 | −1.45 | 27,697.78 | 10,140.63 | Serine |
| | | | | hydroxymethyltransferase (EC |
| | | | | 2.1.2.1) |
| Solyc12g088390 | −1.44 | 1,073.56 | 394.57 | Zinc-finger protein |
| Solyc01g009080 | −1.44 | 211.29 | 77.73 | FHA domain-containing |
| | | | | protein |
| Solyc04g064670 | −1.44 | 428.48 | 157.82 | 23 kDa subunit of oxygen |
| | | | | evolving system of |
| | | | | photosystem II (23 kDa |
| | | | | thylakoid membrane protein) |
| | | | | (OEC 23 kDa subunit) |
| Solyc06g083190 | −1.44 | 4,381.86 | 1,620.13 | Peptidylprolyl isomerase (EC |
| | | | | 5.2.1.8) |
| Solyc02g079720 | −1.43 | 108.34 | 40.07 | EF-hand domain-containing |
| | | | | protein |
| Solyc02g020940 | −1.43 | 2,296.16 | 849.64 | Glyceraldehyde-3 -phosphate |
| | | | | dehydrogenase (EC 1.2.1.—) |
| Solyc01g088090 | −1.43 | 2,068.03 | 766.02 | AB hydrolase-1 domain- |
| | | | | containing protein |
| Solyc02g063510 | −1.43 | 247.29 | 91.62 | Uncharacterized protein |
| Solyc12g006140 | −1.43 | 429.31 | 159.33 | Chlorophyll a-b binding |
| | | | | protein, chloroplastic |
| Solyc06g053150 | −1.43 | 124.97 | 46.39 | Adenosinetriphosphatase (EC |
| | | | | 3.6.1.3) |
| Solyc09g083060 | −1.43 | 1,121.22 | 416.26 | RING-type E3 ubiquitin |
| | | | | transferase (EC 2.3.2.27) |
| Solyc11g069220 | −1.43 | 119.09 | 44.30 | MLO-like protein |
| Solyc09g092640 | −1.43 | 407.71 | 151.67 | Uncharacterized protein |
| Solyc05g010470 | −1.43 | 2,815.41 | 1,048.14 | WD_REPEATS_REGION |
| | | | | domain-containing protein |
| Solyc06g063370 | −1.42 | 10,194.70 | 3,798.45 | Chlorophyll a-b binding |
| | | | | protein, chloroplastic |
| Solyc10g076500 | −1.42 | 195.62 | 72.98 | LRRNT_2 domain-containing |
| | | | | protein |
| Solyc04g079660 | −1.42 | 759.36 | 283.47 | Uncharacterized protein |
| Solyc06g063060 | −1.42 | 378.75 | 141.45 | Uncharacterized protein |
| Solyc06g005090 | −1.42 | 66.79 | 24.95 | LOB domain-containing |
| | | | | protein |
| Solyc01g008130 | −1.42 | 361.69 | 135.19 | Uncharacterized protein |
| Solyc01g107080 | −1.42 | 43.79 | 16.39 | Uncharacterized protein |
| Solyc03g116570 | −1.42 | 1,550.77 | 580.91 | Uncharacterized protein |
| Solyc06g066530 | −1.42 | 186.68 | 69.98 | Uncharacterized protein |
| Solyc11g006030 | −1.41 | 310.85 | 116.69 | RING-type E3 ubiquitin |
| | | | | transferase (EC 2.3.2.27) |
| Solyc03g033790 | −1.41 | 626.56 | 235.36 | Adenosinetriphosphatase (EC |
| | | | | 3.6.1.3) |
| Solyc02g092110 | −1.41 | 249.77 | 93.90 | Phytosulfokine-beta |
| Solyc01g102610 | −1.41 | 464.70 | 174.97 | FAD-binding FR-type domain- |
| | | | | containing protein |
| Solyc05g051530 | −1.41 | 1,264.97 | 476.63 | ATP-binding cassette |
| | | | | transporter |
| Solyc11g012360 | −1.41 | 982.54 | 370.87 | Tonoplast dicarboxylate |
| | | | | transporter |
| Solyc02g093580 | −1.41 | 157.56 | 59.49 | Pectate lyase (EC 4.2.2.2) |
| Solyc01g044365 | −1.40 | 107.35 | 40.54 | Mannosyltransferase (EC |
| | | | | 2.4.1.—) |
| Solyc12g089020 | −1.40 | 57.22 | 21.63 | Protein kinase domain- |
| | | | | containing protein |
| Solyc04g077310 | −1.40 | 452.53 | 171.31 | Receptor-like serine/threonine- |
| | | | | protein kinase (EC 2.7.11.1) |
| Solyc07g052790 | −1.40 | 514.23 | 194.77 | NB-ARC domain-containing |
| | | | | protein |
| Solyc06g076250 | −1.40 | 1,485.37 | 563.63 | Uncharacterized protein |
| Solyc10g084400 | −1.40 | 4,340.37 | 1,650.12 | Protein-serine/threonine |
| | | | | phosphatase (EC 3.1.3.16) |
| Solyc12g044260 | −1.39 | 220.31 | 83.82 | Hydroxyphenylpyruvate |
| | | | | reductase |
| Solyc10g006150 | −1.39 | 242.86 | 92.40 | Uncharacterized protein |
| Solyc11g068620 | −1.39 | 4,178.67 | 1,592.76 | NAC domain-containing |
| | | | | protein |
| Solyc03g007860 | −1.39 | 256.17 | 97.82 | Uncharacterized protein |
| Solyc09g089780 | −1.39 | 113.72 | 43.44 | Fe2OG dioxygenase domain- |
| | | | | containing protein |
| Solyc09g009550 | −1.39 | 71.41 | 27.29 | AB hydrolase-1 domain- |
| | | | | containing protein |
| Solyc10g008660 | −1.39 | 342.46 | 131.11 | Malonyltransferase |
| Solyc08g079870 | −1.38 | 13,820.86 | 5,302.62 | Uncharacterized protein |
| Solyc03g033460 | −1.38 | 312.66 | 120.00 | SAM_MT_RS_MB_NOP |
| | | | | domain-containing protein |
| Solyc02g078400 | −1.38 | 10,013.82 | 3,849.76 | Putative allantoinase 1-like |
| Solyc01g067120 | −1.38 | 1,142.40 | 439.50 | cullin-1-like isoform X2 |
| Solyc10g085880 | −1.38 | 510.72 | 196.50 | UDP-glycosyltransferase 73C4 |
| | | | | (S1UGT73C4) (EC 2.4.1.—) |
| Solyc10g006230 | −1.37 | 15,077.11 | 5,814.86 | Chlorophyll a-b binding |
| | | | | protein, chloroplastic |
| Solyc10g085030 | −1.37 | 1,457.03 | 562.20 | Uncharacterized protein |
| Solyc03g117890 | −1.37 | 3,963.84 | 1,533.70 | Uncharacterized protein |
| Solyc02g088610 | −1.37 | 1,031.64 | 399.60 | Heat shock protein |
| Solyc05g015470 | −1.37 | 1,373.26 | 532.02 | Uncharacterized protein |
| Solyc02g079540 | −1.37 | 599.08 | 232.16 | Receptor-like serine/threonine- |
| | | | | protein kinase (EC 2.7.11.1) |
| Solyc04g054190 | −1.37 | 890.82 | 345.52 | ABC1 domain-containing |
| | | | | protein |
| Solyc05g053760 | −1.36 | 1,138.66 | 442.32 | J domain-containing protein |
| Solyc04g015750 | −1.36 | 3,090.02 | 1,201.61 | Magnesium chelatase (EC |
| | | | | 6.6.1.1) |
| Solyc01g080460 | −1.36 | 10,104.42 | 3,931.93 | Pyruvate, phosphate dikinase |
| | | | | (EC2.7.9.1) |
| Solyc05g052600 | −1.36 | 6,697.77 | 2,607.99 | FBPase domain-containing |
| | | | | protein |
| Solyc12g017480 | −1.36 | 259.84 | 101.21 | Uncharacterized protein |
| Solyc06g050140 | −1.36 | 60.33 | 23.53 | Uncharacterized protein |
| Solyc04g024780 | −1.36 | 278.67 | 108.74 | Uncharacterized protein |
| Solyc09g097910 | −1.36 | 615.01 | 240.00 | Uncharacterized protein |
| Solyc02g076980 | −1.36 | 2,118.68 | 827.57 | Uncharacterized protein |
| Solyc01g095900 | −1.35 | 3,367.26 | 1,316.86 | Uncharacterized protein |
| Solyc04g009860 | −1.35 | 1,974.14 | 772.35 | Fe2OG dioxygenase domain- |
| | | | | containing protein |
| Solyc11g044250 | −1.35 | 531.22 | 207.85 | Uncharacterized protein |
| Solyc02g063250 | −1.35 | 67.87 | 26.56 | Protein-methionine-S-oxide |
| | | | | reductase (EC 1.8.4.11) |
| Solyc03g119200 | −1.35 | 714.00 | 279.55 | Dipeptide epimerase (EC |
| | | | | 5.1.1.—) |
| Solyc01g079790 | −1.35 | 1,081.74 | 424.24 | Glucose-1-phosphate |
| | | | | adenylyltransferase (EC |
| | | | | 2.7.7.27) (ADP-glucose |
| | | | | pyrophosphorylase) |
| Solyc07g042250 | −1.35 | 2,580.68 | 1,012.57 | Chaperonin 21 |
| Solyc04g009910 | −1.35 | 59.06 | 23.18 | Protein kinase domain- |
| | | | | containing protein |
| Solyc04g049360 | −1.35 | 517.28 | 203.35 | Aminodeoxychorismate |
| | | | | synthase, chloroplastic (ADC |
| | | | | synthase) (EC 2.6.1.85) (P- |
| | | | | aminobenzoic acid synthase) |
| | | | | (PABA synthase) (Para- |
| | | | | aminobenzoate synthase) |
| Solyc04g081970 | −1.35 | 14,286.16 | 5,619.99 | Thioredoxin domain- |
| | | | | containing protein |
| Solyc02g080080 | −1.35 | 1,336.03 | 525.92 | Uncharacterized protein |
| Solyc08g065190 | −1.34 | 2,251.54 | 886.72 | Uncharacterized protein |
| Solyc06g071790 | −1.34 | 5,301.43 | 2,098.62 | Elongation factor Tu |
| Solyc12g009780 | −1.34 | 782.44 | 309.86 | LRRNT_2 domain-containing |
| | | | | protein |
| Solyc09g091580 | −1.34 | 1,084.46 | 429.54 | Putative ABC1 family protein- |
| | | | | like |
| Solyc08g016670 | −1.33 | 817.21 | 324.18 | Calcyclin-binding protein |
| Solyc10g018590 | −1.33 | 584.02 | 232.05 | PAP_fibrillin domain- |
| | | | | containing protein |
| Solyc10g018300 | −1.33 | 4,245.79 | 1,687.55 | Transketolase (EC 2.2.1.1) |
| Solyc11g006040 | −1.33 | 172.08 | 68.41 | Protein kinase domain- |
| | | | | containing protein |
| Solyc07g009443 | −1.33 | 349.82 | 139.13 | Receptor-like serine/threonine- |
| | | | | protein kinase (EC 2.7.11.1) |
| Solyc11g067160 | −1.33 | 1,441.10 | 573.20 | Putative ovule protein |
| Solyc09g089640 | −1.33 | 873.31 | 347.43 | Uncharacterized protein |
| Solyc06g008620 | −1.33 | 78.21 | 31.14 | Uncharacterized protein |
| Solyc01g102850 | −1.33 | 338.25 | 134.99 | ADP-ribosyl cyclase/cyclic |
| | | | | ADP-ribose hydrolase (EC |
| | | | | 3.2.2.6) |
| Solyc06g073180 | −1.32 | 1,733.70 | 692.95 | Putative fructokinase 2-like |
| Solyc03g059260 | −1.32 | 5,236.08 | 2,094.93 | PDZ domain-containing |
| | | | | protein |
| Solyc02g094150 | −1.32 | 583.31 | 233.74 | YrdC-like domain-containing |
| | | | | protein |
| Solyc04g150104 | −1.32 | 126.35 | 50.64 | Putative kirola-like (Fragment) |
| Solyc11g011180 | −1.32 | 521.42 | 208.99 | LRRNT_2 domain-containing |
| | | | | protein |
| Solyc03g013240 | −1.31 | 478.79 | 192.92 | Uncharacterized protein |
| Solyc07g047850 | −1.31 | 5,931.94 | 2,390.65 | Chlorophyll a-b binding |
| | | | | protein 4, chloroplastic (LHCII |
| | | | | type I CAB-4) (UHCP) |
| Solyc02g080540 | −1.31 | 5,315.80 | 2,146.50 | Uncharacterized protein |
| Solyc01g096620 | −1.31 | 161.05 | 65.12 | Uncharacterized protein |
| Solyc10g079810 | −1.31 | 245.24 | 99.17 | Uncharacterized protein |
| Solyc12g044950 | −1.31 | 793.24 | 320.88 | Fatty acid desaturase |
| Solyc10g080670 | −1.31 | 10,654.45 | 4,310.08 | Uncharacterized protein |
| Solyc07g009440 | −1.30 | 229.30 | 93.00 | Receptor-like serine/threonine- |
| | | | | protein kinase (EC 2.7.11.1) |
| Solyc09g014860 | −1.30 | 3,148.61 | 1,278.17 | Uncharacterized protein |
| Solyc05g026490 | −1.30 | 405.45 | 164.87 | Phosphoglucomutase (alpha-D- |
| | | | | glucose-1,6-bisphosphate- |
| | | | | dependent) (EC 5.4.2.2) |
| Solyc12g100010 | −1.29 | 184.11 | 75.12 | Uncharacterized protein |
| Solyc05g007880 | −1.29 | 189.72 | 77.44 | Dof-type domain-containing |
| | | | | protein |
| Solyc11g006740 | −1.29 | 86.72 | 35.41 | F-box domain-containing |
| | | | | protein |
| Solyc10g078440 | −1.29 | 2,156.88 | 881.08 | VQ domain-containing protein |
| Solyc02g090890 | −1.29 | 5,292.44 | 2,163.59 | Zeaxanthin epoxidase, |
| | | | | chloroplastic (EC 1.14.15.21) |
| Solyc11g012850 | −1.29 | 952.50 | 389.47 | Chlorophyll synthase |
| Solyc04g007980 | −1.29 | 5,434.75 | 2,224.42 | Fe2OG dioxygenase domain- |
| | | | | containing protein |
| Solyc05g005460 | −1.29 | 4,092.85 | 1,677.00 | Copia LTR rider |
| Solyc02g093860 | −1.29 | 3,906.93 | 1,600.91 | Amino acid transporter |
| Solyc07g006770 | −1.29 | 609.60 | 249.83 | Protein kinase domain- |
| | | | | containing protein |
| Solyc07g017800 | −1.29 | 1,758.75 | 721.67 | Uncharacterized protein |
| Solyc08g080650 | −1.28 | 1,599.56 | 656.55 | Uncharacterized protein |
| Solyc07g006420 | −1.28 | 397.44 | 163.30 | Uncharacterized protein |
| Solyc04g074050 | −1.28 | 173.78 | 71.47 | Protein kinase domain- |
| | | | | containing protein |
| Solyc09g018890 | −1.28 | 313.83 | 129.21 | Uncharacterized protein |
| Solyc10g076480 | −1.28 | 1,113.11 | 458.84 | Ammonium transporter |
| Solyc05g053850 | −1.27 | 563.11 | 232.74 | SP5G |
| Solyc11g069790 | −1.27 | 4,233.67 | 1,752.67 | Uncharacterized protein |
| Solyc07g008590 | −1.27 | 497.87 | 206.15 | LRRNT_2 domain-containing |
| | | | | protein |
| Solyc05g005290 | −1.27 | 327.96 | 135.81 | Poly [ADP-ribose] polymerase |
| | | | | (PARP) (EC 2.4.2.—) |
| Solyc01g080900 | −1.27 | 72.62 | 30.10 | Uncharacterized protein |
| Solyc07g053140 | −1.27 | 1,418.42 | 588.24 | B box-type domain-containing |
| | | | | protein |
| Solyc02g082900 | −1.27 | 2,285.25 | 948.16 | Str_synth domain-containing |
| | | | | protein |
| Solyc10g080570 | −1.27 | 207.70 | 86.23 | RING-type E3 ubiquitin |
| | | | | transferase (EC 2.3.2.27) |
| Solyc09g009540 | −1.27 | 1,378.78 | 573.06 | AB hydrolase-1 domain- |
| | | | | containing protein |
| Solyc05g053550 | −1.27 | 962.92 | 400.40 | Chaicone synthase (EC |
| | | | | 2.3.1.74) |
| Solyc01g109460 | −1.27 | 9,843.78 | 4,093.77 | Polyol/monosaccharide |
| | | | | transporter 4 |
| Solyc04g025530 | −1.27 | 281.21 | 116.99 | Glutamate decarboxylase (EC |
| | | | | 4.1.1.15) |
| Solyc02g077040 | −1.27 | 18,847.98 | 7,841.85 | Phytophthora-inhibited |
| | | | | protease 1 |
| Solyc12g094650 | −1.26 | 2,234.98 | 929.98 | Uncharacterized protein |
| Solyc04g072000 | −1.26 | 408.55 | 170.07 | Chitin-binding type-1 domain- |
| | | | | containing protein |
| Solyc02g092920 | −1.26 | 3,318.56 | 1,381.86 | Heavy metal cation transport |
| | | | | atpase |
| Solyc09g082860 | −1.26 | 1,728.78 | 719.98 | Sulfate adenylyltransferase (EC |
| | | | | 2.7.7.4) |
| Solyc01g099910 | −1.26 | 308.85 | 128.64 | AB hydrolase-1 domain- |
| | | | | containing protein |
| Solyc11g066890 | −1.26 | 685.07 | 285.59 | Arogenate dehydratase (EC |
| | | | | 4.2.1.91) |
| Solyc04g071340 | −1.26 | 320.08 | 133.54 | D-fructose-1,6-bisphosphate 1- |
| | | | | phosphohydrolase (EC |
| | | | | 3.1.3.11) |
| Solyc12g042600 | −1.26 | 789.46 | 329.90 | Glycosyltransferase (EC 2.4.1.—) |
| Solyc05g050380 | −1.26 | 5,865.79 | 2,452.80 | Cyclic nucleotide-gated |
| | | | | calmodulin-binding ion |
| | | | | channel |
| Solyc01g005520 | −1.26 | 1,640.06 | 686.65 | Uncharacterized protein |
| Solyc01g086660 | −1.26 | 209.45 | 87.74 | Uncharacterized protein |
| Solyc12g006840 | −1.25 | 1,216.15 | 509.77 | Receptor-like serine/threonine- |
| | | | | protein kinase (EC 2.7.11.1) |
| Solyc03g111940 | −1.25 | 115.11 | 48.26 | Uncharacterized protein |
| Solyc11g005190 | −1.25 | 71.60 | 30.02 | Ubiquitin ligase protein cop 1 |
| Solyc05g012850 | −1.25 | 1,595.24 | 669.53 | Uncharacterized protein |
| Solyc06g066680 | −1.25 | 118.83 | 49.89 | Mitochondrial |
| | | | | camitine/acylcamitine carrier |
| | | | | protein |
| Solyc04g006940 | −1.25 | 1,000.15 | 419.95 | Phospholipid-transporting |
| | | | | ATPase (EC 7.6.2.1) |
| Solyc07g007660 | −1.25 | 1,800.68 | 756.34 | Uncharacterized protein |
| Solyc01g073890 | −1.25 | 700.26 | 294.70 | Uncharacterized protein |
| Solyc01g105190 | −1.25 | 226.73 | 95.42 | Ubiquinol oxidase (EC |
| | | | | 1.10.3.11) |
| Solyc03g117870 | −1.25 | 2,029.16 | 855.79 | 4-coumarate--CoA ligase (EC |
| | | | | 6.2.1.12) |
| Solyc01g105120 | −1.25 | 3,231.42 | 1,362.99 | Uncharacterized protein |
| Solyc05g050350 | −1.25 | 382.96 | 161.54 | Cyclic nucleotide-binding |
| | | | | domain-containing protein |
| Solyc02g161350 | −1.25 | 81.68 | 34.45 | Receptor protein kinase |
| Solyc12g015630 | −1.24 | 135.94 | 57.36 | A2L_zn_ribbon domain- |
| | | | | containing protein |
| Solyc04g058170 | −1.24 | 89.24 | 37.69 | Uncharacterized protein |
| Solyc06g075530 | −1.24 | 144.25 | 61.01 | Putative ovule protein |
| Solyc09g090510 | −1.24 | 1,150.36 | 487.41 | Amino_oxidase domain- |
| | | | | containing protein |
| Solyc03g063560 | −1.24 | 8,248.35 | 3,496.61 | Glutamine amidotransferase |
| | | | | type-2 domain-containing |
| | | | | protein |
| Solyc03g096070 | −1.23 | 107.46 | 45.70 | Phosphoinositide |
| | | | | phospholipase C (EC 3.1.4.11) |
| Solyc01g102390 | −1.23 | 331.13 | 140.84 | Germin-like protein |
| Solyc07g040950 | −1.23 | 1,407.50 | 599.28 | VOC domain-containing |
| | | | | protein |
| Solyc09g091970 | −1.23 | 410.92 | 175.06 | Uncharacterized protein |
| Solyc04g048900 | −1.23 | 6,187.83 | 2,639.39 | Calreticulin |
| Solyc04g079420 | −1.23 | 5,079.22 | 2,166.59 | RPW8 domain-containing |
| | | | | protein |
| Solyc01g007990 | −1.23 | 894.10 | 381.77 | Uncharacterized protein |
| Solyc12g045030 | −1.22 | 11,392.55 | 4,874.23 | Short-chain type |
| | | | | dehydrogenasereductase |
| Solyc11g008530 | −1.22 | 799.40 | 342.03 | Uncharacterized protein |
| Solyc05g046300 | −1.22 | 2,826.64 | 1,209.87 | Uncharacterized protein |
| Solyc04g007490 | −1.22 | 636.59 | 272.75 | Uncharacterized protein |
| Solyc11g071990 | −1.22 | 164.95 | 70.75 | Pyr_redox_2 domain- |
| | | | | containing protein |
| Solyc12g009200 | −1.22 | 443.39 | 190.97 | Chlorophyll a-b binding |
| | | | | protein, chloroplastic |
| Solyc01g005410 | −1.21 | 1,267.39 | 546.26 | Uncharacterized protein |
| Solyc04g076730 | −1.21 | 112.51 | 48.51 | Uncharacterized protein |
| Solyc12g007010 | −1.21 | 1,417.55 | 611.47 | fe-S cluster assembly factor |
| | | | | HCF101, chloroplastic-like |
| | | | | isoform X2 |
| Solyc10g083330 | −1.21 | 532.56 | 229.79 | Uncharacterized protein |
| Solyc01g090430 | −1.21 | 699.18 | 301.85 | Uncharacterized protein |
| Solyc04g040120 | −1.21 | 157.77 | 68.15 | Uncharacterized protein |
| Solyc05g008960 | −1.21 | 279.57 | 120.88 | Uncharacterized protein |
| Solyc08g075470 | −1.21 | 391.87 | 169.56 | Uncharacterized protein |
| Solyc09g005000 | −1.21 | 1,665.23 | 721.03 | Clade VI lectin receptor kinase |
| Solyc08g006470 | −1.21 | 647.41 | 280.39 | C2H2-type domain-containing |
| | | | | protein |
| Solyc07g006370 | −1.21 | 1,531.45 | 663.83 | Uncharacterized protein |
| Solyc04g054950 | −1.21 | 142.02 | 61.57 | Uncharacterized protein |
| Solyc08g080050 | −1.20 | 4,193.02 | 1,822.13 | Uncharacterized protein |
| Solyc08g078950 | −1.20 | 5,212.43 | 2,265.43 | Uncharacterized protein |
| Solyc07g006140 | −1.20 | 1,531.43 | 666.13 | Uncharacterized protein |
| Solyc08g081700 | −1.20 | 152.84 | 66.56 | Uncharacterized protein |
| Solyc03g117590 | −1.20 | 14,192.40 | 6,186.97 | Heat shock protein binding |
| | | | | protein |
| Solyc01g095320 | −1.19 | 18,651.63 | 8,148.29 | BAG domain-containing |
| | | | | protein |
| Solyc01g108800 | −1.19 | 611.13 | 267.13 | Uncharacterized protein |
| Solyc01g096660 | −1.19 | 1,836.54 | 803.17 | Uncharacterized protein |
| Solyc02g070280 | −1.19 | 1,673.15 | 731.97 | AA_permease_C domain- |
| | | | | containing protein |
| Solyc06g063190 | −1.19 | 1,374.88 | 602.58 | Uncharacterized protein |
| Solyc05g051710 | −1.19 | 205.27 | 90.03 | Uncharacterized protein |
| Solyc01g163210 | −1.19 | 96.76 | 42.45 | Uncharacterized protein |
| Solyc07g043320 | −1.19 | 5,856.47 | 2,573.00 | Uncharacterized protein |
| Solyc08g005700 | −1.19 | 100.44 | 44.13 | Uncharacterized protein |
| Solyc08g082640 | −1.18 | 5,568.60 | 2,449.30 | Uncharacterized protein |
| Solyc02g082450 | −1.18 | 167.17 | 73.56 | Uncharacterized protein |
| Solyc02g031790 | −1.18 | 274.07 | 120.63 | Protein kinase domain- |
| | | | | containing protein |
| Solyc03g118850 | −1.18 | 260.45 | 114.68 | Putative ovule protein |
| Solyc07g056280 | −1.18 | 423.39 | 186.46 | WRKY16 protein |
| Solyc03g095620 | −1.18 | 2,382.17 | 1,049.50 | uncharacterized protein |
| | | | | LOC104247214 |
| Solyc07g056670 | −1.18 | 108.32 | 47.75 | Gibberellin 2-oxidase 2 |
| Solyc10g075160 | −1.18 | 901.20 | 397.71 | Ferredoxin |
| Solyc03g082690 | −1.18 | 1,936.66 | 854.82 | RING-type E3 ubiquitin |
| | | | | transferase (EC 2.3.2.27) |
| Solyc04g072230 | −1.18 | 915.83 | 404.51 | Protein kinase domain- |
| | | | | containing protein |
| Solyc04g079350 | −1.18 | 1,001.67 | 442.94 | Uncharacterized protein |
| Solyc03g114450 | −1.18 | 1,548.65 | 685.05 | Rhodanese domain-containing |
| | | | | protein |
| Solyc12g011450 | −1.17 | 4,300.15 | 1,905.23 | Chlorophyll a-b binding |
| | | | | protein, chloroplastic |
| Solyc07g063600 | −1.17 | 2,361.86 | 1,047.83 | Chlorophyll a-b binding |
| | | | | protein, chloroplastic |
| Solyc06g073320 | −1.17 | 14,250.50 | 6,331.66 | GDP-L-galactose |
| | | | | phosphorylase 1 |
| Solyc03g116870 | −1.17 | 1,302.37 | 578.70 | Uncharacterized protein |
| Solyc09g011010 | −1.17 | 411.65 | 183.15 | One helix protein |
| Solyc04g009850 | −1.17 | 353.04 | 157.12 | Fe2OG dioxygenase domain- |
| | | | | containing protein |
| Solyc10g007070 | −1.17 | 698.73 | 311.04 | Phytocyanin domain- |
| | | | | containing protein |
| Solyc05g009070 | −1.17 | 2,968.85 | 1,321.67 | PPM-type phosphatase |
| | | | | domain-containing protein |
| Solyc03g121420 | −1.17 | 1,261.24 | 561.94 | Uncharacterized protein |
| Solyc11g012325 | −1.17 | 81.02 | 36.11 | NA |
| Solyc05g047680 | −1.16 | 743.74 | 331.72 | cytochrome P450 78A3-like |
| Solyc12g010380 | −1.16 | 839.07 | 374.76 | ATP:AMP phosphotransferase |
| | | | | (EC2.7.4.3) |
| Solyc07g021630 | −1.16 | 236.41 | 105.61 | Pol |
| Solyc12g087870 | −1.16 | 1,576.24 | 704.15 | Uncharacterized protein |
| Solyc05g053210 | −1.16 | 2,903.82 | 1,298.05 | Non-specific serine/threonine |
| | | | | protein kinase (EC 2.7.11.1) |
| Solyc11g030730 | −1.16 | 639.17 | 285.82 | Uncharacterized protein |
| Solyc07g065160 | −1.16 | 217.38 | 97.29 | Pirin |
| Solyc03g093690 | −1.16 | 5,736.59 | 2,569.84 | Protein-methionine-S-oxide |
| | | | | reductase (EC 1.8.4.11) |
| Solyc07g053130 | −1.15 | 919.79 | 413.17 | Receptor-like serine/threonine- |
| | | | | protein kinase (EC 2.7.11.1) |
| Solyc11g013500 | −1.15 | 193.54 | 86.97 | Uncharacterized protein |
| Solyc03g034070 | −1.15 | 199.87 | 89.84 | Putative anti-Muellerian |
| | | | | hormone type-2 receptor-like |
| | | | | (Fragment) |
| Solyc09g082630 | −1.15 | 1,201.64 | 540.13 | 1,2-dihydroxy-3-keto-5- |
| | | | | methylthiopentene dioxygenase |
| | | | | (EC 1.13.11.54) (Acireductone |
| | | | | dioxygenase (Fe(2+)- |
| | | | | requiring)) (ARD) (Fe-ARD) |
| Solyc11g042640 | −1.15 | 1,627.50 | 731.62 | Rieske domain-containing |
| | | | | protein |
| Solyc01g087750 | −1.15 | 1,110.39 | 499.62 | Uncharacterized protein |
| Solyc10g006755 | −1.15 | 149.29 | 67.20 | B box-type domain-containing |
| | | | | protein |
| Solyc11g011240 | −1.15 | 282.57 | 127.20 | Uncharacterized protein |
| Solyc05g024260 | −1.15 | 116.99 | 52.69 | Bidirectional sugar transporter |
| | | | | SWEET |
| Solyc05g012590 | −1.15 | 1,999.62 | 900.87 | ANK_REP_REGION domain- |
| | | | | containing protein |
| Solyc11g007980 | −1.15 | 1,050.23 | 473.23 | Uncharacterized protein |
| Solyc03g031920 | −1.15 | 11,666.30 | 5,258.41 | Uncharacterized protein |
| Solyc05g008980 | −1.15 | 501.13 | 225.89 | Uncharacterized protein |
| Solyc08g080130 | −1.15 | 2,892.60 | 1,306.75 | Phospholipase D (EC 3.1.4.4) |
| Solyc01g060180 | −1.15 | 1,941.83 | 877.75 | Uncharacterized protein |
| Solyc03g080190 | −1.14 | 2,193.09 | 993.51 | Fe2OG dioxygenase domain- |
| | | | | containing protein |
| Solyc02g072160 | −1.14 | 2,191.36 | 992.81 | Uncharacterized protein |
| Solyc05g008070 | −1.14 | 256.52 | 116.22 | NB-ARC domain-containing |
| | | | | protein |
| Solyc02g031990 | −1.14 | 367.58 | 166.74 | VQ domain-containing protein |
| Solyc12g088870 | −1.14 | 480.19 | 217.85 | Uncharacterized protein |
| Solyc03g096050 | −1.14 | 134.41 | 61.00 | Fe2OG dioxygenase domain- |
| | | | | containing protein |
| Solyc07g042830 | −1.14 | 1,061.60 | 481.84 | Starch synthase, |
| | | | | chloroplastic/amyloplastic (EC |
| | | | | 2.4.1.—) |
| Solyc11g065740 | −1.14 | 599.84 | 272.33 | Uncharacterized protein |
| Solyc03g093540 | −1.14 | 2,626.09 | 1,193.41 | AP2/ERF domain-containing |
| | | | | protein |
| Solyc05g050500 | −1.14 | 1,373.83 | 624.56 | Uncharacterized protein |
| Solyc04g015460 | −1.14 | 386.05 | 175.69 | Receptor-like serine/threonine- |
| | | | | protein kinase (EC 2.7.11.1) |
| Solyc05g009040 | −1.13 | 3,320.38 | 1,511.99 | Uncharacterized protein |
| Solyc06g072460 | −1.13 | 489.81 | 223.06 | Uncharacterized protein |
| Solyc10g079860 | −1.13 | 1,740.32 | 793.04 | Uncharacterized protein |
| Solyc07g056420 | −1.13 | 8,113.75 | 3,701.14 | Glutathione S-transferase-like |
| | | | | protein |
| Solyc03g006490 | −1.13 | 874.76 | 399.35 | DUF3700 domain-containing |
| | | | | protein |
| Solyc10g049710 | −1.13 | 4,779.03 | 2,181.92 | ATP-dependent Clp protease |
| | | | | proteolytic subunit |
| Solyc04g072070 | −1.13 | 964.39 | 440.35 | WRKY transcription factor |
| Solyc08g076450 | −1.13 | 3,169.55 | 1,448.95 | Uncharacterized protein |
| Solyc10g009570 | −1.13 | 985.19 | 450.57 | Uncharacterized protein |
| Solyc08g077370 | −1.13 | 2,080.13 | 951.61 | Uncharacterized protein |
| Solyc02g089530 | −1.13 | 140.65 | 64.44 | Putative ovule protein |
| Solyc03g025450 | −1.13 | 1,916.69 | 878.50 | Receptor protein kinase |
| Solyc02g070940 | −1.12 | 6,929.09 | 3,177.44 | Chlorophyll a-b binding |
| | | | | protein 1B, chloroplastic |
| | | | | (LHCII type I CAB-1B) |
| | | | | (LHCP) |
| Solyc10g006330 | −1.12 | 632.42 | 290.05 | C2 NT-type domain-containing |
| | | | | protein |
| Solyc03g112970 | −1.12 | 117.28 | 53.80 | PMEI domain-containing |
| | | | | protein |
| Solyc01g097240 | −1.12 | 4,443.49 | 2,038.68 | Pathogenesis-related protein P2 |
| Solyc01g067660 | −1.12 | 2,723.26 | 1,250.60 | Beta-amylase (EC 3.2.1.2) |
| Solyc01g068490 | −1.12 | 542.85 | 249.37 | Uncharacterized protein |
| Solyc08g078780 | −1.12 | 863.37 | 396.77 | Uncharacterized protein |
| Solyc04g082420 | −1.12 | 3,249.65 | 1,494.51 | Uncharacterized protein |
| Solyc04g077320 | −1.12 | 346.79 | 159.58 | Receptor-like serine/threonine- |
| | | | | protein kinase (EC 2.7.11.1) |
| Solyc01g006520 | −1.12 | 747.58 | 344.12 | Uncharacterized protein |
| Solyc01g005040 | −1.12 | 350.04 | 161.15 | CASP-like protein |
| Solyc07g055660 | −1.12 | 1,056.16 | 486.65 | Uncharacterized protein |
| Solyc08g066790 | −1.12 | 509.33 | 234.72 | Phospholipase D (EC 3.1.4.4) |
| Solyc08g068850 | −1.12 | 106.65 | 49.17 | Uncharacterized protein |
| Solyc11g071780 | −1.11 | 137.25 | 63.38 | Uncharacterized protein |
| Solyc08g007970 | −1.11 | 1,710.03 | 789.60 | C2 NT-type domain-containing |
| | | | | protein |
| Solyc03g045070 | −1.11 | 421.07 | 194.67 | Ammonium transporter 1 |
| | | | | member 3 (LeAMT1;3) |
| Solyc07g008630 | −1.11 | 187.01 | 86.48 | Receptor-like protein EIX2 |
| | | | | (EIX receptor 2) |
| Solyc09g097960 | −1.11 | 170.44 | 78.81 | Uncharacterized protein |
| Solyc01g097340 | −1.11 | 346.79 | 160.40 | GDP-mannose 3′,5′-epimerase |
| | | | | (EC 5.1.3.18) (GDP-mannose |
| | | | | 3,5-epimerase) |
| Solyc02g065190 | −1.11 | 297.48 | 137.88 | Uncharacterized protein |
| Solyc09g075020 | −1.11 | 11,058.73 | 5,127.86 | Uncharacterized protein |
| Solyc05g010530 | −1.10 | 404.44 | 188.03 | Wall-associated kinase |
| Solyc08g081030 | −1.10 | 3,684.83 | 1,715.06 | Uncharacterized protein |
| Solyc04g049380 | −1.10 | 590.91 | 275.25 | DUF4283 domain-containing |
| | | | | protein |
| Solyc08g078760 | −1.10 | 244.99 | 114.13 | Uncharacterized protein |
| Solyc03g097820 | −1.10 | 129.70 | 60.44 | Basic helix-loop-helix |
| Solyc10g055680 | −1.10 | 1,763.45 | 822.20 | Methyltransferase (EC 2.1.1.—) |
| Solyc10g084070 | −1.10 | 157.31 | 73.35 | Uncharacterized protein |
| Solyc01g107900 | −1.10 | 6,695.37 | 3,122.42 | Diacylglycerol O- |
| | | | | acyltransferase (EC 2.3.1.20) |
| Solyc02g092700 | −1.10 | 2,033.71 | 948.66 | Putative ovule protein |
| Solyc02g085950 | −1.10 | 6,308.33 | 2,942.80 | Ribulose-bisphosphate |
| | | | | carboxylase (EC 4.1.1.39) |
| Solyc05g009930 | −1.10 | 3,730.79 | 1,743.85 | Uncharacterized protein |
| Solyc03g119700 | −1.10 | 2,311.37 | 1,081.08 | Putative ovule protein |
| | | | | (Fragment) |
| Solyc09g091030 | −1.10 | 5,961.82 | 2,788.61 | Beta-amylase (EC 3.2.1.2) |
| Solyc12g044280 | −1.09 | 505.40 | 236.67 | Uncharacterized protein |
| Solyc08g079180 | −1.09 | 1,843.64 | 864.23 | Elongation factor G, |
| | | | | chloroplastic (cEF-G) |
| Solyc08g016310 | −1.09 | 993.23 | 465.60 | Uncharacterized protein |
| Solyc04g078660 | −1.09 | 308.02 | 144.50 | Uncharacterized protein |
| Solyc06g063210 | −1.09 | 1,075.50 | 504.57 | Uncharacterized protein |
| Solyc09g075920 | −1.09 | 1,163.60 | 545.91 | Receptor-like serine/threonine- |
| | | | | protein kinase (EC 2.7.11.1) |
| Solyc08g013670 | −1.09 | 2,056.58 | 967.10 | Uncharacterized protein |
| Solyc12g010870 | −1.09 | 1,170.87 | 550.87 | RING-type domain-containing |
| | | | | protein |
| Solyc08g083320 | −1.09 | 2,534.96 | 1,193.10 | Granule-bound starch synthase |
| | | | | (Fragment) |
| Solyc06g066000 | −1.09 | 4,003.57 | 1,886.91 | ATP synthase subunit b′, |
| | | | | chloroplastic |
| Solyc01g096630 | −1.09 | 283.55 | 133.65 | ACI112 |
| Solyc07g063430 | −1.08 | 10,187.36 | 4,809.51 | Uncharacterized protein |
| Solyc08g079170 | −1.08 | 2,755.90 | 1,301.32 | Uncharacterized protein |
| Solyc02g071870 | −1.08 | 1,962.02 | 926.55 | Protein kinase domain- |
| | | | | containing protein |
| Solyc04g078860 | −1.08 | 207.29 | 97.91 | Glutamate receptor |
| Solyc07g008440 | −1.08 | 1,792.79 | 846.86 | Uncharacterized protein |
| Solyc05g056050 | −1.08 | 1,117.99 | 528.34 | Chlorophyll a-b binding |
| | | | | protein, chloroplastic |
| Solyc05g009740 | −1.08 | 139.89 | 66.12 | NB-ARC domain-containing |
| | | | | protein |
| Solyc03g033795 | −1.08 | 878.35 | 415.41 | Adenosinetriphosphatase (EC |
| | | | | 3.6.1.3) |
| Solyc08g079750 | −1.08 | 604.70 | 286.24 | Aminotran_1_2 domain- |
| | | | | containing protein |
| Solyc03g112150 | −1.08 | 1,455.61 | 689.07 | Elongation factor Tu |
| Solyc01g106480 | −1.08 | 4,040.28 | 1,912.80 | Malate dehydrogenase (EC |
| | | | | 1.1.1.37) |
| Solyc06g068970 | −1.08 | 529.65 | 250.77 | Peptidylprolyl isomerase (EC |
| | | | | 5.2.1.8) |
| Solyc03g095650 | −1.08 | 1,589.31 | 753.10 | MLO-like protein |
| Solyc01g096510 | −1.08 | 1,675.07 | 793.92 | VQ domain-containing protein |
| Solyc09g006005 | −1.08 | 5,130.43 | 2,432.96 | Uncharacterized protein |
| Solyc01g106500 | −1.07 | 742.54 | 352.50 | Protein kinase domain- |
| | | | | containing protein |
| Solyc02g070490 | −1.07 | 519.08 | 247.26 | AB hydrolase-1 domain- |
| | | | | containing protein |
| Solyc03g115850 | −1.07 | 155.24 | 74.00 | NAC domain-containing |
| | | | | protein |
| Solyc07g049645 | −1.07 | 1,507.80 | 720.33 | Putative benzyl alcohol O- |
| | | | | benzoyltransferase-like |
| Solyc10g076740 | −1.06 | 647.56 | 309.98 | Isoamyl acetate-hydrolyzing |
| | | | | esterase |
| Solyc05g007640 | −1.06 | 240.31 | 115.04 | Uncharacterized protein |
| Solyc11g007510 | −1.06 | 852.34 | 408.16 | Uncharacterized protein |
| Solyc08g075570 | −1.06 | 3,109.30 | 1,490.79 | Uncharacterized protein |
| Solyc06g068220 | −1.06 | 2,098.59 | 1,007.24 | AB hydrolase-1 domain- |
| | | | | containing protein |
| Solyc05g006440 | −1.06 | 3,261.53 | 1,565.99 | Uncharacterized protein |
| Solyc12g005630 | −1.06 | 7,537.35 | 3,620.96 | Plastoquinol--plastocyanin |
| | | | | reductase (EC 7.1.1.6) |
| Solyc02g081970 | −1.06 | 3,537.91 | 1,700.40 | EGF_CA domain-containing |
| | | | | protein |
| Solyc09g063150 | −1.06 | 250.53 | 120.43 | Uncharacterized protein |
| Solyc12g098910 | −1.06 | 474.93 | 228.31 | Non-specific serine/threonine |
| | | | | protein kinase (EC 2.7.11.1) |
| Solyc04g077360 | −1.06 | 735.25 | 353.57 | Uncharacterized protein |
| Solyc03g111170 | −1.06 | 658.12 | 316.56 | 4-coumarate--CoA ligase (EC |
| | | | | 6.2.1.12) |
| Solyc11g069700 | −1.06 | 6,125.88 | 2,947.46 | Putative elongation factor 1- |
| | | | | alpha-like |
| Solyc11g069940 | −1.06 | 2,168.54 | 1,043.42 | Glutaredoxin domain- |
| | | | | containing protein |
| Solyc01g090650 | −1.05 | 213.56 | 102.80 | Putative chromodomain- |
| | | | | helicase-DNA-binding protein |
| | | | | 1-like |
| Solyc04g082630 | −1.05 | 4,422.45 | 2,128.84 | Glyceraldehyde-3 -phosphate |
| | | | | dehydrogenase (EC 1.2.1.—) |
| Solyc06g053670 | −1.05 | 1,198.30 | 576.89 | Uncharacterized protein |
| Solyc01g010180 | −1.05 | 1,048.62 | 505.34 | Uncharacterized protein |
| Solyc04g081290 | −1.05 | 1,302.90 | 628.17 | Cytokinin riboside 5′- |
| | | | | monophosphate |
| | | | | phosphoribohydrolase (EC |
| | | | | 3.2.2.n1) |
| Solyc01g007890 | −1.05 | 314.73 | 151.87 | Putative zinc finger protein |
| | | | | MAGPIE-like |
| Solyc07g053220 | −1.05 | 3,500.31 | 1,689.15 | Receptor-like serine/threonine- |
| | | | | protein kinase (EC 2.7.11.1) |
| Solyc05g016310 | −1.05 | 787.41 | 380.19 | Peptidase A1 domain- |
| | | | | containing protein |
| Solyc04g040190 | −1.05 | 919.49 | 444.00 | Lycopene beta cyclase, |
| | | | | chloroplastic (EC 5.5.1.19) |
| Solyc05g041200 | −1.05 | 1,504.06 | 726.41 | 4-hydroxyphenylpyruvate |
| | | | | dioxygenase |
| Solyc09g082110 | −1.05 | 226.83 | 109.57 | Uncharacterized protein |
| Solyc01g091840 | −1.05 | 987.70 | 477.37 | Uncharacterized protein |
| Solyc08g008080 | −1.05 | 154.62 | 74.79 | RING-type domain-containing |
| | | | | protein |
| Solyc03g007760 | −1.05 | 663.39 | 321.01 | ATP binding protein |
| Solyc05g010670 | −1.05 | 6,710.05 | 3,251.13 | heat shock protein 90-5, |
| | | | | chloroplastic-like |
| Solyc12g056360 | −1.04 | 530.31 | 257.31 | Uncharacterized protein |
| Solyc11g005630 | −1.04 | 4,350.15 | 2,110.89 | Receptor-like serine/threonine- |
| | | | | protein kinase (EC 2.7.11.1) |
| Solyc06g050930 | −1.04 | 770.73 | 374.17 | Mannosyl-glycoprotein endo- |
| | | | | beta-N-acetylglucosaminidase |
| | | | | (EC 3.2.1.96) |
| Solyc01g099020 | −1.04 | 493.17 | 239.70 | GDSL lipase-like |
| | | | | chlorogenate-dependent |
| | | | | caffeoyltransferase |
| Solyc05g056380 | −1.04 | 155.59 | 75.73 | Uncharacterized protein |
| Solyc07g063930 | −1.04 | 233.84 | 113.85 | MFS domain-containing |
| | | | | protein |
| Solyc05g026550 | −1.04 | 2,141.30 | 1,042.89 | Uncharacterized protein |
| Solyc03g097610 | −1.04 | 813.81 | 396.39 | Bidirectional sugar transporter |
| | | | | SWEET |
| Solyc04g082020 | −1.04 | 1,701.04 | 828.62 | Hop-interacting protein |
| | | | | THI120 |
| Solyc10g085830 | −1.04 | 1,152.01 | 561.31 | Methyltransf_2 domain- |
| | | | | containing protein |
| Solyc07g044860 | −1.04 | 13,013.00 | 6,343.24 | 23 kDa subunit of oxygen |
| | | | | evolving system of |
| | | | | photosystem II (23 kDa |
| | | | | thylakoid membrane protein) |
| | | | | (OEC 23 kDa subunit) |
| Solyc05g041920 | −1.04 | 343.09 | 167.34 | RNase III domain-containing |
| | | | | protein |
| Solyc06g053600 | −1.04 | 425.32 | 207.53 | protein tas-like |
| Solyc02g068820 | −1.03 | 511.13 | 249.53 | Protein kinase domain- |
| | | | | containing protein |
| Solyc12g011280 | −1.03 | 308.01 | 150.43 | Chlorophyll a-b binding |
| | | | | protein, chloroplastic |
| Solyc02g093290 | −1.03 | 1,694.75 | 828.02 | Nicotinate |
| | | | | phosphoribosyltransferase (EC |
| | | | | 6.3.4.21) |
| Solyc07g062500 | −1.03 | 2,379.38 | 1,163.12 | CYP72A58 |
| Solyc10g085100 | −1.03 | 844.79 | 413.45 | Uncharacterized protein |
| Solyc07g049700 | −1.03 | 156.59 | 76.71 | NB-ARC domain-containing |
| | | | | protein |
| Solyc01g103110 | −1.03 | 1,676.21 | 822.04 | Uncharacterized protein |
| Solyc10g081850 | −1.03 | 1,214.36 | 595.75 | Syntaxin |
| Solyc02g086350 | −1.03 | 190.49 | 93.46 | Uncharacterized protein |
| Solyc12g057040 | −1.03 | 1,828.02 | 896.94 | Cryptochrome 1b |
| Solyc01g099690 | −1.03 | 3,835.21 | 1,882.08 | ATP-dependent Clp protease |
| | | | | proteolytic subunit |
| Solyc06g072620 | −1.03 | 162.95 | 80.00 | Bidirectional sugar transporter |
| | | | | SWEET |
| Solyc05g008820 | −1.03 | 567.81 | 278.90 | Phosphatidic acid phosphatase |
| | | | | alpha |
| Solyc03g006250 | −1.02 | 433.17 | 212.92 | Uncharacterized protein |
| Solyc06g011350 | −1.02 | 227.91 | 112.07 | Aquaporin (Plasmamembrane |
| | | | | intrinsic protein 24) |
| Solyc03g115610 | −1.02 | 466.66 | 229.49 | Protein kinase domain- |
| | | | | containing protein |
| Solyc02g080780 | −1.02 | 1,068.05 | 525.28 | Uncharacterized protein |
| Solyc05g013060 | −1.02 | 689.18 | 339.08 | Uncharacterized protein |
| Solyc11g070060 | −1.02 | 202.57 | 99.70 | Uncharacterized protein |
| Solyc01g108630 | −1.02 | 139.43 | 68.64 | Uncharacterized protein |
| Solyc10g007690 | −1.02 | 10,663.05 | 5,250.66 | Chlorophyll a-b binding |
| | | | | protein, chloroplastic |
| Solyc08g066020 | −1.02 | 185.64 | 91.46 | Uncharacterized protein |
| Solyc01g107820 | −1.02 | 7,105.55 | 3,501.70 | Uncharacterized protein |
| Solyc09g020190 | −1.02 | 3,377.00 | 1,664.75 | Uncharacterized protein |
| Solyc05g005150 | −1.02 | 1,265.87 | 624.58 | F-box domain-containing |
| | | | | protein |
| Solyc04g010190 | −1.02 | 628.23 | 310.00 | Uncharacterized protein |
| Solyc06g071070 | −1.02 | 645.36 | 318.73 | Short chain dehydrogenase |
| Solyc03g098210 | −1.02 | 1,130.33 | 558.34 | Cyclic nucleotide-binding |
| | | | | domain-containing protein |
| Solyc04g058110 | −1.01 | 819.55 | 405.54 | Receptor-like serine/threonine- |
| | | | | protein kinase (EC 2.7.11.1) |
| Solyc01g080280 | −1.01 | 7,547.65 | 3,735.02 | Glutamine synthetase (EC |
| | | | | 6.3.1.2) |
| Solyc02g086730 | −1.01 | 1,997.00 | 988.48 | 50S ribosomal protein L12, |
| | | | | chloroplastic |
| Solyc05g006740 | −1.01 | 1,615.29 | 799.66 | Glutathione s-transferase |
| Solyc03g005230 | −1.01 | 6,568.35 | 3,252.23 | SAM_MPBQ_MSBQ_MT |
| | | | | domain-containing protein |
| Solyc02g090980 | −1.01 | 213.58 | 105.76 | Protein kinase domain- |
| | | | | containing protein |
| Solyc04g005610 | −1.01 | 649.41 | 321.70 | NAC domain-containing |
| | | | | protein 2 (S1NAP2) |
| Solyc08g008150 | −1.01 | 156.35 | 77.49 | Uncharacterized protein |
| Solyc03g006620 | −1.01 | 1,113.88 | 552.11 | DUF4408 domain-containing |
| | | | | protein |
| Solyc04g078290 | −1.01 | 4,142.85 | 2,054.83 | Putative isoflavone 2′- |
| | | | | hydroxylase-like (Fragment) |
| Solyc09g090730 | −1.01 | 7,660.74 | 3,801.82 | Ammonium transporter |
| Solyc09g083190 | −1.01 | 963.20 | 478.51 | Uncharacterized protein |
| Solyc08g082280 | −1.01 | 4,042.79 | 2,009.37 | 4-coumarate--CoA ligase (EC |
| | | | | 6.2.1.12) |
| Solyc08g014440 | −1.01 | 276.78 | 137.62 | Uncharacterized protein |
| Solyc08g081455 | −1.01 | 154.68 | 76.92 | Uncharacterized protein |
| Solyc03g078360 | −1.01 | 586.39 | 291.70 | Uncharacterized protein |
| Solyc05g056480 | −1.01 | 870.30 | 432.99 | 2-oxoisovalerate |
| | | | | dehydrogenase subunit alpha 2, |
| | | | | mitochondrial |
| Solyc11g008540 | −1.01 | 761.04 | 378.76 | Uncharacterized protein |
| Solyc09g074610 | −1.01 | 633.54 | 315.34 | DNAJ heat shock N-terminal |
| | | | | domain-containing protein |
| Solyc09g011610 | −1.01 | 255.52 | 127.21 | Putative glutathione S- |
| | | | | transferase-like |
| Solyc06g074200 | −1.01 | 3,272.44 | 1,629.39 | Photosystem I subunit O |
| Solyc12g011150 | −1.00 | 482.19 | 240.33 | Metal ion binding protein |
| Solyc05g054230 | −1.00 | 144.92 | 72.28 | Nudix_hydro domain- |
| | | | | containing protein |
| Solyc07g006000 | −1.00 | 1,156.10 | 576.62 | Putative ovule protein |
| Solyc02g083250 | −1.00 | 2,481.56 | 1,238.14 | CRAL-TRIO domain- |
| | | | | containing protein |
| Solyc02g072470 | −1.00 | 433.69 | 216.42 | O-fucosyltransferase family |
| | | | | protein |
| Solyc01g096190 | −1.00 | 2,155.61 | 1,076.05 | Calcium-transporting ATPase, |
| | | | | endoplasmic reticulum-type |
| | | | | (EC 7.2.2.10) |
| Solyc12g011320 | −1.00 | 155.04 | 77.43 | Uncharacterized protein |
| Solyc11g020100 | −1.00 | 378.05 | 188.93 | NB-ARC domain-containing |
| | | | | protein |
| Solyc12g044940 | −1.00 | 3,297.64 | 1,648.21 | Putative ovule protein |

Table 8 depicts genes whose expression was downregulated by more than a 4-fold decrease in treated plants compared to control plants.

TABLE 8
Downregulated Genes between Treated and Control F3 Tomato
Plants (>4 fold decrease)

| | log2Fold | Control | Treatment | |
|---|---|---|---|---|
| Gene_ID | Change | Expression | Expression | Protein Name |
| Solyc03- | -7.11 | 159.20 | 1.15 | Glycosyltransferase |
| g078500 | | | | (EC 2.4.1.-) |
| Solyc09- | -6.62 | 28.69 | 0.29 | Phosphate transporter |
| g066410 | | | | |
| Solyc03- | -6.23 | 49.00 | 0.65 | SHSP domain- |
| g113930 | | | | containing protein |
| Solyc07- | -5.94 | 57.86 | 0.95 | Uncharacterized |
| g042400 | | | | protein |
| Solyc06- | -5.74 | 1,373.84 | 25.66 | HATPase_c domain- |
| g036290 | | | | containing protein |
| Solyc01- | -5.71 | 201.16 | 3.85 | Cytochrome P450 |
| g094130 | | | | |
| Solyc06- | -5.69 | 293.59 | 5.68 | Putative phosphatase |
| g062550 | | | | |
| Solyc01- | -5.69 | 357.59 | 6.93 | SPX domain- |
| g090890 | | | | containing protein |
| Solyc06- | -5.65 | 709.40 | 14.16 | Putative acid |
| g062540 | | | | phosphatase (Putative |
| | | | | phosphatase) |
| Solyc03- | -5.57 | 71.85 | 1.51 | Small heat stress |
| g123540 | | | | protein class CIII |
| Solyc02- | -4.93 | 8.84 | 0.29 | Uncharacterized |
| g068850 | | | | protein |
| Solyc04- | -4.90 | 108.83 | 3.64 | Uncharacterized |
| g014770 | | | | protein |
| Solyc01- | -4.77 | 964.22 | 35.41 | Uncharacterized |
| g091260 | | | | protein |
| Solyc03- | -4.73 | 1,234.82 | 46.49 | Uncharacterized |
| g115230 | | | | protein |
| Solyc01- | -4.61 | 64.09 | 2.62 | Uncharacterized |
| g073840 | | | | protein |
| Solyc03- | -4.59 | 114.08 | 4.73 | Uncharacterized |
| g059205 | | | | protein |
| Solyc10- | -4.58 | 2,712.88 | 113.21 | TPSI1 (TPSI1 |
| g085850 | | | | protein) |
| Solyc08- | -4.51 | 267.81 | 11.77 | SHSP domain- |
| g062340 | | | | containing protein |
| Solyc07- | -4.48 | 7.29 | 0.33 | Uncharacterized |
| g006670 | | | | protein |
| Solyc06- | -4.43 | 6.25 | 0.29 | Class I small heat |
| g076540 | | | | shock protein 20.1 |
| Solycl2- | -4.33 | 96.09 | 4.76 | Uncharacterized |
| g006720 | | | | protein |
| Solyc09- | -4.31 | 17.28 | 0.87 | HSF_DOMAIN |
| g065660 | | | | domain-containing |
| | | | | protein |
| Solyc09- | -4.27 | 3,153.68 | 163.15 | ATPase_AAA_core |
| g011080 | | | | domain-containing |
| | | | | protein |
| Solyc06- | -4.25 | 279.70 | 14.67 | Copper transporter |
| g005820 | | | | |
| Solyc03- | -4.25 | 5.52 | 0.29 | Uncharacterized |
| g111990 | | | | protein |
| Solyc11- | -4.19 | 5.15 | 0.28 | RING-type domain- |
| g011570 | | | | containing protein |
| Solyc0l- | -4.11 | 15.36 | 0.89 | Cytochrome B561 |
| g005140 | | | | |
| Solyc01- | -4.09 | 264.24 | 15.56 | SHSP domain- |
| g102960 | | | | containing protein |
| Solyc08- | -4.08 | 163.26 | 9.66 | Mitochondrial small |
| g078700 | | | | heat shock protein |
| Solyc05- | -4.06 | 767.35 | 46.13 | SHSP domain- |
| g014280 | | | | containing protein |
| Solyc09- | -4.02 | 146.67 | 9.03 | Uncharacterized |
| g011160 | | | | protein |
| Solyc12- | -3.97 | 4.41 | 0.28 | Nudix hydrolase |
| g015700 | | | | domain-containing |
| | | | | protein |
| Solyc04- | -3.96 | 5.08 | 0.33 | GH10 domain- |
| g162330 | | | | containing protein |
| Solyc09- | -3.94 | 657.34 | 42.86 | SHSP domain- |
| g015000 | | | | containing protein |
| Solyc08- | -3.90 | 4.33 | 0.29 | Aldehyde |
| g007890 | | | | dehydrogenase |
| | | | | (NAD+) |
| Solyc04- | -3.89 | 13.91 | 0.94 | Protein kinase |
| g063370 | | | | domain-containing |
| | | | | protein |
| Solyc03- | -3.74 | 4.37 | 0.33 | Phospholipid- |
| g121810 | | | | transporting ATPase |
| | | | | (EC 7.6.2.1) |
| Solyc12- | -3.72 | 24.45 | 1.85 | NA |
| g006770 | | | | |
| Solyc01- | -3.70 | 7.58 | 0.58 | Uncharacterized |
| g010365 | | | | protein (Fragment) |
| Solyc0l- | -3.67 | 11.02 | 0.86 | Uncharacterized |
| g073860 | | | | protein |
| Solyc05- | -3.62 | 116.01 | 9.46 | Uncharacterized |
| g053150 | | | | protein |
| Solyc11- | -3.57 | 186.81 | 15.76 | Uncharacterized |
| g066100 | | | | protein |
| Solyc07- | -3.54 | 6.65 | 0.57 | Dimer_Tnp_hAT |
| g032140 | | | | domain-containing |
| | | | | protein |
| Solyc07- | -3.52 | 53.52 | 4.67 | Flavin-containing |
| g042430 | | | | monooxygenase (EC |
| | | | | 1.-.-.-) |
| Solyc05- | -3.47 | 369.29 | 33.43 | Phol |
| g010060 | | | | |
| Solyc03- | -3.41 | 821.48 | 77.16 | Acid phosphatase |
| g098010 | | | | (EC 3.1.3.2) |
| | | | | (Fragment) |
| Solyc12- | -3.36 | 91.49 | 8.89 | Putative calcium- |
| g099790 | | | | dependent protein |
| | | | | kinase 17-like |
| Solyc10- | -3.34 | 11.75 | 1.16 | Epimerase domain- |
| g009540 | | | | containing protein |
| Solyc12- | -3.31 | 14.54 | 1.46 | Uncharacterized |
| g005920 | | | | protein |
| Solyc03- | -3.29 | 104.16 | 10.68 | HATPase_c domain- |
| g007890 | | | | containing protein |
| Solyc11- | -3.26 | 75.88 | 7.94 | HPt domain- |
| g070150 | | | | containing protein |
| Solyc08- | -3.26 | 100.86 | 10.55 | Fe2OG dioxygenase |
| g080040 | | | | domain-containing |
| | | | | protein |
| Solyc08- | -3.25 | 24.65 | 2.59 | Na(+)/H(+) antiporter |
| g081820 | | | | |
| Solyc07- | -3.20 | 8.03 | 0.87 | Neprosin domain- |
| g056704 | | | | containing protein |
| Solyc06- | -3.19 | 179.89 | 19.65 | Uncharacterized |
| g065060 | | | | protein |
| Solyc04- | -3.19 | 127.09 | 13.90 | Usp domain- |
| g014600 | | | | containing protein |
| Solyc04- | -3.19 | 967.59 | 106.32 | Heat shock protein 70 |
| g011440 | | | | isoform 3 |
| Solyc10- | -3.19 | 64.35 | 7.07 | SAUR family protein |
| g083320 | | | | |
| Solyc10- | -3.17 | 8.86 | 0.98 | Calmodulin protein |
| g061883 | | | | kinase |
| Solyc12- | -3.14 | 7.92 | 0.90 | WD-repeat protein |
| g006317 | | | | |
| Solyc05- | -3.14 | 69.09 | 7.85 | Uncharacterized |
| g053610 | | | | protein |
| Solyc04- | -3.14 | 10.55 | 1.20 | NA |
| g009870 | | | | |
| Solyc10- | -3.13 | 15.32 | 1.74 | Uncharacterized |
| g083400 | | | | protein |
| Solyc10- | -3.11 | 905.46 | 104.96 | Uncharacterized |
| g078220 | | | | protein |
| Solyc06- | -3.10 | 42.26 | 4.95 | WRKY domain- |
| g070990 | | | | containing protein |
| Solyc12- | -3.08 | 143.98 | 16.99 | Anthocyanin |
| g088170 | | | | acyltransferase |
| Solyc05- | -3.06 | 77.29 | 9.24 | WAT1-related protein |
| g005870 | | | | |
| Solyc08- | -3.05 | 33.48 | 4.05 | PPM-type |
| g065540 | | | | phosphatase domain- |
| | | | | containing protein |
| Solyc10- | -3.04 | 14.92 | 1.81 | Protein kinase |
| g082066 | | | | domain-containing |
| | | | | protein |
| Solyc01- | -3.02 | 32.92 | 4.04 | MFS domain- |
| g080680 | | | | containing protein |
| Solyc02- | -3.02 | 17.34 | 2.14 | Uncharacterized |
| g078150 | | | | protein |
| Solyc06- | -2.99 | 9.15 | 1.15 | Fucosyltransferase |
| g061210 | | | | (EC 2.4.1.-) |
| Solyc12- | -2.97 | 547.08 | 69.79 | Uncharacterized |
| g100250 | | | | protein |
| Solyc01- | -2.97 | 11.74 | 1.50 | Uncharacterized |
| g005880 | | | | protein |
| Solyc08- | -2.95 | 98.77 | 12.77 | Uncharacterized |
| g080530 | | | | protein |
| Solyc12- | -2.95 | 52.96 | 6.86 | Uncharacterized |
| g006760 | | | | protein |
| Solyc12- | -2.93 | 6.60 | 0.86 | Uncharacterized |
| g042965 | | | | protein |
| Solyc04- | -2.93 | 188.93 | 24.79 | Endonuclease (EC |
| g008245 | | | | 2.7.7.49) (Reverse |
| | | | | transcriptase) |
| | | | | (Fragment) |
| Solyc11- | -2.92 | 120.23 | 15.84 | Aminotran_5 domain- |
| g005840 | | | | containing protein |
| Solyc03- | -2.89 | 439.12 | 59.13 | Uncharacterized |
| g120920 | | | | protein |
| Solyc09- | -2.89 | 23.96 | 3.23 | Uncharacterized |
| g007020 | | | | protein |
| Solyc09- | -2.88 | 2,493.69 | 337.93 | Pathogenesis-related |
| g007010 | | | | protein PR1a (P4) |
| Solyc08- | -2.88 | 168.64 | 22.93 | Protein NP24 |
| g080640 | | | | (Fragment) |
| Solyc12- | -2.84 | 10.21 | 1.43 | Uncharacterized |
| g094460 | | | | protein |
| Solyc06- | -2.82 | 8.75 | 1.24 | Uncharacterized |
| g036250 | | | | protein |
| Solyc01- | -2.82 | 541.23 | 76.58 | F-box domain- |
| g099990 | | | | containing protein |
| Solyc04- | -2.82 | 402.93 | 57.23 | SHSP domain- |
| g014480 | | | | containing protein |
| Solyc04- | -2.81 | 9.21 | 1.31 | Phosphatidylinositol |
| g080690 | | | | transfer III |
| Solyc01- | -2.80 | 25.51 | 3.65 | Hcr9-OR2A |
| g014150 | | | | |
| Solyc02- | -2.80 | 38.99 | 5.60 | Cytokinin |
| g085880 | | | | hydroxylase-like |
| Solyc08- | -2.80 | 14.16 | 2.04 | ANK_REP_REGION |
| g021870 | | | | domain-containing |
| | | | | protein |
| Solyc06- | -2.79 | 425.16 | 61.62 | 17.6 kD class I small |
| g076560 | | | | heat shock protein |
| | | | | (Class I small heat |
| | | | | shock protein 17.6) |
| | | | | (Type I small heat |
| | | | | shock protein 17.6 |
| | | | | kDa isoform) |
| Solyc06- | -2.77 | 267.90 | 39.18 | Chaperone protein |
| g011380 | | | | ClpB3, mitochondrial |
| Solyc01- | -2.77 | 881.47 | 129.66 | Uncharacterized |
| g107460 | | | | protein |
| Solyc08- | -2.76 | 79.45 | 11.69 | Uncharacterized |
| g079370 | | | | protein |
| Solyc05- | -2.75 | 436.68 | 64.96 | Stromal antigen |
| g021245 | | | | |
| Solyc04- | -2.73 | 156.27 | 23.50 | Uncharacterized |
| g015120 | | | | protein |
| Solyc03- | -2.72 | 81.94 | 12.46 | Homeobox domain- |
| g034150 | | | | containing protein |
| Solyc02- | -2.71 | 151.33 | 23.18 | FAD-binding PCMH- |
| g070110 | | | | type domain- |
| | | | | containing protein |
| Solyc10- | -2.71 | 319.49 | 48.95 | HTH myb-type |
| g084370 | | | | domain-containing |
| | | | | protein |
| Solyc01- | -2.70 | 2,667.88 | 410.59 | Hcr9-OR3A |
| g009930 | | | | |
| Solyc08- | -2.69 | 178.33 | 27.56 | Beta-amylase (EC |
| g007130 | | | | 3.2.1.2) |
| Solyc11- | -2.66 | 20.60 | 3.26 | C2 domain- |
| g013250 | | | | containing protein |
| Solyc02- | -2.66 | 160.79 | 25.52 | Glutathione |
| g081340 | | | | transferase (EC |
| | | | | 2.5.1.18) |
| Solyc12- | -2.65 | 121.24 | 19.27 | Anthocyanin 5-O- |
| g098590 | | | | glucosyltransferase |
| Solyc10- | -2.65 | 3,968.16 | 633.54 | Uncharacterized |
| g085140 | | | | protein |
| Solyc06- | -2.64 | 408.95 | 65.66 | Uncharacterized |
| g011370 | | | | protein |
| Solyc06- | -2.63 | 17.29 | 2.80 | Zinc transporter |
| g005620 | | | | |
| Solyc09- | -2.62 | 1,381.38 | 225.47 | Glycosyltransferase |
| g098080 | | | | (EC 2.4.1.-) |
| Solyc02- | -2.61 | 163.19 | 26.74 | Uncharacterized |
| g088270 | | | | protein |
| Solyc03- | -2.60 | 5,030.47 | 830.92 | WD_REPEATS_RE |
| g120260 | | | | GION domain- |
| | | | | containing protein |
| Solyc01- | -2.60 | 420.78 | 69.54 | LRRNT_2 domain- |
| g009810 | | | | containing protein |
| Solyc09- | -2.59 | 18.82 | 3.12 | Uncharacterized |
| g073040 | | | | protein |
| Solyc04- | -2.59 | 59.91 | 9.94 | Serpin |
| g079450 | | | | |
| Solyc06- | -2.59 | 28.23 | 4.69 | Uncharacterized |
| g066700 | | | | protein |
| Solyc08- | -2.58 | 716.60 | 119.64 | Putative glycogenin- |
| g078650 | | | | 2-like |
| Solyc11- | -2.57 | 198.01 | 33.33 | Uncharacterized |
| g012120 | | | | protein |
| Solyc11- | -2.54 | 13.19 | 2.26 | Pleiotropic drug |
| g067000 | | | | resistance protein 2 |
| Solyc06- | -2.52 | 8.41 | 1.46 | Uncharacterized |
| g010045 | | | | protein |
| Solyc03- | -2.52 | 55.78 | 9.75 | N-acetyltransferase |
| g025710 | | | | domain-containing |
| | | | | protein |
| Solyc01- | -2.51 | 26.19 | 4.59 | Calcium-transporting |
| g097400 | | | | ATPase (EC 7.2.2.10) |
| Solyc09- | -2.51 | 12.14 | 2.13 | Peptidylprolyl |
| g092690 | | | | isomerase (EC |
| | | | | 5.2.1.8) |
| Solyc12- | -2.51 | 198.01 | 34.82 | Uncharacterized |
| g100240 | | | | protein |
| Solyc02- | -2.48 | 172.45 | 30.84 | Putative |
| g085020 | | | | dihydroflavonol 4- |
| | | | | reductase-like |
| Solyc01- | -2.48 | 10.22 | 1.84 | Protein kinase |
| g103240 | | | | domain-containing |
| | | | | protein |
| Solyc02- | -2.47 | 24.47 | 4.43 | Uncharacterized |
| g077070 | | | | protein |
| Solyc09- | -2.46 | 11.65 | 2.12 | Uncharacterized |
| g005147 | | | | protein |
| Solyc06- | -2.43 | 129.92 | 24.14 | Glutamate receptor |
| g063200 | | | | |
| Solyc02- | -2.42 | 42.61 | 7.97 | Putative |
| g079930 | | | | phosphosulfolactate |
| | | | | synthase-related |
| | | | | protein-like |
| Solyc07- | -2.41 | 167.27 | 31.37 | Protein kinase |
| g005110 | | | | domain-containing |
| | | | | protein |
| Solyc02- | -2.40 | 48.22 | 9.12 | HTH myb-type |
| g150131 | | | | domain-containing |
| | | | | protein |
| Solyc12- | -2.40 | 86.51 | 16.41 | Uncharacterized |
| g010940 | | | | protein |
| Solyc08- | -2.39 | 20.75 | 3.95 | Protein kinase |
| g082460 | | | | domain-containing |
| | | | | protein |
| Solyc04- | -2.39 | 639.66 | 121.96 | CS domain- |
| g072160 | | | | containing protein |
| Solyc03- | -2.38 | 55.56 | 10.65 | Protein |
| g025190 | | | | DETOXIFICATION |
| | | | | (Multidrug and toxic |
| | | | | compound extrusion |
| | | | | protein) |
| Solyc10- | -2.38 | 612.18 | 117.62 | Uncharacterized |
| g079620 | | | | protein |
| Solyc06- | -2.35 | 253.98 | 49.67 | Copia LTR rider |
| g006028 | | | | |
| Solyc12- | -2.35 | 60.40 | 11.84 | Sulfate transporter |
| g043040 | | | | |
| Solyc06- | -2.34 | 964.23 | 190.65 | Beta-carotene |
| g036260 | | | | hydroxylase 1 |
| Solyc05- | -2.34 | 103.20 | 20.43 | HTH myb-type |
| g009720 | | | | domain-containing |
| | | | | protein |
| Solyc07- | -2.34 | 49.61 | 9.83 | Terpene_synth_C |
| g052150 | | | | domain-containing |
| | | | | protein |
| Solyc01- | -2.33 | 64.27 | 12.78 | Uncharacterized |
| g016400 | | | | protein |
| Solyc09- | -2.33 | 347.17 | 69.07 | Caffeoyl-CoA O- |
| g082660 | | | | methyltransferase |
| | | | | (EC 2.1.1.104) |
| Solyc06- | -2.33 | 1,775.76 | 353.74 | Uncharacterized |
| g011400 | | | | protein |
| Solyc11- | -2.32 | 14.56 | 2.91 | F-box domain- |
| g015890 | | | | containing protein |
| Solyc04- | -2.32 | 101.66 | 20.33 | Uncharacterized |
| g016230 | | | | protein |
| Solyc03- | -2.30 | 8,902.63 | 1,805.30 | Glycosyltransferase |
| g078490 | | | | (EC 2.4.1.-) |
| Solyc07- | -2.30 | 272.28 | 55.38 | Bet_v_l domain- |
| g005370 | | | | containing protein |
| Solyc07- | -2.29 | 73.07 | 14.93 | Uncharacterized |
| g055460 | | | | protein |
| Solyc04- | -2.29 | 18.55 | 3.79 | Uncharacterized |
| g079460 | | | | protein |
| Solyc11- | -2.29 | 76.26 | 15.61 | Uncharacterized |
| g007490 | | | | protein |
| Solyc02- | -2.27 | 6,817.55 | 1,414.73 | Plastid lipid |
| g081170 | | | | associated protein |
| | | | | CHRC |
| Solyc06- | -2.27 | 720.70 | 149.58 | uncharacterized |
| g066590 | | | | protein |
| | | | | L0C104222458 |
| Solyc07- | -2.26 | 34.34 | 7.16 | Uncharacterized |
| g044910 | | | | protein |
| Solyc04- | -2.26 | 435.68 | 91.11 | Patatin (EC 3.1.1.-) |
| g079230 | | | | |
| Solyc11- | -2.25 | 630.02 | 132.22 | Uncharacterized |
| g007500 | | | | protein |
| Solyc12- | -2.24 | 161.19 | 34.06 | Na_H_Exchanger |
| g056160 | | | | domain-containing |
| | | | | protein |
| Solyc12- | -2.24 | 10.95 | 2.32 | BHLH domain- |
| g008930 | | | | containing protein |
| Solyc03- | -2.23 | 51.07 | 10.85 | Uncharacterized |
| g097570 | | | | protein |
| Solyc04- | -2.23 | 1,698.21 | 361.50 | Patatin (EC 3.1.1.-) |
| g079210 | | | | |
| Solyc06- | -2.23 | 153.33 | 32.76 | NA |
| g150138 | | | | |
| Solyc10- | -2.22 | 91.10 | 19.50 | Abhydrolase_3 |
| g050880 | | | | domain-containing |
| | | | | protein |
| Solyc08- | -2.22 | 951.95 | 204.01 | Uncharacterized |
| g079900 | | | | protein |
| Solyc03- | -2.22 | 34.44 | 7.40 | Uncharacterized |
| g111993 | | | | protein |
| Solyc05- | | | | Uncharacterized |
| g007940 | -2.22 | 166.43 | 35.78 | protein |
| Solyc01- | -2.20 | 289.50 | 63.07 | Uncharacterized |
| g087270 | | | | protein |
| Solyc01- | -2.18 | 15.34 | 3.38 | ATPase (EC 3.6.-.-) |
| g091870 | | | | (Arsenical pump- |
| | | | | driving ATPase) |
| | | | | (Arsenite-stimulated |
| | | | | ATPase) |
| Solyc12- | -2.18 | 144.93 | 32.09 | WRKY domain- |
| g056750 | | | | containing protein |
| Solyc11- | -2.17 | 163.33 | 36.17 | Uncharacterized |
| g042630 | | | | protein |
| Solyc03- | -2.17 | 48.09 | 10.65 | Small heat shock |
| g082420 | | | | protein, chloroplastic |
| Solyc03- | -2.17 | 13.20 | 2.93 | Uncharacterized |
| g006410 | | | | protein |
| Solyc01- | -2.17 | 59.25 | 13.15 | Uncharacterized |
| g007970 | | | | protein |
| Solyc08- | -2.16 | 33.77 | 7.57 | AAI domain- |
| g078920 | | | | containing protein |
| Solyc02- | -2.16 | 3,471.65 | 778.93 | Uncharacterized |
| g086454 | | | | protein |
| Solyc11- | -2.15 | 151.77 | 34.13 | PKS ER domain- |
| g011340 | | | | containing protein |
| Solyc06- | -2.15 | 495.91 | 111.80 | Protein kinase |
| g048740 | | | | domain-containing |
| | | | | protein |
| Solyc02- | -2.14 | 26.75 | 6.07 | Uncharacterized |
| g066800 | | | | protein |
| Solyc09- | -2.14 | 393.94 | 89.57 | Early light inducible |
| g082690 | | | | protein |
| Solyc12- | -2.13 | 17.16 | 3.92 | Putative cytochrome |
| g044956 | | | | P450 71A1-like |
| Solyc07- | -2.13 | 447.94 | 102.61 | Abhydrolase_3 |
| g040890 | | | | domain-containing |
| | | | | protein |
| Solyc07- | -2.12 | 276.44 | 63.47 | LRRNT_2 domain- |
| g008600 | | | | containing protein |
| Solyc07- | -2.12 | 343.61 | 79.01 | Uncharacterized |
| g056080 | | | | protein |
| Solyc08- | -2.11 | 76.82 | 17.75 | Uncharacterized |
| g150141 | | | | protein |
| Solyc12- | -2.11 | 25.27 | 5.85 | Uncharacterized |
| g042793 | | | | protein |
| Solyc02- | -2.11 | 15.28 | 3.54 | ATP-dependent zinc |
| g081550 | | | | metalloprotease |
| | | | | FTSH 6, chloroplastic |
| Solyc12- | -2.10 | 951.34 | 222.26 | Uncharacterized |
| g100260 | | | | protein |
| Solyc12- | -2.10 | 2,084.98 | 487.86 | Glyceraldehyde-3- |
| g094640 | | | | phosphate |
| | | | | dehydrogenase (EC |
| | | | | 1.2.1.-) |
| Solyc11- | -2.09 | 54.85 | 12.86 | S5A_REDUCTASE |
| g006300 | | | | domain-containing |
| | | | | protein |
| Solyc10- | -2.09 | 32.26 | 7.57 | Gibberellin 2-oxidase |
| g007560 | | | | 3 |
| Solyc01- | -2.09 | 13.57 | 3.19 | SANT domain- |
| g109690 | | | | containing protein |
| Solyc01- | -2.09 | 204.95 | 48.31 | Ankyrin repeat- |
| g088830 | | | | containing protein |
| Solyc12- | -2.08 | 1,798.72 | 424.64 | Uncharacterized |
| g049030 | | | | protein |
| Solyc05- | -2.08 | 25.05 | 5.92 | Nudix hydrolase |
| g016690 | | | | domain-containing |
| | | | | protein |
| Solyc10- | -2.08 | 34,558.06 | 8,168.62 | ATPase_AAA_core |
| g086580 | | | | domain-containing |
| | | | | protein |
| Solyc10- | -2.08 | 3,361.49 | 794.70 | Uncharacterized |
| g083290 | | | | protein |
| Solyc12- | -2.08 | 591.09 | 140.04 | LRRNT 2 domain- |
| g013680 | | | | containing protein |
| Solyc08- | -2.07 | 171.18 | 40.78 | Glutaredoxin domain- |
| g082590 | | | | containing protein |
| Solyc02- | -2.07 | 21,500.30 | 5,124.35 | Uncharacterized |
| g086458 | | | | protein |
| Solyc03- | -2.07 | 616.46 | 147.14 | Uncharacterized |
| g007370 | | | | protein |
| Solyc04- | -2.06 | 26.36 | 6.34 | Uncharacterized |
| g015610 | | | | protein |
| Solyc10- | -2.05 | 371.84 | 89.66 | (+)-neomenthol |
| g017570 | | | | dehydrogenase |
| Solyc05- | -2.05 | 126.44 | 30.64 | Exocyst subunit |
| g054820 | | | | Exo70 family protein |
| Solyc04- | -2.04 | 737.41 | 178.97 | Protein kinase |
| g007380 | | | | domain-containing |
| | | | | protein |
| Solyc11- | -2.04 | 1,151.28 | 279.50 | Fn3_like domain- |
| g044910 | | | | containing protein |
| Solyc08- | -2.04 | 3,855.30 | 936.63 | Uncharacterized |
| g067610 | | | | protein |
| Solyc04- | -2.03 | 302.02 | 73.91 | Uncharacterized |
| g080040 | | | | protein |
| Solyc04- | -2.03 | 44.53 | 10.92 | Metal ion binding |
| g072700 | | | | protein |
| Solyc01- | -2.03 | 46.86 | 11.51 | Uncharacterized |
| g014320 | | | | protein |
| Solyc06- | -2.02 | 19.22 | 4.73 | Uncharacterized |
| g075775 | | | | protein |
| Solyc03- | -2.02 | 11,262.92 | 2,769.84 | Uncharacterized |
| g117950 | | | | protein |
| Solyc03- | -2.02 | 604.99 | 148.95 | Uncharacterized |
| g115060 | | | | protein |
| Solyc08- | -2.02 | 1,729.32 | 427.76 | Uncharacterized |
| g061270 | | | | protein |
| Solyc08- | -2.01 | 59.76 | 14.82 | Uncharacterized |
| g007980 | | | | protein |
| Solyc09- | -2.01 | 36.27 | 9.01 | Uncharacterized |
| g014720 | | | | protein |
| Solyc03- | -2.01 | 2,480.34 | 616.27 | MFS domain- |
| g093140 | | | | containing protein |

### Example 8—Methylation Fingerprint

In order to determine if the irradiation method disclosed in the present application (one hour of red irradiation {between 620-750 nm} for half an hour before sunset and half an hour into the night, for one month) results in methylation alternations, DNA was extracted from F3 tomato plants (line M82) treated with said irradiation protocol and from control plants, and analyzed as follows:

### DNA Extraction and Quality Control

Genomic DNA was extracted from Tomato leaf samples using standard CTAB extraction protocol. The quantification and quality of the genomic DNA was assessed using Nanodrop2000 (Thermo Scientific, USA), Qubit (Thermo Scientific, USA) and agarose gel electrophoresis.

### Immunoprecipitation of Methylated DNA

The immunoprecipitation of DNA was performed using MagMeDIP DNA Immunoprecipitation kit (MagMeDIP, Cat. No.: CO2010020 mc-magme-A10) as per manufacturer's instructions and quantified using Qubit fluorometric assays.

### Library Preparation

MeDIP-Seq libraries were prepared using ThruPLEXPlasma-Seq Kit (Takara Bio, U.S.A.) at Genotypic Technology Pvt. Ltd., Bangalore, India. Briefly, ing of immunoprecipitated or input DNA processed using the MagMeDIP method for immunoprecipitation and purification (Diagenode, Cat #CO2010020) and quantified using Qubitfluorometric assays, was used as template for end-repair and stem-loop adapter ligation, whereby the 5′ ends of the DNA were only ligated with the adapters, while the 3′ ends were left with a nick. The free 3′ ends of the adapter-ligated DNA fragments were subsequently extended for library synthesis, and Illumina-compatible indexes were added through high-fidelity library amplification. Libraries were purified using 1× JetSeq Beads (Bioline, Cat #BIO-68031), followed by library quality control check. Illumina-compatible sequencing libraries were quantified by Qubitfluorometer (Thermo Fisher Scientific, MA, USA) and fragment size distribution was analyzed on Agilent 2200 Tapestation.

### Illumina Sequencing

The libraries were paired-end sequenced on Illumina HiSeq X Ten sequencer for 150 cycles (Illumina, San Diego, USA) following manufacturer's instructions.

### Data Analysis

The MeDIP-Seq analysis was performed by processing the raw data for removal of low-quality data and adaptor sequences. The high-quality reads were considered for alignment with reference genome. Aligned reads were used for identification of enriched regions or methylated regions as peaks and further motifs were identified in the peak regions. Methylated regions were annotated further to identify their genomic locations. This experiment was carried out in triplicates, and was subjected to rigorous and stringent statistical tests and evaluations.

Based on the annotation from the genomic alignments, exons of genes exhibiting methylation patterns were identified. Table 9 shows methylation patterns detected solely in the exons of treated samples (tomato plants exposed to the irradiation protocol disclosed in the present application), which are not detected in the control samples.

TABLE 9
Methylated Exons of Genes of F3 Tomato Plants Treated by The
Irradiation Treatment of The Present Invention

| Annotation | Gene Name | Pathway |
|---|---|---|
| promoter-TSS | Solyc01g104210 | NA |
| (ID = exon: | | |
| Solyc01g104210.3.1.1; | | |
| Parent = mRNA: | | |
| Solyc01g104210.3.1) | | |
| promoter-TSS | Solyc01g105150 | NA |
| (ID = exon: | | |
| Solyc01g105150.4.1.1; | | |
| Parent = mRNA: | | |
| Solyc01g105150.4.1) | | |
| promoter-TSS | Solyc01g107290 | NA |
| (ID = exon: | | |
| Solyc01g107290.3.1.2; | | |
| Parent = mRNA: | | |
| Solyc01g107290.3.1) | | |
| promoter-TSS | Solyc01g111750 | NA |
| (ID = exon: | | |
| Solyc01g111750.4.1.4; | | |
| Parent = mRNA: | | |
| Solyc01g111750.4.1) | | |
| TTS | Solyc01g160170 | NA |
| (ID = exon: | | |
| Solyc01g160170.1.1.1; | | |
| Parent = mRNA: | | |
| Solyc01g160170.1.1) | | |
| TTS | Solyc01g160520 | NA |
| (ID = exon: | | |
| Solyc01g160520.1.1.1; | | |
| Parent = mRNA: | | |
| Solyc01g160520.1.1) | | |
| promoter-TSS | Solyc01g161640 | NA |
| (ID = exon: | | |
| Solyc01g161640.1.1.2; | | |
| Parent = mRNA: | | |
| Solyc01g161640.1.1) | | |
| promoter-TSS | Solyc02g005360 | NA |
| (ID = exon: | | |
| Solyc02g005360.3.1.4; | | |
| Parent = mRNA: | | |
| Solyc02g005360.3.1) | | |
| TTS | Solyc02g014190 | Protein |
| (ID = exon: | | phosphatases and |
| Solyc02g014190.4.1.5; | | associated proteins |
| Parent = mRNA: | | [01009] |
| Solyc02g014190.4.1) | | |
| TTS | Solyc02g014350 | NA |
| (ID = exon: | | |
| Solyc02g014350.4.1.25; | | |
| Parent = mRNA: | | |
| Solyc02g014350.4.1) | | |
| exon | Solyc02g030220 | NA |
| (ID = exon: | | |
| Solyc02g030220.1.1.1; | | |
| Parent = mRNA: | | |
| Solyc02g030220.1.1, | | |
| exon 1 of 1) | | |
| promoter-TSS | Solyc02g031700 | NA |
| (ID = exon: | | |
| Solyc02g031700.3.1.1; | | |
| Parent = mRNA: | | |
| Solyc02g031700.3.1) | | |
| promoter-TSS | Solyc02g062310 | NA |
| (ID = exon: | | |
| Solyc02g062310.1.1.1; | | |
| Parent = mRNA: | | |
| Solyc02g062310.1.1) | | |
| TTS | Solyc02g064850 | NA |
| (ID = exon: | | |
| Solyc02g064850.2.1.1; | | |
| Parent = mRNA: | | |
| Solyc02g064850.2.1) | | |
| promoter-TSS | Solyc02g067270 | Chromosome and |
| (ID = exon: | | associated proteins |
| Solyc02g067270.3.1.2; | | [03036] |
| Parent = mRNA: | | |
| Solyc02g067270.3.1) | | |
| promoter-TSS | Solyc02g078800 | Cytoskeleton proteins |
| (ID = exon: | | [04812] |
| Solyc02g078800.4.1.2; | | |
| Parent = mRNA: | | |
| Solyc02g078800.4.1) | | |
| promoter-TSS | Solyc02g079060 | RNA transport [03013]; |
| (ID = exon: | | Ribosome biogenesis |
| Solyc02g079060.3.1.6; | | [03009]; Translation |
| Parent = mRNA: | | factors [03012] |
| Solyc02g079060.3.1) | | |
| TTS | Solyc02g081500 | NA |
| (ID = exon: | | |
| Solyc02g081500.3.1.3; | | |
| Parent = mRNA: | | |
| Solyc02g081500.3.1) | | |
| promoter-TSS | Solyc02g085790 | Exosome [04147]; |
| (ID = exon: | | Chaperones and folding |
| Solyc02g085790.3.1.2; | | catalysts [03110] |
| Parent = mRNA: | | |
| Solyc02g085790.3.1) | | |
| promoter-TSS | Solyc02g086150 | Translation factors |
| (ID = exon: | | [03012] |
| Solyc02g086150.4.1.9; | | |
| Parent = mRNA: | | |
| Solyc02g086150.4.1) | | |
| promoter-TSS | Solyc02g086190 | Steroid biosynthesis |
| (ID = exon: | | [00100] |
| Solyc02g086190.3.1.1; | | |
| Parent = mRNA: | | |
| Solyc02g086190.3.1) | | |
| promoter-TSS | Solyc02g088750 | NA |
| (ID = exon: | | |
| Solyc02g088750.2.1.1; | | |
| Parent = mRNA: | | |
| Solyc02g088750.2.1) | | |
| promoter-TSS | Solyc02g090530 | NA |
| (ID = exon: | | |
| Solyc02g090530.4.1.1; | | |
| Parent = mRNA: | | |
| Solyc02g090530.4.1) | | |
| TTS | Solyc02g150116 | NA |
| (ID = exon: | | |
| Solyc02g150116.1.1.3; | | |
| Parent = mRNA: | | |
| Solyc02g150116.1.1) | | |
| promoter-TSS | Solyc02g161190 | NA |
| (ID = exon: | | |
| Solyc02g161190.1.1.1; | | |
| Parent = mRNA: | | |
| Solyc02g161190.1.1) | | |
| promoter-TSS | Solyc03g007370 | Transcription |
| (ID = exon: | | machinery |
| Solyc03g007370.3.1.6; | | [03021] |
| Parent = mRNA: | | |
| Solyc03g007370.3.1) | | |
| promoter-TSS | Solyc03g013290 | NA |
| (ID = exon: | | |
| Solyc03g013290.1.1.2; | | |
| Parent = mRNA: | | |
| Solyc03g013290.1.1) | | |
| promoter-TSS | Solyc03g033790 | NA |
| (ID = exon: | | |
| Solyc03g033790.4.1.3; | | |
| Parent = mRNA: | | |
| Solyc03g033790.4.1) | | |
| promoter-TSS | Solyc03g044120 | Phenylalanine |
| (ID = exon: | | metabolism [00360] |
| Solyc03g044120.1.1.1; | | |
| Parent = mRNA: | | |
| Solyc03g044120.1.1) | | |
| promoter-TSS | Solyc03g046440 | NA |
| (ID = exon: | | |
| Solyc03g046440.3.1.2; | | |
| Parent = mRNA: | | |
| Solyc03g046440.3.1) | | |
| exon | Solyc03g082770 | NA |
| (ID = exon: | | |
| Solyc03g082770.1.1.1; | | |
| Parent = mRNA: | | |
| Solyc03g082770.1.1, | | |
| exon 1 of 1) | | |
| TTS | Solyc03g093220 | NA |
| (ID = exon: | | |
| Solyc03g093220.3.1.1; | | |
| Parent = mRNA: | | |
| Solyc03g093220.3.1) | | |
| promoter-TSS | Solyc03g096007 | NA |
| (ID = exon: | | |
| Solyc03g096020.3.1.2; | | |
| Parent = mRNA: | | |
| Solyc03g096020.3.1) | | |
| promoter-TSS | Solyc03g098090 | NA |
| (ID = exon: | | |
| Solyc03g098090.2.1.3; | | |
| Parent = mRNA: | | |
| Solyc03g098090.2.1) | | |
| promoter-TSS | Solyc03g098170 | Exosome [04147]; |
| (ID = exon: | | Cytoskeleton proteins |
| Solyc03g098170.3.1.8; | | [04812] |
| Parent = mRNA: | | |
| Solyc03g098170.3.1) | | |
| promoter-TSS | Solyc03g115610 | NA |
| (ID = exon: | | |
| Solyc03g115610.3.1.1; | | |
| Parent = mRNA: | | |
| Solyc03g115610.3.1) | | |
| TTS | Solyc03g150138 | NA |
| (ID = exon: | | |
| Solyc03g150138.2.1.6; | | |
| Parent = mRNA: | | |
| Solyc03g150138.2.1) | | |
| promoter-TSS | Solyc03g160690 | NA |
| (ID = exon: | | |
| Solyc03g160690.1.1.4; | | |
| Parent = mRNA: | | |
| Solyc03g160690.1.1) | | |
| promoter-TSS | Solyc03g161120 | NA |
| (ID = exon: | | |
| Solyc03g161120.1.1.8; | | |
| Parent = mRNA: | | |
| Solyc03g161120.1.1) | | |
| promoter-TSS | Solyc03g161120 | NA |
| (ID = exon: | | |
| Solyc03g161120.1.1.8; | | |
| Parent = mRNA: | | |
| Solyc03g161120.1.1) | | |
| promoter-TSS | Solyc04g010010 | NA |
| (ID = exon: | | |
| Solyc04g010010.4.1.1; | | |
| Parent = mRNA: | | |
| Solyc04g010010.4.1) | | |
| TTS | Solyc04g016590 | Sulfur metabolism |
| (ID = exon: | | [00920]; Transfer RNA |
| Solyc04g016590.3.1.5; | | biogenesis [03016] |
| Parent = mRNA: | | |
| Solyc04g016590.3.1) | | |
| TTS | Solyc04g024600 | NA |
| (ID = exon: | | |
| Solyc04g024600.1.1.1; | | |
| Parent = mRNA: | | |
| Solyc04g024600.1.1) | | |
| TTS | Solyc04g045420 | Exosome [04147]; |
| (ID = exon: | | Chromosome and |
| Solyc04g045420.1.1.1; | | associated proteins |
| Parent = mRNA: | | [03036]; Chaperones |
| Solyc04g045420.1.1) | | and folding catalysts |
| | | [03110] |
| promoter-TSS | Solyc04g049893 | NA |
| (ID = exon: | | |
| Solyc04g049893.1.1.4; | | |
| Parent = mRNA: | | |
| Solyc04g049893.1.1) | | |
| TTS | Solyc04g051440 | NA |
| (ID = exon: | | |
| Solyc04g051440.1.1.1; | | |
| Parent = mRNA: | | |
| Solyc04g051440.1.1) | | |
| TTS | Solyc04g054260 | NA |
| (ID = exon: | | |
| Solyc04g054260.5.1.2; | | |
| Parent = mRNA: | | |
| Solyc04g054260.5.1) | | |
| TTS | Solyc04g056420 | Phenylpropanoid |
| (ID = exon: | | biosynthesis [00940] |
| Solyc04g056420.1.1.5; | | |
| Parent = mRNA: | | |
| Solyc04g056420.1.1) | | |
| promoter-TSS | Solyc04g080210 | NA |
| (ID = exon: | | |
| Solyc04g080210.1.1.1; | | |
| Parent = mRNA: | | |
| Solyc04g080210.1.1) | | |
| TTS | Solyc04g080290 | NA |
| (ID = exon: | | |
| Solyc04g080290.3.1.1; | | |
| Parent = mRNA: | | |
| Solyc04g080290.3.1) | | |
| TTS | Solyc04g080420 | NA |
| (ID = exon: | | |
| Solyc04g080420.5.1.4; | | |
| Parent = mRNA: | | |
| Solyc04g080420.5.1) | | |
| TTS | Solyc04g080800 | Ribosome biogenesis |
| (ID = exon: | | [03009] |
| Solyc04g080800.4.1.1; | | |
| Parent = mRNA: | | |
| Solyc04g0808004.1) | | |
| TTS | Solyc04g161000 | NA |
| (ID = exon: | | |
| Solyc04g161000.1.1.4; | | |
| Parent = mRNA: | | |
| Solyc04g161000.1.1) | | |
| TTS | Solyc04g162320 | NA |
| (ID = exon: | | |
| Solyc04g162320.1.1.5; | | |
| Parent = mRNA: | | |
| Solyc04g162320.1.1) | | |
| promoter-TSS | Solyc05g005290 | NA |
| (ID = exon: | | |
| Solyc05g005290.4.1.5; | | |
| Parent = mRNA: | | |
| Solyc05g005290.4.1) | | |
| promoter-TSS | Solyc05g006290 | NA |
| (ID = exon: | | |
| Solyc05g006290.2.1.2; | | |
| Parent = mRNA: | | |
| Solyc05g006290.2.1) | | |
| TTS | Solyc05g008260 | Other types of O-glycan |
| (ID = exon: | | biosynthesis [00514]; |
| Solyc05g008260.3.1.8; | | Glycosyltransferases |
| Parent = mRNA: | | [01003] |
| Solyc05g008260.3.1) | | |
| promoter-TSS | Solyc05g008910 | NA |
| (ID = exon: | | |
| Solyc05g008910.3.1.3; | | |
| Parent = mRNA: | | |
| Solyc05g008910.3.1) | | |
| TTS | Solyc05g012325 | NA |
| (ID = exon: | | |
| Solyc05g012325.1.1.1; | | |
| Parent = mRNA: | | |
| Solyc05g012325.1.1) | | |
| promoter-TSS | Solyc05g013990 | Exosome [04147]; |
| (ID = exon: | | Chromosome and |
| Solyc05g013990.3.1.1; | | associated proteins |
| Parent = mRNA: | | [03036]; Chaperones |
| Solyc05g013990.3.1) | | and folding catalysts |
| | | [03110] |
| promoter-TSS | Solyc05g015075 | NA |
| (ID = exon: | | |
| Solyc05g015075.1.1.2; | | |
| Parent = mRNA: | | |
| Solyc05g015075.1.1) | | |
| promoter-TSS | Solyc05g015093 | NA |
| (ID = exon: | | |
| Solyc02g032530.3.1.1; | | |
| Parent = mRNA: | | |
| Solyc02g032530.3.1) | | |
| promoter-TSS | Solyc05g018830 | NA |
| (ID = exon: | | |
| Solyc05g018830.2.1.2; | | |
| Parent = mRNA: | | |
| Solyc05g018830.2.1) | | |
| promoter-TSS | Solyc05g018830 | NA |
| (ID = exon: | | |
| Solyc05g018830.2.1.2; | | |
| Parent = mRNA: | | |
| Solyc05g018830.2.1) | | |
| promoter-TSS | Solyc05g024070 | NA |
| (ID = exon: | | |
| Solyc05g024070.1.1.1; | | |
| Parent = mRNA: | | |
| Solyc05g024070.1.1) | | |
| promoter-TSS | Solyc05g050906 | NA |
| (ID = exon: | | |
| Solyc05g050690.2.1.1; | | |
| Parent = mRNA: | | |
| Solyc05g050690.2.1) | | |
| promoter-TSS | Solyc05g051140 | NA |
| (ID = exon: | | |
| Solyc05g051140.3.1.3; | | |
| Parent = mRNA: | | |
| Solyc05g051140.3.1) | | |
| TTS | Solyc05g052265 | NA |
| (ID = exon: | | |
| Solyc05g052265.1.1.2; | | |
| Parent = mRNA: | | |
| Solyc05g052265.1.1) | | |
| TTS | Solyc05g052880 | NA |
| (ID = exon: | | |
| Solyc05g052880.4.1.1; | | |
| Parent = mRNA: | | |
| Solyc05g052880.4.1) | | |
| TTS | Solyc05g053230 | Plant-pathogen |
| (ID = exon: | | interaction [04626]; |
| Solyc05g053230.3.1.9; | | Protein kinases [01001] |
| Parent = mRNA: | | |
| Solyc05g053230.3.1) | | |
| promoter-TSS | Solyc05g055104 | Glycosyltransferases |
| (ID = exon: | | [01003] |
| Solyc05g055410.3.1.4; | | |
| Parent = mRNA: | | |
| Solyc05g055410.3.1) | | |
| promoter-TSS | Solyc05g161730 | NA |
| (ID = exon: | | |
| Solyc05g161730.1.1.2; | | |
| Parent = mRNA: | | |
| Solyc05g161730.1.1) | | |
| promoter-TSS | Solyc05g162290 | NA |
| (ID = exon: | | |
| Solyc05g162290.1.1.15; | | |
| Parent = mRNA: | | |
| Solyc05g162290.1.1) | | |
| TTS | Solyc06g005800 | mRNA surveillance |
| (ID = exon: | | pathway [03015]; |
| Solyc06g005800.3.1.1; | | Protein phosphatases |
| Parent = mRNA: | | and associated proteins |
| Solyc06g005800.3.1) | | [01009]; Messenger |
| | | RNA biogenesis |
| | | [03019]; Transcription |
| | | machinery [03021]; |
| | | Spliceosome [03041] |
| TTS | Solyc06g008490 | NA |
| (ID = exon: | | |
| Solyc06g008490.3.1.9; | | |
| Parent = mRNA: | | |
| Solyc06g008490.3.1) | | |
| promoter-TSS | Solyc06g016800 | NA |
| (ID = exon: | | |
| Solyc06g016800.3.1.2; | | |
| Parent = mRNA: | | |
| Solyc06g016800.3.1) | | |
| TTS | Solyc06g048620 | NA |
| (ID = exon: | | |
| Solyc06g048620.3.1.2; | | |
| Parent = mRNA: | | |
| Solyc06g048620.3.1) | | |
| promoter-TSS | Solyc06g050260 | NA |
| (ID = exon: | | |
| Solyc06g050260.1.1.2; | | |
| Parent = mRNA: | | |
| Solyc06g050260.1.1) | | |
| promoter-TSS | Solyc06g053280 | NA |
| (ID = exon: | | |
| Solyc06g053280.2.1.8; | | |
| Parent = mRNA: | | |
| Solyc06g053280.2.1) | | |
| promoter-TSS | Solyc06g060380 | Chromosome and |
| (ID = exon: | | associated proteins |
| Solyc06g060380.3.1.14; | | [03036] |
| Parent = mRNA: | | |
| Solyc06g060380.3.1) | | |
| promoter-TSS | Solyc06g063420 | Transcription |
| (ID = exon: | | machinery [03021] |
| Solyc06g063420.5.1.3; | | |
| Parent = mRNA: | | |
| Solyc06g063420.5.1) | | |
| exon | Solyc06g065720 | NA |
| (ID = exon: | | |
| Solyc06g065720.1.1.1; | | |
| Parent = mRNA: | | |
| Solyc06g065720.1.1, | | |
| exon 1 of 1) | | |
| TTS | Solyc06g066430 | NA |
| (ID = exon: | | |
| Solyc06g066430.3.1.1; | | |
| Parent = mRNA: | | |
| Solyc06g066430.3.1) | | |
| promoter-TSS | Solyc06g072520 | NA |
| (ID = exon: | | |
| Solyc06g072520.2.1.3; | | |
| Parent = mRNA: | | |
| Solyc06g072520.2.1) | | |
| promoter-TSS | Solyc06g072840 | NA |
| (ID = exon: | | |
| Solyc06g072840.3.1.1; | | |
| Parent = mRNA: | | |
| Solyc06g072840.3.1) | | |
| promoter-TSS | Solyc06g073580 | NA |
| (ID = exon: | | |
| Solyc06g073580.5.1.1; | | |
| Parent = mRNA: | | |
| Solyc06g073580.5.1) | | |
| promoter-TSS | Solyc06g082890 | Transfer RNA |
| (ID = exon: | | biogenesis [03016] |
| Solyc06g082890.4.1.9; | | |
| Parent = mRNA: | | |
| Solyc06g082890.4.1) | | |
| TTS | Solyc06g083930 | Transcription factors |
| (ID = exon: | | [03000] |
| Solyc06g083930.2.1.1; | | |
| Parent = mRNA: | | |
| Solyc06g083930.2.1) | | |
| promoter-TSS | Solyc06g084430 | Exosome [04147]; |
| (ID = exon: | | Chromosome and |
| Solyc06g084430.4.1.1; | | associated proteins |
| Parent = mRNA: | | [03036] |
| Solyc06g084430.4.1) | | |
| TTS | Solyc07g005630 | NA |
| (ID = exon: | | |
| Solyc07g005630.3.1.2; | | |
| Parent = mRNA: | | |
| Solyc07g005630.3.1) | | |
| TTS | Solyc07g008570 | Others [99999] |
| (ID = exon: | | |
| Solyc07g008570.3.1.11; | | |
| Parent = mRNA: | | |
| Solyc07g008570.3.1) | | |
| promoter-TSS | Solyc07g008810 | NA |
| (ID = exon: | | |
| Solyc07g008810.1.1.1; | | |
| Parent = mRNA: | | |
| Solyc07g008810.1.1) | | |
| TTS | Solyc07g017220 | NA |
| (ID = exon: | | |
| Solyc07g017220.3.1.1; | | |
| Parent = mRNA: | | |
| Solyc07g017220.3.1) | | |
| promoter-TSS | Solyc07g032027 | NA |
| (ID = exon: | | |
| Solyc07g032027.1.1.4; | | |
| Parent = mRNA: | | |
| Solyc07g032027.1.1) | | |
| TTS | Solyc01g005860 | NA |
| (ID = exon: | | |
| Solyc01g005860.2.1.1; | | |
| Parent = mRNA: | | |
| Solyc01g005860.2.1) | | |
| promoter-TSS | Solyc01g005950 | Membrane trafficking |
| (ID = exon: | | [04131]; Protein |
| Solyc01g005950.4.1.11; | | phosphatases and |
| Parent = mRNA: | | associated proteins |
| Solyc01g005950.4.1) | | [01009]; Messenger |
| | | RNA biogenesis |
| | | [03019]; Autophagy - |
| | | other [04136]; Exosome |
| | | [04147]; mRNA |
| | | surveillance pathway |
| | | [03015]; Chromosome |
| | | and associated proteins |
| | | [03036] |
| promoter-TSS | Solyc01g006900 | NA |
| (ID = exon: | | |
| Solyc01g006900.3.1.3; | | |
| Parent = mRNA: | | |
| Solyc01g006900.3.1) | | |
| exon | Solyc01g007860 | DNA repair and |
| (ID = exon: | | recombination proteins |
| Solyc01g007860.3.1.4; | | [03400] |
| Parent = mRNA: | | |
| Solyc01g007860.3.1, | | |
| exon 1 of 1) | | |
| TTS | Solyc01g008050 | Glycan |
| (ID = exon: | | metabolism [99986] |
| Solyc01g008050.4.1.2; | | |
| Parent = mRNA. | | |
| Solyc01g008050.4.1) | | |
| promoter-TSS | Solyc01g008420 | NA |
| (ID = exon: | | |
| Solyc01g008420.3.1.1; | | |
| Parent = mRNA: | | |
| Solyc01g008420.3.1) | | |
| promoter-TSS | Solyc01g008550 | NA |
| (ID = exon: | | |
| Solyc01g008550.4.1.6; | | |
| Parent = mRNA: | | |
| Solyc01g008550.4.1) | | |
| promoter-TSS | Solyc01g012760 | Pantothenate and CoA |
| (ID = exon: | | biosynthesis [00770]; |
| Solyc01g012760.1.1.4; | | Glycolysis/ |
| Parent = mRNA: | | Gluconeogenesis |
| Solyc01g012760.1.1) | | [00010]; Tryptophan |
| | | metabolism [00380]; |
| | | beta-Alanine |
| | | metabolism [00410]; |
| | | Fatty acid degradation |
| | | [00071]; Limonene and |
| | | pinene degradation |
| | | [00903]; Arginine and |
| | | proline metabolism |
| | | [0033 |
| TTS | Solyc01g014830 | NA |
| (ID = exon: | | |
| Solyc01g014830.1.1.1; | | |
| Parent = mRNA: | | |
| Solyc01g014830.1.1) | | |
| promoter-TSS | Solyc01g020357 | NA |
| (ID = exon: | | |
| Solyc01g020357.1.1.1; | | |
| Parent = mRNA: | | |
| Solyc01g020357.1.1) | | |
| promoter-TSS | Solyc01g056830 | Membrane trafficking |
| (ID = exon: | | [04131]; Exosome |
| Solyc01g056830.3.1.4; | | [04147]; SNARE |
| Parent = mRNA: | | interactions in vesicular |
| Solyc01g056830.3.1) | | transport [04130] |
| promoter-TSS | Solyc01g057703 | NA |
| (ID = exon: | | |
| Solyc01g057703.1.1.2; | | |
| Parent = mRNA: | | |
| Solyc01g057703.1.1) | | |
| promoter-TSS | Solyc01g058673 | NA |
| (ID = exon: | | |
| Solyc01g058673.2.1.1; | | |
| Parent = mRNA: | | |
| Solyc01g058673.2.1) | | |
| TTS | Solyc01g065647 | NA |
| (ID = exon: | | |
| Solyc01g065647.1.1.6; | | |
| Parent = mRNA: | | |
| Solyc01g065647.1.1) | | |
| TTS | Solyc01g067380 | NA |
| (ID = exon: | | |
| Solyc01g067380.4.1.1; | | |
| Parent = mRNA: | | |
| Solyc01g067380.4.1) | | |
| promoter-TSS | Solyc01g068270 | NA |
| (ID = exon: | | |
| Solyc01g068270.3.1.2; | | |
| Parent = mRNA: | | |
| Solyc01g068270.3.1) | | |
| promoter-TSS | Solyc01g081490 | NA |
| (ID = exon: | | |
| Solyc01g081490.3.1.1; | | |
| Parent = mRNA: | | |
| Solyc01g081490.3.1) | | |
| promoter-TSS | Solyc01g088800 | NA |
| (ID = exon: | | |
| Solyc01g088800.4.1.2; | | |
| Parent = mRNA: | | |
| Solyc01g088800.4.1) | | |
| promoter-TSS | Solyc01g090430 | NA |
| (ID = exon: | | |
| Solyc01g090430.3.1.2; | | |
| Parent = mRNA: | | |
| Solyc01g090430.3.1) | | |
| promoter-TSS | Solyc01g091890 | PATHWAY: tRNA |
| (ID = exon: | | modification; 5- |
| Solyc01g091890.3.1.4; | | methoxycarbonylmethyl- |
| Parent = mRNA: | | 2-thiouridine-tRNA |
| Solyc01g091890.3.1) | | biosynthesis. |
| | | {ECO: 0000256\|HAMA |
| | | P-Rule: MF_03048, |
| | | ECO: 0000256\| |
| | | RuleBase: RU361182}.; |
| | | Sulfur relay system [04122]; |
| | | Transfer RNA |
| | | biogenesis [03016]; |
| | | Ubiquitin system |
| | | [04121] |
| TTS | Solyc01g094705 | NA |
| (ID = exon: | | |
| Solyc01g094705.1.1.1; | | |
| Parent = mRNA: | | |
| Solyc01g094705.1.1) | | |
| exon | Solyc01g098140 | NA |
| (ID = exon: | | |
| Solyc01g098110.5.1.14; | | |
| Parent = mRNA: | | |
| Solyc01g098110.5.1, | | |
| exon 1 of 1) | | |
| TTS | Solyc07g032140 | NA |
| (ID = exon: | | |
| Solyc07g032140.3.1.1; | | |
| Parent = mRNA: | | |
| Solyc07g032140.3.1) | | |
| TTS | Solyc07g043040 | NA |
| (ID = exon: | | |
| Solyc07g043040.1.1.1; | | |
| Parent = mRNA: | | |
| Solyc07g043040.1.1) | | |
| promoter-TSS | Solyc07g049740 | PATHWAY: |
| (ID = exon: | | Glycolipid |
| Solyc07g049740.4.1.2; | | biosynthesis; lipid |
| Parent = mRNA: | | IV(A) biosynthesis; |
| Solyc07g049740.4.1) | | lipid IV(A) from (3R)- |
| | | 3- |
| | | hydroxytetradecanoyl- |
| | | [acyl-carrier-protein] |
| | | and UDP-N-acetyl- |
| | | alpha-D-glucosamine: |
| | | step 5/6. |
| | | {ECO: 0000256\|ARBA: |
| | | ARBA00004901}.; |
| | | Glycosyltransferases |
| | | [01003]; |
| | | Lipopolysacchar |
| promoter-TSS | Solyc07g052050 | Spliceosome [03041]; |
| (ID = exon: | | Spliceosome [03040] |
| Solyc07g052050.3.1.1; | | |
| Parent = mRNA: | | |
| Solyc07g052050.3.1) | | |
| promoter-TSS | Solyc07g052490 | NA |
| (ID = exon: | | |
| Solyc07g052490.4.1.3; | | |
| Parent = mRNA: | | |
| Solyc07g052490.4.1) | | |
| promoter-TSS | Solyc07g054650 | NA |
| (ID = exon: | | |
| Solyc07g054650.1.1.1; | | |
| Parent = mRNA: | | |
| Solyc07g054650.1.1) | | |
| promoter-TSS | Solyc07g055550 | NA |
| (ID = exon: | | |
| Solyc07g055550.2.1.3; | | |
| Parent = mRNA: | | |
| Solyc07g055550.2.1) | | |
| promoter-TSS | Solyc07g063130 | NA |
| (ID = exon: | | |
| Solyc07g063130.4.1.8; | | |
| Parent = mRNA: | | |
| Solyc07g063130.4.1) | | |
| promoter-TSS | Solyc07g065790 | NA |
| (ID = exon: | | |
| Solyc07g065790.2.1.1; | | |
| Parent = mRNA: | | |
| Solyc07g065790.2.1) | | |
| promoter-TSS | Solyc07g150113 | NA |
| (ID = exon: | | |
| Solyc07g150113.1.1.1; | | |
| Parent = mRNA: | | |
| Solyc07g150113.1.1) | | |
| promoter-TSS | Solyc07g161660 | NA |
| (ID = exon: | | |
| Solyc07g161660.1.1.3; | | |
| Parent = mRNA: | | |
| Solyc07g161660.1.1) | | |
| TTS | Solyc08g007190 | NA |
| (ID = exon: | | |
| Solyc08g007190.3.1.1; | | |
| Parent = mRNA: | | |
| Solyc08g007190.3.1) | | |
| promoter-TSS | Solyc08g016275 | NA |
| (ID = exon: | | |
| Solyc08g016275.2.1.11; | | |
| Parent = mRNA: | | |
| Solyc08g016275.2.1) | | |
| promoter-TSS | Solyc08g023280 | DNA repair and |
| (ID = exon: | | recombination proteins |
| Solyc08g023280.4.1.2; | | [03400]; Messenger |
| Parent = mRNA: | | RNA biogenesis |
| Solyc08g023280.4.1) | | [03019]; Chromosome |
| | | and associated proteins |
| | | [03036]; Homologous |
| | | recombination [03440]; |
| | | Ubiquitin system |
| | | [04121] |
| promoter-TSS | Solyc08g061980 | Transporters [02000] |
| (ID = exon: | | |
| Solyc08g061980.4.1.7; | | |
| Parent = mRNA: | | |
| Solyc08g061980.4.1) | | |
| TTS | Solyc08g063055 | NA |
| (ID = exon: | | |
| Solyc08g063055.1.1.3; | | |
| Parent = mRNA: | | |
| Solyc08g063055.1.1) | | |
| promoter-TSS | Solyc08g068120 | DNA repair and |
| (ID = exon: | | recombination proteins |
| Solyc08g068110.1.1.5; | | [03400] |
| Parent = mRNA: | | |
| Solyc08g068110.1.1) | | |
| promoter-TSS | Solyc08g074895 | NA |
| (ID = exon: | | |
| Solyc08g074910.3.1.1; | | |
| Parent = mRNA: | | |
| Solyc08g074910.3.1) | | |
| promoter-TSS | Solyc08g074910 | NA |
| (ID = exon: | | |
| Solyc08g074910.3.1.1; | | |
| Parent = mRNA: | | |
| Solyc08g074910.3.1) | | |
| promoter-TSS | Solyc08g078120 | NA |
| (ID = exon: | | |
| Solyc08g078120.2.1.4; | | |
| Parent = mRNA: | | |
| Solyc08g078120.2.1) | | |
| promoter-TSS | Solyc08g078160 | NA |
| (ID = exon: | | |
| Solyc08g078160.3.1.2; | | |
| Parent = mRNA: | | |
| Solyc08g078160.3.1) | | |
| promoter-TSS | Solyc08g078580 | NA |
| (ID = exon: | | |
| Solyc08g078580.3.1.1; | | |
| Parent = mRNA: | | |
| Solyc08g078580.3.1) | | |
| promoter-TSS | Solyc08g082550 | NA |
| (ID = exon: | | |
| Solyc08g082550.1.1.2; | | |
| Parent = mRNA: | | |
| Solyc08g082550.1.1) | | |
| TTS | Solyc08g082550 | NA |
| (ID = exon: | | |
| Solyc08g082550.1.1.2; | | |
| Parent = mRNA: | | |
| Solyc08g082550.1.1) | | |
| promoter-TSS | Solyc08g082550 | NA |
| (ID = exon: | | |
| Solyc08g082550.1.1.2; | | |
| Parent = mRNA: | | |
| Solyc08g082550.1.1) | | |
| TTS | Solyc08g082550 | NA |
| (ID = exon: | | |
| Solyc08g082550.1.1.2; | | |
| Parent = mRNA: | | |
| Solyc08g082550.1.1) | | |
| TTS | Solyc08g160540 | NA |
| (ID = exon: | | |
| Solyc08g160540.1.1.3; | | |
| Parent = mRNA: | | |
| Solyc08g160540.1.1) | | |
| promoter-TSS | Solyc08g161050 | Mitochondrial |
| (ID = exon: | | biogenesis [03029]; |
| Solyc08g161050.1.1.1; | | DNA replication |
| Parent = mRNA: | | proteins [03032] |
| Solyc08g161050.1.1) | | |
| exon | Solyc08g161890 | NA |
| (ID = exon: | | |
| Solyc08g161890.1.1.1; | | |
| Parent = mRNA: | | |
| Solyc08g161890.1.1, | | |
| exon 1 of 1) | | |
| TTS | Solyc09g005035 | NA |
| (ID = exon: | | |
| Solyc09g005035.1.1.3; | | |
| Parent = mRNA: | | |
| Solyc09g005035.1.1) | | |
| TTS | Solyc09g005730 | NA |
| (ID = exon: | | |
| Solyc09g005730.4.1.3; | | |
| Parent = mRNA: | | |
| Solyc09g005730.4.1) | | |
| promoter-TSS | Solyc09g009370 | Membrane trafficking |
| (ID = exon: | | [04131] |
| Solyc09g009370.4.1.44; | | |
| Parent = mRNA: | | |
| Solyc09g009370.4.1) | | |
| TTS | Solyc09g010210 | Starch and sucrose |
| (ID = exon: | | metabolism [00500] |
| Solyc09g010210.3.1.7; | | |
| Parent = mRNA: | | |
| Solyc09g010210.3.1) | | |
| promoter-TSS | Solyc09g010995 | Enzymes with EC |
| (ID = exon: | | numbers [99980] |
| Solyc09g010995.1.1.2; | | |
| Parent = mRNA: | | |
| Solyc09g010995.1.1) | | |
| TTS | Solyc09g018690 | Messenger RNA |
| (ID = exon: | | biogenesis [03019]; |
| Solyc09g018690.4.1.11; | | DNA repair and |
| Parent = mRNA: | | recombination proteins |
| Solyc09g018690.4.1) | | [03400]; RNA |
| | | degradation [03018] |
| TTS | Solyc09g031570 | NA |
| (ID = exon: | | |
| Solyc09g031570.1.1.1; | | |
| Parent = mRNA: | | |
| Solyc09g031570.1.1) | | |
| TTS | Solyc09g055830 | NA |
| (ID = exon: | | |
| Solyc09g055830.1.1.1; | | |
| Parent = mRNA: | | |
| Solyc09g055830.1.1) | | |
| exon | Solyc09g059245 | NA |
| (ID = exon: | | |
| Solyc09g059245.1.1.1; | | |
| Parent = mRNA: | | |
| Solyc09g059245.1.1, | | |
| exon 1 of 1) | | |
| promoter-TSS | Solyc09g064775 | NA |
| (ID = exon: | | |
| Solyc09g064775.2.1.2; | | |
| Parent = mRNA: | | |
| Solyc09g064775.2.1) | | |
| promoter-TSS | Solyc09g075360 | Starch and sucrose |
| (ID = exon: | | metabolism [00500] |
| Solyc09g075360.5.1.1; | | |
| Parent = mRNA: | | |
| Solyc09g075360.5.1) | | |
| TTS | Solyc09g083130 | NA |
| (ID = exon: | | |
| Solyc09g083130.3.1.1; | | |
| Parent = mRNA: | | |
| Solyc09g083130.3.1) | | |
| promoter-TSS | Solyc09g090870 | NA |
| (ID = exon: | | |
| Solyc09g090870.3.1.4; | | |
| Parent = mRNA: | | |
| Solyc09g090870.3.1) | | |
| promoter-TSS | Solyc09g091280 | Transcription factors |
| (ID = exon: | | [03000] |
| Solyc09g091280.4.1.1; | | |
| Parent = mRNA: | | |
| Solyc09g091280.4.1) | | |
| TTS | Solyc09g160080 | Transcription factors |
| (ID = exon: | | [03000] |
| Solyc09g160080.1.1.1; | | |
| Parent = mRNA: | | |
| Solyc09g160080.1.1) | | |
| promoter-TSS | Solyc09g161010 | Transcription factors |
| (ID = exon: | | [03000] |
| Solyc09g161010.1.1.8; | | |
| Parent = mRNA: | | |
| Solyc09g161010.1.1) | | |
| promoter-TSS | Solyc10g006840 | NA |
| (ID = exon: | | |
| Solyc10g006840.5.1.1; | | |
| Parent = mRNA: | | |
| Solyc10g006840.5.1) | | |
| promoter-TSS | Solyc10g007660 | NA |
| (ID = exon: | | |
| Solyc10g007660.3.1.1; | | |
| Parent = mRNA: | | |
| Solyc10g007660.3.1) | | |
| promoter-TSS | Solyc10g007870 | NA |
| (ID = exon: | | |
| Solyc10g007870.3.1.2; | | |
| Parent = mRNA: | | |
| Solyc10g007870.3.1) | | |
| TTS | Solyc10g012090 | Transporters [02000] |
| (ID = exon: | | |
| Solyc10g012090.2.1.2; | | |
| Parent = mRNA: | | |
| Solyc10g012090.2.1) | | |
| TTS | Solyc10g018193 | Transcription factors |
| (ID = exon: | | [03000] |
| Solyc10g018193.1.1.1; | | |
| Parent = mRNA: | | |
| Solyc10g018193.1.1) | | |
| TTS | Solyc10g036640 | NA |
| (ID = exon: | | |
| Solyc10g036640.3.1.2; | | |
| Parent = mRNA: | | |
| Solyc10g036640.3.1) | | |
| TTS | Solyc10g039430 | NA |
| (ID = exon: | | |
| Solyc10g039430.3.1.9; | | |
| Parent = mRNA: | | |
| Solyc10g039430.3.1) | | |
| promoter-TSS | Solyc10g044986 | NA |
| (ID = exon: | | |
| Solyc10g044986.1.1.4; | | |
| Parent = mRNA: | | |
| Solyc10g044986.1.1) | | |
| promoter-TSS | Solyc10g049710 | Peptidases and |
| (ID = exon: | | inhibitors [01002] |
| Solyc10g049710.2.1.1; | | |
| Parent = mRNA: | | |
| Solyc10g049710.2.1) | | |
| promoter-TSS | Solyc10g050150 | NA |
| (ID = exon: | | |
| Solyc10g050150.1.1.1; | | |
| Parent = mRNA: | | |
| Solyc10g050150.1.1) | | |
| promoter-TSS | Solyc10g050777 | NA |
| (ID = exon: | | |
| Solyc10g050777.1.1.1; | | |
| Parent = mRNA: | | |
| Solyc10g050777.1.1) | | |
| promoter-TSS | Solyc10g052490 | Enzymeswith EC |
| (ID = exon: | | numbers [99980] |
| Solyc10g052490.2.1.1; | | |
| Parent = mRNA: | | |
| Solyc10g052490.2.1) | | |
| TTS | Solyc10g052590 | NA |
| (ID = exon: | | |
| Solyc10g052590.1.1.1; | | |
| Parent = mRNA: | | |
| Solyc10g052590.1.1) | | |
| TTS | Solyc10g054270 | NA |
| (ID = exon: | | |
| Solyc10g054270.3.1.1; | | |
| Parent = mRNA: | | |
| Solyc10g054270.3.1) | | |
| promoter-TSS | Solyc10g062160 | NA |
| (ID = exon: | | |
| Solyc10g062160.3.1.11; | | |
| Parent = mRNA: | | |
| Solyc10g062160.3.1) | | |
| promoter-TSS | Solyc10g073353 | MAPK signaling |
| (ID = exon: | | pathway - plant |
| Solyc10g073353.1.1.3; | | [04016]; Amino sugar |
| Parent = mRNA: | | and nucleotide sugar |
| Solyc10g073353.1.1) | | metabolism [00520] |
| promoter-TSS | Solyc10g074570 | Plant-pathogen |
| (ID = exon: | | interaction [04626]; |
| Solyc10g074570.2.1.2; | | Protein kinases [01001] |
| Parent = mRNA: | | |
| Solyc10g074570.2.1) | | |
| promoter-TSS | Solyc10g076230 | Phenylpropanoid |
| (ID = exon: | | biosynthesis [00940] |
| Solyc10g076230.3.1.1; | | |
| Parent = mRNA: | | |
| Solyc10g076230.3.1) | | |
| TTS | Solyc10g079795 | NA |
| (ID = exon: | | |
| Solyc10g079795.1.1.2; | | |
| Parent = mRNA: | | |
| Solyc10g079795.1.1) | | |
| promoter-TSS | Solyc10g081470 | NA |
| (ID = exon: | | |
| Solyc10g081470.2.1.10; | | |
| Parent = mRNA: | | |
| Solyc10g081470.2.1) | | |
| promoter-TSS | Solyc10g084850 | Ubiquitin system |
| (ID = exon: | | [04121] |
| Solyc10g084850.2.1.7; | | |
| Parent = mRNA: | | |
| Solyc10g084850.2.1) | | |
| TTS | Solyc10g085550 | PATHWAY: |
| (ID = exon: | | Carbohydrate |
| Solyc10g085550.3.1.17; | | degradation; glycolysis; |
| Parent = mRNA: | | pyruvate from |
| Solyc10g085550.3.1) | | D-glyceraldehyde |
| | | 3-phosphate: step 4/5. |
| | | {ECO: 0000256\|ARBA: |
| | | ARBA00005031}.; |
| | | Glycolysis/ |
| | | Gluconeogenesis |
| | | [00010]; Messenger |
| | | RNA biogenesis |
| | | [03019]; RNA |
| | | degradation [03018]; |
| | | Exosome [04147] |
| TTS | Solyc10g161410 | NA |
| (ID = exon: | | |
| Solyc10g161410.1.1.1; | | |
| Parent = mRNA: | | |
| Solyc10g161410.1.1) | | |
| TTS | Solyc10g162260 | NA |
| (ID = exon: | | |
| Solyc10g162260.1.1.1; | | |
| Parent = mRNA: | | |
| Solyc10g162260.1.1) | | |
| TTS | Solyc10g162300 | NA |
| (ID = exon: | | |
| Solyc10g162300.1.1.1; | | |
| Parent = mRNA: | | |
| Solyc10g162300.1.1) | | |
| promoter-TSS | Solyc10g162750 | NA |
| (ID = exon: | | |
| Solyc10g162750.1.1.1; | | |
| Parent = mRNA: | | |
| Solyc10g162750.1.1) | | |
| promoter-TSS | Solyc11g006750 | NA |
| (ID = exon: | | |
| Solyc11g006750.1.1.2; | | |
| Parent = mRNA: | | |
| Solyc11g006750.1.1) | | |
| promoter-TSS | Solyc11g008450 | NA |
| (ID = exon: | | |
| Solyc11g008450.2.1.1; | | |
| Parent = mRNA: | | |
| Solyc11g008450.2.1) | | |
| promoter-TSS | Solyc11g010900 | Transfer RNA |
| (ID = exon: | | biogenesis [03016] |
| Solyc11g010900.2.1.1; | | |
| Parent = mRNA: | | |
| Solyc11g010900.2.1) | | |
| TTS | Solyc11g020580 | NA |
| (ID = exon: | | |
| Solyc11g020580.1.1.2; | | |
| Parent = mRNA: | | |
| Solyc11g020580.1.1) | | |
| promoter-TSS | Solyc11g022390 | Transfer RNA |
| (ID = exon: | | biogenesis [030161 |
| Solyc11g022390.2.1.1; | | |
| Parent = mRNA: | | |
| Solyc11g022390.2.1) | | |
| exon | Solyc11g022465 | NA |
| (ID = exon: | | |
| Solyc11g022465.1.1.2; | | |
| Parent = mRNA: | | |
| Solyc11g022465.1.1, | | |
| exon 1 of 1) | | |
| promoter-TSS | Solyc11g030387 | NA |
| (ID = exon: | | |
| Solyc11g030387.1.1.4; | | |
| Parent = mRNA: | | |
| Solyc11g030387.1.1) | | |
| TTS | Solyc11g033280 | Spliceosome [03041]; |
| (ID = exon: | | Spliceosome [03040]; |
| Solyc11g033280.2.1.1; | | Messenger RNA |
| Parent = mRNA: | | biogenesis [03019]; |
| Solyc11g033280.2.1) | | RNA transport [03013]; |
| | | Translation factors |
| | | [03012]; Ribosome |
| | | biogenesis [03009]; |
| | | mRNA surveillance |
| | | pathway [03015] |
| TTS | Solyc11g044920 | Plant-pathogen |
| (ID = exon: | | interaction [04626] |
| Solyc11g044910.2.1.1; | | |
| Parent = mRNA: | | |
| Solyc11g044910.2.1) | | |
| promoter-TSS | Solyc11g045210 | NA |
| (ID = exon: | | |
| Solyc11g045210.2.1.8; | | |
| Parent = mRNA: | | |
| Solyc11g045210.2.1) | | |
| promoter-TSS | Solyc11g045210 | NA |
| (ID = exon: | | |
| Solyc11g045210.2.1.8; | | |
| Parent = mRNA: | | |
| Solyc11g045210.2.1) | | |
| promoter-TSS | Solyc11g067250 | DNA repair and |
| (ID = exon: | | recombination proteins |
| Solyc11g067250.4.1.2; | | [03400]; DNA |
| Parent = mRNA: | | replication proteins |
| Solyc11g067250.4.1) | | [03032]; Chromosome |
| | | and associated proteins |
| | | [03036]; Base excision |
| | | repair [03410] |
| promoter-TSS | Solyc11g069000 | Exosome [04147]; |
| (ID = exon: | | Chromosome and |
| Solyc11g069000.2.1.10; | | associated proteins |
| Parent = mRNA: | | [03036]; Chaperones |
| Solyc11g069000.2.1) | | and folding catalysts |
| | | [03110] |
| TTS | Solyc11g069050 | PATHWAY: |
| (ID = exon: | | Phytoalexin |
| Solyc11g069050.2.1.2; | | biosynthesis; 3,4′,5- |
| Parent = mRNA: | | trihydroxystilbene |
| Solyc11g069050.2.1) | | biosynthesis; 3,4′,5- |
| | | trihydroxystilbene from |
| | | trans-4-coumarate: step |
| | | 1/2. |
| | | {ECO: 0000256\|ARBA: |
| | | ARBA00004930}.; |
| | | Phenylpropanoid |
| | | biosynthesis [00940]; |
| | | Ubiquinone and other |
| | | terpenoid-quinone |
| | | biosynthe |
| promoter-TSS | Solyc11g069330 | NA |
| (ID = exon: | | |
| Solyc11g069330.1.1.1; | | |
| Parent = mRNA: | | |
| Solyc11g069330.1.1) | | |
| promoter-TSS | Solyc11g161150 | NA |
| (ID = exon: | | |
| Solyc11g161150.1.1.9; | | |
| Parent = mRNA: | | |
| Solyc11g161150.1.1) | | |
| promoter-TSS | Solyc12g006380 | NA |
| (ID = exon: | | |
| Solyc12g006380.2.1.1; | | |
| Parent = mRNA: | | |
| Solyc12g006380.2.1) | | |
| promoter-TSS | Solyc12g008470 | NA |
| (ID = exon: | | |
| Solyc12g008470.2.1.3; | | |
| Parent = mRNA: | | |
| Solyc12g008470.2.1) | | |
| promoter-TSS | Solyc12g008570 | Transfer RNA |
| (ID = exon: | | biogenesis [03016]; |
| Solyc12g008570.3.1.1; | | Amino acid related |
| Parent = mRNA: | | enzymes [01007]; |
| Solyc12g008570.3.1) | | Mitochondrial |
| | | biogenesis [03029]; |
| | | Aminoacyl-tRNA |
| | | biosynthesis [00970] |
| promoter-TSS | Solyc12g008840 | NA |
| (ID = exon: | | |
| Solyc12g008840.2.1.1; | | |
| Parent = mRNA: | | |
| Solyc12g008840.2.1) | | |
| promoter-TSS | Solyc12g009060 | Exosome [04147]; |
| (ID = exon: | | Membrane trafficking |
| Solyc12g009060.2.1.1; | | [04131]; Endocytosis |
| Parent = mRNA: | | [04144] |
| Solyc12g009060.2.1) | | |
| promoter-TSS | Solyc12g011023 | Enzymes with EC |
| (ID = exon: | | numbers [99980] |
| Solyc12g011023.1.1.2; | | |
| Parent = mRNA: | | |
| Solyc12g011023.1.1) | | |
| promoter-TSS | Solyc12g013810 | Chaperones and folding |
| (ID = exon: | | catalysts [03110] |
| Solyc12g013810.2.1.2; | | |
| Parent = mRNA: | | |
| Solyc12g013810.2.1) | | |
| promoter-TSS | Solyc12g017250 | Photosynthesis [00195]; |
| (ID = exon: | | Photosynthesis proteins |
| Solyc12g017250.2.1.5; | | [00194] |
| Parent = mRNA: | | |
| Solyc12g017250.2.1) | | |
| TTS | Solyc12g040500 | Membrane trafficking |
| (ID = exon: | | [04131]; Peroxisome |
| Solyc12g040500.2.1.3; | | [04146] |
| Parent = mRNA: | | |
| Solyc12g040500.2.1) | | |
| TTS | Solyc12g040790 | Glycerophospholipid |
| (ID = exon: | | metabolism [00564] |
| Solyc12g040790.2.1.2; | | |
| Parent = mRNA: | | |
| Solyc12g040790.2.1) | | |
| promoter-TSS | Solyc12g044220 | Structural |
| (ID = exon: | | proteins [99992] |
| Solyc12g044220.3.1.15; | | |
| Parent = mRNA: | | |
| Solyc12g044220.3.1) | | |
| promoter-TSS | Solyc12g044400 | NA |
| (ID = exon: | | |
| Solyc12g044400.3.1.13; | | |
| Parent = mRNA: | | |
| Solyc12g044400.3.1) | | |
| promoter-TSS | Solyc12g077623 | NA |
| (ID = exon: | | |
| Solyc12g077623.2.1.10; | | |
| Parent = mRNA: | | |
| Solyc12g077623.2.1) | | |
| promoter-TSS | Solyc12g096030 | Transporters [02000] |
| (ID = exon: | | |
| Solyc12g096030.1.1.1; | | |
| Parent = mRNA: | | |
| Solyc12g096030.1.1) | | |
| promoter-TSS | Solyc12g096120 | NA |
| (ID = exon: | | |
| Solyc12g096120.2.1.4; | | |
| Parent = mRNA: | | |
| Solyc12g096120.2.1) | | |
| TTS | Solyc12g160410 | NA |
| (ID = exon: | | |
| Solyc12g160410.1.1.1; | | |
| Parent = mRNA: | | |
| Solyc12g160410.1.1) | | |
| TTS | Solyc12g160480 | NA |
| (ID = exon: | | |
| Solyc12g160480.1.1.1; | | |
| Parent = mRNA: | | |
| Solyc12g160480.1.1) | | |
| TTS | Solyc12g161800 | NA |
| (ID = exon: | | |
| Solyc12g161800.1.1.4; | | |
| Parent = mRNA: | | |
| Solyc12g161800.1.1) | | |

Table 10 shows methylation patterns detected solely in the exons of control samples. Since these specific methylation fingerprints are not detected in the same exons from treated F3 tomato plants, it is highly plausible that the irradiation treatment regulates or leads to demethylation events in the same genes of treated tomato plants.

TABLE 10
Methylated Exons of Genes of Control F3 Tomato Plants

| Annotation | Gene Name | Pathway |
|---|---|---|
| promoter-TSS | Solyc00g160460 | NA |
| (ID = exon: | | |
| Solyc00g160460.1.1.2; | | |
| Parent = mRNA: | | |
| Solyc00g160460.1.1) | | |
| promoter-TSS | Solyc01g005220 | NA |
| (ID = exon: | | |
| Solyc01g005220.3.1.4; | | |
| Parent = mRNA: | | |
| Solyc01g005220.3.1) | | |
| promoter-TSS | Solyc01g005950 | Membrane |
| (ID = exon: | | trafficking [04131]; |
| Solyc01g005960.3.1.1; | | Protein phosphatases |
| Parent = mRNA: | | and associated |
| Solyc01g005960.3.1) | | proteins [01009]; |
| | | Messenger RNA |
| | | biogenesis [03019]; |
| | | Autophagy - other |
| | | [04136]; Exosome |
| | | [04147]; mRNA |
| | | surveillance |
| | | pathway [03015]; |
| | | Chromosome and |
| | | associated proteins |
| | | [03036] |
| promoter-TSS | Solyc01g008180 | NA |
| (ID = exon: | | |
| Solyc01g008180.3.1.6; | | |
| Parent = mRNA: | | |
| Solyc01g008180.3.1) | | |
| promoter-TSS | Solyc01g008420 | NA |
| (ID = exon: | | |
| Solyc01g008420.3.1.2; | | |
| Parent = mRNA: | | |
| Solyc01g008420.3.1) | | |
| promoter-TSS | Solyc01g009380 | NA |
| (ID = exon: | | |
| Solyc01g009380.3.1.1; | | |
| Parent = mRNA: | | |
| Solyc01g009380.3.1) | | |
| promoter-TSS | Solyc01g016530 | NA |
| (ID = exon: | | |
| Solyc01g016540.4.1.4; | | |
| Parent = mRNA: | | |
| Solyc01g016540.4.1) | | |
| promoter-TSS | Solyc01g016540 | NA |
| (ID = exon: | | |
| Solyc01g016540.4.1.4; | | |
| Parent = mRNA: | | |
| Solyc01g016540.4.1) | | |
| promoter-TSS | Solyc01g067090 | NA |
| (ID = exon: | | |
| Solyc01g067090.4.1.3; | | |
| Parent = mRNA: | | |
| Solyc01g067090.4.1) | | |
| promoter-TSS | Solyc01g068130 | NA |
| (ID = exon: | | |
| Solyc01g068130.4.1.4; | | |
| Parent = mRNA: | | |
| Solyc01g068130.4.1) | | |
| promoter-TSS | Solyc01g073985 | Plant-pathogen |
| (ID = exon: | | interaction [04626] |
| Solyc01g073985.1.1.1; | | |
| Parent = mRNA: | | |
| Solyc01g073985.1.1) | | |
| TTS | Solyc01g079590 | NA |
| (ID = exon: | | |
| Solyc01g079600.4.1.1; | | |
| Parent = mRNA: | | |
| Solyc01g079600.4.1) | | |
| promoter-TSS | Solyc01g080700 | NA |
| (ID = exon: | | |
| Solyc01g080700.4.1.1; | | |
| Parent = mRNA: | | |
| Solyc01g080700.4.1) | | |
| promoter-TSS | Solyc01g081600 | NA |
| (ID = exon: | | |
| Solyc01g081600.3.1.2; | | |
| Parent = mRNA: | | |
| Solyc01g081600.3.1) | | |
| promoter-TSS | Solyc01g086975 | NA |
| (ID = exon: | | |
| Solyc01g086975.1.1.6; | | |
| Parent = mRNA: | | |
| Solyc01g086975.1.1) | | |
| promoter-TSS | Solyc01g087040 | NA |
| (ID = exon: | | |
| Solyc01g087040.2.1.1; | | |
| Parent = mRNA: | | |
| Solyc01g087040.2.1) | | |
| promoter-TSS | Solyc01g090230 | NA |
| (ID = exon: | | |
| Solyc01g090230.3.1.18; | | |
| Parent = mRNA: | | |
| Solyc01g090230.3.1) | | |
| TTS | Solyc01g094705 | NA |
| (ID = exon: | | |
| Solyc01g094705.1.1.2; | | |
| Parent = mRNA: | | |
| Solyc01g094705.1.1) | | |
| TTS | Solyc01g101180 | PATHWAY: |
| (ID = exon: | | Secondary |
| Solyc01g101180.5.1.6; | | metabolite |
| Parent = mRNA: | | biosynthesis; |
| Solyc01g101180.5.1) | | terpenoid |
| | | biosynthesis.; |
| | | Enzymes with EC |
| | | numbers [99980] |
| TTS | Solyc01g103240 | MAPK signaling |
| (ID = exon: | | pathway - plant |
| Solyc01g103240.4.1.1; | | [04016]; Protein |
| Parent = mRNA: | | kinases [01001] |
| Solyc01g103240.4.1) | | |
| TTS | Solyc01g103240 | MAPK signaling |
| (ID = exon: | | pathway - plant |
| Solyc01g103240.4.1.1; | | [04016]; Protein |
| Parent = mRNA: | | kinases [01001] |
| Solyc01g103240.4.1) | | |
| TTS | Solyc01g104210 | NA |
| (ID = exon: | | |
| Solyc01g104210.3.1.1; | | |
| Parent = mRNA: | | |
| Solyc01g104210.3.1) | | |
| TTS | Solyc01g110200 | NA |
| (ID = exon: | | |
| Solyc01g110200.2.1.4; | | |
| Parent = mRNA: | | |
| Solyc01g110200.2.1) | | |
| promoter-TSS | Solyc01g111140 | Spliceosome |
| (ID = exon: | | [03041] |
| Solyc01g111140.3.1.1; | | |
| Parent = mRNA: | | |
| Solyc01g111140.3.1) | | |
| promoter-TSS | Solyc01g112370 | NA |
| (ID = exon: | | |
| Solyc01g112370.4.1.1; | | |
| Parent = mRNA: | | |
| Solyc01g112370.4.1) | | |
| promoter-TSS | Solyc01g160350 | NA |
| (ID = exon: | | |
| Solyc01g160350.1.1.2; | | |
| Parent = mRNA: | | |
| Solyc01g160350.1.1) | | |
| TTS | Solyc02g005395 | Tryptophan |
| (ID = exon: | | metabolism [00380] |
| Solyc02g005395.2.1.2; | | |
| Parent = mRNA: | | |
| Solyc02g005395.2.1) | | |
| TTS | Solyc02g020920 | NA |
| (ID = exon: | | |
| Solyc02g020920.1.1.2; | | |
| Parent = mRNA: | | |
| Solyc02g020920.1.1) | | |
| promoter-TSS | Solyc02g062920 | Spliceosome |
| (ID = exon: | | [03040]; |
| Solyc02g062920.3.1.1; | | Spliceosome |
| Parent = mRNA: | | [03041] |
| Solyc02g062920.3.1) | | |
| promoter-TSS | Solyc02g063410 | NA |
| (ID = exon: | | |
| Solyc02g063410.3.1.1; | | |
| Parent = mRNA: | | |
| Solyc02g063410.3.1) | | |
| TTS | Solyc02g063440 | NA |
| (ID = exon: | | |
| Solyc02g063440.3.1.3; | | |
| Parent = mRNA: | | |
| Solyc02g063440.3.1) | | |
| promoter-TSS | Solyc02g067310 | NA |
| (ID = exon: | | |
| Solyc02g067310.4.1.1; | | |
| Parent = mRNA: | | |
| Solyc02g067310.4.1) | | |
| promoter-TSS | Solyc02g068250 | NA |
| (ID = exon: | | |
| Solyc02g068250.4.1.2; | | |
| Parent = mRNA: | | |
| Solyc02g068250.4.1) | | |
| promoter-TSS | Solyc02g070270 | Transport [99977] |
| (ID = exon: | | |
| Solyc02g070270.2.1.1; | | |
| Parent = mRNA: | | |
| Solyc02g070270.2.1) | | |
| promoter-TSS | Solyc02g080460 | Cytoskeleton |
| (ID = exon: | | proteins [04812]; |
| Solyc02g080460.3.1.1; | | Chromosome and |
| Parent = mRNA: | | associated proteins |
| Solyc02g080460.3.1) | | [03036] |
| TTS | Solyc02g081030 | Transporters |
| (ID = exon: | | [02000] |
| Solyc02g081030.4.1.1; | | |
| Parent = mRNA: | | |
| Solyc02g081030.4.1) | | |
| TTS | Solyc02g081083 | NA |
| (ID = exon: | | |
| Solyc02g081083.1.1.1; | | |
| Parent = mRNA: | | |
| Solyc02g081083.1.1) | | |
| promoter-TSS | Solyc02g084113 | NA |
| (ID = exon: | | |
| Solyc02g084113.1.1.1; | | |
| Parent = mRNA: | | |
| Solyc02g084113.1.1) | | |
| promoter-TSS | Solyc02g085790 | Exosome [04147]; |
| (ID = exon: | | Chaperones and |
| Solyc02g085790.3.1.1; | | folding catalysts |
| Parent = mRNA: | | [03110] |
| Solyc02g085790.3.1) | | |
| promoter-TSS | Solyc02g086150 | Translation factors |
| (ID = exon: | | [03012] |
| Solyc02g086150.4.1.8; | | |
| Parent = mRNA: | | |
| Solyc02g086150.4.1) | | |
| promoter-TSS | Solyc02g087250 | NA |
| (ID = exon: | | |
| Solyc02g087250.4.1.17; | | |
| Parent = mRNA: | | |
| Solyc02g087250.4.1) | | |
| promoter-TSS | Solyc02g087250 | NA |
| (ID = exon: | | |
| Solyc02g087250.4.1.17; | | |
| Parent = mRNA: | | |
| Solyc02g087250.4.1) | | |
| promoter-TSS | Solyc02g090310 | NA |
| (ID = exon: | | |
| Solyc02g090310.1.1.1; | | |
| Parent = mRNA: | | |
| Solyc02g090310.1.1) | | |
| promoter-TSS | Solyc02g090630 | NA |
| (ID = exon: | | |
| Solyc02g090630.4.1.1; | | |
| Parent = mRNA: | | |
| Solyc02g090630.4.1) | | |
| promoter-TSS | Solyc02g092760 | NA |
| (ID = exon: | | |
| Solyc02g092760.3.1.1; | | |
| Parent = mRNA: | | |
| Solyc02g092760.3.1) | | |
| TTS | Solyc02g092940 | NA |
| (ID = exon: | | |
| Solyc02g092940.3.1.2; | | |
| Parent = mRNA: | | |
| Solyc02g092940.3.1) | | |
| promoter-TSS | Solyc03g007790 | NA |
| (ID = exon: | | |
| Solyc03g007790.3.1.1; | | |
| Parent = mRNA: | | |
| Solyc03g007790.3.1) | | |
| TTS | Solyc03g013620 | Oxidative |
| (ID = exon: | | phosphorylation |
| Solyc03g013620.1.1.1; | | [00190] |
| Parent = mRNA: | | |
| Solyc03g013620.1.1) | | |
| promoter-TSS | Solyc03g034200 | Messenger RNA |
| (ID = exon: | | biogenesis [03019] |
| Solyc03g034200.3.1.8; | | |
| Parent = mRNA: | | |
| Solyc03g034200.3.1) | | |
| TTS | Solyc03g044930 | NA |
| (ID = exon: | | |
| Solyc03g044930.3.1.1; | | |
| Parent = mRNA: | | |
| Solyc03g044930.3.1) | | |
| promoter-TSS | Solyc03g045000 | NA |
| (ID = exon: | | |
| Solyc03g045000.1.1.1; | | |
| Parent = mRNA: | | |
| Solyc03g045000.1.1) | | |
| promoter-TSS | Solyc03g046440 | NA |
| (ID = exon: | | |
| Solyc03g046440.3.1.1; | | |
| Parent = mRNA: | | |
| Solyc03g046440.3.1) | | |
| promoter-TSS | Solyc03g093440 | Other glycan |
| (ID = exon: | | degradation [00511] |
| Solyc03g093440.4.1.1; | | |
| Parent = mRNA: | | |
| Solyc03g093440.4.1) | | |
| TTS | Solyc03g094150 | NA |
| (ID = exon: | | |
| Solyc03g094150.1.1.1; | | |
| Parent = mRNA: | | |
| Solyc03g094150.1.1) | | |
| TTS | Solyc03g094173 | NA |
| (ID = exon: | | |
| Solyc03g094173.2.1.2; | | |
| Parent = mRNA: | | |
| Solyc03g094173.2.1) | | |
| promoter-TSS | Solyc03g097707 | Starch and sucrose |
| (ID = exon: | | metabolism [00500] |
| Solyc03g097707.1.1.2; | | |
| Parent = mRNA: | | |
| Solyc03g097707.1.1) | | |
| promoter-TSS | Solyc03g098170 | Exosome [04147]; |
| (ID = exon: | | Cytoskeleton |
| Solyc03g098170.3.1.9; | | proteins [04812] |
| Parent = mRNA: | | |
| Solyc03g098170.3.1) | | |
| promoter-TSS | Solyc03g098310 | Glutathione |
| (ID = exon: | | metabolism [00480]; |
| Solyc03g098310.1.1.1; | | Arginine and proline |
| Parent = mRNA: | | metabolism [00330] |
| Solyc03g098310.1.1) | | |
| promoter-TSS | Solyc03g111000 | Carbon fixation in |
| (ID = exon: | | photosynthetic |
| Solyc03g111000.3.1.3; | | organisms [00710]; |
| Parent = mRNA: | | Membrane |
| Solyc03g111000.3.1) | | trafficking [04131]; |
| | | Glycolysis/ |
| | | Gluconeogenesis |
| | | [00010]; Exosome |
| | | [04147] |
| promoter-TSS | Solyc03g111000 | Carbon fixation in |
| (ID = exon: | | photosynthetic |
| Solyc03g111000.3.1.3; | | organisms [00710]; |
| Parent = mRNA: | | Membrane |
| Solyc03g111000.3.1) | | trafficking [04131]; |
| | | Glycolysis/ |
| | | Gluconeogenesis |
| | | [00010]; Exosome |
| | | [04147] |
| promoter-TSS | Solyc03g111390 | NA |
| (ID = exon: | | |
| Solyc03g111390.4.1.1; | | |
| Parent = mRNA: | | |
| Solyc03g111390.4.1) | | |
| promoter-TSS | Solyc03g113190 | NA |
| (ID = exon: | | |
| Solyc03g113190.1.1.1; | | |
| Parent = mRNA: | | |
| Solyc03g113190.1.1) | | |
| promoter-TSS | Solyc03g114940 | Cytochrome P450 |
| (ID = exon: | | [00199] |
| Solyc03g114940.3.1.2; | | |
| Parent = mRNA: | | |
| Solyc03g114940.3.1) | | |
| promoter-TSS | Solyc03g114940 | Cytochrome P450 |
| (ID = exon: | | [00199] |
| Solyc03g114940.3.1.2; | | |
| Parent = mRNA: | | |
| Solyc03g114940.3.1) | | |
| promoter-TSS | Solyc03g115610 | NA |
| (ID = exon: | | |
| Solyc03g115610.3.1.2; | | |
| Parent = mRNA: | | |
| Solyc03g115610.3.1) | | |
| promoter-TSS | Solyc04g014450 | Enzymes with EC |
| (ID = exon: | | numbers [99980] |
| Solyc04g014450.2.1.10; | | |
| Parent = mRNA: | | |
| Solyc04g014450.2.1) | | |
| TTS | Solyc04g014855 | NA |
| (ID = exon: | | |
| Solyc04g014853.2.1.4; | | |
| Parent = mRNA: | | |
| Solyc04g014853.2.1) | | |
| promoter-TSS | Solyc04g015100 | Synthesis and |
| (ID = exon: | | degradation of |
| Solyc04g015100.3.1.1; | | ketone bodies |
| Parent = mRNA: | | [00072]; Pyruvate |
| Solyc04g015100.3.1) | | metabolism [00620]; |
| | | Glyoxylate and |
| | | dicarboxylate |
| | | metabolism [00630]; |
| | | Valine, leucine and |
| | | isoleucine |
| | | degradation [00280]; |
| | | Fatty acid |
| | | degradation [00071]; |
| | | Exosome [04147]; |
| | | Terpenoid backbone |
| | | biosy |
| promoter-TSS | Solyc04g024530 | Cytoskeleton |
| (ID = exon: | | proteins [04812]; |
| Solyc04g024530.3.1.9; | | Membrane |
| Parent = mRNA: | | trafficking [04131]; |
| Solyc04g024530.3.1) | | Exosome [04147] |
| promoter-TSS | Solyc04g049780 | NA |
| (ID = exon: | | |
| Solyc04g049780.3.1.1; | | |
| Parent = mRNA: | | |
| Solyc04g049780.3.1) | | |
| promoter-TSS | Solyc04g054250 | NA |
| (ID = exon: | | |
| Solyc04g054250.4.1.1; | | |
| Parent = mRNA: | | |
| Solyc04g054250.4.1) | | |
| TTS | Solyc04g075000 | NA |
| (ID = exon: | | |
| Solyc04g075000.1.1.1; | | |
| Parent = mRNA: | | |
| Solyc04g075000.1.1) | | |
| promoter-TSS | Solyc04g076390 | Transfer RNA |
| (ID = exon: | | biogenesis [03016]; |
| Solyc04g076390.3.1.1; | | Aminoacyl-tRNA |
| Parent = mRNA: | | biosynthesis |
| Solyc04g076390.3.1) | | [00970]; Amino acid |
| | | related enzymes |
| | | [01007] |
| promoter-TSS | Solyc04g077550 | NA |
| (ID = exon: | | |
| Solyc04g077550.1.1.1; | | |
| Parent = mRNA: | | |
| Solyc04g077550.1.1) | | |
| promoter-TSS | Solyc04g078310 | Chromosome and |
| (ID = exon: | | associated proteins |
| Solyc04g078310.3.1.1; | | [03036]; DNA |
| Parent = mRNA: | | replication proteins |
| Solyc04g078310.3.1) | | [03032] |
| TTS | Solyc04g080890 | NA |
| (ID = exon: | | |
| Solyc04g080890.4.1.1; | | |
| Parent = mRNA: | | |
| Solyc04g080890.4.1) | | |
| promoter-TSS | Solyc04g081350 | Transcription factors |
| (ID = exon: | | [03000] |
| Solyc04g081350.3.1.13; | | |
| Parent = mRNA: | | |
| Solyc04g081350.3.1) | | |
| promoter-TSS | Solyc05g010800 | DNA repair and |
| (ID = exon: | | recombination |
| Solyc05g010800.3.1.5; | | proteins [03400]; |
| Parent = mRNA: | | Base excision repair |
| Solyc05g010800.3.1) | | [03410] |
| promoter-TSS | Solyc05g012530 | NA |
| (ID = exon: | | |
| Solyc05g012530.1.1.1; | | |
| Parent = mRNA: | | |
| Solyc05g012530.1.1) | | |
| promoter-TSS | Solyc05g012770 | NA |
| (ID = exon: | | |
| Solyc05g012770.3.1.1; | | |
| Parent = mRNA: | | |
| Solyc05g012770.3.1) | | |
| promoter-TSS | Solyc05g014052 | NA |
| (ID = exon: | | |
| Solyc05g014052.1.1.1; | | |
| Parent = mRNA: | | |
| Solyc05g014052.1.1) | | |
| promoter-TSS | Solyc05g014260 | Plant hormone |
| (ID = exon: | | signal transduction |
| Solyc05g014260.3.1.1; | | [04075] |
| Parent = mRNA: | | |
| Solyc05g014260.3.1) | | |
| promoter-TSS | Solyc05g015075 | NA |
| (ID = exon: | | |
| Solyc05g015075.1.1.1; | | |
| Parent = mRNA: | | |
| Solyc05g015075.1.1) | | |
| TTS | Solyc05g015490 | NA |
| (ID = exon: | | |
| Solyc05g015490.5.1.2; | | |
| Parent = mRNA: | | |
| Solyc05g015490.5.1) | | |
| promoter-TSS | Solyc05g015795 | NA |
| (ID = exon: | | |
| Solyc05g015795.2.1.3; | | |
| Parent = mRNA: | | |
| Solyc05g015795.2.1) | | |
| promoter-TSS | Solyc05g018590 | NA |
| (ID = exon: | | |
| Solyc05g018590.3.1.9; | | |
| Parent = mRNA: | | |
| Solyc05g018590.3.1) | | |
| TTS | Solyc05g021530 | NA |
| (ID = exon: | | |
| Solyc05g021530.3.1.1; | | |
| Parent = mRNA: | | |
| Solyc05g021530.3.1) | | |
| promoter-TSS | Solyc05g041470 | NA |
| (ID = exon: | | |
| Solyc05g041470.2.1.1; | | |
| Parent = mRNA: | | |
| Solyc05g041470.2.1) | | |
| promoter-TSS | Solyc05g047640 | Membrane |
| (ID = exon: | | trafficking [04131] |
| Solyc05g047640.3.1.3; | | |
| Parent = mRNA: | | |
| Solyc05g047640.3.1) | | |
| TTS | Solyc05g050510 | NA |
| (ID = exon: | | |
| Solyc05g050510.3.1.1; | | |
| Parent = mRNA: | | |
| Solyc05g050510.3.1) | | |
| TTS | Solyc05g050510 | NA |
| (ID = exon: | | |
| Solyc05g050510.3.1.1; | | |
| Parent = mRNA: | | |
| Solyc05g050510.3.1) | | |
| promoter-TSS | Solyc05g051140 | NA |
| (ID = exon: | | |
| Solyc05g051140.3.1.2; | | |
| Parent = mRNA: | | |
| Solyc05g051140.3.1) | | |
| promoter-TSS | Solyc05g051140 | NA |
| (ID = exon: | | |
| Solyc05g051140.3.1.2; | | |
| Parent = mRNA: | | |
| Solyc05g051140.3.1) | | |
| TTS | Solyc05g051425 | NA |
| (ID = exon: | | |
| Solyc05g051425.3.1.5; | | |
| Parent = mRNA: | | |
| Solyc05g051425.3.1) | | |
| promoter-TSS | Solyc05g053380 | NA |
| (ID = exon: | | |
| Solyc05g053380.5.1.2; | | |
| Parent = mRNA: | | |
| Solyc05g053380.5.1) | | |
| promoter-TSS | Solyc05g054440 | NA |
| (ID = exon: | | |
| Solyc05g054440.5.1.2; | | |
| Parent = mRNA: | | |
| Solyc05g054440.5.1) | | |
| promoter-TSS | Solyc05g054720 | NA |
| (ID = exon: | | |
| Solyc05g054720.3.1.1; | | |
| Parent = mRNA: | | |
| Solyc05g054720.3.1) | | |
| promoter-TSS | Solyc06g008530 | Mitochondrial |
| (ID = exon: | | biogenesis [03029]; |
| Solyc06g008530.4.1.1; | | Messenger RNA |
| Parent = mRNA: | | biogenesis [03019]; |
| Solyc06g008530.4.1) | | Membrane |
| | | trafficking [04131]; |
| | | Cytoskeleton |
| | | proteins [04812] |
| promoter-TSS | Solyc06g048630 | NA |
| (ID = exon: | | |
| Solyc06g048630.3.1.4; | | |
| Parent = mRNA: | | |
| Solyc06g048630.3.1) | | |
| TTS | Solyc06g048813 | NA |
| (ID = exon: | | |
| Solyc06g048813.1.1.4; | | |
| Parent = mRNA: | | |
| Solyc06g048813.1.1) | | |
| promoter-TSS | Solyc06g051650 | Enzymes with EC |
| (ID = exon: | | numbers [99980] |
| Solyc06g051650.4.1.3; | | |
| Parent = mRNA: | | |
| Solyc06g051650.4.1) | | |
| promoter-TSS | Solyc06g053635 | NA |
| (ID = exon: | | |
| Solyc06g053635.2.1.1; | | |
| Parent = mRNA: | | |
| Solyc06g053635.2.1) | | |
| TTS | Solyc06g054260 | Photosynthesis |
| (ID = exon: | | proteins [00194]; |
| Solyc06g054270.3.1.1; | | Photosynthesis |
| Parent = mRNA: | | [00195] |
| Solyc06g054270.3.1) | | |
| TTS | Solyc06g062375 | NA |
| (ID = exon: | | |
| Solyc06g062375.2.1.1; | | |
| Parent = mRNA: | | |
| Solyc06g062375.2.1) | | |
| promoter-TSS | Solyc06g066240 | NA |
| (ID = exon: | | |
| Solyc06g066240.5.1.2; | | |
| Parent = mRNA: | | |
| Solyc06g066240.5.1) | | |
| promoter-TSS | Solyc06g072910 | NA |
| (ID = exon: | | |
| Solyc06g072910.2.1.1; | | |
| Parent = mRNA: | | |
| Solyc06g072910.2.1) | | |
| promoter-TSS | Solyc06g073250 | NA |
| (ID = exon: | | |
| Solyc06g073250.1.1.1; | | |
| Parent = mRNA: | | |
| Solyc06g073250.1.1) | | |
| promoter-TSS | Solyc06g082010 | NA |
| (ID = exon: | | |
| Solyc06g082010.4.1.2; | | |
| Parent = mRNA: | | |
| Solyc06g082010.4.1) | | |
| promoter-TSS | Solyc07g006090 | Enzymes with EC |
| (ID = exon: | | numbers [99980] |
| Solyc07g006090.3.1.2; | | |
| Parent = mRNA: | | |
| Solyc07g006090.3.1) | | |
| promoter-TSS | Solyc07g006100 | Membrane |
| (ID = exon: | | trafficking [04131] |
| Solyc07g006100.4.1.10; | | |
| Parent = mRNA: | | |
| Solyc07g006100.4.1) | | |
| promoter-TSS | Solyc07g027020 | NA |
| (ID = exon: | | |
| Solyc07g027020.4.1.9; | | |
| Parent = mRNA: | | |
| Solyc07g027020.4.1) | | |
| TTS | Solyc07g042880 | Cytochrome P450 |
| (ID = exon: | | [00199] |
| Solyc07g042880.1.1.3; | | |
| Parent = mRNA: | | |
| Solyc07g042880.1.1) | | |
| promoter-TSS | Solyc07g052050 | Spliceosome |
| (ID = exon: | | [03041]; |
| Solyc07g052050.3.1.2; | | Spliceosome |
| Parent = mRNA: | | [03040] |
| Solyc07g052050.3.1) | | |
| TTS | Solyc07g053420 | Ubiquitin system |
| (ID = exon: | | [04121] |
| Solyc07g053420.5.1.1; | | |
| Parent = mRNA: | | |
| Solyc07g053420.5.1) | | |
| promoter-TSS | Solyc07g055730 | NA |
| (ID = exon: | | |
| Solyc07g055730.4.1.4; | | |
| Parent = mRNA: | | |
| Solyc07g055730.4.1) | | |
| TTS | Solyc07g056420 | Transporters |
| (ID = exon: | | [02000]; Glutathione |
| Solyc07g056420.4.1.2; | | metabolism [00480] |
| Parent = mRNA: | | |
| Solyc07g056420.4.1) | | |
| promoter-TSS | Solyc07g062547 | NA |
| (ID = exon: | | |
| Solyc07g062560.3.1.1; | | |
| Parent = mRNA: | | |
| Solyc07g062560.3.1) | | |
| promoter-TSS | Solyc07g064060 | NA |
| (ID = exon: | | |
| Solyc07g064060.3.1.9; | | |
| Parent = mRNA: | | |
| Solyc07g064060.3.1) | | |
| TTS | Solyc07g065563 | Transcription factors |
| (ID = exon: | | [03000] |
| Solyc07g065563.1.1.3; | | |
| Parent = mRNA: | | |
| Solyc07g065563.1.1) | | |
| TTS | Solyc07g161660 | NA |
| (ID = exon: | | |
| Solyc07g161660.1.1.2; | | |
| Parent = mRNA: | | |
| Solyc07g161660.1.1) | | |
| TTS | Solyc08g006860 | NA |
| (ID = exon: | | |
| Solyc08g006860.3.1.7; | | |
| Parent = mRNA: | | |
| Solyc08g006860.3.1) | | |
| promoter-TSS | Solyc08g008590 | Ubiquitin system |
| (ID = exon: | | [04121]; Ribosome |
| Solyc08g008590.4.1.2; | | [03011]; Ubiquitin |
| Parent = mRNA: | | mediated proteolysis |
| Solyc08g008590.4.1) | | [04120]; Ribosome |
| | | [03010] |
| promoter-TSS | Solyc08g016275 | NA |
| (ID = exon: | | |
| Solyc08g016275.2.1.10; | | |
| Parent = mRNA: | | |
| Solyc08g016275.2.1) | | |
| promoter-TSS | Solyc08g023280 | DNA repair and |
| (ID = exon: | | recombination |
| Solyc08g023280.4.1.1; | | proteins [03400]; |
| Parent = mRNA: | | Messenger RNA |
| Solyc08g023280.4.1) | | biogenesis [03019]; |
| | | Chromosome and |
| | | associated proteins |
| | | [03036]; |
| | | Homologous |
| | | recombination |
| | | [03440]; Ubiquitin |
| | | system [04121] |
| promoter-TSS | Solyc08g061180 | NA |
| (ID = exon: | | |
| Solyc08g061180.1.1.1; | | |
| Parent = mRNA: | | |
| Solyc08g061180.1.1) | | |
| TTS | Solyc08g062930 | NA |
| (ID = exon: | | |
| Solyc08g062930.3.1.6; | | |
| Parent = mRNA: | | |
| Solyc08g062930.3.1) | | |
| promoter-TSS | Solyc08g065880 | Messenger RNA |
| (ID = exon: | | biogenesis [03019] |
| Solyc08g065880.3.1.2; | | |
| Parent = mRNA: | | |
| Solyc08g065880.3.1) | | |
| promoter-TSS | Solyc08g067020 | Oxidative |
| (ID = exon: | | phosphorylation |
| Solyc08g067020.5.1.1; | | [00190] |
| Parent = mRNA: | | |
| Solyc08g067020.5.1) | | |
| promoter-TSS | Solyc08g068090 | NA |
| (ID = exon: | | |
| Solyc08g068090.3.1.1; | | |
| Parent = mRNA: | | |
| Solyc08g068090.3.1) | | |
| promoter-TSS | Solyc08g077040 | NA |
| (ID = exon: | | |
| Solyc08g077040.3.1.1; | | |
| Parent = mRNA: | | |
| Solyc08g077040.3.1) | | |
| TTS | Solyc08g161890 | NA |
| (ID = exon: | | |
| Solyc08g161890.1.1.1; | | |
| Parent = mRNA: | | |
| Solyc08g161890.1.1) | | |
| TTS | Solyc09g008770 | NA |
| (ID = exon: | | |
| Solyc09g008770.3.1.2; | | |
| Parent = mRNA: | | |
| Solyc09g008770.3.1) | | |
| TTS | Solyc09g009600 | Others [99999] |
| (ID = exon: | | |
| Solyc09g009600.3.1.1; | | |
| Parent = mRNA: | | |
| Solyc09g009600.3.1) | | |
| promoter-TSS | Solyc09g011630 | Transporters |
| (ID = exon: | | [02000]; Glutathione |
| Solyc09g011630.3.1.1; | | metabolism [00480] |
| Parent = mRNA: | | |
| Solyc09g011630.3.1) | | |
| promoter-TSS | Solyc09g012000 | NA |
| (ID = exon: | | |
| Solyc09g012000.1.1.1; | | |
| Parent = mRNA: | | |
| Solyc09g012000.1.1) | | |
| promoter-TSS | Solyc09g018340 | NA |
| (ID = exon: | | |
| Solyc09g018340.2.1.1; | | |
| Parent = mRNA: | | |
| Solyc09g018340.2.1) | | |
| promoter-TSS | Solyc09g018650 | Purine metabolism |
| (ID = exon: | | [00230] |
| Solyc09g018650.3.1.3; | | |
| Parent = mRNA: | | |
| Solyc09g018650.3.1) | | |
| promoter-TSS | Solyc09g056200 | NA |
| (ID = exon: | | |
| Solyc09g056200.3.1.2; | | |
| Parent = mRNA: | | |
| Solyc09g056200.3.1) | | |
| TTS | Solyc09g057630 | NA |
| (ID = exon: | | |
| Solyc09g057630.3.1.1; | | |
| Parent = mRNA: | | |
| Solyc09g057630.3.1) | | |
| promoter-TSS | Solyc09g058993 | Transcription factors |
| (ID = exon: | | [03000] |
| Solyc09g058993.1.1.1; | | |
| Parent = mRNA: | | |
| Solyc09g058993.1.1) | | |
| TTS | Solyc09g059990 | NA |
| (ID = exon: | | |
| Solyc09g059990.1.1.1; | | |
| Parent = mRNA: | | |
| Solyc09g059990.1.1) | | |
| promoter-TSS | Solyc09g063150 | Transporters |
| (ID = exon: | | [02000]; Glutathione |
| Solyc09g063140.4.1.4; | | metabolism [00480] |
| Parent = mRNA: | | |
| Solyc09g063140.4.1) | | |
| TTS | Solyc09g074510 | NA |
| (ID = exon: | | |
| Solyc09g074510.3.1.5; | | |
| Parent = mRNA: | | |
| Solyc09g074510.3.1) | | |
| promoter-TSS | Solyc09g074910 | Transporters |
| (ID = exon: | | [02000] |
| Solyc09g074910.1.1.1; | | |
| Parent = mRNA: | | |
| Solyc09g074910.1.1) | | |
| promoter-TSS | Solyc09g090090 | Protein kinases |
| (ID = exon: | | [01001] |
| Solyc09g090090.2.1.3; | | |
| Parent = mRNA: | | |
| Solyc09g090090.2.1) | | |
| TTS | Solyc09g092235 | NA |
| (ID = exon: | | |
| Solyc09g092235.2.1.1; | | |
| Parent = mRNA: | | |
| Solyc09g092235.2.1) | | |
| TTS | Solyc09g160760 | NA |
| (ID = exon: | | |
| Solyc09g160760.1.1.2; | | |
| Parent = mRNA: | | |
| Solyc09g160760.1.1) | | |
| TTS | Solyc10g009010 | NA |
| (ID = exon: | | |
| Solyc10g009010.5.1.1; | | |
| Parent = mRNA: | | |
| Solyc10g009010.5.1) | | |
| TTS | Solyc10g012245 | NA |
| (ID = exon: | | |
| Solyc10g012245.1.1.1; | | |
| Parent = mRNA: | | |
| Solyc10g012245.1.1) | | |
| TTS | Solyc10g045525 | NA |
| (ID = exon: | | |
| Solyc10g045525.2.1.3; | | |
| Parent = mRNA: | | |
| Solyc10g045525.2.1) | | |
| promoter-TSS | Solyc10g055540 | NA |
| (ID = exon: | | |
| Solyc10g055540.2.1.2; | | |
| Parent = mRNA: | | |
| Solyc10g055540.2.1) | | |
| TTS | Solyc10g079060 | Sesquiterpenoid and |
| (ID = exon: | | triterpenoid |
| Solyc10g079060.2.1.1; | | biosynthesis |
| Parent = mRNA: | | [00909]; Steroid |
| Solyc10g079060.2.1) | | biosynthesis |
| | | [00100]; |
| | | Prenyltransferases |
| | | [01006] |
| promoter-TSS | Solyc10g080600 | NA |
| (ID = exon: | | |
| Solyc10g080600.3.1.1; | | |
| Parent = mRNA: | | |
| Solyc10g080600.3.1) | | |
| promoter-TSS | Solyc10g080890 | NA |
| (ID = exon: | | |
| Solyc10g080890.2.1.1; | | |
| Parent = mRNA: | | |
| Solyc10g080890.2.1) | | |
| promoter-TSS | Solyc10g081850 | Membrane |
| (ID = exon: | | trafficking [04131]; |
| Solyc10g081850.3.1.1; | | Exosome [04147]; |
| Parent = mRNA: | | SNARE interactions |
| Solyc10g081850.3.1) | | in vesicular |
| | | transport [04130] |
| promoter-TSS | Solyc10g081890 | NA |
| (ID = exon: | | |
| Solyc10g081890.2.1.2; | | |
| Parent = mRNA: | | |
| Solyc10g081890.2.1) | | |
| promoter-TSS | Solyc10g084400 | Transporters |
| (ID = exon: | | [02000]; Glutathione |
| Solyc10g084400.2.1.9; | | metabolism [00480] |
| Parent = mRNA: | | |
| Solyc10g084400.2.1) | | |
| promoter-TSS | Solyc10g085480 | Ribosome [03010]; |
| (ID = exon: | | Ribosome [03011] |
| Solyc10g085480.2.1.1; | | |
| Parent = mRNA: | | |
| Solyc10g085480.2.1) | | |
| promoter-TSS | Solyc10g086180 | PATHWAY: |
| (ID = exon: | | Phenylpropanoid |
| Solyc10g086180.2.1.2; | | metabolism; trans- |
| Parent = mRNA: | | cinnamate |
| Solyc10g086180.2.1) | | biosynthesis; trans- |
| | | cinnamate from L- |
| | | Phenylalanine: step |
| | | 1/1. |
| | | {ECO: 0000256\|AR |
| | | BA: ARBA00005138, |
| | | ECO: 0000256\|Rule |
| | | Base: RU003955}.; |
| | | Phenylalanine |
| | | metabolism [00360]; |
| | | Phenylpropanoid |
| | | biosynthesis [00940] |
| promoter-TSS | Solyc10g086500 | PATHWAY: Plant |
| (ID = exon: | | hormone |
| Solyc10g086500.1.1.1; | | biosynthesis; |
| Parent = mRNA: | | brassinosteroid |
| Solyc10g086500.1.1) | | biosynthesis.; |
| | | Brassinosteroid |
| | | biosynthesis [00905] |
| promoter-TSS | Solyc10g161110 | NA |
| (ID = exon: | | |
| Solyc10g161110.1.1.3; | | |
| Parent = mRNA: | | |
| Solyc10g161110.1.1) | | |
| promoter-TSS | Solyc11g012580 | NA |
| (ID = exon: | | |
| Solyc11g012580.2.1.1; | | |
| Parent = mRNA: | | |
| Solyc11g012580.2.1) | | |
| promoter-TSS | Solyc11g012880 | Transporters |
| (ID = exon: | | [02000] |
| Solyc11g012880.2.1.1; | | |
| Parent = mRNA: | | |
| Solyc11g012880.2.1) | | |
| promoter-TSS | Solyc11g017210 | NA |
| (ID = exon: | | |
| Solyc11g017220.1.1.1; | | |
| Parent = mRNA: | | |
| Solyc11g017220.1.1) | | |
| promoter-TSS | Solyc11g020215 | NA |
| (ID = exon: | | |
| Solyc11g020215.1.1.1; | | |
| Parent = mRNA: | | |
| Solyc11g020215.1.1) | | |
| promoter-TSS | Solyc11g022390 | Transfer RNA |
| (ID = exon: | | biogenesis [03016] |
| Solyc11g022390.2.1.2; | | |
| Parent = mRNA: | | |
| Solyc11g022390.2.1) | | |
| promoter-TSS | Solyc11g065610 | Membrane |
| (ID = exon: | | trafficking [04131] |
| Solyc11g065610.1.1.2; | | |
| Parent = mRNA: | | |
| Solyc11g065610.1.1) | | |
| promoter-TSS | Solyc11g069680 | NA |
| (ID = exon: | | |
| Solyc11g069680.1.1.1; | | |
| Parent = mRNA: | | |
| Solyc11g069680.1.1) | | |
| promoter-TSS | Solyc11g072330 | NA |
| (ID = exon: | | |
| Solyc11g072330.2.1.1; | | |
| Parent = mRNA: | | |
| Solyc11g072330.2.1) | | |
| promoter-TSS | Solyc12g007070 | Transcription factors |
| (ID = exon: | | [03000] |
| Solyc12g007070.2.1.1; | | |
| Parent = mRNA: | | |
| Solyc12g007070.2.1) | | |
| promoter-TSS | Solyc12g007110 | NA |
| (ID = exon: | | |
| Solyc12g007110.2.1.1; | | |
| Parent = mRNA: | | |
| Solyc12g007110.2.1) | | |
| TTS | Solyc12g009170 | RNA degradation |
| (ID = exon: | | [03018]; Messenger |
| Solyc12g009170.2.1.2; | | RNA biogenesis |
| Parent = mRNA: | | [03019] |
| Solyc12g009170.2.1) | | |
| TTS | Solyc12g009170 | RNA degradation |
| (ID = exon: | | [03018]; Messenger |
| Solyc12g009170.2.1.2; | | RNA biogenesis |
| Parent = mRNA: | | [03019] |
| Solyc12g009170.2.1) | | |
| promoter-TSS | Solyc12g011023 | Enzymes with EC |
| (ID = exon: | | numbers [99980] |
| Solyc12g011023.1.1.3; | | |
| Parent = mRNA: | | |
| Solyc12g011023.1.1) | | |
| promoter-TSS | Solyc12g014240 | NA |
| (ID = exon: | | |
| Solyc12g014240.4.1.1; | | |
| Parent = mRNA: | | |
| Solyc12g014240.4.1) | | |
| TTS | Solyc12g089385 | NA |
| (ID = exon: | | |
| Solyc12g089385.1.1.1; | | |
| Parent = mRNA: | | |
| Solyc12g089385.1.1) | | |
| TTS | Solyc12g089385 | NA |
| (ID = exon: | | |
| Solyc12g089385.1.1.1; | | |
| Parent = mRNA: | | |
| Solyc12g089385.1.1) | | |
| TTS | Solyc12g096370 | Chromosome and |
| (ID = exon: | | associated proteins |
| Solyc12g096380.1.1.1; | | [03036]; Membrane |
| Parent = mRNA: | | trafficking [04131] |
| Solyc12g096380.1.1) | | |
| promoter-TSS | Solyc12g098620 | NA |
| (ID = exon: | | |
| Solyc12g098620.2.1.1; | | |
| Parent = mRNA: | | |
| Solyc12g098620.2.1) | | |
| TTS | Solyc12g160940 | NA |
| (ID = exon: | | |
| Solyc12g160940.1.1.2; | | |
| Parent = mRNA: | | |
| Solyc12g160940.1.1) | | |
| promoter-TSS | Solyc12g162010 | NA |
| (ID = exon: | | |
| Solyc12g162010.1.1.1; | | |
| Parent = mRNA: | | |
| Solyc12g162010.1.1) | | |

The above examples, tables (Tables 1-10) and experimental results clearly indicate that tomato plants exposed to the irradiation treatment of the present disclosure (one hour of irradiation for half an hour before sunset and half an hour into the night, for one month) exhibit improved inheritable yield-related traits, such as plant height, inflorescence number, plant weight and germination percentage, as well as transcriptomic and methylation differences, which are highly distinguishable from the control plant population.

1. A method for producing a non-genetically modified (non-GM) tomato plant and/or a harvestable part thereof, exhibiting at least one inheritable improved trait as compared to a control population of plants of the same plant cultivar or line, the method comprising the steps of:
(a) providing a population of a cultivar or line of tomato seeds or plants;
(b) exposing said population (a) of a cultivar or line of tomato plants to a predetermined light treatment by irradiating with artificial light, partly in the presence of ambient daylight, every day for a duration of 2 to 6 weeks from sowing, preferably for 30 days from sowing, wherein the predetermined light treatment consists of irradiating with artificial red light of wavelength in the range of from about 600 nm to about 700 nm for a period of about 60 minutes, starting about 30 min. before sunset in the presence of daylight, and continuing about 30 minutes after sunset;
(c) growing the tomato plants in greenhouses for 2-3 months after the end of stage (b), then collecting, counting and weighing the tomatoes and collecting their seeds;
(d) monitoring at least one improved trait of the tomato plants at the end of step (b) and step (c) and comparing to a control population;
(e) selecting at the end of the treatment of step (b) the plants exhibiting at least one improved trait as compared to a control population and selecting again at the end of the growing stage of step (c) the plants exhibiting at least one improved trait as compared to a control population; wherein the number of plants selected in the first generation is from about 1% to about 4%, preferably from about 2% to about 3% of the initial plant population provided (a); and
(f) propagating by sowing the tomato seeds at least one subsequent generation of the plants exhibiting at least one improved trait as compared to a control population;
thereby obtaining a tomato crop plant and/or harvestable part thereof exhibiting at least one improved trait, wherein the improved trait is inheritable for at least one more generation, preferably for two or more generations, more preferably for 3 to 5 generations, and even more preferably for 6 to 8 generations, further wherein said tomato crop plant and/or harvestable part thereof exhibits transcriptomic or methylation fingerprints distinguishable from said control population.

2. The method of claim 1, further comprising repeating steps (b) to (f) of claim 1 on 1-7 subsequent generations of said tomato plants, preferably at least two subsequent generations.

3. The method of claim 1, wherein the at least one improved inheritable trait being monitored is selected from the group consisting of tomato yield, tomato number, tomato weight, tomato shelf life, seed number, seed weight, plant height, main stem width, stem thickness, plant biomass, number of stems, number of secondary stems, stress resistance, pest resistance, virus resistance, drought tolerance, herbicide tolerance, delayed senescence, modified color, photosynthesis efficiency, nitrogen concentration in leaves and any combination thereof.

4. The method of claim 3, wherein the at least one improved inheritable trait is an increased tomato yield of at least 2% and up to 800%, or more particularly 2%, 5%, 10%, 20%, 30%, 40%, 50%, 60%, 70%, 80%, 90%, 100%, 200%, 300%, 400%, 500%, 600%, 700%, 800% or higher, as compared to the control population of the same tomato cultivar or line.

5. The method of claim 1, wherein the at least one inheritable improved trait is the result of an epigenetic effect.

6. The method of claim 1, wherein applied to plants or harvestable plant parts selected from the group consisting of tomatoes, seeds, seedlings, tissue cultures, calluses, meristems, regenerable cells, protoplasts, potted seedlings, adult plants and any combination thereof.

7. The method of claim 1, wherein said step (b) of irradiating with artificial light is applied at a periodicity selected from the group consisting of during sunset, before sunset, after sunset and any combination thereof.

8. The method of claim 1, wherein applied to tomato cultivars, plant lines or plant types selected from the group consisting of M82, Adoration, Alicante, Azoychka, Beefsteak, Better Boy, Big Beef, Big Rainbow, Blaby Special, Black Krim, Brandy wine, Campari, Celebrity, Cherokee Purple, Canario, Early Girl, Enchantment, Ferris Wheel, Flamenco, Fourth of July, Garden Peach, Gardener's delight, German Johnson, Giulietta F1, Granadero, Great White, Green Zebra, Hillbilly, Japanese, Black Trifele, Jersey Boy, Jubilee, Juliet, Kumato, Lilian's Yellow, McDreamy, Matt's Wild Cherry, Micro Tom, Moneymaker, Monterosa, Montserrat, Mortgage Lifter, Mr. Stripey, Pantano Romanesco, Paul Robeson, Plum Tomato, Pumpkin Tomato, Raf Tomato, Rebellion, Red Currant, Roma, Rosa de Barbastro, Rutgers Tomato, San Marzano, Sasha Altai, Stupice, Tiny Tim, Traveller, Arkansas Traveler, Cherry Bambelo, Cherrry Nebula, Santorini, Super Sweet, Tomaccio, Yellow Pear, White Queen and any combination thereof.

9. The method of claim 1, wherein applied to a genetically modified (GM) plant.

10. A tomato plant or a harvestable tomato plant part, preferably a tomato fruit, having at least one improved inheritable trait, wherein obtained by the method of claim 1.

11. A tomato plant or a harvestable tomato plant part, preferably a tomato fruit, exhibiting at least one improved trait compared to a control population of tomato plants of the same cultivar or line, wherein produced by the method of claim 1, further wherein said tomato plant or a harvestable tomato plant part exhibits transcriptomic or methylation fingerprints distinguishable from said control population of plants of the same plant cultivar or line.

12. A tomato plant exhibiting at least one improved trait compared to a control population of tomato plants of the same cultivar or line, wherein produced by the method of claim 1 and wherein said tomato plant preserves its at least one improved trait for at least one subsequent generation, preferably for two or more generations, more preferably for 3 to 5 generations, and even more preferably for 6 to 8 generations, without exposure to additional light treatment.

13. The tomato plant or harvestable plant part according to any one of claims 10-12, wherein selected from the group consisting of tomatoes, seeds, seedlings, tissue cultures, calluses, meristems, regenerable cells, protoplasts, potted seedlings, adult plants and any combination thereof.

14. The plant or harvestable tomato plant part of according to any one of claims 10-12, wherein produced by irradiating with artificial light of red wavelengths.

15. The plant or harvestable tomato plant part of according to any one of claims 10-12, wherein the at least one improved inheritable trait is a yield characteristic selected from the group consisting of tomato number, tomato weight, seeds number, seeds weight, plant height, main stem width, stem thickness, plant biomass, number of stems, number of secondary stems, photosynthesis efficiency, nitrogen concentration in leaves and any combination thereof.

16. The plant or harvestable tomato plant part of claim 15, wherein said inheritable improved trait is an increased tomato yield from about 2% to about 800% or more, as compared to a control population of the same cultivar or line.

17. The plant or harvestable plant part of claim 1, wherein the population of a cultivar or line of tomato seeds or plants provided in claim 1(a) is a non-genetically modified (non-GM) plant population.

18. The plant or harvestable plant part of claim 1, wherein the population of a cultivar or line of tomato seeds or plants provided in claim 1(a) is a genetically modified (GM) plant population.

19. The harvestable tomato part according to any ones of claims 10-12, wherein it is a tomato exhibiting as improved trait an improved room temperature (17-27° C.) shelf life of at least 45 days after harvesting, as compared to about 10 days for an untreated control tomato.

20. A method of screening for a tomato plant cultivar or line amenable to exhibiting at least one inheritable improved trait by applying the method of claim 1 and selecting the best plants exhibiting at least one inheritable improved trait.
